(19) United States
(12) Patent Application Publication
(10) Pub. No.: US 2025/0273346 A1
(43) Pub. Date: Aug. 28, 2025

(54) IDENTIFICATION AND METHODS OF TREATMENT OF BREAST CANCER

(71) Applicant: PRELUDE CORPORATION, Laguna Hills, CA (US)

(72) Inventors: Axel Emanuel Stenmark Tullberg, Göteborg (SE); Erik Carl Viktor Holmberg, Asperö (SE); Per Oswald Karlsson, Göteborg (SE); Troy M. Bremer, Irvine, CA (US)

(21) Appl. No.: 18/998,333

(22) PCT Filed: Jul. 25, 2023
(86) PCT No.: PCT/US2023/070978

§ 371 Date: Jan. 24, 2025

Related U.S. Application Data
(60) Provisional application No. 63/496,608, filed on Apr. 17, 2023.
(60) Provisional application No. 63/479,049, filed on Jan. 9, 2023.
(60) Provisional application No. 63/477,078, filed on Dec. 23, 2022.
(60) Provisional application No. 63/369,500, filed on Jul. 26, 2022.

(51) Int. Cl. G16H 50/50; G16B 25/10; G16B 40/20

(52) U.S. Cl. CPC G16H 50/50; G16B 25/10; G16B 40/20

(57) ABSTRACT

Methods for treating and/or prognosing breast cancer are provided. The method includes determining an Immunescore (IS) for gene sets selected from an immunological signature gene set, wherein the IS is based on an immunological model, determining a Proliferative Index (PI) for gene sets selected from an oncogenic signature gene set, wherein the PI is based on a tumor-intrinsic model; and integrating the IS and the PI into an Integrated model to stratify subjects and identify a high-risk clinical group. The immunological model is trained (IS) in basal and HER2+ tumors, and the tumor-intrinsic model (PI) is trained in immune-depleted tumors.

### BACKGROUND

The present technology generally relates to a quantification of tumor aggressiveness, and more particularly, the quantification of tumor aggressiveness, immune activity, and patient age for predicting prognosis and radiotherapy (i.e., radiation therapy) benefit, which can thereafter be used for individualized treatment when required.

### REFERENCE TO SEQUENCE LISTING

The present application is being filed along with a Sequence Listing in electronic format. The Sequence Listing is provided as a file entitled “2023-07-15_Sequence_Listing-PRLUD018WO.xml”, which was created and last modified on Jul. 15, 2023, which is approximately 13,712 bytes in size. The information in the electronic Sequence Listing is hereby incorporated by reference in its entirety.

### SUMMARY

The methods disclosed herein each have several aspects, no single one of which is solely responsible for their desirable attributes. Without limiting the scope of the claims, some prominent features will now be discussed briefly. Numerous other embodiments are also contemplated, including embodiments that have fewer, additional, and/or different components, steps, features, objects, benefits, and advantages. The components, aspects, and steps may also be arranged and ordered differently. After considering this discussion, and particularly after reading the section entitled “Detailed Description”, one will understand how the features of the devices and methods disclosed herein provide advantages over other known devices and methods.

Provided herein is a method that includes a final model that is based on the quantification of tumor aggressiveness (measured by Proliferative Index), immune activity (measured by Immunescore), and patient age. In some embodiments, the final model is effective at predicting prognosis and radiotherapy (RT) benefit. However, the groups created are heterogeneous; e.g., a “moderate-risk” score can include patients with aggressive tumors (high Proliferative Index) with a strong immune activity (high Immunescore) or patients with less aggressive tumors (moderate Proliferative Index) who do not benefit from an antitumoral immune response. By also analyzing the Proliferative Index and Immunescore individually (these are always calculated for the Final model), a further understanding of the specific biology of the tumor is gained which allows health providers/clinicians to further individualize the treatment. This can have implications for other types of therapy (e.g., immunotherapy; a moderate Final model score with high Immunescore and Proliferative Index are excellent candidates for immunotherapy). Therefore, the model according to some non-limiting embodiments of the present disclosure provides information about the prognosis and RT benefit and providing information about the individual tumor biology, which can most likely guide treatment with immunotherapy, chemotherapy, and other types of targeted therapies. Analyses indicate that it is very prognostic for distant metastases as well, which makes it very useful for stratifying patients according to the benefit from chemotherapy in a non-limiting example.

Also provided herein is a method for treating breast cancer, the method including determining an Immunescore (IS) based on an immunological model comprised of genes from immunological gene sets; determining a Proliferative Index (PI) based on a tumor-intrinsic model comprised of genes from tumor-intrinsic gene sets; and integrating the IS and the PI into an Integrated model; integrating the Integrated model and an age of a subject into a Final model to identify if the subject falls into a specific-risk clinical group; and providing an appropriate therapy to the subject based on the specific-risk clinical group, wherein the immunological model is trained in basal and HER2⁺ tumors, and wherein the tumor intrinsic model is trained in immune-depleted tumors. In some embodiments, the immunological model includes immunological gene sets. In some embodiments, the method further includes performing a meta-analysis of the prognostic effect of each of the immunological gene sets.

In some embodiments, the immunological gene sets include C7 and any immune-related gene sets from the H, C1, C2, C3, C4, C5, C6 or C8 categories (e.g., identified using any of the keywords “LYMPHOCYTE|T_CELL|PD1|PD-1|PDL1|PD-L1|LAG3|CHECKPOINT_RECEPTOR|B_CELL|PERFORIN|GRANZYME|NK_CELL|CD 8|CYTOTOXIC”). In some embodiments, the method further includes collinearity filtering to remove highly correlated gene sets.

In some embodiments, the IS determining step includes training an elastic net for tumors; and optionally, in some embodiments, the PI determining step includes training the elastic net for tumors, the tumors including immune-depleted tumors. In some embodiments, the PI determining step includes training an elastic net for tumors, the tumors including immune-depleted tumors.

In some embodiments, the method further includes ranking, based on Rho, genes in the gene sets selected in the determining the IS step; and selecting one or more of the ranked genes. In some embodiments, the method further includes ranking, based on a Prognostic Score, genes in the gene sets selected in the determining the PI step, wherein the Prognostic Score includes Rho/Pmeta; and excluding the ranked genes having a Rho from the Prognostic Score of <0.2. In some embodiments, the method further includes combining the Integrated model and an age of a subject into a final model to output a risk score; and recommending one or more treatment plans based on the risk score.

In some embodiments, the tumor-intrinsic model (termed Proliferative Index) includes H, C2, and/or C6 gene sets. In some embodiments, the final model includes extraction of genes included in the IS and the PI; performing, in a first cohort, a meta-analysis of a prognostic effect of each of the extracted genes; performing, in a second cohort, a selection of stably expressed genes across cores and tissue types; ranking of the extracted genes; selecting all of the extracted genes from the IS and the PI. In some embodiments, a number of the extracted genes from the IS is less than 23 per gene set. In some embodiments, a number of the extracted genes from the PI is less than 60 per gene set.

In some embodiments, the final model includes extraction of genes included in the IS and the PI; performing, in a first cohort, a meta-analysis of a prognostic effect of each of the extracted genes; performing, in a second cohort, a selection of stably expressed genes across cores and tissue types; ranking of the extracted genes; selecting a top 23 of the extracted genes per gene set from the IS; selecting a top 60 of the extracted genes per gene set from the PI. In some embodiments, a number of the extracted genes per gene set from the IS is ≥23. In some embodiments, a number of the extracted genes per gene set from the PI is ≥than 60.

In some embodiments, the first cohort includes a training cohort. In some embodiments, the second cohort includes the SweBCG91-RT cohort (or another similar cohort). In some embodiments, the SweBCG91-RT cohort passed RNA, cDNA, and microarray quality control. In some embodiments, the SweBCG91-RT cohort is treated with: breast-conscrving surgery and included in a multivariablc analysis; or breast-conscrving surgery and radiotherapy and included in a multivariable analysis.

In some embodiments, the risk score is a moderate risk score that includes a high PI that is indicative of aggressive tumors and a high IS that is indicative of strong immune activity. In some embodiments, the moderate risk score corresponds to grade III tumors that include PD-1^{High}/PD-L1^{High}/TILs^{High}. In some embodiments, the treatment plain includes immunotherapy. In some embodiments, the treatment plan includes de-intensifying one or more treatments to the grade III tumors if the IS and the PI are high. In some embodiments, the risk score is a high-risk score that includes a moderate/low IS and a high PI. In some embodiments, the high risk score corresponds to grade III tumors that include PD-1^{Low}/PD-L1^{Low}/TILs^{Low}. In some embodiments, the high risk score corresponds to grade I/II tumors that include PD-1^{High}/PD-L1^{High}/TILs^{High}.

In some embodiments, the treatment plan is intensified. In some embodiments, the intensified treatment plan includes treating the subject with intensified radiotherapy that includes a dose of at least one of: 67 Gy or more, add a boosting dose to a standard recommended treatment for the subject when the standard recommended treatment does not include a boosting dose, increase a boosting dose beyond the standard amount for the subject, increase the fraction dose on a per fraction basis above the standard for the subject, increase the number of fractions of a recommended dose above the standard for the subject. In some embodiments, the intensified treatment plan denotes at least one of: intensified radiotherapy treatment, systemic therapy, mastectomy, the additional use of a sensitizer to another therapy; a therapy above a level set by at least one of: the NCCN, ESMO, ESTRO, Clinical Practice Recommendations Australia, and/or NICE guidelines for the subject's remaining indicators, or any combination thereof.

Also provided herein is a method for treating breast cancer, the method including determining an IS based on an immunological model comprised of genes from immunological gene sets; determining a PI based on a tumor-intrinsic model comprised of genes from tumor-intrinsic gene sets; integrating the IS and the PI into an Integrated model; integrating the Integrated model and an age of a subject into a Final model to identify if the subject falls into a specific-risk clinical group based on a risk score from the Final model; and intensifying a treatment plan to the subject when the risk score for the subject is a high risk score, the treatment plan including treating the subject with intensified radiotherapy including a dose of at least one of: 67 Gy or more, add a boosting dose to a standard recommended treatment for the subject when the standard recommended treatment does not include a boosting dose, increase a boosting dose beyond the standard amount for the subject, increase the fraction dose on a per fraction basis above the standard for the subject, increase the number of fractions of a recommended dose above the standard for the subject. In some embodiments, the intensified treatment plan denotes at least one of: intensified radiotherapy treatment, systemic therapy, mastectomy, the additional use of a sensitizer to another therapy; a therapy above a level set by at least one of: the NCCN, ESMO, ESTRO, Clinical Practice Recommendations Australia, and/or NICE guidelines for the subject's remaining indicators, or any combination thereof. In some embodiments, the immunological model is trained in basal and HER2+ tumors. In some embodiments, the tumor intrinsic model is trained in immune-depleted tumors. In some embodiments, the high risk score groups the subject into a high-risk clinical group.

Also provided herein is a method for treating breast cancer, the method including determining expression levels of genes that are included in a model; adjusting the expression levels of the genes by scaling or normalizing to a background population of representative tumors; summing the adjusted expression levels of the genes to determine enrichment scores of gene sets included in the model, wherein the gene sets include the genes; standardizing the enrichment scores by comparing the enrichment scores to the background population; using IS and PI models to calculate IS and PI respectively; standardizing the IS and PI scores by comparing the IS and PI scores to the background population; using an integrated model to calculate an integrated score; standardizing the integrated score by comparing the score to a representative background population; using a final model to determine a risk score of a subject, wherein the final model includes an age of the subject; comparing the risk score of the subject to the representative background population to determine if the risk score of subject falls into a risk category, the risk category including a low-risk group, a medium risk group, or a high risk group; and providing a therapy to the subject based on the risk category of the subject.

In some embodiments, the subject is part of the high risk group, and the therapy provided to the subject is RT intensification. In some embodiments, the subject is part of the low risk group, and the therapy provided to the subject is RT de-intensification. In some embodiments, the subject is part of the medium risk group, and the therapy provided to the subject is standard RT. In some embodiments, the background population is age-matched.

Also provided herein is a method for treating breast cancer, the method including determining an Immunescore (IS) from the tumor sample based on an immunological model comprised of genes from immunological gene sets; determining a Proliferative Index (PI) from the tumor sample based on a tumor-intrinsic model comprised of genes from tumor-intrinsic gene sets; and integrating the IS and the PI into an Integrated model; integrating the Integrated model and an age of a subject into a Final model to identify if the subject falls into a specific-risk clinical group; and providing an appropriate therapy to the subject based on the specific-risk clinical group.

Also provided herein is a method for treating breast cancer, the method including identifying relevant gene sets from immunological gene sets and tumor-intrinsic gene sets; selecting a plurality of the relevant gene sets; creating an IS and a PI from the selected gene sets; integrating the IS and the PI into an integrated model; integrating the integrated model and an age of a subject to create a Final model; identifying, by the Final model, if the subject falls into a specific-risk clinical group; and providing an appropriate therapy to the subject based on the specific-risk clinical group. In some embodiments, the IS corresponds to the immunological gene sets. In some embodiments, the PI corresponds to the tumor-intrinsic gene sets.

Also provided herein is a method for treating breast cancer, the method including determining a tumor aggressivity, the tumor aggressivity including a histological grade of a tumor from at least a portion of a sample of the tumor provided by a subject, wherein the histological grade includes Grade I, Grade II, and Grade III; classifying the sample, when determined as a Grade I tumor, as low-risk; classifying the sample, when determined as a Grade III tumor, as high-risk; determining the tumor aggressivity of the sample, when determined as a Grade II tumor, the tumor aggressivity further including a Proliferative Index score of the sample; classifying the Grade II tumor as: a) high-risk when the Proliferative Index score is greater or equal to a median score of a background population of Grade III tumors, or b) low-risk when the Proliferative Index score is less than the median score of the background population of Grade III tumors; determining an Immunescore of the sample, a level of tumor infiltrating lymphocytes (TILs) in the sample, a level of checkpoint molecules in the sample, or any combination thereof to determine an immunological activity; integrating the tumor aggressivity and the immunological activity by training an elastic net having an interaction term between the tumor aggressivity and the immunological activity, determining a treatment plan based on an integration of tumor aggressivity and immunological activity. In some embodiments, the Proliferative Index score is based on expression of a rust group of genes. In some embodiments, the Immunescore is based on expression of a second group of genes. In some embodiments, the checkpoint molecules include programmed cell death protein-1 (PD-1) and programmed death-ligand 1 (PD-L1). In some embodiments, the immunological activity is determined to be: i) active when the TILs score is ≥10%, and the checkpoint molecules score is ≥1% of lymphocytes with positive staining for PD-1 or PD-L1, or ii) inactive when the TILs score is less than 10%, and the checkpoint molecules score is less than 1% of lymphocytes with positive staining for both PD-1 and PD-L1. In some embodiments, the active immunological activity indicates an activated immune infiltrate. In some embodiments, the inactive immunological activity indicates an inactive immune infiltrate. In some embodiments, the first group of genes includes one or more genes listed in Table 6. In some embodiments, the second group of genes includes one or more genes listed in Table 4. In some embodiments, the treatment plan includes: c) standard radiotherapy, or radiotherapy omission, when the sample is classified as the high-risk Grade II tumor and the immunological activity is active; d) radiotherapy intensification when sample is classified as the high-risk Grade II tumor and the immunological activity is inactive; e) radiotherapy intensification when the sample is classified as the low-risk Grade II tumor and the immunological activity is active; or f) radiotherapy de-intensification, or radiotherapy omission, when the sample is classified as the low-risk Grade II tumor and the immunological activity is inactive.

Also provided herein is a method for treating breast cancer, the method including: determining tumor aggressivity, the tumor aggressivity including a histological grade of a tumor from at least a portion of a sample of the tumor provided by a subject, wherein the histological grade includes Grade I, Grade II, and Grade III; classifying the sample, when determined as a Grade I tumor, as low-risk; classifying the sample, when determined as a Grade III tumor, as high-risk; determining the tumor aggressivity of the sample, when determined as a Grade II tumor, the tumor aggressivity further including a Proliferative Index score of the sample; classifying the Grade II tumor as: a) high-risk when the Proliferative Index score is between 60th to 95th percentile compared to a background population of representative Grade II tumors, or b) low-risk when the Proliferative Index score is below 60th percentile to the background population of representative Grade II tumors; determining an Immunescore of the sample, a level of TILs in the sample, a level of checkpoint molecules in the sample, or any combination thereof to determine an immunological activity; integrating the tumor aggressivity and immunological activity by training an elastic net having an interaction term between the tumor aggressivity and the immunological activity, determining a treatment plan based on the tumor aggressivity, the immunological activity, and the interaction term. In some embodiments, the Proliferative Index score is based on expression of a first group of genes. In some embodiments, the Immunescore is based on expression of a second group of genes. In some embodiments, the checkpoint molecules include PD-1 and PD-L1. In some embodiments, the immunological activity is determined to be: i) active when the TILs score is ≥10%, and the checkpoint molecules score is ≥1% of lymphocytes with positive staining for PD-1 or PD-L1; or ii) inactive when the TILs score is less than 10%, and the checkpoint molecules score is less than 1% of lymphocytes with positive staining for both PD-1 and PD-L1, or when the TILs score is ≥10%, and the checkpoint molecules score is less than 1% of lymphocytes with positive staining for both PD-1 and PD-L1. In some embodiments, the active immunological activity indicates an activated immune infiltrate. In some embodiments, the inactive immunological activity indicates an inactive immune infiltrate. In some embodiments, the first group of genes includes one or more genes listed in Table 6. In some embodiments, the second group of genes includes one or more genes listed in Table 4. In some embodiments, the treatment plan includes: c) standard radiotherapy, or radiotherapy omission, when the sample is classified as the high-risk Grade II tumor and the immunological activity is active; d) radiotherapy intensification when sample is classified as the high-risk Grade II tumor and the immunological activity is inactive; e) the radiotherapy intensification when the sample is classified as the low-risk Grade II tumor and the immunological activity is active; f) radiotherapy de-intensification, or the radiotherapy omission, when the sample is classified as the low-risk Grade II tumor and the immunological activity is inactive; g) the radiotherapy intensification when the sample is classified as a Grade ITT tumor and the immunological activity is inactive; h) radiotherapy de-intensification, or the radiotherapy omission, when the sample is classified as a Grade III tumor and the immunological activity is active; i) the radiotherapy intensification when the sample is classified as a Grade I tumor and the immunological activity is active; or j) radiotherapy de-intensification, or the radiotherapy omission, when the sample is classified as a Grade I tumor and the immunological activity is inactive.

Also provided herein is a method for treating breast cancer, the method including subtyping a tumor from at least a portion of a sample of the tumor provided by a subject, wherein the subtype includes Luminal A, Luminal B, HER2⁺, and triple negative; determining a tumor aggressivity of the sample, when the sample is a Luminal A tumor or a Luminal B tumor, the tumor aggressivity including a Proliferative Index score of the tumor sample; classifying the sample, when the sample is subtyped as the Luminal A tumor, as: a) high-risk when the Proliferative Index score is between 60th to 95th percentile compared to a background population of representative Luminal A tumors, or b) low-risk when the Proliferative Index score is below 60th percentile to the background population of representative Luminal A tumors; classifying the sample, when the sample is subtyped as the Luminal B tumor, as: c) high-risk, when the Proliferative Index score is between 60th to 95th percentile compared to a background population of representative Luminal B tumors, or d) low-risk when the Proliferative Index score is below 60th percentile to the background population of representative Luminal B tumors; determining an immunological activity including scoring an Immunescore of the sample, scoring a level of TILs in the sample, scoring a level of checkpoint molecules in the sample, or any combination thereof; integrating the tumor aggressivity and the immunological activity by training an elastic net having an interaction term between the tumor aggressivity and the immunological activity, determining a treatment plan based on the integrated tumor aggressivity, immunological activity, and the interaction term. In some embodiments, the scoring of the Proliferative Index is based on expression of a first group of genes. In some embodiments, the scoring of the Immunescore is based on expression of a second group of genes. In some embodiments, the checkpoint molecules include PD-1 and PD-L1. In some embodiments, the immunological activity is determined to be: i) active when the TILs score is ≥10%, and either of the checkpoint molecules score is ≥1%, or ii) inactive when the TILs score is less than 10%, and/or the checkpoint molecules score is less than 1%. In some embodiments, the active immunological activity indicates an activated immune infiltrate. In some embodiments, the inactive immunological activity indicates an inactive immune infiltrate. In some embodiments, the first group of genes includes one or more genes listed in Table 6. In some embodiments, the second group of genes includes one or more genes listed in Table 4. In some embodiments, the treatment plan includes e) radiotherapy omission when the tumor sample is classified as the low-risk Luminal A tumor, f) standard radiotherapy or the radiotherapy omission when the sample is classified as the high-risk Luminal B tumor and the immunological activity is active; or g) radiotherapy intensification when tumor sample is classified as the high-risk Luminal B tumor and the immunological activity is inactive.

Also provided herein is a method for treating breast cancer, including: determining a tumor aggressivity of a tumor from at least a portion of a sample of the tumor provided by a subject, wherein the tumor aggressivity includes a Proliferative Index that classifies the tumor as: a) high-risk when a Proliferative Index score of the sample is between 60th to 95th percentile compared to a background population of representative tumors, or b) low-risk when the Proliferative Index score of the sample is below 60th percentile compared to the background population of representative tumors; determining an immunological activity of the tumor based on an Immunescore of the sample; integrating the tumor aggressivity and immunological activity based on an interaction term between the tumor aggressivity and the immunological activity; and determine a treatment plan based on the tumor aggressivity, immunological activity, and the interaction term.

Also provided herein is a method for treating breast cancer, including: determining a tumor aggressivity of a tumor from at least a portion of a sample of the tumor provided by a subject, wherein the tumor aggressivity includes a Proliferative Index score that classifies the tumor as: a) high-risk when the Proliferative Index score of the sample is greater or equal to a median score of a background population of representative tumors, or b) low-risk when the Proliferative Index score of the sample is less than the median score of the background population of representative tumors; determining an immunological activity of the tumor based on an Immunescore of the sample; and integrating the tumor aggressivity and immunological activity based on an interaction term between the tumor aggressivity and the immunological activity; and determining a treatment plan based on the tumor aggressivity, immunological activity, and the interaction term.

In some embodiments, when the tumor is classified as high-risk and the immunological activity is active, the treatment plan includes standard radiotherapy or radiotherapy omission. In some embodiments, when the tumor is classified as high-risk and the immunological activity is inactive, the treatment plan includes radiotherapy intensification. In some embodiments, when the tumor is classified as low-risk and the immunological activity is active, the treatment plan includes radiotherapy intensification. In some embodiments, when the tumor is classified as low-risk and the immunological activity is inactive, the treatment plan includes radiotherapy de-intensification.

In some embodiments, the integrating step includes training an elastic net having an interaction term between the tumor aggressivity and the immunological activity. In some embodiments, a high Proliferative Index score indicates an aggressive tumor. In some embodiments, when the sample is classified as the aggressive tumor and the immunological activity is inactive, the treatment includes radiotherapy intensification.

In some embodiments, the high Proliferative Index score includes a Proliferative Index of the sample being at least in the 60th percentile compared to the background population of representative tumors. In some embodiments, a high Proliferative Index score indicates an aggressive tumor. In some embodiments, when the sample is classified as the aggressive tumor and the immunological activity is inactive, the treatment includes radiotherapy intensification. In some embodiments, the high Proliferative Index score includes a Proliferative Index of the sample being greater or equal to the median score of a background population of representative tumors.

In some embodiments, the scoring of the Proliferative Index is based on expression of a first group of genes. In some embodiments, the first group of genes includes one or more genes listed in Table 6. In some embodiments, the scoring of the Immunescore is based on the expression of a second group of genes. In some embodiments, the second group of genes includes one or more genes listed in Table 4.

In some embodiments, the determining of the immunological activity further includes scoring TILs in the sample. In some embodiments, the active immunological activity includes an activated tumor infiltrate. In some embodiments, the activated tumor infiltrate includes a TILs score of ≥10%. In some embodiments, the step of determining of the immunological activity further includes measuring expression of one or more checkpoint molecules. In some embodiments, the step of determining the immunological activity further includes measuring expression of one or more checkpoint molecules. In some embodiments, the checkpoint molecules include PD-1 and PD-L1.

In some embodiments, the active immunological activity includes an activated tumor infiltrate. In some embodiments, the activated tumor infiltrate includes a positive staining for the expression of one or more of the checkpoint molecules. In some embodiments, the step of determining of the immunological activity further includes scoring TILs in the sample. In some embodiments, the activated tumor infiltrate further includes a TILs score of ≥10%. In some embodiments, the inactive immunological activity includes a TILs score of <10%.

In some embodiments, the integrating step is performed by training an elastic net having the interaction term between the tumor aggressivity and the immunological activity. In some embodiments, the tumor aggressivity further includes a histological grade of the sample of the tumor. In some embodiments, the histological grade of the sample of the tumor is determined as a Grade II tumor. In some embodiments, the method further includes determining a subtype of the tumor sample, wherein the subtype includes Luminal A, Luminal B, HER2⁺, and triple negative prior to determining the tumor aggressivity. In some embodiments, the subtype is Luminal A. In some embodiments, the subtype is Luminal B.

Also provided herein is a method for treating breast cancer, the method including: determining a histological grade of a tumor from at least a sample of the tumor provided by a subject, wherein the histological grade for the tumor includes Grade I, Grade II, or Grade III; assigning a tumor aggressivity of the sample as: a) low-risk when the sample is determined as a Grade I tumor; b) high-risk when the sample is determined as a Grade III tumor, c) low-risk when the sample is determined as a Grade I tumor and a Proliferative Index score of the sample is less than a median score of a background population of tumors; or d) high-risk when the sample is determined as the Grade II tumor and a Proliferative Index score of the sample is greater or equal to a median score of the background population of tumors; classifying an immunological activity as active or inactive, the classifying including determining: e) an Immunescore of the sample; f) a level of TILs in the sample; g) a level of checkpoint molecules in the sample; or h) any combination of e)-f); integrating the tumor aggressivity and the immunological activity using interaction term to determine the benefit of a treatment plan.

In some embodiments, the tumor is classified as high-risk and the immunological activity is active, the treatment plan includes standard radiotherapy or radiotherapy omission. In some embodiments, when the tumor is classified as high-risk and the immunological activity is inactive, the treatment plan includes radiotherapy intensification. In some embodiments, when the tumor is classified as low-risk and the immunological activity is active, the treatment plan includes radiotherapy intensification. In some embodiments, when the tumor is classified as low-risk and the immunological activity is inactive, the treatment plan includes radiotherapy de-intensification.

In some embodiments, the integrating step includes training an elastic net having the interaction term between the tumor aggressivity and the immunological activity. In some embodiments, a high Proliferative Index score indicates an aggressive tumor. In some embodiments, when the sample is classified as the aggressive tumor and the immunological activity is inactive, the treatment includes radiotherapy intensification. In some embodiments, the score of the Proliferative Index is based on expression of a first group of genes. In some embodiments, the first group of genes includes one or more genes listed in Table 6. In some embodiments, the Immunescore is based on expression of a second group of genes. In some embodiments, the second group of genes includes one or more genes listed in Table 4. In some embodiments, the checkpoint molecules include PD-1 and PD-L1.

In some embodiments, the immunological activity is active when: f) is ≥10%, and g) is ≥1% of at least one of the checkpoint molecules. In some embodiments, the active immunological activity indicates an activated tumor infiltrate. In some embodiments, the activated tumor infiltrate includes a positive staining for the expression of one or more of the checkpoint molecules. In some embodiments, the immunological activity is inactive when: f) is <10%, and/or g) is <1% for both checkpoint molecules.

Also provided herein is a method for treating breast cancer including: supplying a sample of a tumor; receiving a treatment based on an analysis of the sample, the analysis including: determining a tumor aggressivity of the tumor from the sample; determining an immunological activity of the tumor from sample; and integrating the tumor aggressivity and the immunological activity based on an interaction term. In some embodiments, the tumor aggressivity includes a Proliferative Index score that classifies the tumor as high-risk or low-risk. In some embodiments, the immunological activity includes: a) an Immunescore of the sample; b) a level of TILs in the sample; c) a level of checkpoint molecules in the sample; or d) any combination of a)-c).

In some embodiments, the tumor is classified as: e) high-risk when the Proliferative Index score of the sample is above a threshold between the 60th to 95th percentile compared to a background population of representative tumors, or f) low-risk when the Proliferative Index score of the sample is below a threshold below the 60th percentile compared to the background population of representative tumors. In some embodiments, the tumor is classified as: g) high-risk when the Proliferative Index score of the sample is greater or equal to a median score of a background population of representative tumors, or h) low-risk when the Proliferative Index score of the sample is less than the median score of the background population of representative tumors. In some embodiments, when the tumor is classified as high-risk and the immunological activity is active, the treatment plan includes standard radiotherapy or radiotherapy omission. In some embodiments, when the tumor is classified as high-risk and the immunological activity is inactive, the treatment plan includes radiotherapy intensification. In some embodiments, when the tumor is classified as low-risk and the immunological activity is active, the treatment plan includes radiotherapy intensification. In some embodiments, the tumor is classified as low-risk and the immunological activity is inactive, the treatment plan includes radiotherapy de-intensification.

In some embodiments, a high Proliferative Index score indicates the tumor as aggressive. In some embodiments, when the tumor is indicated as aggressive, the treatment includes radiotherapy intensification. In some embodiments, the active immunological activity includes an activated tumor infiltrate. In some embodiments, the activated tumor infiltrate includes a TILs score of ≥10%. In some embodiments, the inactivated tumor infiltrate includes a TILs score of <10%. In some embodiments, the checkpoint molecules include PD-1 and PD-L1. In some embodiments, the checkpoint molecules include PD-1 and PD-L1. In some embodiments, the activated tumor infiltrate further includes a positive staining for the expression of one or more of the checkpoint molecules.

In some embodiments, the tumor aggressivity further includes a histological grade of the sample of the tumor. In some embodiments, the histological grade includes Grade I, Grade II, and Grade III. In some embodiments, the tumor analysis further includes determining a subtype of the tumor sample, wherein the subtype includes Luminal A, Luminal B, HER2⁺, and triple negative/basal prior to determining the tumor aggressivity. In some embodiments, the subtype is Luminal A. In some embodiments, the subtype is Luminal B.

In some embodiments, the scoring of the Proliferative Index is based on expression of a first group of genes. In some embodiments, the first group of genes includes one or more genes listed in Table 6. In some embodiments, the Immunescore is based on expression of a second group of genes. In some embodiments, the second group of genes includes one or more genes listed in Table 4. In some embodiments, the integrating step includes training an elastic net having the interaction term between the tumor aggressivity and the immunological activity. In some embodiments, the analysis further includes stratification by pre- and postmenopausal status. In some embodiments, the premenopausal status includes female subjects that are <55 years. In some embodiments, the postmenopausal status includes female subjects that are ≥55 years of age. In some embodiments, the background population is age-matched.

In some embodiments, a method of treating a subject is provided, the method including determining a Proliferative Index based on a level of one or more of the genes in Table 6; determining an Immunescore based on expression of one or more of the genes in Table 4; combining the Proliferative Index and Immunescore and optionally factoring in age of the subject, to determine if the subject will respond to a cancer therapy, and administering the cancer therapy if the Proliferative Index and Immunescore and optionally the age of the subject indicates that the therapy will be successful. In some embodiments, a) there is a high-risk when a Proliferative Index score of the sample is above a threshold between 60th to 95th percentile compared to a background population of representative tumors, or b) there is a low-risk when the Proliferative Index score of the sample is below a threshold below the 60th percentile compared to the background population of representative tumors; c) one combines the Proliferative Index and Immunescore based on an interaction term between the two; and d) one determines the cancer therapy based on the Proliferative Index, Immunescore, and the interaction term. In some embodiments, the background population is age-matched.

In some embodiments, a method of treating a subject is provided, the method including factoring in a level of one or more genes from Table 6; factoring in a level of one or more genes from Table 4; and factoring in an age of the subject, thereby determining if the subject should receive standard radiation therapy, radiotherapy intensification, radiotherapy de-intensification or radiotherapy omission.

In some embodiments, the expression of one or more genes for the IS and/or PI is measured from a tissue sample from the subject. In some embodiments, the sample comprises formalin fixed paraffin embedded tissue sample. In some embodiments, the sample does not comprise TILs. In some embodiments, the analysis does not measure TIL level. In some embodiments, the sample comprises a formalin fixed paraffin embedded tissue sample. In some embodiments, the sample is collected prior to a treatment of the subject for the cancer. In some embodiments, the sample is collected prior to a therapeutic surgery of the subject for the cancer. In some embodiments, age is used as a factor in determining the appropriate therapy or the treatment. In some embodiments, a subject that is younger than 50 is considered young and a subject that is 50 or older is considered old.

In some embodiments, a method of determining a therapy for the treatment of cancer is provided, the method including determining an Immunescore of a core biopsy without determining a level of TILs surrounding a tumor from which the core biopsy was obtained, using the Immunescore to determine an amount of radiotherapy to administer to a subject, without factoring in the level of TILs. In some embodiments, one treats the subject with neoadjuvant immunotherapy.

In some embodiments, the subject is 55 years or older. In some embodiments, the subject is older than 55 years in age. In some embodiments, the subject is 65 years or older. In some embodiments, the subject is older than 65 years. In some embodiments, the subject is 55 years or younger. In some embodiments, the subject is less than 55 years in age. In some embodiments, the subject is less than 50 years of age. In some embodiments, the subject is between 50 and 65 years of age. In some embodiments, the stromal TILS around the tumor are not used in and/or available for analysis or used as part of the method.

In some embodiments, a method of predicting the effectiveness of a cancer therapy is provided, the method including analyzing a sample for a presence of one or more of the genes in Table 4 and/or one or more of the genes in Table 6, wherein a variation in a level of the gene indicates an effectiveness of the cancer therapy. In some embodiments, the method further includes administering a therapy appropriate for the subject based on the analysis of the genes in one or more of Tables 4 and/or 6.

In some embodiments, a method of predicting the effectiveness of a cancer therapy is provided, a method for treating breast cancer is provided the method including determining a histological grade of a tumor from at least a sample of the tumor provided by a subject, wherein the histological grade for the tumor includes Grade I, Grade II, or Grade III; assigning a tumor aggressivity of the sample as: a) low-risk when the sample is determined as a Grade I tumor, b) low-risk when the sample is determined as a Grade II tumor and a Proliferative Index score of the sample is less than a median score of a background population of high-risk tumors; c) high-risk when the sample is determined as the Grade II tumor and a Proliferative Index score of the sample is greater or equal to a median score of the background population of high-risk tumors; or d) high-risk when the sample is determined as: i) a Grade III tumor, ii) the Proliferative Index score of the sample is greater or equal to a median score of the background population of high-risk tumors, iii) ER-negative, iv) HER2-positive, v) the level of Ki67 is high, vi) mutational burden is high, or vii) any combination of i)-vi); classifying an immunological activity includes a level of TILs in the sample; and integrating the tumor aggressivity and the immunological activity using an interaction term to determine a treatment plan.

In some embodiments, the level of Ki67 is ≥10%. In some embodiments, the level of Ki67 is ≥20%. In some embodiments, the level of Ki67 is ≥30%. In some embodiments, the mutational burden is ≥5 mutations per genomic megabase (mut/MB). In some embodiments, the mutational burden is ≥7 mut/MB. In some embodiments, the mutational burden is ≥10 mut/MB.

In some embodiments, a method for treating breast cancer is provided, the method including determining a histological grade of a tumor from at least a sample of the tumor provided by a subject, wherein the histological grade for the tumor includes Grade I, Grade II, or Grade III; assigning a tumor aggressivity of the sample; classifying an immunological activity includes a level of TILs in the sample; and integrating the tumor aggressivity and the immunological activity using an interaction term to determine a treatment plan.

In some embodiments, the assigning of the tumor aggressivity as high-risk includes determining the sample as: i) a Grade III tumor, ii) the Proliferative Index score of the sample is greater or equal to a median score of the background population of tumors, iii) ER-negative, iv) HER2-positive, v) the level of Ki67 is high, vi) mutational burden is high, or vii) any combination of i)-vi). In some embodiments, the level of Ki67 is ≥10%. In some embodiments, the level of Ki67 is ≥20%. In some embodiments, the level of Ki67 is ≥30%. In some embodiments, the mutational burden is ≥5 mut/MB. In some embodiments, the mutational burden is ≥7 mut/MB. In some embodiments, the mutational burden is ≥10 mut/MB.

In some embodiments, the assigning of the tumor aggressivity as high-risk comprises determining the sample as the Grade II tumor and a Proliferative Index score of the sample is greater or equal to a median score of the background population of tumors. In some embodiments, the tumor aggressivity is high-risk. In some embodiments, the level of TILs in the sample is in a range of 50% to 90%, and the treatment plan comprises radiotherapy omission. In some embodiments, the level of TILs in the sample is in a range of 10% to 49%, and the treatment plan comprises boost omission.

In some embodiments, the classifying of the immunological activity further includes a level of checkpoint molecules in the sample. In some embodiments, the checkpoint molecules include PD-1 and PD-L1. In some embodiments, the level of PD-1 is ≥1%. In some embodiments, the level of PD-L1 is ≥1%. In some embodiments, the level of TILs is ≥10%. In some embodiments, the treatment plan comprises RT omission. In some embodiments, the level of PD-1 is <1%. In some embodiments, the level of PD-L1 is <1%. In some embodiments, the level of TILs is ≥10%. In some embodiments, the treatment plan comprises RT boost omission.

In some embodiments, the subject is 55 years or older. In some embodiments, the subject is older than 55 years in age. In some embodiments, the subject is 65 years or older. In some embodiments, the subject is older than 65 years. In some embodiments, the subject is 55 years or younger. In some embodiments, the subject is less than 55 years in age. In some embodiments, the subject is between 50 and 65 years of age. In some embodiments, the subject is less than 50 years of age. In some embodiments, the background population is age-matched. In some embodiments, the sample comprises a core biopsy.

In some embodiments, a method for treating breast cancer is provided, the method including determining a tumor-intrinsic aggressivity of a sample provided by a subject, the tumor-intrinsic aggressivity comprising a Proliferative Index of the sample, a histological grade of the sample, a level of Ki67 from the sample, HER2 expression of the sample, ER-status of the sample, or a combination thereof; determining an immune infiltrate of the sample, the immune infiltrate comprising an Immunescore of the sample, a level of TILs of the sample, a level of PD-1 of the sample, a level of PD-L1 of the sample, or a combination thereof; and providing a treatment based on the tumor-intrinsic aggressivity and the immune infiltrate In some embodiments, the subject is 65 years of age or older.

In some embodiments, a tumor-intrinsic risk is high when: the Proliferative Index is above a 60th percentile compared to a background population of representative patients and tumors; the histological grade is 3; the histological is 2 and the Proliferative Index is equal to or above a median of a background population of grade 3 tumors; the HER2 expression is amplified; the ER-status of the sample is negative; or a combination thereof.

In some embodiments, the immune infiltrate is activated when: the Immunescore is above a threshold of 60th percentile compared to the background population of representative tumors; the level of TILs in the sample is greater than 10%; the level of PD-1 in the sample is greater than 1%; the level of PD-L1 in the sample is greater than 1%; or a combination thereof.

In some embodiments, the tumor-intrinsic risk is high and the immune activation inactive, and the treatment comprises RT boost. In some embodiments, the tumor-intrinsic risk is high, the immune activation inactive, and the subject does not have a co-morbidity, and the treatment comprises chemotherapy intensification. In some embodiments, the treatment further comprises immunotherapy omission. In some embodiments, the sample is ER-positive, and the treatment further comprises endocrine therapy. In some embodiments, the tumor-intrinsic risk is high, the immune activation inactive, and the subject does not have a co-morbidity, and the treatment comprises immunotherapy. In some embodiments, the tumor-intrinsic risk is high, the immune activation inactive, the subject does not have a co-morbidity, and the sample is ER-positive, and the treatment comprises endocrine therapy. In some embodiments, the tumor-intrinsic risk is not high, the immune infiltrate is not activated, the subject does not have a co-morbidity, and the treatment comprises RT omission. In some embodiments, the treatment further comprises chemotherapy omission or de-escalation. In some embodiments, the treatment further comprises endocrine therapy.

In some embodiments, the tumor-intrinsic risk is not high, the immune infiltrate is not activated, the subject does not have a co-morbidity, and the treatment comprises chemotherapy omission or de-escalation. In some embodiments, the tumor-intrinsic risk is not high, the immune infiltrate is not activated, the subject does not have a co-morbidity, and the treatment comprises endocrine therapy. In some embodiments, the tumor-intrinsic risk is not high, the immune infiltrate is activated, and the subject does not have a co-morbidity, and the treatment comprises RT. In some embodiments, the treatment further comprises chemotherapy. In some embodiments, the treatment further comprises endocrine therapy.

In some embodiments, the tumor-intrinsic risk is not high, the immune infiltrate is activated, and the subject does not have a co-morbidity, and the treatment comprises chemotherapy escalation. In some embodiments, the tumor-intrinsic risk is not high, the immune infiltrate is activated, and the subject does not have a co-morbidity, and the treatment comprises endocrine therapy.

In some embodiments, the subject has a co-morbidity. In some embodiments, the co-morbidity includes coronary heart disease, heart failure, chronic obstructive pulmonary disease, previous stroke (ischemic or hemorrhagic), uncontrolled hypertension, diabetes mellitus, osteoporosis, one or more other cancers, or a combination thereof. In some embodiments, the treatment comprises chemotherapy omission and RT de-escalation.

In some embodiments, a method for treating breast cancer is provided, the method including building an Integrated model from a sample provided by a subject to generate an Integrated score, wherein the Integrated model includes a Proliferative Index and an Immunescore; and building a Final model based on the Integrated model and an age of the subject to generate a Final score, providing a treatment based on an Integrated/Final score comprising the Integrated Score or the Final Score. In some embodiments, the subject is 65 years old or older.

In some embodiments, the integrated/Final score is high when the Integrated score or the Final Score is above a 60th percentile of a representative background population of patients and tumors. In some embodiments, the background population is age-matched. In some embodiments, the background population is age-matched with the subject. In some embodiments, the method further comprising determining an immune infiltrate of the sample, the immune infiltrate comprising the Immunescore of the sample. In some embodiments, the immune infiltrate further comprises a level of TILs of the sample, a level of PD-1 of the sample, a level of PD-L1 of the sample, or a combination thereof. In some embodiments, the immune infiltrate is activated when: the Immunescore is above a threshold of 60th percentile compared to the background population of representative tumors; the level of TILs in the sample is greater than 10%; the level of PD-1 in the sample is greater than 1%; the level of PD-L1 in the sample is greater than 1%; or a combination thereof. In some embodiments, the immune infiltrate is activated when the Immunescore is above a threshold of 60th percentile compared to the background population of representative patients and tumors. In some embodiments, the background population is age-matched. In some embodiments, the background population is age-matched with the subject.

In some embodiments, the Integrated/Final score is high, and the treatment comprises RT boost. In some embodiments, the Integrated/Final score is not high and the immune infiltrate is active, and the treatment comprises chemotherapy omission or chemotherapy de-escalation. In some embodiments, the treatment further comprises immunotherapy. In some embodiments, the sample is ER-positive, and the treatment further comprises endocrine therapy. In some embodiments, the Integrated/Final score is high and the immune infiltrate is inactive, and the treatment comprises immunotherapy omission. In some embodiments, the Integrated/Final score is not high and the immune infiltrate is activated, and the treatment comprises RT omission. In some embodiments, the treatment further comprises chemotherapy omission or de-escalation. In some embodiments, the treatment further comprises endocrine therapy. In some embodiments, the Integrated/Final score is not high and the immune infiltrate is activated, and the treatment comprises standard RT. In some embodiments, the treatment further comprises chemotherapy escalation. In some embodiments, the Integrated/Final score is not high and the immune infiltrate is activated, and the treatment comprises chemotherapy escalation. In some embodiments, the treatment further comprises endocrine therapy.

In some embodiments, a method for treating breast cancer is provided, the method including determining a tumor-intrinsic aggressivity of a sample provided by a subject, the tumor-intrinsic aggressivity comprising a Proliferative Index of the sample, a histological grade of the sample, a level of Ki67 from the sample, HER2 expression of the sample, ER-status of the sample, or a combination thereof; determining an immune infiltrate of the sample, the immune infiltrate comprising an Immunescore of the sample, a level of TILs of the sample, a level of PD-1 of the sample, a level of PD-L1 of the sample, or a combination thereof; and providing a treatment based on the tumor-intrinsic aggressivity and the immune infiltrate, wherein the subject is less than 55 years of age.

In some embodiments, a method for treating breast cancer is provided, the method including building an Integrated model from a sample provided by a subject to generate an Integrated score, wherein the Integrated model includes a Proliferative Index and an Immunescore; and building a Final model based on the Integrated model and an age of the subject to generate a Final score, providing a treatment based on an Integrated/Final score comprising the Integrated score or the Final score, wherein the subject is less than 55 years old.

In some embodiments, the Integrated/Final score is less than a 5th percentile of a representative background population of patients and tumors. In some embodiments, the treatment comprises a RT de-escalation. In some embodiments, the Integrated/Final score is greater than a 50th percentile of a representative background population of patients and tumors. In some embodiments, the treatment comprises RT escalation compared to standard RT. In some embodiments, the RT escalation comprises RT boost. In some embodiments, the Integrated/Final score is greater than 95th percentile of a representative background population of patients and tumors. In some embodiments, the treatment comprises surgical escalation. In some embodiments, the surgical escalation comprises mastectomy. In some embodiments, the surgical escalation further comprises wider resection margins. In some embodiments, the treatment further comprises systemic therapy escalation. In some embodiments, the treatment comprises systemic therapy escalation. In some embodiments, the background population is age-matched. In some embodiments, the background population is age-matched with the subject.

In some embodiments, a method for treating breast cancer is provided, the method including building an Integrated model from a sample provided by a subject to generate an Integrated score, wherein the Integrated model includes a Proliferative Index and an Immunescore; and building a Final model based on the Integrated model and an age of the subject to generate a Final score, providing a treatment based on an Integrated/Final score comprising the Integrated score or the Final score, wherein the subject is greater than 55 years old. In some embodiments, the Integrated/Final score is above a 70th percentile of a representative background population of patients and tumors. In some embodiments, the treatment comprises RT escalation. In some embodiments, the RT escalation comprises RT boost. In some embodiments, the Integrated/Final score is above an 85th percentile of a representative background population of patients and tumors. In some embodiments, the treatment comprises mastectomy. In some embodiments, the treatment comprises escalating systemic therapy. In some embodiments, the background population is age-matched. In some embodiments, the background population is age-matched with the subject.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1 is a schematic diagram showing a method according to some non-limiting embodiments of the present disclosure.

FIG. 2 is a schematic diagram continuing the method of FIG. 1 according to some non-limiting embodiments of the present disclosure.

FIGS. 3A and 3B show results from the different methods for the genes in the Immunescore according to some non-limiting embodiments of the present disclosure, wherein FIG. 3A illustrates ipsilateral breast tumor recurrence (local recurrence, IBTR) risk among grade III tumors depending on N genes per biological process (sometimes also called gene set throughout this application), and methods; and FIG. 3B illustrates the interaction with Proliferative Index depending on N genes, and methods.

FIGS. 4A and 4B show results from the different methods for the genes in the Proliferative Index according to some non-limiting embodiments of the present disclosure, wherein FIG. 4A illustrates the IBTR risk of Proliferative Index depending on N genes per biological process, and methods; and FIG. 4B illustrates the interaction with Immunescore depending on N genes per biological process, and methods.

FIG. 5 illustrates the process of using the tool according to some non-limiting embodiments of the present disclosure.

FIG. 6 depicts results according to some non-limiting embodiments of the present disclosure when the model is used on all tumor types in patients from the SweBCG91RT cohort who are divided into groups based on quartiles.

FIG. 7 depicts results according to some non-limiting embodiments of the present disclosure when the model is used on high-risk tumor types patients from the SweBCG91RT cohort with grade III tumors divided into groups based on tertiles.

FIG. 8 is a schematic diagram of the SweBCG91RT cohort.

FIGS. 9A and 9B depict the cumulative IBTR in the SweBCG91RT cohort depending on Immunescore, Proliferative Index and radiotherapy.

FIG. 10 depicts the cumulative incidence of IBTR in the Sjöström and Servant cohorts depending on Immunescore and Proliferative Index. The tumor-intrinsic model, Proliferative Index, could predict the prognostic effect from the immunological model, Immunescore. With high Proliferative Index values, Immunescore was strongly prognostic. However, with low Proliferative Index values, trends toward the opposite were observed.

FIGS. 11A, 11B, 11C, 11D, and 11E depict the cumulative incidence of IBTR among patients aged <70 with grade III tumors or patients aged <60 depending on RT and on a model integrating immunological and tumor-intrinsic qualities. In FIGS. 11A and 11B, high-risk patients (<60 years of age or <70 years with histological grade III) were dichotomized based on a model that integrates Immunescore and Proliferative Index to predict the prognostic effect of an immune infiltrate based on tumor-intrinsic characteristics. In FIGS. 11C, 11D, and 11E, high-risk patients (<60 years of age or <70 years with histological grade III) were stratified into tertiles based on a model that integrates Immunescore and Proliferative Index to predict the prognostic effect of an immune infiltrate based on tumor-intrinsic characteristics. The prognosis and benefit from radiotherapy were analyzed to determine whether combining immunological biomarkers and tumor-intrinsic characteristics may improve radiotherapy individualization.

FIG. 12 is a schematic diagram illustrating a method according to some non-limiting embodiments of the disclosure.

FIG. 13 is a schematic diagram illustrating a method according to some non-limiting embodiments of the disclosure.

FIG. 14 is a schematic diagram illustrating a method according to some non-limiting embodiments of the disclosure.

FIG. 15 depicts a comparison of the correlations of Immunescore, ESTIMATE, and xCell with immunohistochemistry measurements of tumor-infiltrating lymphocytes.

FIG. 16 depicts a correlation between Proliferative Index and Ki67 in the SweBCG91RT cohort. The Ki67 data was evaluated by a board-certified pathologist on tissue microarrays.

FIG. 17 depicts prognostic and RT predictive effect of the Integrated score in the SweBCG91RT cohort.

FIG. 18 depicts prognostic effect of the Integrated model among postmenopausal patients (less than 55 years of age) depending on RT.

FIG. 19 depicts prognostic effect of the Integrated model among postmenopausal patients (55 years or older) depending on RT.

FIG. 20 is a consort diagram of included patients.

FIG. 21 are flow charts for the classification of tumors into low- and high-risk tumor-intrinsic groups as well as of immune infiltrates as activated or inactivated, according to some embodiments. The median score of grade III tumors was used as the cut-off to classify grade II tumors as low- or high-risk.

FIG. 22 is a flow chart of the training of the immunological model (Immunescore), the tumor-intrinsic model (Proliferative Index), and the Integrated model according to some embodiments. Gene sets from the molecular signatures database were used to create a model measuring immunological activity and immunomodulatory tumor-intrinsic factors. These were merged to create a model considering the interaction between the antitumoral immune response and tumor-intrinsic immunomodulatory factors.

FIGS. 23A, 23B, 23C, and 23D are graphs showing cumulative incidences among high- and low-risk tumors with and without an activated immune response.

FIG. 24 shows a negative staining of a sample.

FIGS. 25A and 25B show a positive staining of a sample indicating 1-9% of lymphocytes with FIG. 25B showing an enhance resolution of an area from FIG. 25A as indicated by Box B.

FIG. 26 shows a sample having tumor infiltrating lymphocytes (TILs) present.

FIG. 27 is a consort diagram of included patients.

FIGS. 28A, 28B, and 28C depict cumulative incidences of IBTR in the high-risk group with increasing levels of TILs.

FIG. 29 is a flow chart showing a method according to some non-limiting embodiments of the present disclosure.

FIG. 30 is a flow chart showing systemic therapy according to some non-limiting embodiments of the present disclosure.

FIG. 31 is a flow chart showing radiotherapy according to some non-limiting embodiments of the present disclosure.

### DETAILED DESCRIPTION

All patents, applications, published applications and other publications referred to herein are incorporated herein by reference to the referenced material and in their entireties.

The local immune infiltrate's influence on tumor progression is closely linked to tumor-intrinsic factors. Genomically unstable, or highly proliferating, tumors with a high tumor mutational burden benefit more from an immune infiltrate. Provided herein are methods to integrate immunological and tumor-intrinsic factors to identify clinically high-risk patients who may be candidates for de-escalation of radiotherapy (RT). In some embodiments, the method also allows for the upgrading clinically low-/moderate risk tumors into high-risk tumors (with appropriate therapy thereto when desired). In some embodiments, the sample which is analyzed can be from a core biopsy (and/or lack an analysis of stromal TILs around the tumor and/or the sample is obtained prior to neo-adjuvant and/or adjuvant therapy). In some embodiments, a method is provided that employs one or more of the genes provided herein (such as one or more in Tables 4 and/or 6), where a variation in the gene (e.g., transcript level) indicates an effectiveness of the potential cancer therapy, and optionally, one can thereafter administer an appropriate therapy to the subject to match the specific gene arrangement.

### Definitions

The term “and/or” shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a nucleic acid molecule” includes single or plural nucleic acid molecules and is considered equivalent to the phrase “comprising at least one nucleic acid molecule.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. Unless otherwise specified, the definitions provided herein control when the present definitions may be different from other possible definitions.

As used herein, the term “gene set” denotes a collection of genes where the common denominator of the genes in a particular gene set is the involvement in a specific biological process. Gene sets can be accessed at the Molecular Signatures Database (www.gsea-msigdb.org/gsea/msigdb/). Without being bound by theory, one may regard gene sets as surrogates of specific biological processes. All of the genes sets and their associated genes mentioned herein are incorporated by reference in their entirety. Any of the gene sets and any of their associated genes may be modified, or even discarded, based on feature selection criteria employed by any of the methods disclosed herein.

As used herein, the term “gene” means nucleic acid in the genome of a subject capable of being expressed to produce a mRNA in addition to intervening intronic sequences and in addition to regulatory regions that control the expression of the gene, e.g., a promoter or fragment thereof.

As used herein, the term “Immunescore” (IS) denotes the name of the model that captures immune activity according to some embodiments. As used herein, Immunescore is interchangeable with the term “Cytotoxic Score” (CS).

As used herein, the term “Proliferative Index” (PI) denotes the name of the model that capture aggressive tumor-intrinsic qualities according to some embodiments. As used herein, Proliferative Index is interchangeable with the term “Genomic Index” (GI).

As used herein, the term “Integrated model” denotes the model that combines the Immunescore and Proliferative Index models according to some embodiments.

As used herein, the term “final model” denotes the model that combines the Integrated model with the age of the patient according to some embodiments. In some embodiments, age is included as a continuous variable in the final model.

As used herein, the term “subject” encompasses any animal including humans, preferably a mammal. Exemplary subjects include but are not limited to humans, primates, livestock (e.g. sheep, cows, horses, donkeys, pigs), companion animals (e.g. dogs, cats), laboratory test animals (e.g. mice, rabbits, rats, guinea pigs, hamsters), captive wild animals (e.g. fox, deer). Preferably the mammal is a human or primate. More preferably the mammal is a human. “Subject” and “patient” are used interchangeably herein.

As used herein, the term “diagnosis”, and variants thereof, such as, but not limited to “diagnose” or “diagnosing” shall include, but not be limited to, a primary diagnosis of a clinical state or any primary diagnosis of a clinical state. Furthermore, this term denotes the process of identifying a disease by its signs, symptoms and results of various tests. The conclusion reached through that process is also called “a diagnosis.” Forms of testing commonly performed include biopsy for the collection of the tumor. In some embodiments, the prognosis can be a high or low likelihood of a subsequent (within the next 10 years, 15, or 20 years) invasive breast cancer event.

As used herein, the term “prognosis” denotes an outcome or course of a disease. In some embodiments provided herein, the phrase, when used in the context of a person already having invasive breast cancer, denotes the likelihood that a subject having the invasive breast cancer will go on (within a following ten, fifteen, or twenty year period) to have a subsequent ipsilateral invasive breast cancer event after surgical removal of the primary tumor. The outcome can include a) the likelihood of an ipsilateral breast event, b) the likelihood of an ipsilateral breast event in a particular amount of time (e.g., 1, 2, 3 or 5 years), c) the likelihood that a particular therapy (e.g., radiation) will prevent an ipsilateral breast event, d) an optimal treatment to help prevent an ipsilateral event that matches the severity of the most likely event, or e) combinations thereof.

As used herein, the term “breast tumor” denotes a neoplastic condition of breast tissue that can be benign or malignant. The term “tumor” is synonymous with “neoplasm” and “lesion”. Exemplary breast tumors include invasive breast cancer, ductal carcinoma in situ (DCIS), lobular carcinoma in situ (LCIS), and atypical ductal hyperplasia (ADH).

As used herein, the term “cancer” denotes a malignant neoplasm that has undergone characteristic anaplasia with loss of differentiation, increased rate of growth, invasion of surrounding tissue, and is capable of metastasis. The term “cancer” shall be taken to include a disease that is characterized by uncontrolled growth of cells within a subject, such as, but not limited to, invasive breast cancer. In some embodiments, invasion of the surrounding tissue is the invasion of the basement membrane.

As used herein, the term “intraductal lesion” refers to tumors that are confined to the interior of the mammary ducts and are, therefore, not invasive breast cancers. Exemplary intraductal lesions include ADH and DCIS.

As used herein, ADH is a neoplastic intraductal (non-invasive) lesion characterized by proliferation of evenly distributed, monomorphic mammary epithelial cells.

As used herein, DCIS is a neoplastic intraductal (non-invasive) lesion characterized by increased mammary epithelial proliferation with subtle to marked cellular atypia. DCIS has been divided into grades (low, intermediate, and high) based on factors such as nuclear atypia, intraluminal necrosis, mitotic activity etc. Low-grade DCIS and ADH are morphologically identical, and ADH is distinguished from DCIS based on the extent of the lesion, as determined by its size and/or the number of involved ducts. DCIS is initially typically diagnosed from a tissue biopsy triggered by a suspicious finding (e.g., microcalcifications, unusual mass, tissue distortion or asymmetry, etc.) on a mammogram and/or ultrasound imaging test. It may be from routine screening imaging or, more rarely, from diagnostic imaging triggered by a positive physical examination (e.g., a palpable mass, nipple discharge, skin change, etc.) or by a significant change in a previously identified mass.

Cellular proliferation in DCIS is confined to the milk ducts. If the proliferating cells have invaded through the basement membrane of the myoepithelial cell (MEC) layer lining the duct, thus appearing in the surrounding stroma, then the lesion is considered an invasive breast cancer, even if DCIS is also present. In some cases, the invasion is very minimal (microinvasion) or the only evidence of invasion is disruption of the MEC layer (e.g., by observing discontinuities in MEC-specific protein marker stains such as SMMHC and/or p63). Typically, these microinvasive cases are treated as invasive breast cancers, although there is some controversy in the treatment of these cases.

Recurrence rates in DCIS with current treatments are difficult to estimate. However, it is likely that about 20% of patients who receive lumpectomies without any further treatment would experience recurrence events within 10 years, approximately evenly split between DCIS and invasive events, while <2% of patients who receive mastectomies would experience recurrence. Standard of care with lumpectomy is to receive adjuvant radiation therapy (RT). Several randomized clinical trials provide evidence that adjuvant radiation therapy following lumpectomy reduces recurrence risk by approximately half for both DCIS and invasive event types, and that current clinical and pathologic assessment techniques cannot identify a low-risk sub-group in which there is no benefit from radiation therapy.

As used herein, LCIS is non-invasive lesion that originates in mammary terminal duct-lobular units generally composed of small and often loosely cohesive cells. When it has spread into the ducts, it can be differentiated from DCIS based on morphology and/or marker stains.

As used herein, the term “invasive breast cancer” denotes that the neoplastic (tumor) cells have invaded through the epithelial basement membrane. This distinguishes invasive breast cancer from other hyperplastic (ductal hyperplasia) or dysplastic (atypical ductal hyperplasia, ADH) or non-invasive neoplastic (DCIS, LCIS) breast lesions which are characterized by an intact (non-invaded) basement membrane. It can be divided into stages (I, IIA, IIB, IIA, IIB, and IV). In some embodiments, any of the methods provided herein can be applied to invasive breast cancer to determine the success of radiotherapy for preventing an invasive breast cancer recurrence. In some embodiments, any of the methods provided herein can be applied to DCIS to determine the success of radiotherapy for preventing a DCIS cancer recurrence.

As used herein, the term “radiation therapy” (RT) denotes a therapy that involves or includes some form of radiation in an amount that is therapeutic to the subject.

As used herein, the terms “RT boost” or “boost,” the latter when used within the context of RT, includes an additional/extra dose of radiation applied to a site. The site may be a tumor bed. The site may be an initial tumor site. The site may be any tissue or location requiring the additional or extra dose of radiation. The additional/extra dose may be simultaneously integrated/concomitant (i.e., given together with the standard radiotherapy) or sequential (i.e., given after standard radiotherapy). The additional/extra dose of radiation may be at least 10 Gy if given sequentially or at least 5 Gy if simultaneously integrated/concomitant. The additional/extra dose of radiation may be no more than 16 Gy. The additional/extra dose may be provided using external beam radiotherapy, internal beam radiotherapy/brachytherapy, stereotactic radiotherapy, or intraoperative radiotherapy.

Surgery is a treatment for a breast tumor and is frequently involved in diagnosis. The type of surgery depends upon how widespread the tumor is when diagnosed (the tumor stage), as well as the type and grade of tumor.

The terms “treatment” and “therapy” as provided herein are used interchangeably and does not require the complete or 100% curing of the subject. Instead, it encompasses the broader concept or delaying the onset of one or more symptoms, extending the life and/or quality of life of the subject, reducing the severity of one or more symptoms, etc.

“Standard of care” or “standard” as used herein, with reference to a therapy or treatment, have the ordinary and customary meaning to one of ordinary skill in the art in view of the present disclosure. In some embodiments, standard of care denotes a therapy or treatment option recommended for a patient under a guideline such as that provided by NCCN, ESMO, ESTRO, ASTRO, Clinical Practice Recommendations Australia, or NICE guideline, and optionally, any one or more of the respective guidelines as of June 2022. In some embodiments, a therapy recommended under standard of care for a patient does not take into account guidance provided by analysis of markers as disclosed herein.

The terms “standard radiation therapy” and “standard radiotherapy” are used interchangeably herein and denote a therapy that involves or includes some form of radiation in an amount that is therapeutic to the subject under the current standard of care for breast cancer. In some embodiments, the standard of care is any one that is provided in NCCN, ESMO, ESTRO, ASTRO, Clinical Practice Recommendations Australia, or NICE guideline, and optionally, any one or more of the respective guidelines as of June 2022. In some embodiments, the standard of care is any one of those provided in Tables 14 and 15.

As used herein, the term “radiotherapy de-intensification” includes radiotherapy omission (i.e., not giving any radiotherapy at all) or simply reducing the radiation dose or the number of fractions of a given dose fractionation scheme.

As used herein, the term “local recurrence” denotes that a recurrence is in the operated breast. As used herein, local recurrence is interchangeable with ipsilateral breast tumor recurrence (IBTR).

As used herein, the term “Regional recurrence” denotes that a recurrence is in regional lymph nodes (axillary, supraclavicular, infraclavicular, intrapectoral or internal mammary lymph nodes).

As used herein, the term “distant metastasis” refers to all other recurrences outside the above types of recurrences (local or regional). In other words, distant metastasis refers to recurrences in all other tissues of the body.

### Methods

A vast amount (around 10,000) of pre-existing gene sets were tested in an extensive training cohort to identify and carefully select biological processes associated with antitumoral immune activation and tumor aggressiveness. This was done by calculating the enrichment of each gene set in each tumor according to the method by Barbie, D. A., et al., Systematic RNA interference reveals that oncogenic KRAS-driven cancers require TBK1. Nature, 2009. 462 (7269): 108-12. This means that the expression levels of the genes included in a given gene set are ranked in relation to all other genes by a specific formula (empirical cumulative distribution function). In this way, one can determine whether the specific gene set is enriched in the tumor or not. The gene sets were then analyzed in a meta-analysis of breast cancer training cohorts and the most prognostic gene sets were selected.

To further improve the above method, two models were created by combining the most prognostic different gene sets: one for immune activation, which is named the Immunescore (an example of which is shown in Table 4); and one for tumor aggressiveness, which is named the Proliferative Index (an example of which is shown in Table 6). This was done by the method known as elastic net: a penalized linear regression method. Without being bound by theory, it is thought that the Immunescore and Proliferative Index strongly influence each other, and therefore, integrating them created a new model called Integrated model. This was done by training a new elastic net with an interaction term between Immunescore and Proliferative Index after having calculated, centered and scaled Immunescore and Proliferative Index, respectively, within each training cohort after which the cohorts were merged and the Integrated model was fitted using the formula (Immunescore+Proliferative Index)\hat{ }2.

The model (also called the “Final Model”) is based on the quantification of tumor aggressiveness (measured by Proliferative Index), immune activity (measured by Immunescore), and patient age. The final model is effective at predicting prognosis and RT benefit. However, the groups created are heterogeneous; e.g., a “moderate-risk” score can include patients with aggressive tumors (high Proliferative Index) with a strong immune activity (high Immunescore) or patients with less aggressive tumors (moderate Proliferative Index) who do not benefit from an antitumoral immune response. Aggressive tumors (high PI) with a rich immune infiltrate (high IS) would correspond to a moderate risk score in terms of absolute risk of recurrence (i.e., when comparing this to all tumors, also including low-risk tumors). However, compared to high-risk tumors based on clinical characteristics [e.g., histological grade III and/or ER negative tumors and/or young patients (e.g., with an age <55 years)] this would be considered a low risk score. Similarly, grade III tumors with PD-1^{high}/PD-L1^{High}/TILs^{High} would also correspond to a low risk score when compared to other grade III tumors. But when compared to all tumors (including low-risk tumors), they would probably be considered as a moderate risk score (since grade III in itself is an inherent marker of poor prognosis but an active immune response downgrades this risk from high to a moderate/low risk of recurrence). Grade I/II tumors with a high IS, with a moderate risk score from the final model, or with PD-1^{high}/PD-L1^{High}/TILs^{High} would be considered high-risk tumors when compared to other grade I/II tumors. However, when compared to all tumors (including grade III tumors which are more high-risk), they would be considered to have a moderate risk of recurrence. Table 13 provides additional disclosure on methods other than tumor grade that can be used to define the risk groups.

By also analyzing the Proliferative Index and Immunescore individually (these are always calculated for the Final model), the specific biology of the tumor can be understood which allows for further individualization of the treatment. This can have implications for other types of therapy (e.g., immunotherapy; a moderate Final model score with high Immunescore and Proliferative Index are excellent candidates for immunotherapy). Therefore, the model provides information about the prognosis and RT benefit and as well as provides information about the individual tumor biology, which can most likely guide treatment with immunotherapy, chemotherapy, and other types of targeted therapies. Having a high risk of recurrence may also indicate that the patient derives a benefit from mastectomy over breast-conserving surgery. This model is also very prognostic for distant metastases as well, which makes it very useful for stratifying patients according to the benefit from chemotherapy for example. Since Proliferative Index quantifies tumor aggressiveness, it can be used to estimate the benefit from chemotherapy among patients with clinically low-risk tumors (e.g., Luminal A tumors).

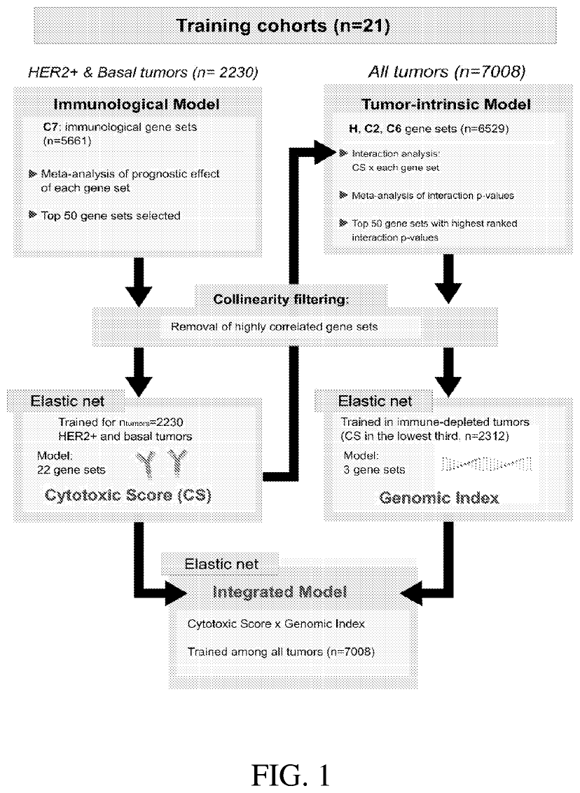

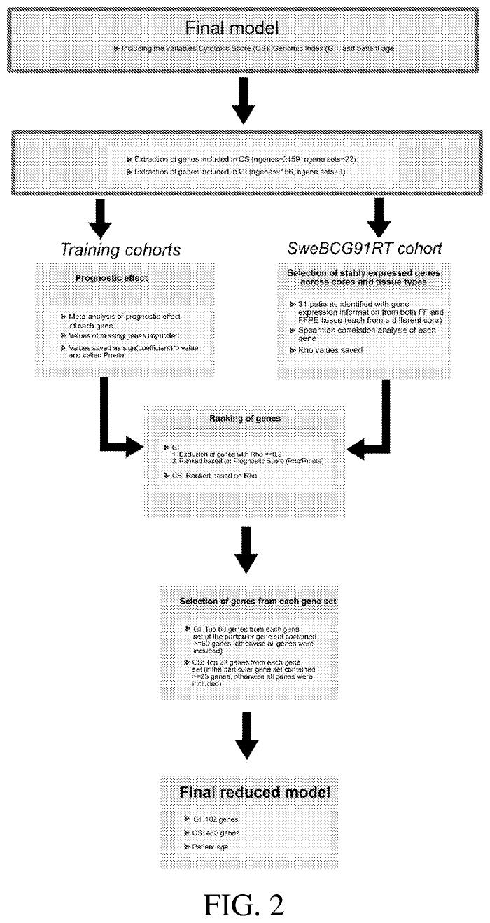

To further improve the Integrated model, one last model was trained to include the patient's age with local recurrence as the endpoint and named the Final Model. This was done in the public cohort called Servant. FIG. 1 describes the whole method up to this step. In some embodiments, FIG. 1 describes the creation of the model where all redundant biological information are filtered out and a distillate of the most important biological processes (as represented by gene sets) is isolated, thereby allowing for the creation of a model based on the interaction between genomic instability and immune activity. It can be said that FIG. 2 is the continuation of FIG. 1 (i.e., the rest of this section of FIG. 1 is illustrated in FIG. 2). FIG. 2 shows how the most useful genes of each gene set included in the model (shown in FIG. 1) were identified, and FIG. 2 also shows the process of removing all redundant genes by taking into account the correlation between different tissue types (fresh-frozen vs. formalin-fixed paraffin-embedded), the heterogeneity of expression over different tumor cores, and the prognostic effect of each individual gene.

The models described above thus use all genes included in each gene set for their calculations (a total of up to about 3,000 genes). A tool needs to be created that can be effectively implemented in the clinic, there is a need to reduce the number of genes significantly and exclude about 90-95% of all genes. This requires great accuracy as reducing the number of genes makes the model less stable. For this tool, genes were chosen that:

Point 1. Profiles well in formalin-fixed paraffin-embedded tissue (which is the type of tumor tissue preparation used in the clinic) as this tissue type introduces significant non-biological variance.

Point 2. Shows low heterogeneity across different tumor cores (high heterogeneity makes the tool insecure and necessitates sampling of multiple cores to be reliable).

Point 3. Provides the most biological information (for example, some genes are more prognostic than others; these are the most prognostic genes that a health practitioner/clinician would desire to find).

Points 1 and 2. Genes are profiled much worse in formalin-fixed paraffin-embedded (FFPE) than in fresh-frozen (FF) due to the mRNA being fragmented in FFPE. Also, cores are less reliable than, for example, whole sections (which evaluate a larger area of the tumor). To overcome this problem, 31 tumor samples were collected with a core from both fresh-frozen material and from FFPE material. By analyzing how genes correlated between the two different cores and tissue types derived from the same tumor, it was possible to determine which genes showed adequate replicability in FFPE tissue in another core. All genes were ranked by rho value which is a measure of this correlation. The analysis was performed for tumors from the SweBCG91RT cohort (Table 1) for which additional fresh-frozen samples with gene expression information were available.

Point 3. To identify the genes that provide the most information, a meta-analysis of all genes was performed (which were included in the different gene sets included in the final models) in the training cohort as shown in Table 3. After, genes were ranked by their prognostic value. For the genes from gene sets from the Immunescore, the analysis was performed for HER2 and basal tumors because the prognostic signal from immunological biomarkers is most robust among these tumors (a sample of these genes are listed in Table 4). All tumors were used for the ranking of genes from Proliferative Index based on prognosis (a sample of these genes are listed in Table 6).

TABLE 1
Clinical characteristics of the SweBCG91RT cohort

| | | No radiotherapy (n = | | Radiotherapy (n = | |
|---|---|---|---|---|---|
| Variable | | 402) | | 362) | |
| Subtype | Luminal A | 222 | (55.8%) | 198 | (55.5%) |
| | Luminal B | 112 | (28.1%) | 104 | (29.1%) |
| | HER2 | 25 | (6.3%) | 29 | (8.1%) |
| | positive | | | | |
| | Triple | 39 | (9.8%) | 26 | (7.3%) |
| | negative | | | | |
| | Missing | 4 | | 5 | |
| ER status | Negative | 51 | (12.7%) | 38 | (10.6%) |
| | Positive | 350 | (87.3%) | 321 | (89.4%) |
| | Missing | 1 | | 3 | |
| | Negative | 106 | (26.4%) | 100 | (27.9%) |
| PgR status | Positive | 295 | (73.6%) | 259 | (72.1%) |
| | Missing | 1 | | 3 | |
| | I | 48 | (12.1%) | 56 | (15.8%) |
| | II | 240 | (60.3%) | 217 | (61.3%) |
| Histological | III | 110 | (27.6%) | 81 | (22.9%) |
| grade | Missing | 4 | | 8 | |
| | No | 368 | (91.5%) | 341 | (94.2%) |
| Endocrine | Yes | 34 | (8.5%) | 21 | (5.8%) |
| therapy | Missing | 0 | | 0 | |
| | No | 396 | (98.5%) | 358 | (98.9%) |
| Chemotherapy | Yes | 6 | (1.5%) | 4 | (1.1%) |
| | Missing | 0 | | 0 | |
| Age (years) | Age | 59 (range: | | 59 (range: | |
| | (years)* | 33-78) | | 31-78) | |
| | Missing | 0 | | 0 | |
| Tumor size | Tumor | 12 | | 12 | |
| (mm) | size | | | | |
| | (mm)* | | | | |
| | Missing | 3 | | 2 | |
| | Stage I | 332 | (83.2%) | 317 | (88.1%) |
| Stage | Stage IIA | 67 | (16.8%) | 43 | (11.9%) |
| | Missing | 3 | | 2 | |
| Events | IBTR | 81 | (20.1%) | 33 | (9.1%) |
| | within 10 | | | | |
| | years | | | | |
| | Competing | 57 | (14.2%) | 58 | (16.0%) |
| | event | | | | |
| | within 10 | | | | |
| | years | | | | |
| | No event | 264 | (65.7%) | 271 | (74.9%) |
| | Missing | 0 | | 0 | |
| LN status | Positive | 0 | | 0 | |
| | Negative | 402 | (100%) | 362 | (100%) |

| | | |
|---|---|---|
| Missing | 0 | 0 |

### Selection of Genes

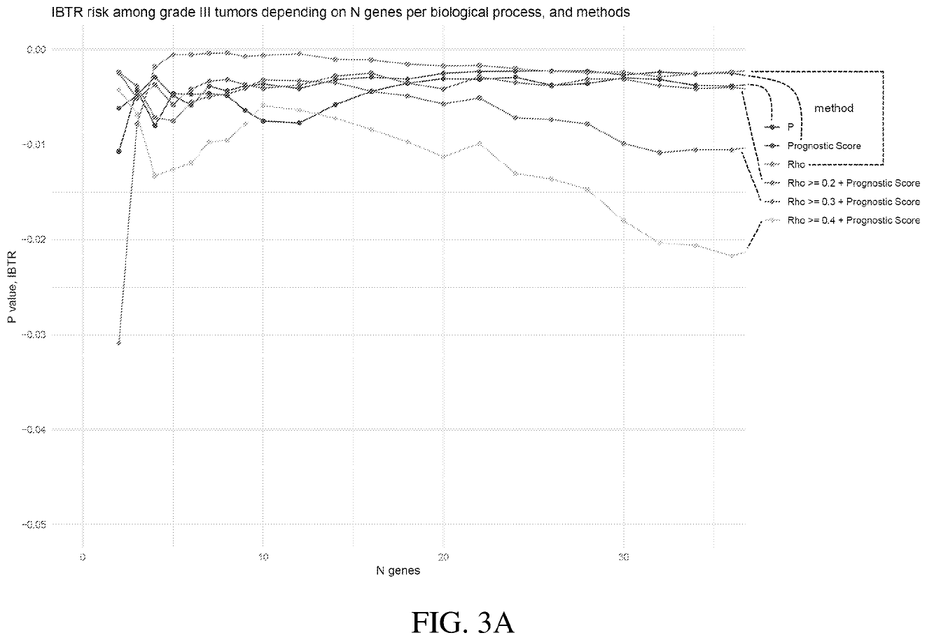

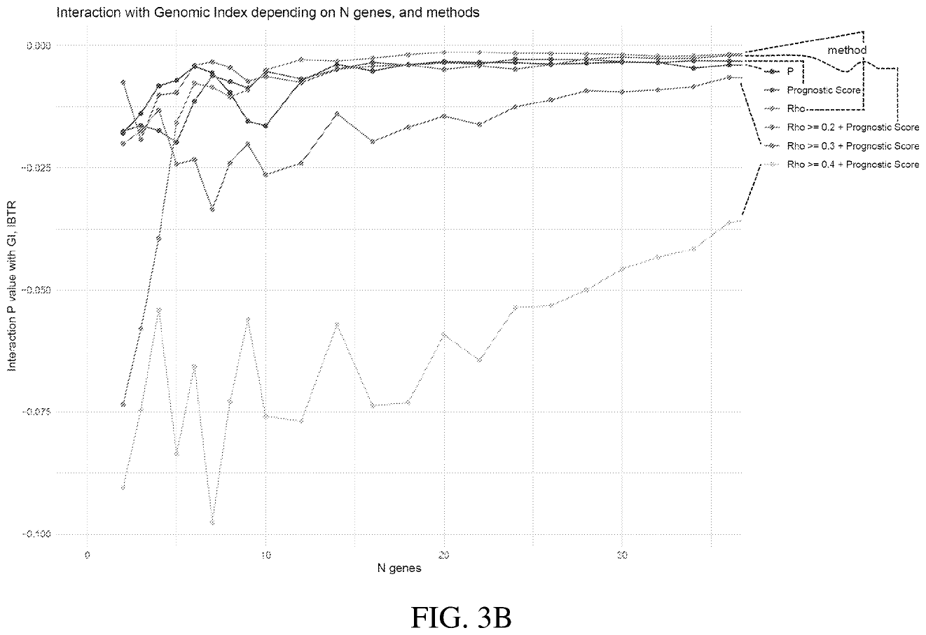

To determine the optimal method for gene selection and to identify the optimal number of genes for the model to work, different methods for gene selection were evaluated. These methods are illustrated in FIGS. 3A-B and 4A-B. The methods are as follows:

Rho: The genes are ranked according to the correlation between fresh-frozen and FFPE tissue and then the number of included genes were varied (from highest correlation and down)

P: The genes are ranked according to meta-analysis p-value from public datasets (e.g., the training cohort), values are imputed when a gene was missing in a dataset. For immune signature genes, the prognostic effect in HER2 and basal tumors was tested; for PI index genes, the prognostic effect among all tumors was tested. Genes are selected from the lowest p-value and increase.

Prognostic score: A value was created that takes into account both the correlation between FFPE vs fresh frozen and the prognostic effect in public datasets through the calculation Rho/P.

Prognostic score+rho>X: Here, first a threshold value on correlation (Rho) (0.2, 0.3, or 0.4, for example) is set and then the remaining genes based on the score are ranked.

For evaluating the methods, instead of the GSEA method (from Barbie et al, 2009), which uses all genes and calculates the enrichment of gene sets, genes have been standardized and then the mean value of the genes in each gene set was calculated. In addition, gene sets are no longer used, rather a smaller subset of the genes is used instead, which differs from the GSEA method. The mean value of the genes is then multiplied by the respective coefficient in the original models. In some embodiments, the values of the genes are first standardized to that of housekeeping genes, and then the mean values are calculated for the genes corresponding to each coefficient. In some embodiments, the housekeeping genes include one or more genes shown in Table 2 below. In some embodiments, the housekeeping genes include one or more of the genes shown in Table 2 below (e.g., 2, 3, 4, 5, 6, 7, 8, 9, 10, 11 or more of the genes below).

TABLE 2
Housekeeping Genes

| Gene | | Chromo- |
|---|---|---|
| symbol | Gene name | some |
| A4galt | alpha 1,4-galactosyltransferase | chr7 |
| Actb | Actin, beta | chr12 |
| B2m | beta-2 microglobulin | chr3 |
| Cck | Cholecystokinin | chr8 |
| Cry2 | Cryptochrome 2 (photolyase-like) | chr3 |
| Csnk1g2 | Casein kinase 1, gamma 2 | chr7 |
| Decr1 | 2,4-dienoyl CoA reductase 1, mitochondrial | chr5 |
| Dimt1 | DIM1 dimethyladenosine transferase 1 homolog | chr2 |
| | (S. cerevisiae) | |
| Eef1a1 | Eukaryotic translation elongation factor 1 alpha 1 | chr8 |
| Farp1 | FERM, RhoGEF (Arhgef) and pleckstrin domain | chr15 |
| | protein 1 (chondrocyte-derived) | |
| Fpgs | Folylpolyglutamate synthase | chr3 |
| Gapdh | Glyceraldehyde-3-phosphate dehydrogenase | chr4 |
| Gins2 | GINS complex subunit 2 (Psf2 homolog) | chr19 |
| Gusb | Glucuronidase, beta | chr12 |
| Hmbs | Hydroxymethylbilane synthase | chr8 |
| Hprt1 | Hypoxanthine phosphoribosyltransferase 1 | chrX |
| Hsp90ab1 | Heat shock protein 90 alpha (cytosolic), class B | chr9 |
| | member 1 | |
| Mapre2 | Microtubule-associated protein, RP/EB family, | chr18 |
| | member 2 | |
| Pex16 | Peroxisomal biogenesis factor 16 | chr3 |
| Pgk1 | Phosphoglycerate kinase 1 | chrX |
| Polr2a | Polymerase (RNA) II (DNA directed) | chr10 |
| | polypeptide A | |
| Ppia | Peptidylprolyl isomerase A (cyclophilin A) | chr14 |
| Ppib | Peptidylprolyl isomerase B | chr8 |
| Pum1 | Pumilio RNA-binding family member 1 | chr5 |
| Rpl4 | Ribosomal protein L4 | chr8 |
| Rplp2 | Ribosomal protein, large P2 | chr1 |
| Sdha | Succinate dehydrogenase complex, subunit A, | chr1 |
| | flavoprotein (Fp) | |
| Srsf4 | Serine/arginine-rich splicing factor 4 | chr5 |
| Tbp | TATA box binding protein | chr1 |
| Tfrc | Transferrin receptor | chr11 |
| Trap1 | TNF receptor-associated protein 1 | chr10 |
| Ubc | Ubiquitin C | chr12 |
| Ywhag | Tyrosine 3-monooxygenase/tryptophan 5- | chr12 |
| | monooxygenase activation protein, gamma | |
| Ywhaz | Tyrosine 3-monooxygenase/tryptophan 5- | chr7 |
| | monooxygenase activation protein, zeta | |

FIGS. 3A and 3B show the different methods for the genes in the Immunescore. The X-axis (N genes) indicates how many of the highest-ranked genes are selected from each gene set. The y-axis in FIG. 3A shows the p-value and the direction of the association (negative value=prognostically favorable, positive value=prognostically unfavorable) of the methods of grade III tumors (wherein it is expected that the model to be prognostically unfavorable). The y-axis in the FIG. 3B shows the p-value and the direction of the interaction test with the original Proliferative Index for the different methods (a negative value as close to zero as possible is preferred). In both FIGS. 3A and 3B, it is seen that the Rho method tends to work best and that less than 5 genes per gene set gives a steep decrease in usefulness. 23 genes per signature (i.e., N genes=23) were included; if the gene set includes >23 genes, the 23 highest ranked are selected, otherwise, all are selected. Further, these methods may evaluate several hundred genes per gene set. In some embodiments, the genes are ranked based on a combination of the prognostic effect in the meta-analysis of the first cohort and the Rho in the second cohort.

In some embodiments, removing genes not sufficiently prognostic and then ranking the remaining genes based on Rho (a measure of correlation) is performed. For Immunescore, this is accomplished by removing genes from immunological gene sets with a coefficient having the opposite sign of the gene set coefficient of the corresponding gene set in the Immunescore model (e.g., if the coefficient of a gene set in the Immunescore model is negative while the coefficient of a gene included in the same gene set is positive) and removing genes having a p-value above a certain threshold (e.g., 0.05 or 0.1 or 0.2 or 0.3) in a meta-analysis of HER2+ and Basal tumors. For Proliferative Index, this is accomplished by removing genes from tumor-intrinsic gene sets with a coefficient going having the opposite sign of the gene set coefficient of the corresponding gene set in the Proliferative Index model and removing genes having a p-value above a certain threshold (e.g., 0.05 or 0.1 or 0.2 or 0.3) in a meta-analysis of all subtypes. Thereafter, ranking the genes from highest to lowest Rho and selecting a top number (designated N) of genes from each gene set.

In some embodiments, a composite score is created which integrates the prognostic effect and Rho value and then all genes are ranked based on this score. This could be accomplished by: 1) Multiplying the meta-analysis coefficient sign (which is negative if the gene is associated with a favorable prognosis and positive if the gene is associated with an unfavorable prognosis) with the meta-analysis p-value. The analysis is performed among HER2+ and Basal tumors for genes included in gene sets included in Immunescore and among all subtypes for genes included in gene sets included in Proliferative Index; 2) Scaling and transforming the above variable while keeping the sign. This could be accomplished by standardizing the variable to have a mean of 0 and a standard deviation of 1; 3) Multiplying the above variable with the rho value; optionally, the rho value can be scaled and transformed to have a mean of 0 and a standard deviation of 1; 4) For each gene set, multiplying the above created variable with the sign of the gene set coefficient in the model (if the coefficient is negative, the variable is multiplied by −1 and if the coefficient is positive, the variable is multiplied by 1). Thereafter, ranking the variable from highest to lowest and selecting a top number (designated N) of genes from each gene set.

In some embodiments, genes with an insufficient correlation (i.e., a Rho value below a certain threshold, for example 0.3) are removed and then the remaining genes are ranked based on the prognostic effect. This is accomplished by: 1) Multiplying the meta-analysis coefficient sign (which is negative if the gene is associated with a favorable prognosis and positive if the gene is associated with an unfavorable prognosis) with the meta-analysis p-value. The analysis is performed among HER2+ and Basal tumors for genes included in gene sets included in Immunescore and among all subtypes for genes included in gene sets included in Proliferative Index; 2) Scaling and transforming the above variable while keeping the sign, this could be accomplished by standardizing the variable to have a mean of 0 and a standard deviation of 1; 3) For each gene set, multiplying the above created variable with the sign of the gene set coefficient in the model (if the coefficient is negative, the variable is multiplied by −1 and if the coefficient is positive, the variable is multiplied by 1). Thereafter, ranking the variable from highest to lowest and selecting a top number (designated N) of genes from each gene set.

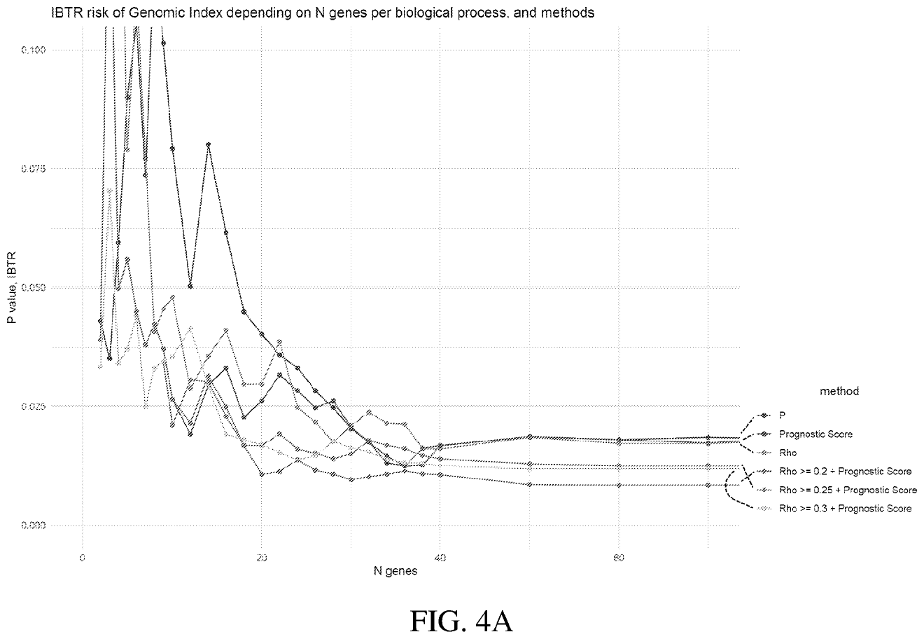

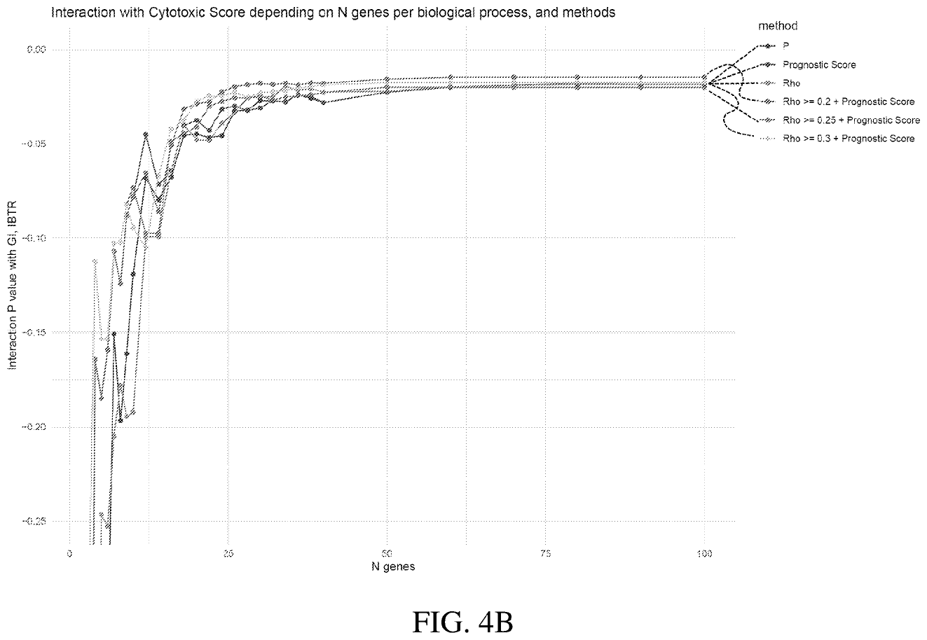

FIGS. 4A and 4B show the different methods for the genes in the Proliferative Index. The x-axis (N genes) indicates, as for FIGS. 3A and 3B, how many of the highest-ranked genes from each gene set are selected. The y-axis in FIG. 4A shows the p-value and the direction of the association (negative value=prognostically favorable, positive value=prognostically unfavorable) of the methods (where the model is expected to be prognostically unfavorable; a positive value as close to 0 as possible is desired). The y-axis in FIG. 4B shows the p-value of the interaction test and the direction with the original Immunescore for the different methods (where a negative value as close to 0 as possible is desired). In both FIGS. 4A and 4B, it is seen that the method called “Rho>=0.2+Prognostic Score” or the Rho method tend to work best and that fewer than 25 genes per gene set give a steep decrease in usability. As shown in FIG. 2, the Prognostic Score includes Rho/Pmeta. Here, 60 genes per signature (i.e., N genes=60) were chosen; if the gene set contains >60 genes, the 60 highest ranked are selected, otherwise, all are selected. Where Pmeta is the score based on the meta analysis of prognostic effect of each gene. In some embodiments, the 5 highest-ranked genes from each gene sets were selected. In some embodiments, the 100 highest-ranked genes from each gene sets were selected. In some embodiments, any number from 5 to 100 of the highest-ranked genes from each gene set were selected.

Two main points from FIGS. 4A and 4B are that, without the present disclosure, it is not apparent which method is best (determined by extensive experimentation), and not all combinations of genes work equally well, as indicated by the steeply decreasing performance as the number of included genes is severely limited.

### The Final Model

Using the calculations and specified genes (shown in Tables 4, 5, 6, and 7) and the outlined methods above, a model that includes 552 genes can be created, which integrates immunological, tumor-intrinsic, and clinical (patient age) information.

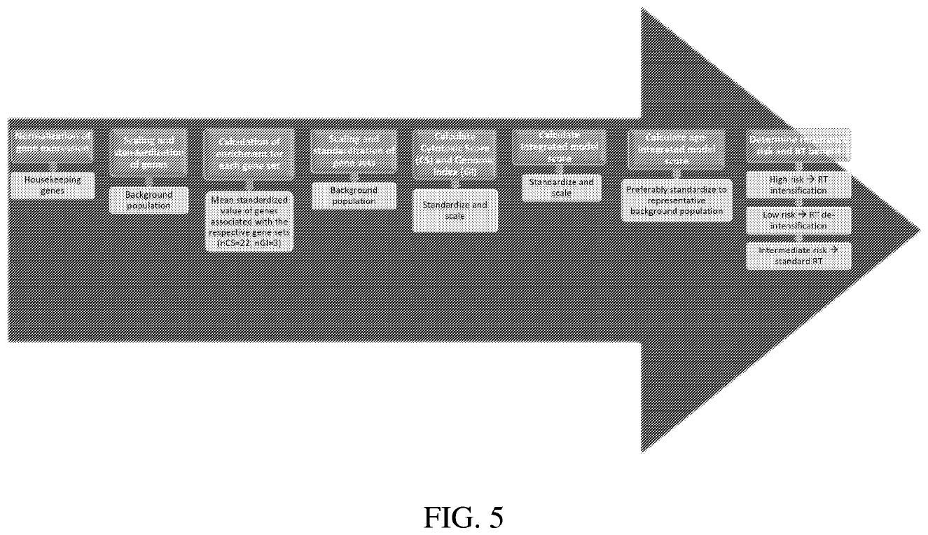

FIG. 5 illustrates the process of using the tool. In general, it can be said that the model's calculations work best when the values for the constituent genes are standardized and scaled. This means that the value is, for example, related to a representative background population where the mean is 0, and the standard deviation is 1. This step is preferably done for each gene and for the models (Immunescore, Proliferative Index, Integrated model) that lead to the final model (Final model).

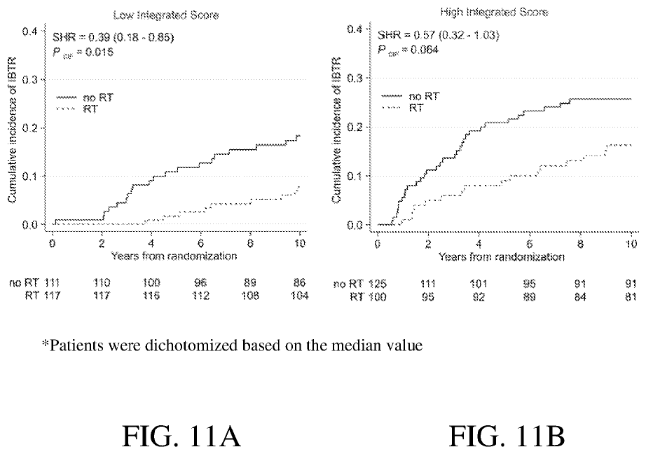

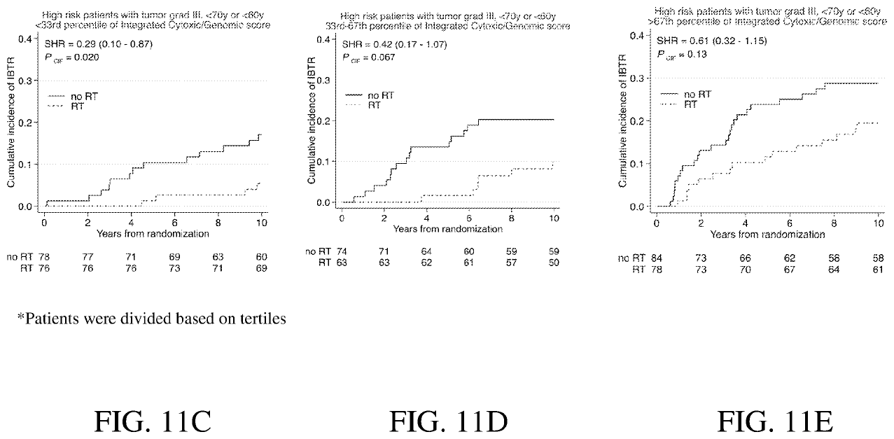

As shown in FIG. 5, in some embodiments, the expression levels of the genes included in the model are determined. Different methods are available to determine these expression levels, the methods including, but not limited to, microarray, RNA-seq, PCR, qPCR (with normalization), Nanostring, etc. Preferably, normalizing the expression levels uses any of the housekeeping genes, or combination thereof, shown in Table 2. Normalization of the expression levels is not limited to using housekeeping genes, as other normalization methods are contemplated. In some embodiments, the genes are scaled compared to a background population of representative tumors. The enrichment of the gene sets (representing different biological pathways) that are included in the model is calculated by summing the (normalized and standardized) values of the genes in the respective included gene sets. The calculated enrichment scores are standardized by comparing a patient's sample to a representative background population. Thereafter, an immunological model and a tumor-intrinsic model are used to calculate IS and PI respectively. The scores of the IS and PI are standardized by comparing to a representative background population. The integrated model is employed to calculate an integrated score (as shown in FIG. 11) and then further standardized by comparing to a representative background population. A final model is used, which also includes patient age, to determine a final risk score. The patient's score is compared to a representative background population to determine if the patient falls into the low-/medium-/high-risk group and providing a suitable therapy based on the patient's risk group.

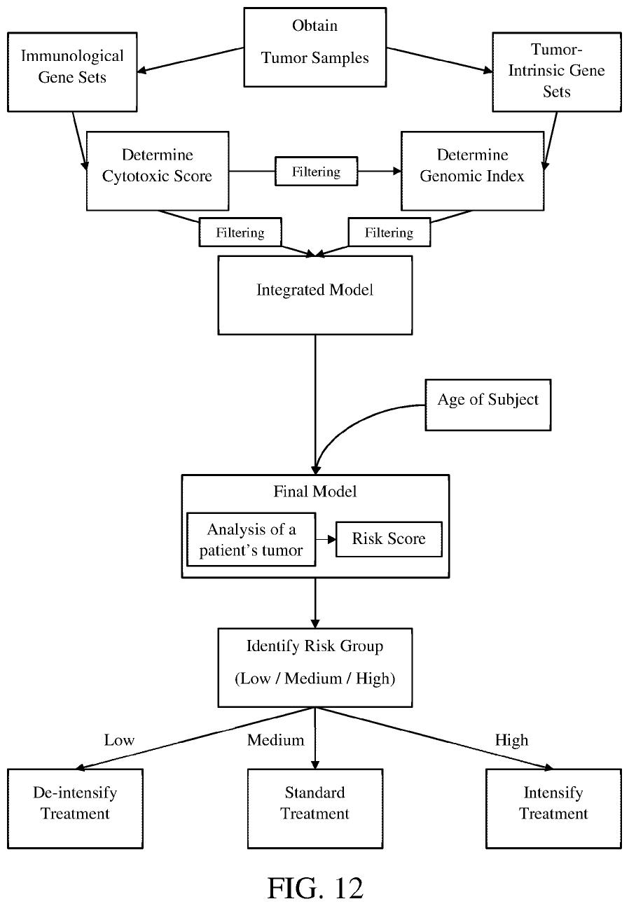

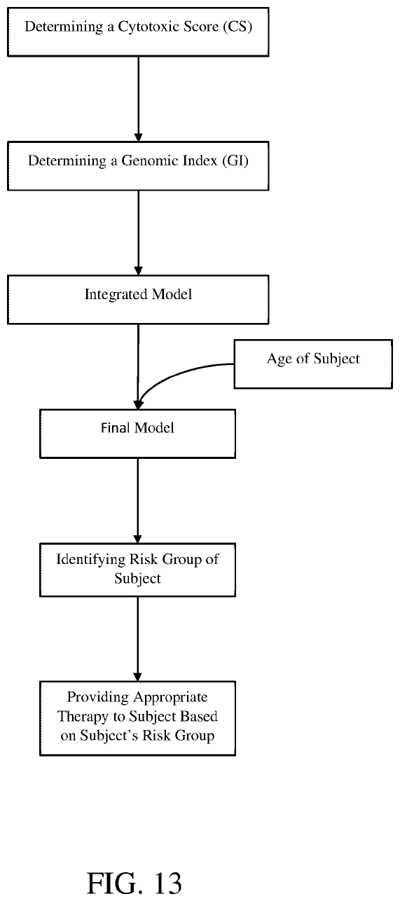

In some embodiments, the method can be depicted as in FIG. 12, and can include one or more of the steps being repeated or additional steps being added in between each of the noted steps. In some embodiments, the method can be depicted as in FIG. 13, and can include one or more of the steps being repeated or additional steps being added in between each of the noted steps.

### General Method

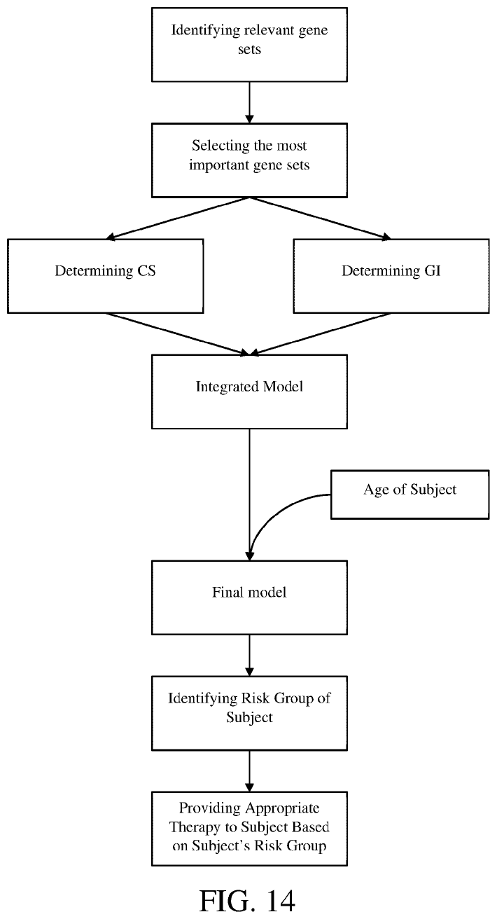

In some embodiments, as shown in FIG. 14, the general steps for the respective tumor-intrinsic and immunological models include: identifying relevant gene sets (e.g., immunological gene sets for IS and tumor-intrinsic gene sets for PI); selecting the most important gene sets; creating IS and PI models; integrating the IS and PI models into an Integrated model; and integrating the Integrated model with a patient age to create a Final model. Once these steps are done, then the best-performing genes in each of the selected gene sets are filtered out to a final list of selected genes, which reduces the number of genes in each gene set in the Final model to improve the clinical utility of the method. Following the selection, the Final model can be used to identify which risk group a subject falls into using the final list of the selected genes from each respective gene set. The gene set scores can be calculated using the mean of the selected genes, which are preferably normalized to one or more housekeeping genes shown in Table 2. In some embodiments, the Final model does not change depending on the subject (i.e., the process of creating this model is not repeated for each subject). It is contemplated that one or more of the method steps may be repeated or additional steps added from any other disclosed methods between each of the noted steps. It is also contemplated that slight permutations in the creation of the Final model may achieve similarly useful results.

Several permutations are possible, which include, but ae not limited to, adjusting the number of gene sets selected, adjusting the threshold for correlation filtering, adjusting the proportion of patients with the lowest IS where PI was trained, adjusting the number of highest-ranked genes from each gene set, and using housekeeping genes as controls. In some embodiments, the number of selected gene sets are adjusted. In some embodiments, the top 5 gene sets are selected. In some embodiments, the top 50 gene sets are selected. In some embodiments, the top 100 gene sets are selected. In some embodiments, the top 200 gene sets are selected. In some embodiments, the gene sets selected may range from the top 5 gene sets to the top 200 gene sets. In some embodiments, a Spearman rho of 0.7 is used as the threshold for correlation/collinearity filtering. In some embodiments, a Spearman rho of 0.5 is used as the threshold for correlation/collinearity filtering. In some embodiments, a Spearman rho of 0.9 is used as the threshold for correlation/collinearity filtering. In some embodiments, a Spearman rho that ranges from 0.5 to 0.9 is used as the threshold for correlation/collinearity filtering. In some embodiments, a proportion of patients included those with the lowest 33% IS where PI was trained. In some embodiments, a proportion of patients included those with the lowest 5% IS where PI was trained. In some embodiments, a proportion of patients included those with the lowest 50% IS where PI was trained. In some embodiments, a proportion of patients included those having a range of the lowest 5% IS to the lowest 50% IS, where PI was trained.

Regarding the number of highest-ranked genes from each gene set, in some embodiments, the top 23 genes from each gene set for IS were used in the general method. In some embodiments, the top 5 genes from each gene set for IS were used in the general method. In some embodiments, the top 100 genes from each gene set for IS were used in the general method. In some embodiments, the top genes ranged from the top 5 genes to the top 100 genes from each gene set for IS were used in the general method. In some embodiments, the top 60 genes from each gene set for PI were used in the general method. In some embodiments, the top 5 genes from each gene set for PI were used in the general method. In some embodiments, the top 100 genes from each gene set for PI were used in the general method. In some embodiments, the top genes ranged from the top 5 genes to the top 100 genes from each gene set for PI were used in the general method.

### Distinct Groups

The groups that are created with the method correspond to groups that have been attempted to identify using histological grade and PD-1/PD-L1/TILs. TIL referring to tumor infiltrating lymphocytes.

“Programmed death protein 1”, “PD-1”, “PD1”, “PDCD1” are used interchangeably herein and refers to the gene or gene product of PDCD1. In some embodiments, PD-1 is human PD-1. In some embodiments, PD-1 protein has the amino acid sequence as shown in SEQ ID NO:1, with or without the signal peptide (underlined).

| (SEQ ID NO: 1) | |
|---|---|
| 1 | mqipqapwpv vwavlqlgwr pgwfldspdr pwnpptfspa llvvtegdna tftcsfsnts |
| 61 | esfvlnwyrm spsnqtdkla afpedrsqpg qdcrfrvtql pngrdfhmsv vrarrndsgt |
| 121 | ylcgaislap kaqikeslra elrvterrae vptahpspsp rpagqfqtlv vgvvggllgs |
| 181 | lvllvwvlav icsraargti garrtgqplk edpsavpvfs vdygeldfqw rektpeppvp |
| 241 | cvpeqteyat ivfpsgmgts sparrgsadg prsaqplrpe dghcswpl |

“PD-L1” and “PDL1” are used interchangeably herein and refers to the gene or gene product of CD274. In some embodiments, PD-L1 is human PD-L1. In some embodiments, PD-L1 protein has the amino acid sequence as shown in SEQ ID NO:2, with or without the signal peptide (underlined).

| (SEQ ID NO: 2 | |
|---|---|
| 1 | mrifavfifm tywhllnaft vtvpkdlyvv eygsnmtiec kfpvekqldl aalivyweme |
| 61 | dkniiqfvhg eedlkvqhss yrqrarllkd qlslgnaalq itdvklqdag vyrcmisygg |
| 121 | adykritvkv napynkinqr ilvvdpvtse heltcqaegy pkaeviwtss dhqvlsgktt |
| 181 | ttnskreekl fnvtstlrin tttneifyct frrldpeenh taelvipelp lahppnerth |
| 241 | lvilgaillc lgvaltfifr lrkgrmmdvk kcgiqdtnsk kqsdthleet |

In some embodiments, PD-L1 protein has the amino acid sequence as shown in SEQ ID NO:3, with or without the signal peptide (underlined).

| (SEQ ID NO: 3) | |
|---|---|
| 1 | mrifavfifm tywhllnapy nkinqrilvv dpvtsehelt cqaegypkae viwtssdhqv |
| 61 | lsgkttttns kreeklfnvt stlrintttn eifyctfrrl dpeenhtael vipelplahp |
| 121 | pnerthlvil gaillclgva ltfifrlrkg rmmdvkkcgi qdtnskkqsd thleet |

In some embodiments, PD-L1 mRNA has the nucleotide sequence as shown in SEQ ID NO:4, or a processed form thereof:

| (SEQ ID NO: 4) | |
|---|---|
| 1 | agttctgcgc agcttcccga ggctccgcac cagccgcgct tctgtccgcc tgcagggcat |
| 61 | tccagaaaga tgaggatatt tgctgtcttt atattcatga cctactggca tttgctgaac |
| 121 | gcatttactg tcacggttcc caaggaccta tatgtggtag agtatggtag caatatgaca |
| 181 | attgaatgca aattcccagt agaaaaacaa ttagacctgg ctgcactaat tgtctattgg |
| 241 | gaaatggagg ataagaacat tattcaattt gtgcatggag aggaagacct gaaggttcag |
| 301 | catagtagct acagacagag ggcccggctg ttgaaggacc agctctccct gggaaatgct |
| 361 | gcacttcaga tcacagatgt gaaattgcag gatgcagggg tgtaccgctg catgatcagc |
| 421 | tatggtggtg ccgactacaa gcgaattact gtgaaagtca atgccccata caacaaaatc |
| 481 | aaccaaagaa ttttggttgt ggatccagtc acctctgaac atgaactgac atgtcaggct |
| 541 | gagggctacc ccaaggccga agtcatctgg acaagcagtg accatcaagt cctgagtggt |
| 601 | aagaccacca ccaccaattc caagagagag gagaagcttt tcaatgtgac cagcacactg |
| 661 | agaatcaaca caacaactaa tgagattttc tactgcactt ttaggagatt agatcctgag |
| 721 | gaaaaccata cagctgaatt ggtcatccca gaactacctc tggcacatcc tccaaatgaa |
| 781 | aggactcact tggtaattct gggagccatc ttattatgcc ttggtgtagc actgacattc |
| 841 | atcttccgtt taagaaaagg gagaatgatg gatgtgaaaa aatgtggcat ccaagataca |
| 901 | aactcaaaga agcaaagtga tacacatttg gaggagacgt aatccagcat tggaacttct |
| 961 | gatcttcaag cagggattct caacctgtgg tttaggggtt catcggggct gagcgtgaca |
| 1021 | agaggaagga atgggcccgt gggatgcagg caatgtggga cttaaaaggc ccaagcactg |
| 1081 | aaaatggaac ctggcgaaag cagaggagga gaatgaagaa agatggagtc aaacagggag |
| 1141 | cctggaggga gaccttgata ctttcaaatg cctgaggggc tcatcgacgc ctgtgacagg |
| 1201 | gagaaaggat acttctgaac aaggagcctc caagcaaatc atccattgct catcctagga |
| 1261 | agacgggttg agaatcccta atttgagggt cagttcctgc agaagtgccc tttgcctcca |
| 1321 | ctcaatgcct caatttgttt tctgcatgac tgagagtctc agtgttggaa cgggacagta |
| 1381 | tttatgtatg agtttttcct atttattttg agtctgtgag gtcttcttgt catgtgagtg |
| 1441 | tggttgtgaa tgatttcttt tgaagatata ttgtagtaga tgttacaatt ttgtcgccaa |
| 1501 | actaaacttg ctgcttaatg atttgctcac atctagtaaa acatggagta tttgtaaggt |
| 1561 | gcttggtctc ctctataact acaagtatac attggaagca taaagatcaa accgttggtt |
| 1621 | gcataggatg tcacctttat ttaacccatt aatactctgg ttgacctaat cttattctca |
| 1681 | gacctcaagt gtctgtgcag tatctgttcc atttaaatat cagctttaca attatgtggt |
| 1741 | agcctacaca cataatctca tttcatcgct gtaaccaccc tgttgtgata accactatta |
| 1801 | ttttacccat cgtacagctg aggaagcaaa cagattaagt aacttgccca aaccagtaaa |
| 1861 | tagcagacct cagactgcca cccactgtcc ttttataata caatttacag ctatatttta |
| 1921 | ctttaagcaa ttcttttatt caaaaaccat ttattaagtg cccttgcaat atcaatcgct |
| 1981 | gtgccaggca ttgaatctac agatgtgagc aagacaaagt acctgtcctc aaggagctca |
| 2041 | tagtataatg aggagattaa caagaaaatg tattattaca atttagtcca gtgtcatagc |
| 2101 | ataaggatga tgcgagggga aaacccgagc agtgttgcca agaggaggaa ataggccaat |
| 2161 | gtggtctggg acggttggat atacttaaac atcttaataa tcagagtaat tttcatttac |
| 2221 | aaagagaggt cggtacttaa aataaccctg aaaaataaca ctggaattcc ttttctagca |
| 2281 | ttatatttat tcctgatttg cctttgccat ataatctaat gcttgtttat atagtgtctg |
| 2341 | gtattgttta acagttctgt cttttctatt taaatgccac taaattttaa attcatacct |
| 2401 | ttccatgatt caaaattcaa aagatcccat gggagatggt tggaaaatct ccacttcatc |
| 2461 | ctccaagcca ttcaagtttc ctttccagaa gcaactgcta ctgcctttca ttcatatgtt |
| 2521 | cttctaaaga tagtctacat ttggaaatgt atgttaaaag cacgtatttt taaaattttt |
| 2581 | ttcctaaata gtaacacatt gtatgtctgc tgtgtacttt gctattttta tttattttag |
| 2641 | tgtttcttat atagcagatg gaatgaattt gaagttccca gggctgagga tccatgcctt |
| 2701 | ctttgtttct aagttatctt tcccatagct tttcattatc tttcatatga tccagtatat |
| 2761 | gttaaatatg tcctacatat acatttagac aaccaccatt tgttaagtat ttgctctagg |
| 2821 | acagagtttg gatttgttta tgtttgctca aaaggagacc catgggctct ccagggtgca |
| 2881 | ctgagtcaat ctagtcctaa aaagcaatct tattattaac tctgtatgac agaatcatgt |
| 2941 | ctggaacttt tgttttctgc tttctgtcaa gtataaactt cactttgatg ctgtacttgc |
| 3001 | aaaatcacat tttctttctg gaaattccgg cagtgtacct tgactgctag ctaccctgtg |
| 3061 | ccagaaaagc ctcattcgtt gtgcttgaac ccttgaatgc caccagctgt catcactaca |
| 3121 | cagccctcct aagaggcttc ctggaggttt cgagattcag atgccctggg agatcccaga |
| 3181 | gtttcctttc cctcttggcc atattctggt gtcaatgaca aggagtacct tggctttgcc |
| 3241 | acatgtcaag gctgaagaaa cagtgtctcc aacagagctc cttgtgttat ctgtttgtac |
| 3301 | atgtgcattt gtacagtaat tggtgtgaca gtgttctttg tgtgaattac aggcaagaat |
| 3361 | tgtggctgag caaggcacat agtctactca gtctattcct aagtcctaac tcctccttgt |
| 3421 | ggtgttggat ttgtaaggca ctttatccct tttgtctcat gtttcatcgt aaatggcata |
| 3481 | ggcagagatg atacctaatt ctgcatttga ttgtcacttt ttgtacctgc attaatttaa |
| 3541 | taaaatattc ttatttattt tcttacttgg tacaccagca tgtccatttt cttgtttatt |
| 3601 | ttgtgtttaa taaaatgttc agtttaacat ccca |

In some embodiments, PD-L1 mRNA has the nucleotide sequence as shown in SEQ ID NO:5, or a processed form thereof:

| (SEQ ID NO: 5) | |
|---|---|
| 1 | agttctgcgc agcttcccga ggctccgcac cagccgcgct tctgtccgcc tgcagggcat |
| 61 | tccagaaaga tgaggatatt tgctgtcttt atattcatga cctactggca tttgctgaac |
| 121 | gccccataca acaaaatcaa ccaaagaatt ttggttgtgg atccagtcac ctctgaacat |
| 181 | gaactgacat gtcaggctga gggctacccc aaggccgaag tcatctggac aagcagtgac |
| 241 | catcaagtcc tgagtggtaa gaccaccacc accaattcca agagagagga gaagcttttc |
| 301 | aatgtgacca gcacactgag aatcaacaca acaactaatg agattttcta ctgcactttt |
| 361 | aggagattag atcctgagga aaaccataca gctgaattgg tcatcccaga actacctctg |
| 421 | gcacatcctc caaatgaaag gactcacttg gtaattctgg gagccatctt attatgcctt |
| 481 | ggtgtagcac tgacattcat cttccgttta agaaaaggga gaatgatgga tgtgaaaaaa |
| 541 | tgtggcatcc aagatacaaa ctcaaagaag caaagtgata cacatttgga ggagacgtaa |
| 601 | tccagcattg gaacttctga tcttcaagca gggattctca acctgtggtt taggggttca |
| 661 | tcggggctga gcgtgacaag aggaaggaat gggcccgtgg gatgcaggca atgtgggact |
| 721 | taaaaggccc aagcactgaa aatggaacct ggcgaaagca gaggaggaga atgaagaaag |
| 781 | atggagtcaa acagggagcc tggagggaga ccttgatact ttcaaatgcc tgaggggctc |
| 841 | atcgacgcct gtgacaggga gaaaggatac ttctgaacaa ggagcctcca agcaaatcat |
| 901 | ccattgctca tcctaggaag acgggttgag aatccctaat ttgagggtca gttcctgcag |
| 961 | aagtgccctt tgcctccact caatgcctca atttgttttc tgcatgactg agagtctcag |
| 1021 | tgttggaacg ggacagtatt tatgtatgag tttttcctat ttattttgag tctgtgaggt |
| 1081 | cttcttgtca tgtgagtgtg gttgtgaatg atttcttttg aagatatatt gtagtagatg |
| 1141 | ttacaatttt gtcgccaaac taaacttgct gcttaatgat ttgctcacat ctagtaaaac |
| 1201 | atggagtatt tgtaaggtgc ttggtctcct ctataactac aagtatacat tggaagcata |
| 1261 | aagatcaaac cgttggttgc ataggatgtc acctttattt aacccattaa tactctggtt |
| 1321 | gacctaatct tattctcaga cctcaagtgt ctgtgcagta tctgttccat ttaaatatca |
| 1381 | gctttacaat tatgtggtag cctacacaca taatctcatt tcatcgctgt aaccaccctg |
| 1441 | ttgtgataac cactattatt ttacccatcg tacagctgag gaagcaaaca gattaagtaa |
| 1501 | cttgcccaaa ccagtaaata gcagacctca gactgccacc cactgtcctt ttataataca |
| 1561 | atttacagct atattttact ttaagcaatt cttttattca aaaaccattt attaagtgcc |
| 1621 | cttgcaatat caatcgctgt gccaggcatt gaatctacag atgtgagcaa gacaaagtac |
| 1681 | ctgtcctcaa ggagctcata gtataatgag gagattaaca agaaaatgta ttattacaat |
| 1741 | ttagtccagt gtcatagcat aaggatgatg cgaggggaaa acccgagcag tgttgccaag |
| 1801 | aggaggaaat aggccaatgt ggtctgggac ggttggatat acttaaacat cttaataatc |
| 1861 | agagtaattt tcatttacaa agagaggtcg gtacttaaaa taaccctgaa aaataacact |
| 1921 | ggaattcctt ttctagcatt atatttattc ctgatttgcc tttcccatat aatctaatgc |
| 1981 | ttgtttatat agtgtctggt attgtttaac agttctgtct tttctattta aatgccacta |
| 2041 | aattttaaat tcataccttt ccatgattca aaattcaaaa gatcccatgg gagatggttg |
| 2101 | gaaaatctcc acttcatcct ccaagccatt caagtttcct ttccagaagc aactgctact |
| 2161 | gcctttcatt catatgttct tctaaagata gtctacattt ggaaatgtat gttaaaagca |
| 2221 | cgtattttta aaattttttt cctaaatagt aacacattgt atgtctgctg tgtactttgc |
| 2281 | tatttttatt tattttagtg tttcttatat agcagatgga atgaatttga agttcccagg |
| 2341 | gctgaggatc catgccttct ttgtttctaa gttatctttc ccatagcttt tcattatctt |
| 2401 | tcatatgatc cagtatatgt taaatatgtc ctacatatac atttagacaa ccaccatttg |
| 2461 | ttaagtattt gctctaggac agagtttgga tttgtttatg tttgctcaaa aggagaccca |
| 2521 | tgggctctcc agggtgcact gagtcaatct agtcctaaaa agcaatctta ttattaactc |
| 2581 | tgtatgacag aatcatgtct ggaacttttg ttttctgctt tctgtcaagt ataaacttca |
| 2641 | ctttgatgct gtacttgcaa aatcacattt tctttctgga aattccggca gtgtaccttg |
| 2701 | actgctagct accctgtgcc agaaaagcct cattcgttgt gcttgaaccc ttgaatgcca |
| 2761 | ccagctgtca tcactacaca gccctcctaa gaggcttcct ggaggtttcg agattcagat |
| 2821 | gccctgggag atcccagagt ttcctttccc tcttggccat attctggtgt caatgacaag |
| 2881 | gagtaccttg gctttgccac atgtcaaggc tgaagaaaca gtgtctccaa cagagctcct |
| 2941 | tgtgttatct gtttgtacat gtgcatttgt acagtaattg gtgtgacagt gttctttgtg |
| 3001 | tgaattacag gcaagaattg tggctgagca aggcacatag tctactcagt ctattcctaa |
| 3061 | gtcctaactc ctccttgtgg tgttggattt gtaaggcact ttatcccttt tgtctcatgt |
| 3121 | ttcatcgtaa atggcatagg cagagatgat acctaattct gcatttgatt gtcacttttt |
| 3181 | gtacctgcat taatttaata aaatattctt atttattttg ttacttggta caccagcatg |
| 3241 | tccattttct tgtttatttt gtgtttaata aaatgttcag tttaacatcc ca |

One group includes a high-risk refractory group that corresponds to grade III PD-1^{Low}/PD-L1^{Low}/TILs^{Low} group, which reflects the high risk scores of the final model. In one method, the high risk score of the final model is independent of other clinical characteristics. In another method, tumors are even more effectively identified by combining histological grade (or other clinical markers of tumor aggressivity, such as ER-negativity or premenopausal age) with the model, e.g., histological grade III tumors with high risk scores. In some embodiments, the high risk score in the final model is combined with moderate/low Immunescore and a high Proliferative Index score. In some embodiments, the high risk score in the final model is combined with moderate/low Immunescore and a high Proliferative Index score; this method relies entirely on in-house developed models.

Another group includes an immune-activated group that corresponds to grade III PD-1^{High}/PD-L1^{High}/TILs^{High} group, which reflects low/medium risk scores of the final model where Proliferative Index and Immunescores are high. In one method, they have a medium risk score in the final model. These tumors are most effectively identified by combining histological grade (or other clinical markers of tumor aggressivity) with the model, e.g., histological grade III tumors with low/medium scores. In another method, they have high Immunescore and high Proliferative Index. These tumors show up as a medium/low score in the final model. Looking at the Proliferative Index and immunescore separately is helpful as lower-risk tumors may also show a moderate/low score in the final model. Low-risk tumors have instead a low/moderate score for Proliferative Index. This has implications for what treatment should be chosen for moderate-score tumors.

Yet another group includes a low-risk refractory group that corresponds to grade I/II PD-1^{High}/PD-L1^{High}/TILs^{High} group, which reflects the moderate/low risk scores of the final model. Those who are grouped under a moderate/low risk score need intensified treatment similar to the grade/II PD-1^{High}/PD-L1^{High}/TILs^{High} group from the prior disclosures (PCT/US2021/032080 filed on May 21, 2022 and PCT/US2022/022934 filed on Mar. 31, 2022, all of which are incorporated by reference herein). In one method, the group includes the moderate risk score of the final model and a moderate Proliferative Index score. This group can be differentiated from the “Immune-activated group” by its lower Proliferative Index score. Usually, this group will also have a moderate or high Immunescore. In another method, combining histological grade (or other clinical markers of tumor low tumor aggressivity) with the model, e.g., histological grade I/II tumors with moderate scores of the Final model or histological grade I/II tumors with moderate/high Immunescore.

Yet another group includes a low-risk immunological group that corresponds to grade I/II PD-1^{High}/PD-L1^{High}/TILs^{High} group, which reflect moderate/low risk scores of the final model. These tumors need intensified treatment similar to the grade I/II PD-1^{High}/PD-L1^{High}/TILs^{High} group from the prior applications. In one method, the method yields low/moderate risk scores from the final model with a low/moderate Proliferative Index and a moderate/high Immunescore. In another method, the method combines histological grade (or other clinical markers of tumor low tumor aggressivity) with the model, e.g., histological grade I/II tumors with moderate risk scores in the final model and preferably high Immunescores.

The intensified treatment is more aggressive than the standard radiotherapy treatment, as provided herein. In some embodiments, the intensified treatment includes radiotherapy treatment (e.g., intensified radiotherapy). In some embodiments, the radiotherapy treatment is whole breast external radiotherapy, partial breast radiotherapy or brachytherapy or a combination thereof. In some embodiments, the radiotherapy treatment (e.g., intensified radiotherapy) includes using a biologically effective dose (BED) of about 73 Gy or more, e.g., about 78 Gy or more, about 83 Gy or more, about 87 Gy or more, about 93 Gy or more, about 97 Gy or more, about 100 Gy or more, or about 104 Gy or more, about 111 Gy or more, or about 133 Gy or more. In some embodiments, the radiotherapy treatment includes using a suitable biologically effective dose (BED) with a tumor alpha/beta ratio of 5 or less, 4 or less, 3 or less, or 2 or less. In some embodiments, the radiotherapy treatment includes using a BED of 73 Gy or more with a tumor alpha/beta ratio of 5. In some embodiments, the radiotherapy treatment includes using a BED of 78 Gy or more with a tumor alpha/beta ratio of 4. In some embodiments, the radiotherapy treatment includes using a BED of 87 Gy or more with a tumor alpha/beta ratio of 3. In some embodiments, the radiotherapy treatment includes using a BED of 104 Gy or more with a tumor alpha/beta ratio of 2. In some embodiments, the radiotherapy treatment uses a BED of 93 Gy or more with a tumor alpha/beta ratio of 5. In some embodiments, the radiotherapy treatment uses a BED of 100 Gy or more with a tumor alpha/beta ratio of 4. In some embodiments, the radiotherapy treatment uses a BED of 111 Gy or more with a tumor alpha/beta ratio of 3. In some embodiments, the radiotherapy treatment uses a BED of 133 Gy or more with a tumor alpha/beta ratio of 2 if the patient is recommended a boosting dose according to guidelines. In some embodiments, BED is defined as a measure of a true biological dose delivered by a combination of dose per fraction (d) and number of fractions (n) to a tissue characterized by a specific radiosensitivity (alpha/beta ratio): BED=n×d (1+d/(α/β)), where α (alpha) is the linear dose damage response and β (beta) is the quadratic dose response in tissue. Without being bound by theory, the alpha/beta ratio generally indicates how resistant a cell or tissue is to radiation damage.

In some embodiments, the intensified treatment plan includes treating the subject with intensified radiotherapy comprising a dose of at least one of: 67 Gy or more, add a boosting dose to a standard recommended treatment for the subject when the standard recommended treatment does not include a boosting dose, increase a boosting dose beyond the standard amount for the subject, increase the fraction dose on a per fraction basis above the standard for the subject, increase the number of fractions of a recommended dose above the standard for the subject. In some embodiments, the intensified treatment plan denotes at least one of: intensified radiotherapy treatment, systemic therapy, mastectomy, the additional use of a sensitizer to another therapy; a therapy above a level set by at least one of: the NCCN, ESMO, ESTRO, Clinical Practice Recommendations Australia, and/or NICE guidelines for the subject's remaining indicators, or any combination thereof as provided in Tables 14 and 15.

In any method of the present disclosure, in some embodiments, the method includes, for a subject with no boost otherwise recommended (e.g., recommended as the standard of care, or without the guidance provided by analysis of the biomarkers herein), the intensified radiotherapy treatment is whole breast external radiotherapy, partial breast radiotherapy or brachytherapy or a combination thereof, with a biologically effective dose of (BED) of 73 Gy or more with a tumor alpha/beta ratio of 5, or a BED of 78 Gy or more with a tumor alpha/beta ratio of 4, or a BED of 87 Gy or more with a tumor alpha/beta ratio of 3, or a BED of 104 Gy or more with a tumor alpha/beta ratio of 2. In some embodiments, for a subject with a boost otherwise recommended, the intensified radiotherapy treatment is one or more of whole breast external radiotherapy, partial breast radiotherapy or brachytherapy or a combination thereof, with a biologically effective dose of (BED) of 93 Gy or more with a tumor alpha/beta ratio of 5, or a BED of 100 Gy or more with a tumor alpha/beta ratio of 4, or a BED of 111 Gy or more with a tumor alpha/beta ratio of 3, or a BED of 133 Gy or more with a tumor alpha/beta ratio of 2 for patients who are recommended a boost according to the current guidelines.

In some embodiments, an intensified treatment, e.g., intensified radiotherapy, or more aggressive treatment is augmented in one or more relevant aspects of the treatment compared to the standard of care therapy. In some embodiments, the exposure of the subject to the therapeutic agent (e.g., radiation, antibody, cytotoxic agent, etc.) is increased in a meaningful way, e.g., to achieve better prognosis, compared to the standard of care therapy in the intensified or more aggressive treatment. In some embodiments, the length of exposure to a therapeutic agent (e.g., radiation, antibody, cytotoxic agent, etc.) is increased compared to the standard of care therapy in the intensified or more aggressive treatment. In some embodiments, the amount of exposure to a therapeutic agent (e.g., radiation, antibody, cytotoxic agent, etc.) is increased compared to the standard of care therapy in the intensified or more aggressive treatment. In some embodiments, the number and/or frequency of exposure to a therapeutic agent (e.g., radiation, antibody, cytotoxic agent, etc.) is increased compared to the standard of care therapy in the intensified or more aggressive treatment. In some embodiments, the intensified or more aggressive treatment is increased in at least one aspect (e.g., at least one of length, amount, number, frequency of exposure to the therapeutic agent) compared to the standard of care therapy by at least 1%, 2%, 3%, 4%, 5%, 10%, 15%, 20%, 25%, 30%, 35%, 40%, 50%, 60%, 70%, 80%, 90%, or more, or by any percentage within a range defined by any two of the preceding values.

Yet another group includes a low-risk favorable group that corresponds to grade I/II PD-1^{Low}/PD-L1^{Low}/TILs^{Low} group, which reflect low risk scores of the final model. In some embodiments, low risk scores in the final model are independent of other clinical characteristics. In some embodiments, low risk scores in the final model are combined with a low Proliferative Index score and a low Immunescore. In some embodiments, grade I/II tumors (and/or have other markers of a favorable prognosis such as ER/PgR positivity, older age (>60/70 years), low Ki67, HER2 negative etc.) and a low risk score from the final model with a low Immunescore and a low Proliferative Index score.

### Performance in the SweBCG91RT Cohort

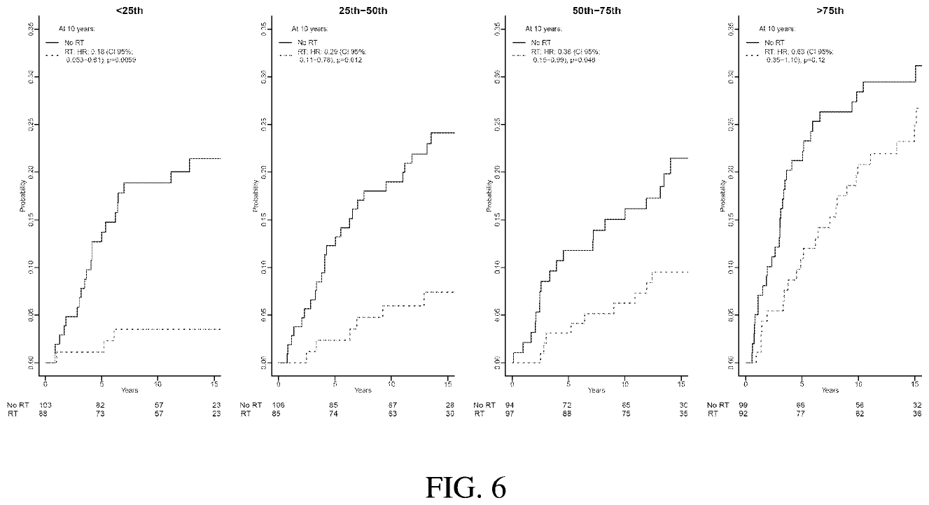

The final model can be used on all tumor types, as illustrated in FIG. 6, including all patients from the SweBCG91RT cohort. The patients are divided into groups based on quartiles (<25^{th} means below the 25^{th} percentile, etc.). High risk scores (preferably in relation to a background population) would lead to treatment intensification (with similar formulations as for the grade III PD-1^{Low}/PD-L1^{Low}/TILs^{Low} group, and the grade I/II PD-1^{High}/PD-L1^{High}/TILs^{High} group). A high risk score is a score compared to a background population of representative tumors and patients. The threshold for the high risk score may be anywhere above the 60th percentile (e.g., the 67th percentile, the 75th percentile, the 80th percentile, etc.). A low risk score would lead to treatment de-intensification (with similar formulations as for the grade III PD-1^{High}/PD-L1^{High}/TILs^{High} group, and the grade I/II PD-1^{Low}/PD-L1^{Low}/TILs^{Low} group). A low risk score is a score compared to a background population of representative tumors and patients. The threshold for the low risk score may be anywhere below 40th percentile (e.g., the 5th percentile, the 15th percentile, the 25th percentile, etc.). Medium risk scores would lead standard treatment or, under special circumstances, treatment according to descriptions in the paragraph above. A medium risk score is a score compared to a background population of representative tumors and patients. The threshold for the medium risk score may be an interval anywhere between the 30th and 70th percentile (e.g., the 30th-70th percentile, the 40th-60th percentile, the 45th-65th percentile, etc.). The high, medium, and low risk scores are also summarized in Table 13.

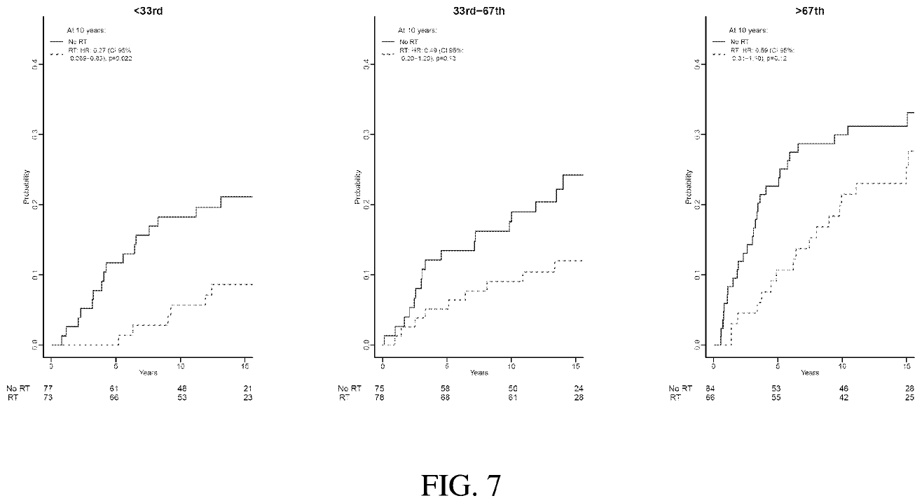

The model can also be used on high-risk tumor types as illustrated in FIG. 7, which includes patients from the SwcBCG91RT cohort with grade III tumors aged <70, and patients aged <60. Patients are divided into groups based on tertiles. High-risk patients can be defined as having grade III tumors, or being aged <60, or having an estrogen receptor-negative tumor, or having a high risk of recurrence according to other prognostic tests. For high-risk patients, the following can be said: High risk scores (preferably in relation to a background population) would lead to treatment intensification (with similar formulations as for the grade III PD-1^{Low}/PD-L1^{Low}/TILs^{Low} group); Low risk scores would lead to treatment de-intensification (with identical formulations as the grade III PD-1^{High}/PD-L1^{High}/TILs^{High} group). The thresholds for the high and low risk scores are similar to that of the high and low risk scores summarized in Table 13.

### Further Distinct Groups

Also provided herein is a method for treating breast cancer, the method including determining a tumor aggressivity, the tumor aggressivity including a histological grade of a tumor from at least a portion of a sample of the tumor provided by a subject, wherein the histological grade includes Grade I, Grade II, and Grade III; classifying the sample, when determined as a Grade I tumor, as low-risk; classifying the sample, when determined as a Grade III tumor, as high-risk; determining the tumor aggressivity of the sample, when determined as a Grade II tumor, the tumor aggressivity further including a Proliferative Index score of the sample. In some embodiments, the Proliferative Index score is based on expression of a first group of genes. In some embodiments, the first group of genes includes one or more genes listed in Table 6. In some embodiments, the method further includes classifying the Grade II tumor as: a) high-risk when the Proliferative Index score is greater or equal to a median score of a background population of Grade III tumors, or b) low-risk when the Proliferative Index score is less than the median score of the background population of Grade III tumors; determining an Immunescore of the sample, a level of tumor infiltrating lymphocytes (TILs) in the sample, a level of checkpoint molecules in the sample, or any combination thereof to determine an immunological activity. In some embodiments, the Immunescore is based on expression of a second group of genes. In some embodiments, the second group of genes includes one or more genes listed in Table 4. In some embodiments, the checkpoint molecules include programmed cell death protein-1 (PD-1) and programmed death-ligand 1 (PD-L1). In some embodiments, the immunological activity is determined to be: i) active when the TILs score is ≥10%, and the checkpoint molecules score is ≥1% (e.g., ≥1% of lymphocytes with positive staining for PD-1 or PD-L1), or ii) inactive when the TILs score is <10%, and/or the checkpoint molecules score is <1% (e.g., <1% of lymphocytes with positive staining for both PD-1 and PD-L1). In some embodiments, the method further includes integrating the tumor aggressivity and the immunological activity by training an elastic net having an interaction term between the tumor aggressivity and the immunological activity, determining a treatment plan based on an integration of tumor aggressivity and immunological activity. In some embodiments, the method further includes classifying the Grade II tumor as high-risk when the Proliferative Index score is greater or equal to a median score of a background population of Grade II tumors. In some embodiments, the method further includes classifying the Grade II tumor as high-risk when the Proliferative Index score is greater or equal to a cut-off above the 67th percentile of a background population of Grade II tumors. In some embodiments, the method further includes classifying the Grade II tumor as low-risk when the Proliferative Index is less than the median score of a background population of Grade II tumors. In some embodiments, the method further includes classifying the Grade II tumor as low-risk when the Proliferative Index is less than a cut-off below the 67th percentile of a background population of Grade II tumors (e.g., the 67th, the 50th, the 33rd percentiles, etc.).

In some embodiments, the active immunological activity indicates an activated immune infiltrate. In some embodiments, the inactive immunological activity indicates an inactive immune infiltrate. In some embodiments, the treatment plan includes standard radiotherapy, or radiotherapy omission, when the sample is classified as the high-risk Grade II tumor and the immunological activity is active. In some embodiments, the treatment plan includes radiotherapy intensification when sample is classified as the high-risk Grade II tumor and the immunological activity is inactive. In some embodiments, the treatment plan includes radiotherapy intensification when the sample is classified as the low-risk Grade II tumor and the immunological activity is active. In some embodiments, the treatment plan includes radiotherapy de-intensification, or radiotherapy omission, when the sample is classified as the low-risk Grade II tumor and the immunological activity is inactive.

Also provided herein is a method for treating breast cancer, the method including: determining tumor aggressivity, the tumor aggressivity including a histological grade of a tumor from at least a portion of a sample of the tumor provided by a subject, wherein the histological grade includes Grade I, Grade II, and Grade III; classifying the sample, when determined as a Grade I tumor, as low-risk; classifying the sample, when determined as a Grade III tumor, as high-risk; determining the tumor aggressivity of the sample, when determined as a Grade II tumor, the tumor aggressivity further including a Proliferative Index score of the sample; classifying the Grade II tumor as: a) high-risk when the Proliferative Index score is above a threshold between the 60th to 95th percentile compared to a background population of representative Grade II tumors, or b) low-risk when the Proliferative Index score is below a threshold between the 30^{th} to 60th percentile to the background population of representative Grade II tumors. In some embodiments, the Grade II tumor is classified as high-risk when the Proliferative Index score is above a threshold that is 60th percentile or higher compared to a background population of representative Grade II tumors. In some embodiments, the method further includes determining an Immunescore of the sample, a level of TILs in the sample, a level of checkpoint molecules in the sample, or any combination thereof to determine an immunological activity; integrating the tumor aggressivity and immunological activity by training an elastic net having an interaction term between the tumor aggressivity and the immunological activity, determining a treatment plan based on the tumor aggressivity, the immunological activity, and the interaction term. In some embodiments, the Proliferative Index score is based on expression of a first group of genes. In some embodiments, the Immunescore is based on expression of a second group of genes. In some embodiments, the checkpoint molecules include PD-1 and PD-L1. In some embodiments, the immunological activity is determined to be: i) active when the TILs score is ≥10%, and the checkpoint molecules score is ≥1% of lymphocytes with positive staining for PD-1 or PD-L1, or ii) inactive when the TILs score is less than 10%, and the checkpoint molecules score is less than 1% of lymphocytes with positive staining for both PD-1 and PD-L1. In some embodiments, the active immunological activity indicates an activated immune infiltrate. In some embodiments, the inactive immunological activity indicates an inactive immune infiltrate. In some embodiments, the first group of genes includes one or more genes listed in Table 6. In some embodiments, the second group of genes includes one or more genes listed in Table 4. In some embodiments, the treatment plan includes: c) standard radiotherapy, or radiotherapy omission, when the sample is classified as the high-risk Grade II tumor and the immunological activity is active; d) radiotherapy intensification when sample is classified as the high-risk Grade II tumor and the immunological activity is inactive; e) the radiotherapy intensification when the sample is classified as the low-risk Grade II tumor and the immunological activity is active; f) radiotherapy de-intensification, or the radiotherapy omission, when the sample is classified as the low-risk Grade II tumor and the immunological activity is inactive; g) the radiotherapy intensification when the sample is classified as a Grade III tumor and the immunological activity is inactive; h) radiotherapy de-intensification, or the radiotherapy omission, when the sample is classified as a Grade III tumor and the immunological activity is active; i) the radiotherapy intensification when the sample is classified as a Grade I tumor and the immunological activity is active; or j) radiotherapy de-intensification, or the radiotherapy omission, when the sample is classified as a Grade I tumor and the immunological activity is inactive.

Also provided herein is a method for treating breast cancer, the method including subtyping a tumor from at least a portion of a sample of the tumor provided by a subject, wherein the subtype includes Luminal A, Luminal B, HER2⁺, and triple negative/basal; determining a tumor aggressivity of the sample, when the sample is a Luminal A tumor or a Luminal B tumor, the tumor aggressivity including a Proliferative Index score of the tumor sample; classifying the sample, when the sample is subtyped as the Luminal A tumor, as: a) high-risk when the Proliferative Index score is above a threshold between 60th to 95th percentile compared to a background population of representative Luminal A tumors, or b) low-risk when the Proliferative Index score is below a threshold below the 60th percentile to the background population of representative Luminal A tumors; classifying the sample, when the sample is subtyped as the Luminal B tumor, as: c) high-risk, when the Proliferative Index score is above a threshold between 60th to 95th percentile compared to a background population of representative Luminal B tumors, or d) low-risk when the Proliferative Index score is below a threshold below the 60th percentile to the background population of representative Luminal B tumors; determining an immunological activity including scoring an Immunescore of the sample, scoring a level of TILs in the sample, scoring a level of checkpoint molecules in the sample, or any combination thereof; integrating the tumor aggressivity and the immunological activity by training an elastic net having an interaction term between the tumor aggressivity and the immunological activity, determining a treatment plan based on the integrated tumor aggressivity, immunological activity, and the interaction term. In some embodiments, the scoring of the Proliferative Index is based on expression of a first group of genes. In some embodiments, the scoring of the Immunescore is based on expression of a second group of genes. In some embodiments, the checkpoint molecules include PD-1 and PD-L1. In some embodiments, the immunological activity is determined to be: i) active when the TILs score is ≥10%, and either of the checkpoint molecules score is ≥1%, or ii) inactive when the TILs score is less than 10%, and/or the checkpoint molecules score is less than 1%. In some embodiments, the active immunological activity indicates an activated immune infiltrate. In some embodiments, the inactive immunological activity indicates an inactive immune infiltrate. In some embodiments, the first group of genes includes one or more genes listed in Table 6. In some embodiments, the second group of genes includes one or more genes listed in Table 4. In some embodiments, the treatment plan includes e) radiotherapy omission when the tumor sample is classified as the low-risk Luminal A tumor, f) standard radiotherapy, radiotherapy de-intensification, or the radiotherapy omission when the sample is classified as the high-risk Luminal B tumor and the immunological activity is active; or g) radiotherapy intensification when tumor sample is classified as the high-risk Luminal B tumor and the immunological activity is inactive.

In some embodiments, the sample, when subtyped as the Luminal A tumor, is classified as high-risk when the Proliferative Index score is above a threshold that is 60th percentile or higher compared to a background population of representative Luminal A tumors. In some embodiments, the sample, when subtyped as the Luminal B tumor, is classified as high-risk when the Proliferative Index score is above a threshold that is 60th percentile or higher compared to a background population of representative Luminal B tumors.

Also provided herein is a method for treating breast cancer, including: determining a tumor aggressivity of a tumor from at least a portion of a sample of the tumor provided by a subject, wherein the tumor aggressivity includes a Proliferative Index that classifies the tumor as: a) high-risk when a Proliferative Index score of the sample is above a threshold between a 60th to 95th percentile compared to a background population of representative tumors, or b) low-risk when the Proliferative Index score of the sample is below a threshold below the 60th percentile compared to the background population of representative tumors; determining an immunological activity of the tumor based on an Immunescore of the sample; integrating the tumor aggressivity and immunological activity based on an interaction term between the tumor aggressivity and the immunological activity; and determine a treatment plan based on the tumor aggressivity, immunological activity, and the interaction term. In some embodiments, the tumor is classified as high-risk, when the Proliferative Index score of the sample is above a threshold that is 60th percentile or higher compared to a background population of representative tumors.

Also provided herein is a method for treating breast cancer, including: determining a tumor aggressivity of a tumor from at least a portion of a sample of the tumor provided by a subject, wherein the tumor aggressivity includes a Proliferative Index score that classifies the tumor as: a) high-risk when the Proliferative Index score of the sample is greater or equal to a median score of a background population of representative tumors, or b) low-risk when the Proliferative Index score of the sample is less than the median score of the background population of representative tumors; determining an immunological activity of the tumor based on an Immunescore of the sample; and integrating the tumor aggressivity and immunological activity based on an interaction term between the tumor aggressivity and the immunological activity; and determining a treatment plan based on the tumor aggressivity, immunological activity, and the interaction term.

In some embodiments, when the tumor is classified as high-risk and the immunological activity is active, the treatment plan includes standard radiotherapy, radiotherapy de-intensification, or radiotherapy omission. In some embodiments, when the tumor is classified as high-risk and the immunological activity is inactive, the treatment plan includes radiotherapy intensification. In some embodiments, when the tumor is classified as low-risk and the immunological activity is active, the treatment plan includes radiotherapy intensification. In some embodiments, when the tumor is classified as low-risk and the immunological activity is inactive, the treatment plan includes radiotherapy de-intensification or radiotherapy omission.

In some embodiments, the integrating step includes training an elastic net having the interaction term between the tumor aggressivity and the immunological activity. In some embodiments, a high Proliferative Index score indicates an aggressive tumor. In some embodiments, when the sample is classified as the aggressive tumor and the immunological activity is inactive, the treatment includes radiotherapy intensification.

In some embodiments, the high Proliferative Index score includes a Proliferative Index of the sample being at least in the 60th percentile compared to the background population of representative tumors. In some embodiments, a high Proliferative Index score indicates an aggressive tumor. In some embodiments, when the sample is classified as the aggressive tumor and the immunological activity is inactive, the treatment includes radiotherapy intensification. In some embodiments, the high Proliferative Index score includes a Proliferative Index of the sample being greater or equal to the median score of a background population of representative tumors.

In some embodiments, the scoring of the Proliferative Index is based on expression of a first group of genes. In some embodiments, the first group of genes includes one or more genes listed in Table 6. In some embodiments, the scoring of the Immunescore is based on the expression of a second group of genes. In some embodiments, the second group of genes includes one or more genes listed in Table 4.

In some embodiments, the determining of the immunological activity further includes scoring TILs in the sample. In some embodiments, the active immunological activity includes an activated tumor infiltrate. In some embodiments, the activated tumor infiltrate includes a TILs score of ≥10%. In some embodiments, the step of determining of the immunological activity further includes measuring expression of one or more checkpoint molecules. In some embodiments, the step of determining the immunological activity further includes measuring expression of one or more checkpoint molecules. In some embodiments, the checkpoint molecules include PD-1 and PD-L1.

In some embodiments, the active immunological activity includes an activated tumor infiltrate. In some embodiments, the activated tumor infiltrate includes a positive staining for the expression of one or more of the checkpoint molecules. In some embodiments, the step of determining of the immunological activity further includes scoring TILs in the sample. In some embodiments, the activated tumor infiltrate further includes a TILs score of ≥10%. In some embodiments, the activated tumor infiltrate further includes a TILs score of ≥10% and a checkpoint molecule score >1%. In some embodiments, the inactive immunological activity includes a TILs score of <10%.

In some embodiments, the integrating step is performed by training an clastic net having the interaction term between the tumor aggressivity and the immunological activity. In some embodiments, the tumor aggressivity further includes a histological grade of the sample of the tumor. In some embodiments, the histological grade of the sample of the tumor is determined as a Grade II tumor. In some embodiments, the method further includes determining a subtype of the tumor sample, wherein the subtype includes Luminal A, Luminal B, HER2⁺, and triple negative/basal prior to determining the tumor aggressivity. In some embodiments, the subtype is Luminal A. In some embodiments, the subtype is Luminal B.

Also provided herein is a method for treating breast cancer, the method including: determining a histological grade of a tumor from at least a sample of the tumor provided by a subject, wherein the histological grade for the tumor includes Grade I, Grade II, or Grade III; assigning a tumor aggressivity of the sample as: a) low-risk when the sample is determined as a Grade I tumor; b) high-risk when the sample is determined as a Grade III tumor, c) low-risk when the sample is determined as a Grade I tumor and a Proliferative Index score of the sample compared to a background population of tumors; or d) high-risk when the sample is determined as the Grade II tumor and a Proliferative Index score of the sample is compared to the background population of tumors; classifying an immunological activity as active or inactive, the classifying including determining: e) an Immunescore of the sample; f) a level of TILs in the sample; g) a level of checkpoint molecules in the sample; or h) any combination of e)-f); integrating the tumor aggressivity and the immunological activity using interaction term to determine the benefit of a treatment plan. In some embodiments, the immunological activity is active when: f) is ≥10%, and g) is ≥1% of at least one of the checkpoint molecules. In some embodiments, the active immunological activity indicates an activated tumor infiltrate. In some embodiments, the activated tumor infiltrate includes a positive staining for the expression of one or more of the checkpoint molecules. In some embodiments, the immunological activity is inactive when: f) is <10%, and/or g) is <1% for both checkpoint molecules. In some embodiments, for c) the Proliferative Index score of the sample is below a threshold between a 20th to 60th percentile of a representative background population of tumors. In some embodiments, for c) the Proliferative Index score of the sample is below the 60th percentile of a representative background population of tumors. In some embodiments, for c) the Proliferative Index score of the sample is less than a median score of the background population of tumors. In some embodiments, for d) the Proliferative Index score of the sample is greater than or equal to the 60th to 95th percentile of the background population of tumors. In some embodiments, for d) the Proliferative Index score of the sample is 60th percentile or higher of the background population of tumors. In some embodiments, for d) the Proliferative Index score of the sample is greater than or equal to the median score of the background population of tumors. In some embodiments, for c) and/or d), the background population of tumors is a background population of Grade II tumors. In some embodiments, for c) and/or d) the background population of tumors is a background population of Grade III tumors. In some embodiments, for c) and/or d) the background population of tumors is a background population of Grade I tumors.

In some embodiments, the tumor is classified as high-risk and the immunological activity is active, the treatment plan includes standard radiotherapy, radiotherapy de-intensification, or radiotherapy omission. In some embodiments, when the tumor is classified as high-risk and the immunological activity is inactive, the treatment plan includes radiotherapy intensification. In some embodiments, when the tumor is classified as low-risk and the immunological activity is active, the treatment plan includes radiotherapy intensification. In some embodiments, when the tumor is classified as low-risk and the immunological activity is inactive, the treatment plan includes radiotherapy de-intensification (including radiotherapy omission).

In some embodiments, the integrating step includes training an elastic net having the interaction term between the tumor aggressivity and the immunological activity. In some embodiments, a high Proliferative Index score indicates an aggressive tumor. In some embodiments, when the sample is classified as the aggressive tumor and the immunological activity is inactive, the treatment includes radiotherapy intensification. In some embodiments, the score of the Proliferative Index is based on expression of a first group of genes. In some embodiments, the first group of genes includes one or more genes listed in Table 6. In some embodiments, the Immunescore is based on expression of a second group of genes. In some embodiments, the second group of genes includes one or more genes listed in Table 4. In some embodiments, the checkpoint molecules include PD-1 and PD-L1.

Also provided herein is a method for treating breast cancer including: supplying a sample of a tumor; receiving a treatment based on an analysis of the sample, the analysis including: determining a tumor aggressivity of the tumor from the sample; determining an immunological activity of the tumor from sample; and integrating the tumor aggressivity and the immunological activity based on an interaction term. In some embodiments, the tumor aggressivity includes a Proliferative Index score that classifies the tumor as high-risk or low-risk. In some embodiments, the immunological activity includes: a) an Immunescore of the sample; b) a level of TILs in the sample; c) a level of checkpoint molecules in the sample; or d) any combination of a)-c).

In some embodiments, the tumor is classified as: e) high-risk when the Proliferative Index score of the sample is above a threshold between 60th to 95th percentile compared to a background population of representative tumors, or f) low-risk when the Proliferative Index score of the sample is below a threshold below the 60th percentile compared to the background population of representative tumors. In some embodiments, the tumor is classified as: g) high-risk when the Proliferative Index score of the sample is greater or equal to a median score of a background population of representative tumors, or h) low-risk when the Proliferative Index score of the sample is less than the median score of the background population of representative tumors. In some embodiments, the tumor is classified as high-risk when the Proliferative Index score of the sample is above a threshold that is 60th percentile or higher compared to a background population of representative tumors. In some embodiments, when the tumor is classified as high-risk and the immunological activity is active, the treatment plan includes standard radiotherapy, radiotherapy de-intensification, or radiotherapy omission. In some embodiments, when the tumor is classified as high-risk and the immunological activity is inactive, the treatment plan includes radiotherapy intensification. In some embodiments, when the tumor is classified as low-risk and the immunological activity is active, the treatment plan includes radiotherapy intensification. In some embodiments, the tumor is classified as low-risk and the immunological activity is inactive, the treatment plan includes radiotherapy de-intensification or omission.

In some embodiments, a high Proliferative Index score indicates the tumor as aggressive. In some embodiments, when the tumor is indicated as aggressive, the treatment includes radiotherapy intensification. In some embodiments, the active immunological activity includes an activated tumor infiltrate. In some embodiments, the activated tumor infiltrate includes a TILs score of ≥10%. In some embodiments, the inactivated tumor infiltrate includes a TILs score of <10%. In some embodiments, the checkpoint molecules include PD-1 and PD-L1. In some embodiments, the checkpoint molecules include PD-1 and PD-L1. In some embodiments, the activated tumor infiltrate further includes a positive staining of at least 1% of lymphocytes for the expression of one or more of the checkpoint molecules.

In some embodiments, the tumor aggressivity further includes a histological grade of the sample of the tumor. In some embodiments, the histological grade includes Grade I, Grade II, and Grade III. In some embodiments, the tumor analysis further includes determining a subtype of the tumor sample, wherein the subtype includes Luminal A, Luminal B, HER2⁺, and triple negative/basal prior to determining the tumor aggressivity. In some embodiments, the subtype is Luminal A. In some embodiments, the subtype is Luminal B.

In some embodiments, the scoring of the Proliferative Index is based on expression of a first group of genes. In some embodiments, the first group of genes includes one or more genes listed in Table 6. In some embodiments, the Immunescore is based on expression of a second group of genes. In some embodiments, the second group of genes includes one or more genes listed in Table 4. In some embodiments, the integrating step includes training an elastic net, the elastic net including the interaction term between the tumor aggressivity and the immunological activity. In some embodiments, the elastic net further includes an age information of a subject that provided the sample. In some embodiments, the analysis further includes stratification by pre- and postmenopausal status. In some embodiments, the premenopausal status includes female subjects that are <55 years. In some embodiments, the postmenopausal status includes female subjects that are ≥55 years of age.

In some embodiments, a method of treating a subject is provided, the method including determining a Proliferative Index based on a level of one or more of the genes from Table 6; determining an Immunescore based on expression of one or more of the genes from Table 4; combining the Proliferative Index and Immunescore and optionally factoring in age of the subject, to determine if the subject will respond to a cancer therapy, and administering the cancer therapy if the Proliferative Index and Immunescore and optionally the age of the subject indicates that the therapy will be successful. In some embodiments, a) there is a high-risk when a Proliferative Index score of the sample is above a threshold between 60th to 95th percentile compared to a background population of representative tumors, or b) there is a low-risk when the Proliferative Index score of the sample is below a threshold below the 60th percentile compared to the background population of representative tumors. In some embodiments, c) one combines the Proliferative Index and Immunescore based on an interaction term between the two; and d) one determines the cancer therapy based on the Proliferative Index, Immunescore, and the interaction term.

In some embodiments, a method of treating a subject is provided, the method including factoring in a level of one or more genes from Table 6; factoring in a level of one or more genes in Table 4; and factoring in an age of the subject, thereby determining if the subject should receive standard radiation therapy, radiotherapy intensification, radiotherapy de-intensification or radiotherapy omission. More specifically, this information must be extracted from Tables 4 and 6. In some embodiments, a method of treating a subject is provided, the method including factoring in a level of one or more genes per gene set (“Gene Set” column) in Table 6 and multiplying with the respective coefficient (“Coefficient” column) in Table 6; and factoring in a level of one or more genes per gene set (“Gene Set” column) in Table 4 and multiplying with the respective coefficient (“Coefficient” column) in Table 4. In some embodiments, the level of one or more genes from Table 6 includes one or more levels of the one or more genes of Table 6, and the level of one or more genes of Table 4 includes one or more levels of the one or more genes of Table 4.

In some embodiments, the expression of one or more genes for the IS and/or PI is measured from the sample from the subject. In some embodiments the sample comprises a tissue sample. In some embodiments, the tissue sample comprises formalin fixed paraffin embedded tissue sample. In some embodiments, the tissue sample comprises or includes essentially of a core biopsy. In some embodiments, the sample does not comprise TILs. In some embodiments, the analysis does not measure TIL level. In some embodiments, the sample consists of a core biopsy. In some embodiments, the sample is collected prior to a treatment of the subject for the cancer. In some embodiments, the sample is collected prior to a therapeutic surgery of the subject for the cancer.

In some embodiments, age is used as a factor in determining the appropriate therapy or the treatment. In some embodiments, wherein a subject that is younger than 50 is considered young and a subject that is 50 or older is considered old. Age is preferably weighted as a continuous variable. It is desirous to use the continuous variable so that no information is lost from dichotomization.

Using age as a continuous variable. The Final model includes patient age and the Integrated score to produce a predicted risk. Each increase in age with one year reduces the risk score by 0.03471772. This can be contrasted against the weight of the Integrated score, where each increase with one standard deviation (compared to a representative background population) increases the risk score by 0.19541174. Both of these numbers are arbitrary and, therefore, do not represent absolute risks. However, it can be concluded that a decrease in age with 0.19541174/0.03471772=5.63 years has the same impact on the predicted risk as an increase in the Integrated score with one standard deviation. The absolute thresholds/cut-offs for defining high- and low-risk groups are obtained by comparing a score to a representative background population, as described herein.

Using age as an ordinal/categorical variable. It would also be possible to use the Integrated score/model or Proliferative Index and combine it with a variable representing age groups. In some embodiments, the age groups could, for example, be presumed pre-/perimenopausal (<55 years), 55-65 years, >=65 years. The Integrated model score or Proliferative Index could then be interpreted differently depending on the age category of the subject. For example, a low Integrated model score and/or Proliferative Index in a woman below 50 years of age may result in a recommendation to omit the RT boost (current guidelines recommend RT boost to young women), while a low Integrated model score and/or Proliferative Index in a woman above 65 years of age may result in a recommendation to omit RT altogether.

In some embodiments, the Integrated model score works in a superior manner for young women (<65 years, <60 years, or even <55 years), while the Proliferative Index works in a superior manner for older women (≥65 years). This is because the Integrated model considers the immune response, and, without being bound by theory, young women are thought to generally have better conditions for mounting an immune response.

In some embodiments, a method of determining a therapy for the treatment of cancer is provided, the method including determining an Immunescore of a core biopsy without determining a level of TILs surrounding a tumor from which the core biopsy was obtained, using the Immunescore to determine an amount of radiotherapy to administer to a subject, without factoring in the level of TILs. In some embodiments, the subject is treated with neoadjuvant immunotherapy. Neoadjuvant immunotherapy would be administered if the subject is believed to benefit from it rather than RT being inadequate. The clinical problem lies in identifying patients who benefit from immunotherapy. Candidates for neoadjuvant immunotherapy could be defined as any of the following, which would indicate that the subject benefits from this therapy: High Proliferative Index; High Proliferative Index+high Immunescore; Medium Proliferative Index+high Immunescore; Low/Medium Integrated model score with a high Proliferative Index and a high Immunescore; Low/Medium Integrated model score with a high Proliferative Index, a high Immunescore, and young age (<55, for example); Low/Medium Final model score with a high Proliferative Index and a high Immunescore; Low/Medium Final model score with a high Proliferative Index, a high Immunescore, and young age (<55, for example).

In some embodiments, one of skill in the art can examine the level of gene expression to determine what type of cancer therapy will be effective and appropriate for a subject. The genes examined can be any one or more in Tables 4 and/or Table 6. The gene level can be compared to a standard level of the gene in a healthy subject in some embodiments.

In some embodiments, the subject is 55 or less than 55. In some embodiments, the subject is more than 55 years of age. In some embodiments, the subject is 55 years or older. In some embodiments, the subject is 65 years or older. In some embodiments, the subject is older than 65 years. In some embodiments, the subject is 55 years or younger. In some embodiments, the subject is less than 55 years in age. In some embodiments, the subject is less than 50 years of age. In some embodiments, the subject is between 50 and 65 years of age. In some embodiments, the age cutoffs noted herein are used as a further variable to define which of the treatment paths one takes (such as in any of the embodiments provided herein, including in any of the flow charts depicted in the figures). In some embodiments, the cutoff is 55 and/or younger and 65 and/or older.

In some embodiments, the Immunescore (IS) can be used when a surgical excision specimen is not available and/or the sample for analysis is from a core biopsy. In some embodiments, this can be, for example, when the stromal TILs around the tumor are not available for analysis. In some embodiments, the tumor can be sampled prior to neo-adjuvant or adjuvant therapy for an accurate prognosis and prediction.

In some embodiments, a method for treating breast cancer is provided, the method including determining a histological grade of a tumor from at least a sample of the tumor provided by a subject, wherein the histological grade for the tumor includes Grade I. Grade II, or Grade III and assigning a tumor aggressivity of the sample. In some embodiments, the tumor aggressivity is assigned as low-risk when the sample is determined as a Grade I tumor. In some embodiments, the tumor aggressivity is assigned as low-risk when the sample is determined as a Grade II tumor and a Proliferative Index score of the sample is less than a median score of a background population of tumors. In some embodiments, the tumor aggressivity is assigned as high-risk when the sample is determined as the Grade II tumor and a Proliferative Index score of the sample is greater or equal to a median score of the background population of tumors. In some embodiments, the tumor aggressivity is assigned as high-risk when the sample is determined as a Grade III tumor. In some embodiments, the tumor aggressivity is assigned as high-risk when the Proliferative Index score of the sample is greater or equal to a median score of the background population of tumors. In some embodiments, the tumor aggressivity is assigned as high-risk when the sample is determined as ER-negative. In some embodiments, the tumor aggressivity is assigned as high-risk when the sample is determined as HER2-positive. In some embodiments, the tumor aggressivity is assigned as high-risk when the level of Ki67 of the sample is high. In some embodiments, when the level of Ki67 is determined from TMAs, a level of Ki67≥10% is considered high. More preferably, in some embodiments, when the level of Ki67 is determined from TMAs, a level of Ki67≥20% is considered high. Most preferably, in some embodiments, when the level of Ki67 is determined from TMAs, a level of Ki67≥30% is considered high. In some embodiments, when the level of Ki67 is determined from whole sections, a level of Ki67≥20% is considered high. More preferably, in some embodiments, when the level of Ki67 is determined from whole sections, a level of Ki67≥25% is considered high. Most preferably, in some embodiments, when the level of Ki67 is determined from whole sections, a level of Ki67≥30% is considered high. In some embodiments, when the level of Ki67 is determined from whole sections, a level of Ki67≤10% is considered low. More preferably, in some embodiments, when the level of Ki67 is determined from whole sections, a level of Ki67≤5% is considered low. When scored on whole sections, Ki67 is preferably scored as a global assessment, i.e., the proportion of tumor cells with positive staining, preferably using automated scoring. In some embodiments, hot spots are used and the values of the different hot spots are averaged. In some embodiments, hot spots include a proportion of tumor cells with positive Ki67 scores in areas with the strongest staining.

In some embodiments, the tumor aggressivity is assigned as high-risk when the mutational burden is high. In some embodiments, ≥5 mutations per genomic megabase (mut/MB) is considered a high mutational burden, more preferably ≥7 mut/MB, most preferably ≥10 mut/MB. The mutational burden thresholds may differ when the mutational burden assay is used for cancers in other organs. In some embodiments, the tumor aggressivity is assigned as high-risk when the sample is determined as a Grade III tumor, the Proliferative Index score of the sample is greater or equal to a median score of the background population of tumors; ER-negative; HER2-positive; the level of Ki67 is high; mutational burden is high; or any combination thereof. Preferably, the treatment of the high-risk group is determined based on immunological factors. High-risk+activated immune infiltrate (as determined by, for example, high TILs and/or high Immunescore and/or high PD-1 and/or high PD-L1, etc.) will lead to de-intensified radiotherapy. This could be radiotherapy boost omission (if a boost is otherwise indicated and immune activation above a moderate threshold is seen) or complete radiotherapy omission (suitable if immunological values are above a high threshold; e.g., any of the following or a combination (preferable) of the following: TILs>=50% and high PD-1/PD-L1, TILs>75%, Immunescore above a high threshold (e.g., the 70th percentile of a representative background population). In addition, certain forms of systemic therapy may be more effective among these tumors (immunotherapy, antiHER2-therapy, chemotherapy, etc.) which may further reduce the need for radiotherapy if provided. High-risk+not activated immune infiltrate may lead to radiotherapy boost and/or intensified systemic therapy.

In some embodiments, the method further includes classifying an immunological activity based on a level of TILs in the sample. In some embodiments, the level of TILs in the sample is in a range of 50% to 90%. In some embodiments, the level of TILs in the sample is in a range of 10% to 49%. In some embodiments, the tumor aggressivity is assigned as high-risk. In some embodiments, integrating high-risk tumor aggressivity and the immunological activity using an interaction term determines a treatment plan. In some embodiments, the treatment plan includes RT omission when the level of TILs in the sample is in a range of 50% to 90%. In some embodiments, the treatment plan comprises RT boost omission when the level of TILs in the sample is in a range of 10% to 49%. Additional factors may be taken into account, such as whether other forms of targeted therapies are available (e.g., anti-HER2 treatment), additional clinical variables (e.g., tumor size, lymph node status, patient age etc.), and patient preferences. In addition, the accuracy is improved by integrating different measures. As such, TILs level between 10-49% could indicate RT boost omission while TILs 10-49%+PD-1/PD-L1>=1% could justify RT omission. Additional measures (PD-1/PD-L1/Immunescore) can up- or downgrade treatment for borderline TILs values (e.g., TILs 10-49%). In one non-limiting example, tumors with TILs>=10% with PD-1/PD-L1 >=1% had a better prognosis than tumors with TILs>=50% without considering PD-1 or PD-L1; furthermore, tumors with TILs>=50% had a better prognosis than tumors with TILs 10-49%. These results indicate that a dose-response relationship exists between TILs levels and the risk of a local recurrence and that PD-1/PD-L1 provides information beyond TILs. The latter is particularly useful in borderline cases. In terms of RT treatment among high-risk tumors, increased levels of TILs will be used to omit RT boost or RT altogether. The optimal boost omission threshold is a level of TILs between 10-49% while the RT omission threshold is somewhere above a level of TILs of 50%. In some embodiments, RT boost omission: level of TILs anywhere above 10%; RT omission: level of TILs anywhere above 50%.

In some embodiments, the classifying of the immunological activity further includes a level of checkpoint molecules in the sample. In some embodiments, the checkpoint molecules include PD-1 and PD-L1. In some embodiments, the level of PD-1 is ≥1%. In some embodiments, the level of PD-L1 is ≥i %. In some embodiments, the level of TILs is ≥10%. In some embodiments, when the levels of PD-1 is ≥1%, PD-L1 is ≥1%, and TILs is ≥10%, then the treatment plan includes RT omission. In some embodiments, the level of PD-1 is <1%. In some embodiments, the level of PD-L1 is <1%, PDL-1 is <1%, and TILs is ≥10%, then the treatment plane includes RT boost omission. With respect to PD-1, the levels represent the proportion of lymphocytes with positive staining for PD-1. With respect to PD-L1, the levels represent the proportion of lymphocytes with positive staining for PD-L1. Staining assays to obtain these levels can be performed on TMAs/cores or whole sections (the latter preferable) by a pathologist or using automated image analysis.

In some embodiments, the sample includes a core biopsy. In some embodiments, the level of TILs is obtained via an automated scoring of whole tissue sections of the sample under high resolution microscopy.

In some embodiments, the method is combined with clinical variables. Principles for integrating additional clinical variables include: (1) The presence of high-risk clinical variables can optionally upgrade a recommendation of treatment de-escalation to standard treatment or upgrade a recommendation of treatment omission (e.g., RT omission or chemotherapy omission) to treatment de-escalation (e.g., RT de-escalation (e.g., reduced dose or number of fractions) or chemotherapy de-escalation. As used herein, chemotherapy de-escalation includes any of the following: a) Switch from dose-dense (decreased interval between each treatment and 8 or more treatments, e.g., an interval of 1-2 weeks instead of 3 for >=8 treatments, a common regimen is Epirubicin+Cyclophosphamide every other week for 4 treatments followed by weekly Paclitaxel for 12 weeks) to standard duration (6-8 treatments with an interval of 3 weeks between each treatment); b) Switch from longer than standard duration (e.g., ≥8 treatments) to standard duration (6-8 treatments with an interval of 3 weeks); c) Switch from standard duration (6-8 treatments with an interval of 3 weeks) to shorter duration (4-6 treatments with an interval of 3 weeks). As used herein, chemotherapy escalation includes any switch in the opposite direction as described in the definition of “chemotherapy de-escalation”. As used herein, “chemotherapy omission” means a switch from any treatment with chemotherapy to complete omission of chemotherapy. As used herein, RT de-escalation includes any of the following: a) Switch from whole-breast radiotherapy (WBRT) to intraoperative radiotherapy (IORT) or accelerated partial breast irradiation (APBI); b) A reduction of the dose or number of fractions; c) Switch from standard RT+boost to standard RT without boost; and d) A meaningful decrease in the Biologically Effective Dose (BED) compared to recommended guidelines. As used herein, RT escalation includes any switch in the opposite direction as described in the definition of “RT de-escalation”. As used herein, RT omission includes a switch from any treatment with RT to complete omission of RT. Of note, measures of tumor aggressiveness related to proliferation (histological grade. Ki67, ER-status) are not included in this notion as the method surprisingly shows such variables are predictive of a favorable prognosis if associated with an activated immune infiltrate; and (2) Absence of high-risk clinical variables will not downgrade the treatment recommendation for the high-risk group without an activated immune infiltrate. The presence of high-risk clinical variables in the low tumor-intrinsic risk group without an activated immune infiltrate can upgrade de-escalated therapy to standard therapy. Non-limiting examples of such variables are: tumor size >=5 cm, no endocrine therapy, extensive lymphovascular invasion. Non-limiting examples of various scenarios are shown in Table 29.

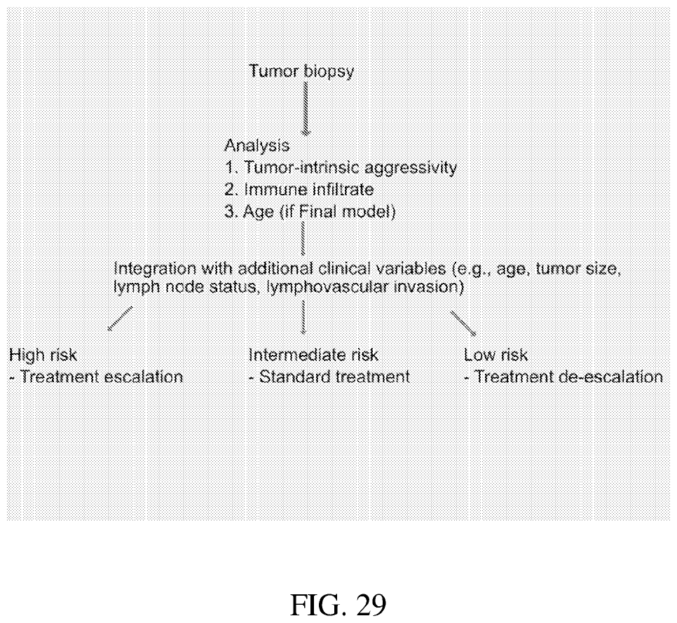

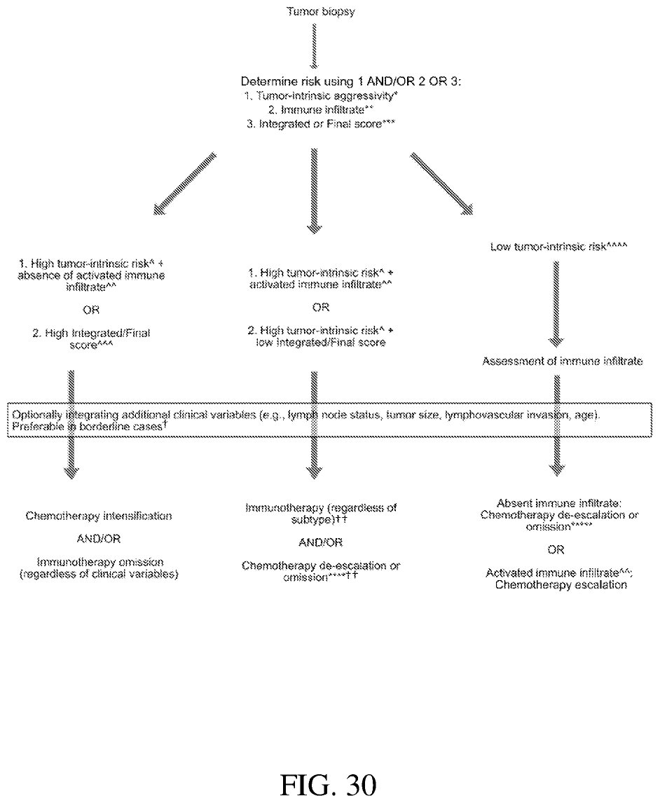

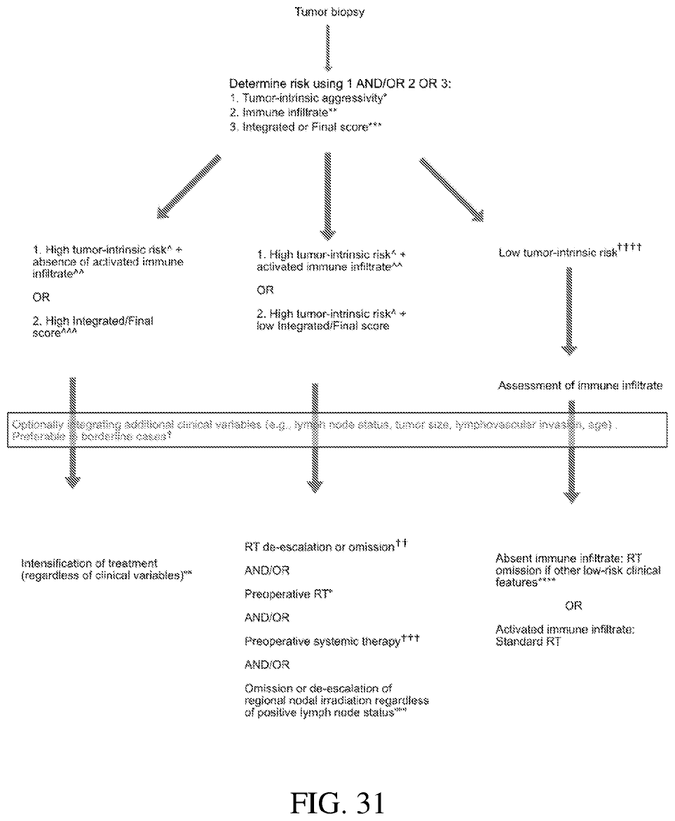

In some embodiments, a general flow chart shown in FIG. 29 illustrates a method with clinical variables. A tumor biopsy is performed and/or obtained, different analysis of the biopsy is conducted to determine tumor-intrinsic aggressivity, immune infiltrate, and age (if Final Model). Integration of the analysis with additional clinical variables (e.g., age, tumor size, lymph node status, lymphovascular invasion) classifies the tumor biopsies as high-, intermediate-, or low-risk as shown, with each leading to treatment escalation, standard treatment, and treatment de-escalation respectively. With respect to age, in some embodiments, young age becomes a less important risk-increasing factor among young subjects with high-risk tumors (determined by tumor-intrinsic aggressivity, point 1) and a rich immune infiltrate (immune infiltrate analysis, point 2). Here, the risk increase from a young age (e.g., <50 years) may be partially or completely offset by a better immune response among such patients. Additionally, the immune response (point 2) becomes less important among older subjects (e.g., >=65 years) with high-risk tumors (point 1) due to a waning immune response with older age. For older subjects, it may be preferred to only use point 1 (e.g., Proliferative Index) with age to determine the appropriate therapy (systemic therapy and radiotherapy shown in FIGS. 30 and 31 respectively). In some embodiments, older subjects, only tumor-intrinsic aggressivity and age are included in a model. In some embodiments, the method can be the method depicted in FIG. 29, in whole or in part, and include going through one of the decision lines, two of the decision lines, or all of the decision lines.

For systemic therapy as shown in FIG. 30, after obtaining or performing a tumor biopsy, one or more assessments are performed to determine a risk level of the tumor. In some embodiments, the assessments include tumor-intrinsic aggressivity, immune infiltrate, integrated or final score, or a combination thereof. After determining the risk level of the tumor, various therapies as illustrated in FIG. 30 may be performed based on the determined risk level of the tumor. In some embodiments, the method can be the method depicted in FIG. 30, in whole or in part, and include going through one of the decision lines, two of the decision lines, or all of the decision lines.

Additional aspects of the systemic therapy illustrated in FIG. 30 are provided. The tumor aggressivity assessment (“*”) includes Proliferative Index, histological grade, Ki67, HER2-status, ER-status. Assessing immune infiltrate (“**”) includes Immunescore, TILs, PD-1, PD-L1. Assessing the integrated or final score (“***”) includes an integrated model combining Proliferative Index and Immunescore, whereas the final model combines Integrated model and age. With respect to the remaining indicators shown in FIG. 30, “****”—Omission possible if no high-risk features: Extensive lymphovascular invasion, lymph node positivity, large tumor (e.g., >=50 mm). Otherwise, chemotherapy de-escalation. “*****”—Omission possible if low-risk features present: lymph node-negativity, older age (e.g., >=65), small tumor (e.g., <20 mm)), endocrine therapy. “\hat{ }”—Any or a combination of the following: 1. Proliferative Index above a threshold above the 60th percentile compared to a background population of representative tumors and patients. 2. Histological grade 3 or histological grade 2 and a Proliferative Index equal to or above the median of a background population of grade 3 tumors. 3. HER2-amplification. 4. High Ki67 (e.g., above a threshold higher than 20% scored as a global assessment). 5. ER-negative tumor. “\hat{ }\hat{ }”—1. Immunescore above a threshold above the 60th percentile compared to a background population of representative tumors and patients. 2. TILs above a threshold above 10%. 3. PD-1 or PD-L1 above 1%. “\hat{ }\hat{ }\hat{ }”—Integrated or Final score above a threshold above the 60th percentile of a representative background population of patients and tumors. “\hat{ }\hat{ }\hat{ }\hat{ }”—Any or a combination of the following: 1. Proliferative Index below a threshold below the 30th percentile compared to a background population of representative tumors and patients. 2. Histological grade 1 or histological grade 2 and a Proliferative Index below the median of a background population of grade 3 tumors. 3. HER2-negativity. 4. Low Ki67 (e.g., above a threshold lower than 20% scored as a global assessment). 5. ER-positive tumor. 6 Integrated or Final score below a threshold below the 40th percentile of a representative background population of patients and tumors and an absent immune infiltrate. “†”—Example of borderline case: intermediate Integrated/Final score/Proliferative Index/global Ki67 scores (in an interval somewhere between the 30th to 70th percentile of a representative background population of tumors and patients) and/or histological grade 2. If a low intermediate score (e.g., in the 40th percentile) and no high-risk features present (positive lymph nodes, tumor size >=20 mm, age <50 years). de-escalation or standard of care. If a high intermediate score (e.g., in the 60th percentile) and high-risk features, standard of care or treatment escalation. Otherwise, standard of care. “††”—Can be given preoperatively together with anti-HER2 and/or endocrine therapy. If pathological complete response is obtained, it can further guide RT de-escalation. Alternatively, can be given preoperatively together with neoadjuvant RT where pathological complete response indicates that postoperative systemic therapy (e.g., chemotherapy) can be de-escalated or omitted.

For radiotherapy shown in FIG. 31, after performing and/or obtaining a tumor biopsy from a patient, one or more assessments are performed to determine a risk level of the tumor. In some embodiments, the assessments include tumor-intrinsic aggressivity, immune infiltrate, integrated or final score, or a combination thereof. After determining the risk level of the tumor, various radiotherapies, or adjustments thereof, as illustrated in FIG. 31 may be performed based on the determined risk level of the tumor. In some embodiments, the method can be the method depicted in FIG. 31, in whole or in part, and include going through one of the decision lines, two of the decision lines, or all of the decision lines.

Additional aspects of the radiotherapy illustrated in FIG. 31 are provided. The tumor aggressivity assessment (“*”) includes Proliferative Index, histological grade, Ki67, ER-status and HER2-status. The tumor aggressivity assessment preferably combines several of the listed assessments. In some embodiments, at least Proliferative Index and histological grade are assessed. Assessing immune infiltrate (“**”) includes Immunescore, TILs, PD-1, PD-L1, or a combination thereof. Assessing the integrated or final score (“***”) includes an integrated model combining Proliferative Index and Immunescore, whereas the final model combines Integrated model and age. With respect to the remaining indicators shown in FIG. 31, “****”—Low-risk features: lymph node-negativity, older age (e.g., >=65), small tumor (e.g., <20 mm)), endocrine therapy. “\hat{ }”—Any of or a combination of the following: 1. Proliferative Index above a threshold above the 60th percentile compared to a background population of representative tumors and patients. 2. Histological grade 3 or histological grade 2 and a Proliferative Index equal to or above the median of a background population of grade 3 tumors. 3. HER2-amplified tumor. 4. High Ki67 (e.g., above a threshold higher than 20% scored as a global assessment). 5. ER-negative tumor. “\hat{ }\hat{ }”—Activated if >=1 of the following (preferably a combination of >=2) (otherwise, absent): 1. Immunescore above a threshold above the 60th percentile compared to a background population of representative tumors and patients. 2. TILs above a threshold above 10%. 3. PD-1 and/or PD-L1 above 1%. “\hat{ }\hat{ }\hat{ }”—Integrated or Final score below a threshold below the 40th percentile of a representative background population of patients and tumors. “†”—Intermediate Integrated/Final score; scores in an interval somewhere between the 30th to 70th percentile of a representative background population of tumors and patients. If a low intermediate score (e.g., in the 40th percentile) and no high-risk features present (positive lymph nodes, tumor size >=20 mm, age <50 years), de-escalation or standard of care; if the tumor size is between 20-50 mm, then the indication to provide standard of care instead of de-escalation increases with increasing tumor size. If a high intermediate score (e.g., in the 60th percentile) and high-risk features, standard of care or treatment escalation. Otherwise, standard of care. “††”—RT de-escalation (e.g., omitting RT boost or reducing the total RT dose or switching from whole-breast radiotherapy to intraoperative radiotherapy or accelerated partial breast irradiation) or omission of RT depends on the degree of activation of the immune infiltrate and the presence of high-risk clinical features. For example, TILs>=50% (preferably combined with PD-1/PD-L1>=1% and/or Immunescore >=a threshold above the 75th percentile of a representative background population of tumors and patients) without high-risk clinical features justifies RT omission, while 10%=<TILs <50% or high-risk clinical features justifies RT de-escalation. May be combined with preoperative systemic therapy (anti-HER2 and/or immunotherapy and/or chemotherapy and/or endocrine therapy) to further guide RT de-escalation. A pathological complete response to preoperative systemic therapy strengthens the indication to omit RT. “†††”—Preoperative systemic therapy can be given as a substitute for postoperative RT. If pathological complete response is obtained, the indication for RT omission is further strengthened. “††††”—All of the following: Low Proliferative Index score (e.g. below a threshold below the 40th percentile of a representative background population of tumors and patients), low histological grade (e.g., grade 1 or grade 2 with low/intermediate Proliferative Index scores (e.g., below the median of a representative background population of grade 3 or grade 2 tumors)), low Ki67 score (e.g., a global score below a threshold below 10%), ER-positive tumor, HER2-negative tumor, low Final or Integrated score (e.g., below a threshold below the 40th percentile of a representative background population of tumors and patients), treated with endocrine therapy. “″”—Preoperative RT can be used as an alternative to de-escalation or omission of postoperative RT. Preferably combined with preoperative systemic therapy (chemotherapy and/or anti-HER2 therapy and/or immunotherapy and/or endocrine therapy). If pathological complete response is obtained, postoperative systemic therapy can be de-escalated. “″″”—If standard RT according to guidelines, add RT boost. If RT boost is already indicated according to guidelines, escalate systemic therapy (primarily chemotherapy) and/or use wider surgical resection margins. “″″″”—If 1-3 positive lymph nodes, omit regional nodal irradiation. If >=4 positive lymph nodes, omit or reduce intensity of regional nodal irradiation.

In some embodiments, the method can be the method depicted in any one or a combination of the methods shown in FIGS. 29, 30, and/or 31. In some embodiments, the method can include going through one of the decision lines, two of the decision lines, three of the decision lines, four of the decision lines, five of the decision lines, six of the decision lines, seven of the decision lines, eight of the decision lines, or nine of the decision lines of FIGS. 29, 30, and/or 31. In some embodiments, a sample is provided by or obtained from a patient. In some embodiments, the sample includes a tumor biopsy. In some embodiments, the tumor biopsy is assessed for which risk level it falls under. In some embodiments, the assessment is performed by a trained practitioner. In some embodiments, the assessment is performed by an electronic device. In some embodiments, the assessment is performed by a trained practitioner in part and by a machine in part. In some embodiments, the assessment includes determining tumor-intrinsic aggressivity. In some embodiments, the assessment further includes determining an immune infiltrate. In some embodiments, the assessment includes determining the risk level by an Integrated model to produce an Integrated Score. In some embodiments, the assessment further includes determining the risk level by a Final Model, which includes an Integrated Score and the age of the patient, to produce a Final Score. In some embodiments, the determining of the tumor-intrinsic aggressivity includes Proliferative Index, histological grade, Ki67, ER-status, HER2-status, or a combination thereof. In some embodiments, the determining of the immune infiltrate includes determining an Immunescore, a level of TILs, a level of PD-1, a level of PD-L1, or a combination thereof. In some embodiments, the Integrated model combines Proliferative Index and Immunescore. In some embodiments, the Final model combines Integrated model and age of the patient. In some embodiments, wherein the risk level of the biopsy is a high tumor-intrinsic risk if the Proliferative Index is above a threshold above the 60th percentile compared to a background population of representative tumors and patients, a histological grade 3 or histological grade 2 and a Proliferative Index equal to or above the median of a background population of grade 3 tumors, a high Ki67, a ER-negative tumor, a HER2-amplified tumor, or a combination thereof. In some embodiments, the high Ki67 is above a threshold higher than 20% scored as a global assessment.

In some embodiments, an activated immune infiltrate of the biopsy is present if the Immunescore is above a threshold above the 60th percentile compared to a background population of representative tumors and patients, the level of TILs is above a threshold above 10%, the level of PD-1 or PD-L1 is above 1%, or a combination thereof. In some embodiments, the Integrated/Final Score is high if the Integrated or Final score threshold is above the 60th percentile of a representative background population of patients and tumors.

In some embodiments, activated immune infiltrate of the biopsy is considered absent if the Immunescore is below a threshold below the 60th percentile compared to a background population of representative tumors and patients, the level of TILs is below a threshold below 10%, the level of PD-1 or PD-L1 is below 1%, or a combination thereof. Preferably, all of these conditions related to determining an absence of the activated immune infiltrate will be present in reaching the determination of the absence of an activated immune infiltrate. A dose-dependent relationship can be seen where the lower the threshold, the stronger the indication to classify the patient as having an absent immune response.

As shown in FIG. 30, in some embodiments, if the biopsy has a high tumor-intrinsic risk and lacks an activated immune infiltrate, or has a high integrated/final score, then chemotherapy intensification and/or immunotherapy omission is provided to the patient. In some embodiments, if the biopsy has a high tumor-intrinsic risk and an activated immune infiltrate, or has a high tumor-intrinsic risk and low integrated/final score, then immunotherapy is administered and/or chemotherapy is de-escalated/omitted. In some embodiments, the method further includes assessment of an immune infiltrate; if the biopsy has a low tumor-intrinsic risk and lacks an activated immune infiltrate, or has a high integrated/final score. In some embodiments, chemotherapy de-escalation/omission is provided to the patient if the immune infiltrate is absent. In some embodiments, chemotherapy is administered to the patient if the immune infiltrate is activated.

As shown in FIG. 31, in some embodiments, if the biopsy has a high tumor-intrinsic risk and lacks an activated immune infiltrate, or has a high integrated/final score, then radiotherapy treatment is intensified for the patient. In some embodiments, if the biopsy has a high tumor-intrinsic risk and an activated immune infiltrate, or has a high tumor-intrinsic risk and low integrated/final score, then the radiotherapy is de-escalated or omitted, and/or preoperative radiotherapy will be provided to the patient, and/or preoperative systemic therapy will be provided to the patient, and/or omission or de-escalation of regional nodal irradiation regardless of previous lymph node status. In some embodiments, the method further includes assessment of an immune infiltrate. In some embodiments, if the biopsy has a low tumor-intrinsic risk and lacks an activated immune infiltrate, then radiotherapy will be omitted from the patient if other low-risk clinical features are present. In some embodiments, the low-risk clinical features include lymph node-negativity, older age (e.g., >=65), small tumor (e.g., <20 mm)), endocrine therapy. In some embodiments, standard radiotherapy is provided to the patient if the immune infiltrate is shown to be activated.

In some embodiments, a method of predicting the effectiveness of a cancer therapy is provided, a method for treating breast cancer is provided the method including determining a histological grade of a tumor from at least a sample of the tumor provided by a subject, wherein the histological grade for the tumor includes Grade I, Grade II, or Grade III; assigning a tumor aggressivity of the sample as: a) low-risk when the sample is determined as a Grade I tumor, b) low-risk when the sample is determined as a Grade II tumor and a Proliferative Index score of the sample is less than a median score of a background population of high-risk tumors; c) high-risk when the sample is determined as the Grade II tumor and a Proliferative Index score of the sample is greater or equal to a median score of the background population of high-risk tumors; or d) high-risk when the sample is determined as: i) a Grade III tumor, ii) the Proliferative Index score of the sample is greater or equal to a median score of the background population of high-risk tumors, iii) ER-negative, iv) HER2-positive, v) the level of Ki67 is high, vi) mutational burden is high, or vii) any combination of i)-vi); classifying an immunological activity includes a level of TILs in the sample; and integrating the tumor aggressivity and the immunological activity using an interaction term to determine a treatment plan.

In some embodiments, the level of Ki67 is ≥10%. In some embodiments, the level of Ki67 is ≥20%. In some embodiments, the level of Ki67 is ≥30%. In some embodiments, the mutational burden is ≥5 mutations per genomic megabase (mut/MB). In some embodiments, the mutational burden is ≥7 mut/MB. In some embodiments, the mutational burden is ≥10 mut/MB.

In some embodiments, a method for treating breast cancer is provided, the method including determining a histological grade of a tumor from at least a sample of the tumor provided by a subject, wherein the histological grade for the tumor includes Grade I, Grade II, or Grade III; assigning a tumor aggressivity of the sample; classifying an immunological activity includes a level of TILs in the sample; and integrating the tumor aggressivity and the immunological activity using an interaction term to determine a treatment plan.

In some embodiments, the assigning of the tumor aggressivity as high-risk includes determining the sample as: i) a Grade III tumor, ii) the Proliferative Index score of the sample is greater or equal to a median score of the background population of tumors, iii) ER-negative, iv) HER2-positive, v) the level of Ki67 is high, vi) mutational burden is high, or vii) any combination of i)-vi). In some embodiments, the level of Ki67 is ≥10%. In some embodiments, the level of Ki67 is ≥20%. In some embodiments, the level of Ki67 is ≥30%. In some embodiments, the mutational burden is ≥5 mut/MB. In some embodiments, the mutational burden is ≥7 mut/MB. In some embodiments, the mutational burden is ≥10 mut/MB.

In some embodiments, the assigning of the tumor aggressivity as high-risk comprises determining the sample as the Grade II tumor and a Proliferative Index score of the sample is greater or equal to a median score of the background population of tumors. In some embodiments, the tumor aggressivity is high-risk. In some embodiments, the level of TILs in the sample is in a range of 50% to 90%, and the treatment plan comprises radiotherapy omission. In some embodiments, the level of TILs in the sample is in a range of 10% to 49%, and the treatment plan comprises boost omission.

In some embodiments, the classifying of the immunological activity further includes a level of checkpoint molecules in the sample. In some embodiments, the checkpoint molecules include PD-1 and PD-L1. In some embodiments, the level of PD-1 is ≥1%. In some embodiments, the level of PD-L1 is ≥1%. In some embodiments, the level of TILs is ≥10%. In some embodiments, the treatment plan comprises RT omission. In some embodiments, the level of PD-1 is <1%. In some embodiments, the level of PD-L1 is <1%. In some embodiments, the level of TILs is ≥10%. In some embodiments, the treatment plan comprises RT boost omission.

In some embodiments, the subject is 55 years or older. In some embodiments, the subject is older than 55 years in age. In some embodiments, the subject is 65 years or older. In some embodiments, the subject is older than 65 years. In some embodiments, the subject is 55 years or younger. In some embodiments, the subject is less than 55 years in age. In some embodiments, the subject is between 50 and 65 years of age. In some embodiments, the subject is less than 50 years of age. In some embodiments, the background population is age-matched. In some embodiments, the sample comprises a core biopsy.

In some embodiments, a method for treating breast cancer is provided, the method including determining a tumor-intrinsic aggressivity of a sample provided by a subject, the tumor-intrinsic aggressivity comprising a Proliferative Index of the sample, a histological grade of the sample, a level of Ki67 from the sample, HER2 expression of the sample, ER-status of the sample, or a combination thereof; determining an immune infiltrate of the sample, the immune infiltrate comprising an Immunescore of the sample, a level of TILs of the sample, a level of PD-1 of the sample, a level of PD-L1 of the sample, or a combination thereof; and providing a treatment based on the tumor-intrinsic aggressivity and the immune infiltrate In some embodiments, the subject is 65 years of age or older.

In some embodiments, a tumor-intrinsic risk is high when: the Proliferative Index is above a 60th percentile compared to a background population of representative patients and tumors; the histological grade is 3; the histological is 2 and the Proliferative Index is equal to or above a median of a background population of grade 3 tumors; the HER2 expression is amplified; the ER-status of the sample is negative; or a combination thereof.

In some embodiments, the immune infiltrate is activated when: the Immunescore is above a threshold of 60th percentile compared to the background population of representative tumors; the level of TILs in the sample is greater than 10%; the level of PD-1 in the sample is greater than 1%: the level of PD-L1 in the sample is greater than 1%; or a combination thereof.

In some embodiments, the tumor-intrinsic risk is high and the immune activation inactive, and the treatment comprises RT boost. In some embodiments, the tumor-intrinsic risk is high, the immune activation inactive, and the subject does not have a co-morbidity, and the treatment comprises chemotherapy intensification. In some embodiments, the treatment further comprises immunotherapy. In some embodiments, the sample is ER-positive, and the treatment further comprises endocrine therapy. In some embodiments, the tumor-intrinsic risk is high, the immune activation inactive, and the subject does not have a co-morbidity, and the treatment comprises immunotherapy. In some embodiments, the tumor-intrinsic risk is high, the immune activation inactive, the subject does not have a co-morbidity, and the sample is ER-positive, and the treatment comprises endocrine therapy. In some embodiments, the tumor-intrinsic risk is not high, the immune infiltrate is not activated, the subject does not have a co-morbidity, and the treatment comprises RT omission. In some embodiments, the treatment further comprises chemotherapy omission or de-escalation. In some embodiments, the treatment further comprises endocrine therapy.

In some embodiments, the tumor-intrinsic risk is not high, the immune infiltrate is not activated, the subject does not have a co-morbidity, and the treatment comprises chemotherapy omission or de-escalation. In some embodiments, the tumor-intrinsic risk is not high, the immune infiltrate is not activated, the subject does not have a co-morbidity, and the treatment comprises endocrine therapy. In some embodiments, the tumor-intrinsic risk is not high, the immune infiltrate is activated, and the subject does not have a co-morbidity, and the treatment comprises RT. In some embodiments, the treatment further comprises chemotherapy. In some embodiments, the treatment further comprises endocrine therapy.

In some embodiments, the tumor-intrinsic risk is not high, the immune infiltrate is activated, and the subject does not have a co-morbidity, and the treatment comprises chemotherapy escalation. In some embodiments, the tumor-intrinsic risk is not high, the immune infiltrate is activated, and the subject does not have a co-morbidity, and the treatment comprises endocrine therapy.

In some embodiments, the subject has a co-morbidity. Some non-limiting examples of the co-morbidity include the co-morbidity includes coronary heart disease, heart failure, chronic obstructive pulmonary disease, previous stroke (ischemic or hemorrhagic), uncontrolled hypertension, diabetes mellitus, osteoporosis, one or more other cancers, or a combination thereof. In some embodiments, the treatment comprises chemotherapy omission and RT de-escalation.

In some embodiments, a method for treating breast cancer is provided, the method including building an Integrated model from a sample provided by a subject to generate an Integrated score, wherein the Integrated model includes a Proliferative Index and an immunescore; and building a Final model based on the Integrated model and an age of the subject to generate a Final score, providing a treatment based on an Integrated/Final score comprising the Integrated Score or the Final Score. In some embodiments, the subject is 65 years old or older.

In some embodiments, the Integrated/Final score is high when the Integrated score or the Final Score is above a 60th percentile of a representative background population of patients and tumors. In some embodiments, the background population is age-matched. In some embodiments, the background population is age-matched with the subject. In some embodiments, the method further comprising determining an immune infiltrate of the sample, the immune infiltrate comprising the Immunescore of the sample. In some embodiments, the immune infiltrate further comprises a level of TILs of the sample, a level of PD-1 of the sample, a level of PD-L1 of the sample, or a combination thereof. In some embodiments, the immune infiltrate is activated when: the Immunescore is above a threshold of 60th percentile compared to the background population of representative tumors; the level of TILs in the sample is greater than 10%; the level of PD-1 in the sample is greater than 1%; the level of PD-L1 in the sample is greater than 1%; or a combination thereof. In some embodiments, the immune infiltrate is activated when the Immunescore is above a threshold of 60th percentile compared to the background population of representative patients and tumors. In some embodiments, the background population is age-matched. In some embodiments, the background population is age-matched with the subject.

In some embodiments, the Integrated/Final score is high, and the treatment comprises RT boost. In some embodiments, the Integrated/Final score is not high and the immune infiltrate is active, and the treatment comprises chemotherapy omission or chemotherapy de-escalation. In some embodiments, the treatment further comprises immunotherapy. In some embodiments, the sample is ER-positive, and the treatment further comprises endocrine therapy. In some embodiments, the Integrated/Final score is high and the immune infiltrate is activated, and the treatment comprises immunotherapy. In some embodiments, the Integrated/Final score is not high and the immune infiltrate is not activated, and the treatment comprises RT omission. In some embodiments, the treatment further comprises chemotherapy omission or de-escalation. In some embodiments, the treatment further comprises endocrine therapy. In some embodiments, the Integrated/Final score is not high and the immune infiltrate is activated, and the treatment comprises standard RT. In some embodiments, the treatment further comprises chemotherapy escalation. In some embodiments, the Integrated/Final score is not high and the immune infiltrate is activated, and the treatment comprises chemotherapy escalation. In some embodiments, the treatment further comprises endocrine therapy.

In some embodiments, a method for treating breast cancer is provided, the method including determining a tumor-intrinsic aggressivity of a sample provided by a subject, the tumor-intrinsic aggressivity comprising a Proliferative Index of the sample, a histological grade of the sample, a level of Ki67 from the sample, HER2 expression of the sample, ER-status of the sample, or a combination thereof; determining an immune infiltrate of the sample, the immune infiltrate comprising an Immunescore of the sample, a level of TILs of the sample, a level of PD-1 of the sample, a level of PD-L1 of the sample, or a combination thereof; and providing a treatment based on the tumor-intrinsic aggressivity and the immune infiltrate, wherein the subject is less than 55 years of age.

In some embodiments, a method for treating breast cancer is provided, the method including building an Integrated model from a sample provided by a subject to generate an Integrated score, wherein the Integrated model includes a Proliferative Index and an Immunescore; and building a Final model based on the Integrated model and an age of the subject to generate a Final score, providing a treatment based on an Integrated/Final score comprising the Integrated score or the Final score, wherein the subject is less than 55 years old.

In some embodiments, the Integrated/Final score is less than a 5th percentile of a representative background population of patients and tumors. In some embodiments, the treatment comprises a RT de-escalation. In some embodiments, the Integrated/Final score is greater than a 50th percentile of a representative background population of patients and tumors. In some embodiments, the treatment comprises RT escalation compared to standard RT. In some embodiments, the RT escalation comprises RT boost. In some embodiments, the Integrated/Final score is greater than 95th percentile of a representative background population of patients and tumors. In some embodiments, the treatment comprises surgical escalation. In some embodiments, the surgical escalation comprises mastectomy. In some embodiments, the surgical escalation further comprises wider resection margins. In some embodiments, the treatment further comprises systemic therapy escalation. In some embodiments, the treatment comprises systemic therapy escalation. In some embodiments, the background population is age-matched. In some embodiments, the background population is age-matched with the subject.

In some embodiments, a method for treating breast cancer is provided, the method including building an Integrated model from a sample provided by a subject to generate an Integrated score, wherein the Integrated model includes a Proliferative Index and an Immunescore; and building a Final model based on the Integrated model and an age of the subject to generate a Final score, providing a treatment based on an Integrated/Final score comprising the Integrated score or the Final score, wherein the subject is greater than 55 years old. In some embodiments, the Integrated/Final score is above a 70th percentile of a representative background population of patients and tumors. In some embodiments, the treatment comprises RT escalation. In some embodiments, the RT escalation comprises RT boost. In some embodiments, the Integrated/Final score is above an 85th percentile of a representative background population of patients and tumors. In some embodiments, the treatment comprises mastectomy. In some embodiments, the treatment comprises escalating systemic therapy. In some embodiments, the background population is age-matched. In some embodiments, the background population is age-matched with the subject.

For patients with high-risk tumors where the tumor-intrinsic risk is high (e.g., high Proliferative Index, high grade tumor, young age, etc.) and there is a high Immunescore/low Integrated Score, prognosis is surprisingly good. Therefore, these patients need de-escalated therapy in comparison to current guidelines. Some non-limiting examples of de-escalated therapy include de-escalating chemotherapy (e.g., chemotherapy omission, no dose-dense chemotherapy, shorter duration of chemotherapy, and the like), omission of RT boost, immunotherapy only, and no prolonged endocrine treatment. In some embodiments, for patients with high-risk tumors where the tumor-intrinsic risk is high (e.g., high Proliferative Index, high grade tumor, young age, etc.) and there is a low Immunescore/high Integrated Score, prognosis is surprisingly poor. Therefore, these patients need escalated therapy. Some non-limiting examples of escalated therapy includes dose-dense chemotherapy, antibody drug conjugates, RT with boot, and prolonged endocrine treatment.

Chemotherapeutic agents used in cancer treatment can be divided into several groups, depending on their mechanism of action. Some chemotherapeutic agents directly damage DNA and RNA. By disrupting replication of the DNA such chemotherapeutics either completely halt replication, or result in the production of nonsense DNA or RNA. This category includes, for example, cisplatin (Platinol®), daunorubicin (Cerubidine®), doxorubicin (Adriamycin®), and etoposide (VePesid®). Another group of cancer chemotherapeutic agents interfere with the formation of nucleotides or deoxyribonucleotides, so that RNA synthesis and cell replication is blocked. Examples of drugs in this class include methotrexate (Abitrexate®), mercaptopurine (Purinethol®), fluorouracil (Adrucil®), and hydroxyurea (Hydrea®). A third class of chemotherapeutic agents effects the synthesis or breakdown of mitotic spindles, and, as a result, interrupt cell division. Examples of drugs in this class include Vinblastine (Velban®), Vincristine (Oncovin®) and taxenes, such as, Pacitaxel (Taxol®), and Tocetaxel (Taxotere®) Tocetaxel is currently approved in the United States to treat patients with locally advanced or metastatic breast cancer after failure of prior chemotherapy, and patients with locally advanced or metastatic non-small cell lung cancer after failure of prior platinum-based chemotherapy. The prediction of patient response to all of these, and other chemotherapeutic agents is specifically within the scope of the present invention.

In some embodiments, chemotherapy includes treatment with a taxane derivative. Taxanes include, without limitation, paclitaxel (Taxol®) and docetaxel (Taxotere®), which are widely used in the treatment of cancer. As discussed above, taxanes affect cell structures called microtubules, which play an important role in cell functions. In normal cell growth, microtubules are formed when a cell starts dividing. Once the cell stops dividing, the microtubules are broken down or destroyed. Taxanes stop the microtubules from breaking down, which blocks cell proliferation.

In some embodiments, chemotherapy includes treatment with an anthracycline derivative, such as, for example, doxorubicin, daunorubicin, and aclacinomycin.

In some embodiments, chemotherapy includes treatment with a topoisomerase inhibitor, such as, for example, camptothecin, topotecan, irinotecan, 20-S-camptothecin, 9-nitro-camptothecin, 9-amino-camptothecin, or GI147211.

Treatment with any combination of these and other chemotherapeutic drugs is specifically contemplated.

Most patients receive chemotherapy immediately following surgical removal of the tumor. This approach is commonly referred to as adjuvant therapy. However, chemotherapy can be administered also before surgery, as so called neoadjuvant treatment. Although the use of neo-adjuvant chemotherapy originates from the treatment of advanced and inoperable breast cancer, it has gained acceptance in the treatment of other types of cancers as well. The efficacy of neoadjuvant chemotherapy has been tested in several clinical trials. In the multi-center National Surgical Adjuvant Breast and Bowel Project B-18 (NSAB B-18) trial (Fisher et al., J. Clin. Oncology 15:2002-2004 (1997); Fisher et al., J. Clin. Oncology 16:2672-2685 (1998)) neoadjuvant therapy was performed with a combination of adriamycin and cyclophosphamide (“AC regimen”). In another clinical trial, neoadjuvant therapy was administered using a combination of 5-fluorouracil, epirubicin and cyclophosphamide (“FEC regimen”) (van Der Hage et al., J. Clin. Oncol. 19:4224-4237 (2001)). Newer clinical trials have also used taxane-containing neoadjuvant treatment regiments. See, e.g. Holmes et al., J. Natl. Cancer Inst. 83:1797-1805 (1991) and Moliterni et al., Seminars in Oncology, 24:S17-10-S-17-14 (1999). For further information about neoadjuvant chemotherapy for breast cancer see, Cleator et al., Endocrine-Related Cancer 9:183-195 (2002).

### Arrangements

Additional non-limiting embodiments of the present disclosure are provided in the following numbered arrangements.

1. A method for treating breast cancer, comprising:
determining an Immunescore (IS) based on an immunological model comprised of genes from immunological gene sets;
determining a Proliferative Index (PI) based on a tumor-intrinsic model comprised of genes from tumor-intrinsic gene sets; and
integrating the IS and the PI into an Integrated model;
integrating the Integrated model and an age of a subject into a Final model to identify if the subject falls into a specific-risk clinical group; and
providing an appropriate therapy to the subject based on the specific-risk clinical group,
wherein the immunological model is trained in basal and HER2⁺ tumors, and
wherein the tumor intrinsic model is trained in immune-depleted tumors.

2. The method of arrangement 1, wherein the immunological model comprises immunological gene sets.

3. The method of arrangement 2, further comprising performing meta-analysis of prognostic effect of each of the immunological gene sets.

4. The method of arrangement 2 or 3, wherein the immunological gene sets comprises C7 and any immune-related gene sets from H, C1, C2, C3, C4, C5, C6, or C8 categories (e.g., identified using any of the keywords “LYMPHOCYTE|T_CELL|PD1|PD-1|PDL1|PD-L1|LAG3|CHECKPOINT_RECEPTOR|B_CELL|PERFORIN|GRANZYME|NK_CELL|CD 8|CYTOTOXIC”).

5. The method of any one of arrangements 1-4, further comprising collinearity filtering to remove highly correlated gene sets.

6. The method of any one of arrangements 1-5, wherein the IS determining step comprises training an elastic net for tumors; and optionally wherein the PI determining step comprises training the elastic net for tumors comprising immune-depleted tumors.

7. The method of arrangement 6, wherein the tumors comprise HER2⁺ tumors and basal tumors.

8. The method of any one of arrangements 1-5, wherein the PI determining step comprises training an elastic net for tumors comprising immune-depleted tumors.

9. The method of arrangement 6, wherein the IS determining step is performed prior to the PI determining step.

10. The method of any one of arrangements 1-8, further comprising:
ranking, based on Rho, genes in the gene sets selected in the determining the IS step; and
selecting one or more of the ranked genes.

11. The method any one of arrangements 1-10, further comprising: ranking, based on Rho or a Prognostic Score, genes in the gene sets selected in the determining the PI step, wherein the Prognostic Score includes Rho/Pmeta; and excluding the ranked genes having a Rho from the Prognostic Score of <0.2.

12. The method of any one of arrangements 1-11, further comprising combining the Integrated model and an age of a subject into a final model to output a risk score; and recommending one or more treatment plans based on the risk score.

13. The method of any one of arrangements 1-12, wherein the tumor-intrinsic model comprises H, C2, and/or C6 gene sets.

14. The method of arrangement 12, wherein the final model comprises the steps of:
extraction of genes included in the IS and the PI;
performing, in a first cohort, a meta-analysis of a prognostic effect of each of the extracted genes;
performing, in a second cohort, a selection of stably expressed genes across cores and tissue types;
ranking of the extracted genes; and
selecting all of the extracted genes from the IS and the PI,
wherein a number of the extracted genes from the IS is less than 23, and
wherein a number of the extracted genes from the PI is less than 60.

15. The method of arrangement 12, wherein the final model comprises the steps of:
extraction of genes included in the IS and the PI;
performing, in a first cohort, a meta-analysis of a prognostic effect of each of the extracted genes;
performing, in a second cohort, a selection of stably expressed genes across cores and tissue types;
ranking of the extracted genes;
selecting a top 23 of the extracted genes from the IS; and
selecting a top 60 of the extracted genes from the PI,
wherein a number of the extracted genes from the IS is ≥23, and
wherein a number of the extracted genes from the PI is ≥than 60.

16. The method of arrangement 14 or 15, wherein the first cohort comprises a training cohort.

17. The method of any one of arrangements 14-16, wherein the second cohort comprises the SweBCG91-RT cohort, or another similar cohort,
wherein the SweBCG91-RT cohort passed RNA, cDNA, and microarray quality control,
wherein the SweBCG91-RT cohort is treated with:
   breast-conserving surgery and included in a multivariable analysis; or
   breast-conserving surgery and radiotherapy and included in a multivariable analysis.

18. The method of arrangement 12, wherein the risk score is a moderate risk score comprising a high PI indicative of aggressive tumors and a high IS indicative of strong immune activity.

19. The method of arrangement 12, wherein the moderate risk score corresponds to grade III tumors comprising PD-1^{High}/PD-L1^{High}/TILs^{High}.

20. The method of arrangement 18 or 19, wherein the treatment plain comprises immunotherapy.

21. The method of arrangement 19 or 20, wherein the IS and PT are high.

22. The method of arrangement 21, wherein the high IS is a value above a threshold in a range of 60th to 90th percentile, and
wherein the high PI is a value above a threshold in a range of 60th to 90th percentile.

23. The method of arrangement 19, the treatment plan comprising de-intensifying one or more treatments to the grade III tumors if the IS and the PI are high.

24. The method of arrangement 23, wherein the high IS is a value above a threshold in a range of 67th to 90th percentile, and
wherein the high PI is a value above a threshold in a range of 67th to 90th percentile.

25. The method of arrangement 12, wherein the PI is high at a value above a threshold in a range of 67th to 90th percentile.

26. The method of arrangement 25, further comprising scoring a level of TILs.

27. The method of arrangement 26, wherein the scored level of TILs is in a range of 50% to 90%, and
wherein the scored level of TILs indicates high-risk tumors that are candidates for radiotherapy omission.

28. The method of arrangement 26, wherein the scored level of TILs is in range of 10% to 49%, and
wherein the scored level of TILs indicates high-risk tumors that are candidates for boost omission.

29. The method of arrangement 12, wherein the risk score is a high-risk score comprising a moderate/low IS and a high PI.

30. The method of arrangement 29, wherein the high risk score corresponds to grade III tumors comprising PD-1^{Low}/PD-L1^{Low}/TILs^{Low}.

31. The method of arrangement 29, wherein the high risk score corresponds to grade I/II tumors comprising PD-1^{High}/PD-L1^{High}/TILs^{High}.

32. The method of any one of arrangements 29-31, wherein the moderate IS is a value below a threshold in a range of 10th to less than 40th percentile,
wherein the low IS is a value below a threshold in a range of 30th to less than 70th percentile, and
wherein the high PI is a value above a threshold in a range of 60th to 90th percentile.

33. The method of arrangement 30 or 31, the treatment plan is intensified.

34. The method of arrangement 33, wherein the intensified treatment plan comprises treating the subject with intensified radiotherapy comprising a dose of at least one of: 67 Gy or more, add a boosting dose to a standard recommended treatment for the subject when the standard recommended treatment does not include a boosting dose, increase a boosting dose beyond the standard amount for the subject, increase the fraction dose on a per fraction basis above the standard for the subject, increase the number of fractions of a recommended dose above the standard for the subject.

35. The method of arrangement 33, wherein the intensified treatment plan denotes at least one of: intensified radiotherapy treatment, systemic therapy, mastectomy, the additional use of a sensitizer to another therapy; a therapy above a level set by at least one of: the NCCN, ESMO, ESTRO, Clinical Practice Recommendations Australia, and/or NICE guidelines for the subject's remaining indicators, or any combination thereof.

36. A method for treating breast cancer, comprising:
determining an IS based on an immunological model comprised of genes from immunological gene sets;
determining a PT based on a tumor-intrinsic model comprised of genes from tumor-intrinsic gene sets;
integrating the IS and the PI into an Integrated model;
integrating the Integrated model and an age of a subject into a Final model to identify if the subject falls into a specific-risk clinical group based on a risk score from the Final model; and
intensifying a treatment plan to the subject when the risk score for the subject is a high risk score, the treatment plan comprising treating the subject with intensified radiotherapy comprising a dose of at least one of: 67 Gy or more, add a boosting dose to a standard recommended treatment for the subject when the standard recommended treatment does not include a boosting dose, increase a boosting dose beyond the standard amount for the subject, increase the fraction dose on a per fraction basis above the standard for the subject, increase the number of fractions of a recommended dose above the standard for the subject,
wherein the intensified treatment plan denotes at least one of: intensified radiotherapy treatment, systemic therapy, mastectomy, the additional use of a sensitizer to another therapy; a therapy above a level set by at least one of: the NCCN, ESMO, ESTRO, Clinical Practice Recommendations Australia, and/or NICE guidelines for the subject's remaining indicators, or any combination thereof,
wherein the immunological model is trained in basal and HER2+ tumors.
wherein the tumor intrinsic model is trained in immune-depleted tumors, and
wherein the high risk score groups the subject into a high-risk clinical group.

37. A method for treating breast cancer, comprising:
determining expression levels of genes that are included in a model;
adjusting the expression levels of the genes by scaling or normalizing to a background population of representative tumors;
summing the adjusted expression levels of the genes to determine enrichment scores of gene sets included in the model, wherein the gene sets include the genes;
standardizing the enrichment scores by comparing the enrichment scores to the background population;
using IS and PT models to calculate IS and PT respectively;
standardizing the IS and PI scores by comparing the IS and PI scores to the background population;
using an integrated model to calculate an integrated score;
standardizing the integrated score by comparing the score to the background population;
using a final model to determine a risk score of a subject, wherein the final model includes an age of the subject;
comparing the risk score of the subject to the background population to determine if the risk score of the subject falls into a risk category, the risk category comprising a low-risk group, a medium risk group, or a high risk group; and
providing a therapy to the subject based on the risk category of the subject.

38. The method of arrangement 37, wherein the subject is part of the high risk group, and
wherein the therapy provided to the subject is RT intensification.

39. The method of arrangement 37, wherein the subject is part of the low risk group, and
wherein the therapy provided to the subject is RT de-intensification or omission.

40. The method of arrangement 37, wherein the subject is part of the medium risk group, and
wherein the therapy provided to the subject is standard RT.

41. The method of any one of arrangements 37-40, wherein the background population is age-matched.

42. A method for treating breast cancer, comprising:
determining an Immunescore (IS) from the tumor sample based on an immunological model comprised of genes from immunological gene sets;
determining a Proliferative Index (PI) from the tumor sample based on a tumor-intrinsic model comprised of genes from tumor-intrinsic gene sets;
integrating the IS and the PT into an Integrated model;
integrating the Integrated model and an age of a subject into a Final model to identify if the subject falls into a specific-risk clinical group; and
providing an appropriate therapy to the subject based on the specific-risk clinical group.

43. A method for treating breast cancer, comprising:
identifying relevant gene sets from immunological gene sets and tumor-intrinsic gene sets;
selecting a plurality of the relevant gene sets;
creating an IS and a PI from the selected gene sets;
integrating the IS and the PI into an integrated model;
integrating the integrated model and an age of a subject to create a Final model;
identifying, by the Final model, if the subject falls into a specific-risk clinical group; and
providing an appropriate therapy to the subject based on the specific-risk clinical group,
wherein the IS corresponds to the immunological gene sets, and
   wherein the PI corresponds to the tumor-intrinsic gene sets.

44. A method for treating breast cancer, comprising:
determining a tumor aggressivity, the tumor aggressivity comprising a histological grade of a tumor from at least a portion of a sample of the tumor provided by a subject, wherein the histological grade includes Grade I, Grade II, and Grade III;
classifying the sample, when determined as a Grade I tumor, as low-risk;
classifying the sample, when determined as a Grade III tumor, as high-risk;
determining the tumor aggressivity of the sample, when determined as a Grade II tumor, the tumor aggressivity further comprising a Proliferative Index score of the sample;
classifying the Grade II tumor as:
   a) high-risk when the Proliferative Index score is greater or equal to a median score of a background population of Grade III tumors, or
   b) low-risk when the Proliferative Index score is less than the median score of the background population of Grade III tumors;
determining an Immunescore of the sample, a level of tumor infiltrating lymphocytes (TILs) in the sample, a level of checkpoint molecules in the sample, or any combination thereof to determine an immunological activity;
integrating the tumor aggressivity and the immunological activity by training an elastic net having an interaction term between the tumor aggressivity and the immunological activity,
determining a treatment plan based on an integration of tumor aggressivity and immunological activity,
wherein the Proliferative Index score is based on expression of a first group of genes,
wherein the Immunescore is based on expression of a second group of genes,
wherein, the checkpoint molecules comprise programmed cell death protein-1 (PD-1) and programmed death-ligand 1 (PD-L1),
wherein the immunological activity is determined to be:
   i) active when the TILs score is ≥10% and the checkpoint molecules score is ≥1% of lymphocytes with positive staining for PD-1 or PD-L1; or
   ii) inactive when the TILs score is less than 10% and the checkpoint molecules score is less than 1% of lymphocytes with positive staining for both PD-1 and PD-L1, or when the TILs score is ≥10%, and the checkpoint molecules score is less than 1% of lymphocytes with positive staining for both PD-1 and PD-L1,
wherein the active immunological activity indicates an activated immune infiltrate,
wherein the inactive immunological activity indicates an inactive immune infiltrate,
wherein the first group of genes comprises one or more genes listed in Table 6,
wherein the second group of genes comprises one or more genes listed in Table 4, and
wherein the treatment plan comprises:
   c) standard radiotherapy, radiotherapy de-intensification, or radiotherapy omission, when the sample is classified as the high-risk Grade II tumor and the immunological activity is active;
   d) radiotherapy intensification when sample is classified as the high-risk Grade II tumor and the immunological activity is inactive;
   e) radiotherapy intensification when the sample is classified as the low-risk Grade II tumor and the immunological activity is active; or
   f) radiotherapy de-intensification, or radiotherapy omission, when the sample is classified as the low-risk Grade II tumor and the immunological activity is inactive.

45. A method for treating breast cancer, comprising:
determining tumor aggressivity, the tumor aggressivity comprising a histological grade of a tumor from at least a portion of a sample of the tumor provided by a subject, wherein the histological grade includes Grade I, Grade II, and Grade III;
classifying the sample, when determined as a Grade I tumor, as low-risk;
classifying the sample, when determined as a Grade III tumor, as high-risk;
determining the tumor aggressivity of the sample, when determined as a Grade II tumor, the tumor aggressivity further comprising a Proliferative Index score of the sample;
classifying the Grade II tumor as:
   a) high-risk when the Proliferative Index score is above a threshold between 60th to 95th percentile compared to a background population of representative Grade II tumors, or
   b) low-risk when the Proliferative Index score is below a threshold below the 60th percentile to the background population of representative Grade II tumors;
determining an Immunescore of the sample, a level of TILs in the sample, a level of checkpoint molecules in the sample, or any combination thereof to determine an immunological activity;
integrating the tumor aggressivity and immunological activity by training an elastic net having an interaction term between the tumor aggressivity and the immunological activity,
determining a treatment plan based on the tumor aggressivity, the immunological activity, and the interaction term,
wherein the Proliferative Index score is based on expression of a first group of genes,
wherein the Immunescore is based on expression of a second group of genes,
wherein, the checkpoint molecules comprise PD-1 and PD-L1,
wherein the immunological activity is determined to be:
   i) active when the TILs score is ≥10% and the checkpoint molecules score is ≥1% of lymphocytes with positive staining for PD-1 or PD-L1, or
   ii) inactive when the TILs score is less than 10% and/or the checkpoint molecules score is less than 1% of lymphocytes with positive staining for both PD-1 and PD-L1,
wherein the active immunological activity indicates an activated immune infiltrate,
wherein the inactive immunological activity indicates an inactive immune infiltrate,
wherein the first group of genes comprises one or more genes listed in Table 6,
wherein the second group of genes comprises one or more genes listed in Table 4, and
wherein the treatment plan comprises:
   c) standard radiotherapy, radiotherapy de-intensification, or radiotherapy omission, when the sample is classified as the high-risk Grade II tumor and the immunological activity is active;
   d) radiotherapy intensification when sample is classified as the high-risk Grade II tumor and the immunological activity is inactive;
   e) the radiotherapy intensification when the sample is classified as the low-risk Grade II tumor and the immunological activity is active;
   f) radiotherapy de-intensification, or the radiotherapy omission, when the sample is classified as the low-risk Grade I tumor and the immunological activity is inactive;
   g) the radiotherapy intensification when the sample is classified as a Grade III tumor and the immunological activity is inactive;
   h) radiotherapy de-intensification, or the radiotherapy omission, when the sample is classified as a Grade III tumor and the immunological activity is active;
   i) the radiotherapy intensification when the sample is classified as a Grade I tumor and the immunological activity is active; or
   j) radiotherapy de-intensification, or the radiotherapy omission, when the sample is classified as a Grade I tumor and the immunological activity is inactive.

46. A method for treating breast cancer, comprising:
subtyping a tumor from at least a portion of a sample of the tumor provided by a subject, wherein the subtype includes Luminal A, Luminal B, HER2⁺, and triple negative/basal;
determining a tumor aggressivity of the sample, when the sample is a Luminal A tumor or a Luminal B tumor, the tumor aggressivity comprising a Proliferative Index score of the tumor sample;
classifying the sample, when the sample is subtyped as the Luminal A tumor, as:
   a) high-risk when the Proliferative Index score is above a threshold between 60th to 95th percentile compared to a background population of representative Luminal A tumors, or
   b) low-risk when the Proliferative Index score is below a threshold below the 60th percentile to the background population of representative Luminal A tumors;
classifying the sample, when the sample is subtyped as the Luminal B tumor, as:
   c) high-risk, when the Proliferative Index score is above a threshold between 60th to 95th percentile compared to a background population of representative Luminal B tumors, or
   d) low-risk when the Proliferative Index score is below a threshold below the 60th percentile to the background population of representative Luminal B tumors;
determining an immunological activity comprising scoring an immunescore of the sample, scoring a level of TILs in the sample, scoring a level of checkpoint molecules in the sample, or any combination thereof;
integrating the tumor aggressivity and the immunological activity by training an elastic net having an interaction term between the tumor aggressivity and the immunological activity,
determining a treatment plan based on the integrated tumor aggressivity, immunological activity, and the interaction term,
wherein the scoring of the Proliferative Index is based on expression of a first group of genes,
wherein the scoring of the Immunescore is based on expression of a second group of genes,
wherein, the checkpoint molecules comprise PD-1 and PD-L1,
wherein the immunological activity is determined to be:
   i) active when the TILs score is ≥10% and either of the checkpoint molecules score is ≥1%, or
   ii) inactive when the TILs score is less than 10% and the checkpoint molecules score is less than 1%,
wherein the active immunological activity indicates an activated immune infiltrate,
wherein the inactive immunological activity indicates an inactive immune infiltrate,
wherein the first group of genes comprises one or more genes listed in Table 6,
wherein the second group of genes comprises one or more genes listed in Table 4, and
wherein the treatment plan comprises:
   e) radiotherapy de-intensification or omission when the tumor sample is classified as the low-risk Luminal A tumor;
   f) standard radiotherapy, radiotherapy de-intensification, or the radiotherapy omission when the sample is classified as the high-risk Luminal B tumor and the immunological activity is active; or
   g) radiotherapy intensification when tumor sample is classified as the high-risk Luminal B tumor and the immunological activity is inactive.

47. A method for treating breast cancer, comprising:
determining a tumor aggressivity of a tumor from at least a portion of a sample of the tumor provided by a subject, wherein the tumor aggressivity comprises a Proliferative Index that classifies the tumor as:
   a) high-risk when a Proliferative Index score of the sample is above a threshold between 60th to 95th percentile compared to a background population of representative tumors, or
   b) low-risk when the Proliferative Index score of the sample is below a threshold below the 60th percentile compared to the background population of representative tumors;
determining an immunological activity of the tumor based on an Immunescore of the sample;
integrating the tumor aggressivity and immunological activity based on an interaction term between the tumor aggressivity and the immunological activity; and
determine a treatment plan based on the tumor aggressivity, immunological activity, and the interaction term.

48. A method for treating breast cancer, comprising:
determining a tumor aggressivity of a tumor from at least a portion of a sample of the tumor provided by a subject, wherein the tumor aggressivity comprises a Proliferative Index score that classifies the tumor as:
   a) high-risk when the Proliferative Index score of the sample is greater or equal to a median score of a background population of representative tumors, or
   b) low-risk when the Proliferative Index score of the sample is less than the median score of the background population of representative tumors;
determining an immunological activity of the tumor based on an Immunescore of the sample; and
integrating the tumor aggressivity and immunological activity based on an interaction term between the tumor aggressivity and the immunological activity; and
determining a treatment plan based on the tumor aggressivity, immunological activity, and the interaction term.

49. The method of arrangement 47 or 48, wherein, when the tumor is classified as high-risk and the immunological activity is active, the treatment plan comprises standard radiotherapy, radiotherapy de-intensification, or radiotherapy omission.

50. The method of arrangement 47 or 48, wherein, when the tumor is classified as high-risk and the immunological activity is inactive, the treatment plan comprises radiotherapy intensification.

51. The method of arrangement 47 or 48, wherein, when the tumor is classified as low-risk and the immunological activity is active, the treatment plan comprises radiotherapy intensification.

52. The method of arrangement 47 or 48, wherein, when the tumor is classified as low-risk and the immunological activity is inactive, the treatment plan comprises radiotherapy de-intensification or radiotherapy omission.

53. The method of any one of arrangements 47-52, wherein the integrating step comprises training an elastic net having the interaction term between the tumor aggressivity and the immunological activity.

54. The method any one of arrangements 47 and 49-53, wherein a high Proliferative Index score indicates an aggressive tumor, and
wherein, when the sample is classified as the aggressive tumor and the immunological activity is inactive, the treatment comprises radiotherapy intensification.

55. The method of arrangement 54, wherein the high Proliferative Index score comprises a Proliferative Index of the sample being at least in the 60th percentile compared to the background population of representative tumors.

56. The method of any one of arrangements 48-53, wherein a high Proliferative Index score indicates an aggressive tumor, and
wherein, when the sample is classified as the aggressive tumor and the immunological activity is inactive, the treatment comprises radiotherapy intensification.

57. The method of arrangement 56, wherein the high Proliferative Index score comprises a Proliferative Index of the sample being greater or equal to the median score of a background population of representative tumors.

58. The method of any one of arrangements 47-57, wherein the scoring of the Proliferative Index is based on expression of a first group of genes.

59. The method of arrangement 58, wherein the first group of genes comprises one or more genes listed in Table 6.

60. The method of any one of arrangements 47-59, wherein the scoring of the Immunescore is based on the expression of a second group of genes.

61. The method of arrangement 60, wherein the second group of genes comprises one or more genes listed in Table 4.

62. The method of any one of arrangements 47-61, wherein the determining of the immunological activity further comprises scoring TILs in the sample.

63. The method of arrangement 62, wherein the active immunological activity comprises an activated tumor infiltrate, and
wherein the activated tumor infiltrate comprises a TILs score of ≥10%.

64. The method of any one of arrangements 47-60, wherein the step of determining of the immunological activity further comprises measuring expression of one or more checkpoint molecules.

65. The method of arrangement 62, wherein the step of determining the immunological activity further comprises measuring expression of one or more checkpoint molecules.

66. The method of arrangement 64 or 65, wherein the checkpoint molecules comprise PD-1 and PD-L1.

67. The method of arrangement 66, wherein the active immunological activity comprises an activated tumor infiltrate, and wherein the activated tumor infiltrate comprises a positive staining for the expression of one or more of the checkpoint molecules.

68. The method of arrangement 67, further comprising wherein the step of determining of the immunological activity further comprises scoring TILs in the sample.

69. The method of arrangement 68, wherein the activated tumor infiltrate further comprises a TILs score of ≥10%.

70. The method of arrangement 62, wherein the inactive immunological activity comprises a TILs score of <10%.

71. The method of any one of arrangements 47-70, wherein the integrating step is performed by training an elastic net having the interaction term between the tumor aggressivity and the immunological activity.

72. The method of any one of arrangements 47-71, wherein the tumor aggressivity further comprises a histological grade of the sample of the tumor.

73. The method of arrangement 72, wherein the histological grade of the sample of the tumor is determined as a Grade II tumor.

74. The method of any one of arrangements 47 and 49-70 further comprising determining a subtype of the tumor sample, wherein the subtype includes Luminal A, Luminal B, HER2⁺, and triple negative/basal prior to determining the tumor aggressivity.

75. The method of arrangement 74, wherein the subtype is Luminal A.

76. The method of arrangement 74, wherein the subtype is Luminal B.

77. A method for treating breast cancer comprising:
determining a histological grade of a tumor from at least a sample of the tumor provided by a subject, wherein the histological grade for the tumor includes Grade I, Grade II, or Grade III;
assigning a tumor aggressivity of the sample as:
   a) low-risk when the sample is determined as a Grade I tumor;
   b) high-risk when the sample is determined as a Grade III tumor;
   c) low-risk when the sample is determined as a Grade II tumor and a Proliferative Index score of the sample is less than a median score of a background population of tumors; or
   d) high-risk when the sample is determined as the Grade II tumor and a Proliferative Index score of the sample is greater or equal to a median score of the background population of tumors;
classifying an immunological activity as active or inactive, the classifying comprising determining:
   e) an Immunescore of the sample;
   f) a level of TILs in the sample;
   g) a level of checkpoint molecules in the sample; or
   h) any combination of e)-f);
integrating the tumor aggressivity and the immunological activity to using an interaction term to determine the benefit of a treatment plan.

78. The method of arrangement 77, wherein, where the tumor is classified as high-risk and the immunological activity is active, the treatment plan comprises standard radiotherapy, radiotherapy de-intensification, or radiotherapy omission.

79. The method of arrangement 77, wherein, when the tumor is classified as high-risk and the immunological activity is inactive, the treatment plan comprises radiotherapy intensification.

80. The method of arrangement 77, wherein, when the tumor is classified as low-risk and the immunological activity is active, the treatment plan comprises radiotherapy intensification.

81. The method of arrangement 77, wherein, when the tumor is classified as low-risk and the immunological activity is inactive, the treatment plan comprises radiotherapy de-intensification or radiotherapy omission.

82. The method of any one of arrangements 77-81, wherein the integrating step comprises training an elastic net having the interaction term between the tumor aggressivity and the immunological activity.

83. The method of any one of arrangements 77-82, wherein a high Proliferative Index score indicates an aggressive tumor, and
wherein, when the sample is classified as the aggressive tumor and the immunological activity is inactive, the treatment comprises radiotherapy intensification.

84. The method of any one of arrangements 77-83, wherein the score of the Proliferative Index is based on expression of a first group of genes.

85. The method of arrangement 84, wherein the first group of genes comprises one or more genes listed in Table 6.

86. The method of any one of arrangements 77-85, wherein the Immunescore is based on expression of a second group of genes.

87. The method of arrangement 86, wherein the second group of genes comprises one or more genes listed in Table 4.

88. The method of any one of arrangements 77-87, wherein the wherein the checkpoint molecules comprise PD-1 and PD-L1.

89. The method of arrangement 88, wherein the immunological activity is active when:
f) is ≥10%; and
g) is ≥1% of at least one of the checkpoint molecules.

90. The method of arrangement 89, wherein the active immunological activity indicates an activated tumor infiltrate.

91. The method of arrangement 90, wherein the activated tumor infiltrate comprises a positive staining for the expression of one or more of the checkpoint molecules.

92. The method of arrangement 88, wherein the immunological activity is inactive when:
f) is <10%; and/or
g) is <1% for both checkpoint molecules.

93. A method for treating breast cancer comprising:
supplying a sample of a tumor;
receiving a treatment based on an analysis of the sample, the analysis comprising:
   determining a tumor aggressivity of the tumor from the sample;
   determining an immunological activity of the tumor from sample; and
   integrating the tumor aggressivity and the immunological activity based on an interaction term,
wherein the tumor aggressivity comprises a Proliferative Index score that classifies the tumor as high-risk or low-risk, and
wherein the immunological activity comprises:
   a) an Immunescore of the sample;
   b) a level of TILs in the sample;
   c) a level of checkpoint molecules in the sample; or
   d) any combination of a)-c).

94. The method of arrangement 93, wherein the tumor is classified as:
e) high-risk when the Proliferative Index score of the sample is above a threshold between 60th to 95th percentile compared to a background population of representative tumors, or
f) low-risk when the Proliferative Index score of the sample is below a threshold below the 60th percentile compared to the background population of representative tumors.

95. The method of arrangement 93, wherein the tumor is classified as:
g) high-risk when the Proliferative Index score of the sample is greater or equal to a median score of a background population of representative tumors, or
h) low-risk when the Proliferative Index score of the sample is less than the median score of the background population of representative tumors.

96. The method of arrangement 94 or 95, wherein, when the tumor is classified as high-risk and the immunological activity is active, the treatment plan comprises standard radiotherapy, radiotherapy de-intensification, or radiotherapy omission.

97. The method of arrangement 94 or 95, wherein, when the tumor is classified as high-risk and the immunological activity is inactive, the treatment plan comprises radiotherapy intensification.

98. The method of arrangement 94 or 95, wherein, when the tumor is classified as low-risk and the immunological activity is active, the treatment plan comprises radiotherapy intensification.

99. The method of arrangement 94 or 95, wherein, when the tumor is classified as low-risk and the immunological activity is inactive, the treatment plan comprises radiotherapy de-intensification or radiotherapy omission.

100. The method of arrangement 94 or 95, wherein a high Proliferative Index score indicates the tumor as aggressive.

101. The method of arrangement 100, wherein, when the tumor is indicated as aggressive, the treatment comprises radiotherapy intensification.

102. The method of any one of arrangements 94-101, wherein the active immunological activity comprises an activated tumor infiltrate, and
wherein the activated tumor infiltrate comprises a TILs score of ≥10%, and
wherein the inactivated tumor infiltrate comprises a TILs score of <10%.

103. The method of any one of arrangements 94-102, wherein the checkpoint molecules comprise PD-1 and PD-L1.

104. The method of arrangement 102, wherein the checkpoint molecules comprise PD-1 and PD-L1, and
wherein the activated tumor infiltrate further comprises a positive staining for the
expression of one or more of the checkpoint molecules.

105. The method of any one of arrangements 93-104, wherein the tumor aggressivity further comprises a histological grade of the sample of the tumor.

106. The method of arrangement 105, wherein the histological grade comprises Grade I, Grade II, and Grade III.

107. The method of any one of arrangements 93-104, wherein the tumor analysis further comprises determining a subtype of the tumor sample, wherein the subtype includes Luminal A, Luminal B, HER2⁺, and triple negative/basal prior to determining the tumor aggressivity.

108. The method of arrangement 107, wherein the subtype is Luminal A.

109. The method of arrangement 108, wherein the subtype is Luminal B.

110. The method of any one of arrangements 93-109, wherein the scoring of the Proliferative Index is based on expression of a first group of genes.

111. The method of arrangement 110, wherein the first group of genes comprises one or more genes listed in Table 6.

112. The method of any one of arrangements 93-111, wherein the Immunescore is based on expression of a second group of genes.

113. The method of arrangement 112, wherein the second group of genes comprises one or more genes listed in Table 4.

114. The method of any one of arrangements 93-113, wherein the integrating step comprises training an elastic net, the elastic net including the interaction term between the tumor aggressivity and the immunological activity.

115. The method of arrangement 114, wherein the elastic net further includes an age information of a subject that provided the sample.

116. The method of any one of arrangements 93-115, wherein the analysis further comprises stratification by pre- and postmenopausal status.

117. The method of arrangement 116, wherein premenopausal status includes female subjects that are <55 years, and
wherein postmenopausal status includes female subjects that are ≥55 years of age.

118. The method of any one of arrangements 44-117, wherein the background population is age-matched.

119. A method of treating a subject comprising:
determining a Proliferative Index based on a level of one or more of the genes in Table 6;
determining an Immunescore based on expression of one or more of the genes in Table 4;
combining the Proliferative Index and Immunescore and optionally factoring in age of the subject, to determine if the subject will respond to a cancer therapy, and administering the cancer therapy if the Proliferative Index and Immunescore and optionally the age of the subject indicates that the therapy will be successful.

120. The method of arrangement 119, wherein:
a) there is a high-risk when a Proliferative Index score of the sample is above a threshold between 60th to 95th percentile compared to a background population of representative tumors, or
b) there is a low-risk when the Proliferative Index score of the sample is below a threshold below the 60th percentile compared to the background population of representative tumors;
c) one combines the Proliferative Index and Immunescore based on an interaction term between the two; and
d) one determines the cancer therapy based on the Proliferative Index, Immunescore, and the interaction term.

121. The method of arrangement 120, wherein the background population is age-matched.

122. A method of treating a subject, comprising:
factoring in a level of one or more genes in Table 6;
factoring in a level of one or more genes in Table 4; and
factoring in an age of the subject, thereby determining if the subject should receive standard radiation therapy, radiotherapy intensification, radiotherapy de-intensification or radiotherapy omission.

123. The method of any one of the preceding arrangements, wherein the expression of one or more genes for the IS and/or PI is measured from the sample from the subject.

124. The method of arrangement 123, or any one of the preceding arrangements, wherein the sample comprises or consists essentially of a core biopsy.

125. The method of arrangement 123, or any one of the preceding arrangements, wherein the sample does not comprise TILs.

126. The method of arrangement 123, or any one of the preceding arrangements, wherein the analysis does not measure TIL level.

127. The method of arrangement 123, or any one of the preceding arrangements, wherein the sample consists of a core biopsy.

128. The method of arrangement 123, or any one of the preceding arrangements, wherein the sample is collected prior to a treatment of the subject for the cancer.

129. The method of arrangement 123, or any one of the preceding arrangements, wherein the sample is collected prior to a therapeutic surgery of the subject for the cancer.

130. The method of any one of the preceding arrangements, wherein age is used as a factor in determining the appropriate therapy or the treatment.

131. The method of arrangement 130, wherein a subject that is younger than 50 is considered young and a subject that is 50 or older is considered old.

132. A method of determining a therapy for the treatment of cancer, the method comprising determining an Immunescore of a core biopsy without determining a level of TILs surrounding a tumor from which the core biopsy was obtained, using the Immunescore to determine an amount of radiotherapy to administer to a subject, without factoring in the level of TILs.

133. The method of any one of the preceding arrangements, wherein one treats the subject with neoadjuvant immunotherapy.

134. The method of any one of the preceding arrangements, wherein the subject is 55 years or older.

135. The method of any one of arrangements 1-133, wherein the subject is older than 55 years in age.

136. The method of arrangement 134 or 135, wherein the subject is 65 years or older.

137. The method of arrangement 134 or 135, wherein the subject is older than 65 years.

138. The method of any one of arrangements 1-133, wherein the subject is 55 years or younger.

139. The method of any one of arrangements 1-133, wherein the subject is less than 55 years in age.

140. The method of arrangement 138 or 139, wherein the subject is less than 50 years of age.

141. The method of any one of arrangements 1-133, wherein the subject is between 50 and 65 years of age.

142. The method of any one of the preceding arrangements, wherein the tumor is sampled prior to neo-adjuvant or adjuvant therapy.

143. The method of any one of the preceding arrangements, wherein the stromal TILS around the tumor are not used in and/or available for analysis or used as part of the method.

144. A method of predicting the effectiveness of a cancer therapy, the method comprising analyzing a sample for a presence of one or more of the genes in Table 4 and/or one or more of the genes in Table 6, wherein a variation in a level of the gene indicates an effectiveness of the cancer therapy.

145. The method of arrangement 144, wherein the method further comprises administering a therapy appropriate for the subject based on the analysis of the genes in one or more of Tables 4 and/or 6.

146. A method for treating breast cancer comprising:
determining a histological grade of a tumor from at least a sample of the tumor provided by a subject, wherein the histological grade for the tumor includes Grade I, Grade II, or Grade III;
assigning a tumor aggressivity of the sample as:
   a) low-risk when the sample is determined as a Grade I tumor;
   b) low-risk when the sample is determined as a Grade II tumor and a Proliferative Index score of the sample is less than a median score of a background population of high-risk tumors;
   c) high-risk when the sample is determined as the Grade II tumor and a Proliferative Index score of the sample is greater or equal to a median score of the background population of high-risk tumors; or
   d) high-risk when the sample is determined as:
      i) a Grade III tumor,
      ii) the Proliferative Index score of the sample is greater or equal to a median score of the background population of high-risk tumors,
      iii) ER-negative,
      iv) HER2-positive,
      v) the level of Ki67 is high,
      vi) mutational burden is high, or
      vii) any combination of i)-vi);
classifying an immunological activity includes a level of TILs in the sample; and
   integrating the tumor aggressivity and the immunological activity using an interaction term to determine a treatment plan.

147. The method of arrangement 146, wherein the level of Ki67 is ≥10%.

148. The method of arrangement 146, wherein the level of Ki67 is ≥20%.

149. The method of arrangement 146, wherein the level of Ki67 is 30%.

150. The method of any one of arrangements 146-149, wherein the mutational burden is ≥5 mutations per genomic megabase (mut/MB).

151. The method of any one of arrangements 146-149, wherein the mutational burden is ≥7 mut/MB.

152. The method of any one of arrangements 146-149, wherein the mutational burden is ≥10 mut/MB.

153. A method for diagnosing breast cancer comprising:
determining a histological grade of a tumor from at least a sample of the tumor provided by a subject, wherein the histological grade for the tumor includes Grade I, Grade II, or Grade III;
assigning a tumor aggressivity of the sample as:
   a) low-risk when the sample is determined as a Grade I tumor,
   b) low-risk when the sample is determined as a Grade II tumor and a Proliferative Index score of the sample is less than a median score of a background population of high-risk tumors;
   c) high-risk when the sample is determined as the Grade II tumor and a Proliferative Index score of the sample is greater or equal to a median score of the background population of high-risk tumors; or
   d) high-risk when the sample is determined as:
      i) a Grade III tumor,
      ii) the Proliferative Index score of the sample is greater or equal to a median score of the background population of high-risk tumors,
      iii) ER-negative,
      iv) HER2-positive,
      v) the level of Ki67 is high,
      vi) mutational burden is high, or
      vii) any combination of i)-vi);
classifying an immunological activity includes a level of TILs in the sample; and
integrating the tumor aggressivity and the immunological activity using an interaction term.

154. The method of arrangement 153, wherein the level of Ki67 is ≥10%.

155. The method of arrangement 153, wherein the level of Ki67 is ≥20%.

156. The method of arrangement 153, wherein the level of Ki67 is ≥30%.

157. The method of any one of arrangements 153-156, wherein the mutational burden is ≥5 mutations per genomic megabase (mut/MB).

158. The method of any one of arrangements 153-156, wherein the mutational burden is ≥7 mut/MB.

159. The method of any one of arrangements 153-156, wherein the mutational burden is ≥10 mut/MB.

160. The method of any one of arrangements 153-159, wherein the classifying of the immunological activity further includes a level of checkpoint molecules in the sample.

161. The method of arrangement 160, wherein the checkpoint molecules include PD-1 and PD-L1.

162. A method for treating breast cancer comprising:
determining a histological grade of a tumor from at least a sample of the tumor provided by a subject, wherein the histological grade for the tumor includes Grade I, Grade II, or Grade III;
assigning a tumor aggressivity of the sample;
classifying an immunological activity includes a level of TILs in the sample; and integrating the tumor aggressivity and the immunological activity using an interaction term to determine a treatment plan.

163. The method of arrangement 162, wherein the assigning of the tumor aggressivity as high-risk comprises determining the sample as:
i) a Grade III tumor,
ii) the Proliferative Index score of the sample is greater or equal to a median score of the background population of tumors,
iii) ER-negative,
iv) HER2-positive,
v) the level of Ki67 is high,
vi) mutational burden is high, or
vii) any combination of i)-vi).

164. The method of arrangement 163, wherein the level of Ki67 is ≥10%.

165. The method of arrangement 163, wherein the level of Ki67 is ≥20%.

166. The method of arrangement 163, wherein the level of Ki67 is ≥30%.

167. The method of any one of arrangements 163-166, wherein the mutational burden is ≥5 mut/MB.

168. The method of any one of arrangements 163-166, wherein the mutational burden is ≥7 mut/MB.

169. The method of any one of arrangements 163-166, wherein the mutational burden is ≥10 mut/MB.

170. The method of arrangement 162, wherein the assigning of the tumor aggressivity as high-risk comprises determining the sample as the Grade II tumor and a Proliferative Index score of the sample is greater or equal to a median score of the background population of tumors.

171. The method of any one of arrangements 146-170, wherein the tumor aggressivity is high-risk.

172. The method of arrangement 171, wherein the level of TILs in the sample is in a range of 50% to 90%, and
wherein the treatment plan comprises radiotherapy omission.

173. The method of arrangement 171, wherein the level of TILs in the sample is in a range of 10% to 49%, and
wherein the treatment plan comprises boost omission.

174. The method of any one of arrangements 146-170, wherein the classifying of the immunological activity further includes a level of checkpoint molecules in the sample.

175. The method of arrangement 174, wherein the checkpoint molecules include PD-1 and PD-L1.

176. The method of arrangement 175, wherein the level of PD-1 is ≥1%.

177. The method of arrangement 176, wherein the level of PD-L1 is ≥1%.

178. The method of arrangement 177, wherein the level of TILs is ≥10%.

179. The method of arrangement 178, wherein the treatment plan comprises RT omission.

180. The method of arrangement 175, wherein the level of PD-1 is <1%.

181. The method of arrangement 180, wherein the level of PD-L1 is <1%.

182. The method of arrangement 181, wherein the level of TILs is ≥10%.

183. The method of arrangement 182, wherein the treatment plan comprises RT boost omission.

184. The method of any one of arrangements 146-183, wherein the subject is 55 years or older.

185. The method of any one of arrangements 146-183, wherein the subject is older than 55 years in age.

186. The method of any one of arrangements 146-183, wherein the subject is 65 years or older.

187. The method of any one of arrangements 146-183, wherein the subject is older than 65 years.

188. The method of any one of arrangements 146-183, wherein the subject is 55 years or younger.

189. The method of any one of arrangements 146-183, wherein the subject is less than 55 years in age.

190. The method of any one of arrangements 146-183, wherein the subject is between 50 and 65 years of age.

191. The method of any one of arrangements 146-183, wherein the subject is less than 50 years of age.

192. The method any one of arrangements 146-191, wherein the background population is age-matched.

193. The method of any one of arrangements 146-192, wherein the sample comprises a core biopsy.

194. A method for treating breast cancer, comprising:
determining a tumor-intrinsic aggressivity of a sample provided by a subject, the tumor-intrinsic aggressivity comprising a Proliferative Index of the sample, a histological grade of the sample, a level of Ki67 from the sample, HER2 expression of the sample, ER-status of the sample, or a combination thereof;
determining an immune infiltrate of the sample, the immune infiltrate comprising an Immunescore of the sample, a level of TILs of the sample, a level of PD-1 of the sample, a level of PD-L1 of the sample, or a combination thereof; and
providing a treatment based on the tumor-intrinsic aggressivity and the immune infiltrate,
wherein the subject is 65 years of age or older.

195. The method of arrangement 194, wherein a tumor-intrinsic risk is high when:
the Proliferative Index is above a 60th percentile compared to a background population of representative patients and tumors;
the histological grade is 3;
the histological is 2 and the Proliferative Index is equal to or above a median of a background population of grade 3 tumors;
the HER2 expression is amplified;
the ER-status of the sample is negative; or
a combination thereof.

196. The method of arrangement 194 or 195, wherein the immune infiltrate is activated when:
the Immunescore is above a threshold of 60th percentile compared to the background population of representative tumors;
the level of TILs in the sample is greater than 10%;
the level of PD-1 in the sample is greater than 1%;
the level of PD-L1 in the sample is greater than 1%; or
a combination thereof.

197. The method of arrangement 196, wherein the tumor-intrinsic risk is high and the immune activation inactive, and
wherein the treatment comprises RT boost.

198. The method of arrangement 196, wherein the tumor-intrinsic risk is high, the immune activation inactive, and the subject does not have a co-morbidity, and
wherein the treatment comprises chemotherapy intensification.

199. The method of arrangement 198, wherein the treatment further comprises immunotherapy omission.

200. The method of arrangement 198 or 199, wherein the sample is ER-positive, and
wherein the treatment further comprises endocrine therapy.

201. The method of arrangement 196, wherein the tumor-intrinsic risk is high, the immune activation inactive, and the subject does not have a co-morbidity, and
wherein the treatment comprises immunotherapy omission.

202. The method of arrangement 196, wherein the tumor-intrinsic risk is high, the immune activation inactive, the subject does not have a co-morbidity, and the sample is ER-positive, and
wherein the treatment comprises endocrine therapy.

203. The method of arrangement 196, wherein the tumor-intrinsic risk is not high, the immune infiltrate is not activated, the subject does not have a co-morbidity, and
wherein the treatment comprises RT omission.

204. The method of arrangement 203, wherein the treatment further comprises chemotherapy omission or de-escalation.

205. The method of arrangement 204, wherein the treatment further comprises endocrine therapy.

206. The method of arrangement 196, wherein the tumor-intrinsic risk is not high, the immune infiltrate is not activated, the subject does not have a co-morbidity, and
wherein the treatment comprises chemotherapy omission or de-escalation.

207. The method of arrangement 196, wherein the tumor-intrinsic risk is not high, the immune infiltrate is not activated, the subject does not have a co-morbidity, and
wherein the treatment comprises endocrine therapy.

208. The method of arrangement 196, wherein the tumor-intrinsic risk is not high, the immune infiltrate is activated, and the subject does not have a co-morbidity, and
wherein the treatment comprises RT.

209. The method of arrangement 208, wherein the treatment further comprises chemotherapy escalation.

210. The method of arrangement 209, wherein the treatment further comprises endocrine therapy.

211. The method of arrangement 196, wherein the tumor-intrinsic risk is not high, the immune infiltrate is activated, and the subject does not have a co-morbidity, and
wherein the treatment comprises chemotherapy escalation.

212. The method of arrangement 196, wherein the tumor-intrinsic risk is not high, the immune infiltrate is activated, and the subject does not have a co-morbidity, and
wherein the treatment comprises endocrine therapy.

213. The method of any one of arrangements 194-196, wherein the subject has a co-morbidity.

214. The method of arrangement 213, wherein the treatment comprises chemotherapy omission and RT de-escalation.

215. The method of any one of arrangements 198-214, wherein the co-morbidity includes coronary heart disease, heart failure, chronic obstructive pulmonary disease, previous stroke (ischemic or hemorrhagic), uncontrolled hypertension, diabetes mellitus, osteoporosis, one or more other cancers, or a combination thereof.

216. A method for treating breast cancer, comprising:
building an Integrated model from a sample provided by a subject to generate an Integrated score, wherein the Integrated model includes a Proliferative Index and an Immunescore; and
building a Final model based on the Integrated model and an age of the subject to generate a Final score,
providing a treatment based on an Integrated/Final score comprising the Integrated Score or the Final Score,
wherein the subject is 65 years old or older.

217. The method of arrangement 216, wherein the Integrated/Final score is high when the Integrated score or the Final Score is above a 60th percentile of a representative background population of patients and tumors.

218. The method of arrangement 217, wherein the background population is age-matched.

219. The method of arrangement 217, wherein the background population is age-matched with the subject.

220. The method of any one of arrangements 216-219, further comprising determining an immune infiltrate of the sample, the immune infiltrate comprising the Immunescore of the sample.

221. The method of arrangement 220, wherein the immune infiltrate further comprises a level of TILs of the sample, a level of PD-1 of the sample, a level of PD-L1 of the sample, or a combination thereof.

222. The method of arrangement 221, wherein the immune infiltrate is activated when:
the Immunescore is above a threshold of 60th percentile compared to the background population of representative tumors;
the level of TILs in the sample is greater than 10%;
the level of PD-1 in the sample is greater than 1%;
the level of PD-L1 in the sample is greater than 1%; or
a combination thereof.

223. The method of arrangement 220, wherein the immune infiltrate is activated when the Immunescore is above a threshold of 60th percentile compared to the background population of representative patients and tumors.

224. The method of arrangement 223, wherein the background population is age-matched.

225. The method of arrangement 223, wherein the background population is age-matched with the subject.

226. The method of any one of arrangements 216-225, wherein the Integrated/Final score is high, and
wherein the treatment comprises RT boost.

227. The method of any one of arrangements 222-225, wherein the Integrated/Final score is not high and the immune infiltrate is active, and
wherein the treatment comprises chemotherapy omission or chemotherapy de-escalation.

228. The method of arrangement 227, wherein the treatment further comprises immunotherapy.

229. The method of arrangement 228, wherein the sample is ER-positive, and
wherein the treatment further comprises endocrine therapy.

230. The method of any one of arrangements 222-225, wherein the Integrated/Final score is high and the immune infiltrate is activated, and
wherein the treatment comprises immunotherapy.

231. The method of any one of arrangements 222-225, wherein the Integrated/Final score is not high and the immune infiltrate is not activated, and
wherein the treatment comprises RT omission.

232. The method of arrangement 231, wherein the treatment further comprises chemotherapy omission or de-escalation.

233. The method of arrangement 232, wherein the treatment further comprises endocrine therapy.

234. The method of any one of arrangements 222-225, wherein the Integrated/Final score is not high and the immune infiltrate is activated, and
wherein the treatment comprises standard RT.

235. The method of arrangement 234, wherein the treatment further comprises chemotherapy escalation.

236. The method of any one of arrangements 222-225, wherein the Integrated/Final score is not high and the immune infiltrate is activated, and
wherein the treatment comprises chemotherapy escalation.

237. The method of any one of arrangements 234-236, wherein the treatment further comprises endocrine therapy.

238. A method for treating breast cancer, comprising:
determining a tumor-intrinsic aggressivity of a sample provided by a subject, the tumor-intrinsic aggressivity comprising a Proliferative Index of the sample, a histological grade of the sample, a level of Ki67 from the sample, HER2 expression of the sample, ER-status of the sample, or a combination thereof;
determining an immune infiltrate of the sample, the immune infiltrate comprising an Immunescore of the sample, a level of TILs of the sample, a level of PD-1 of the sample, a level of PD-L1 of the sample, or a combination thereof; and
providing a treatment based on the tumor-intrinsic aggressivity and the immune infiltrate,
wherein the subject is less than 55 years of age.

239. A method for treating breast cancer, comprising:
building an Integrated model from a sample provided by a subject to generate an Integrated score, wherein the Integrated model includes a Proliferative Index and an Immunescore; and
building a Final model based on the Integrated model and an age of the subject to generate a Final score,
providing a treatment based on an Integrated/Final score comprising the Integrated score or the Final score,
wherein the subject is less than 55 years old.

240. The method of arrangement 239, wherein the Integrated/Final score is less than a 5th percentile of a representative background population of patients and tumors.

241. The method of arrangement 240, wherein the treatment comprises a RT de-escalation.

242. The method of arrangement 239, wherein the Integrated/Final score is greater than a 50th percentile of a representative background population of patients and tumors.

243. The method of arrangement 242, wherein the treatment comprises RT escalation compared to standard RT.

244. The method of arrangement 243, wherein the RT escalation comprises RT boost.

245. The method of arrangement 239, wherein the Integrated/Final score is greater than 95th percentile of a representative background population of patients and tumors.

246. The method of arrangement 245, wherein the treatment comprises surgical escalation.

247. The method of arrangement 246, wherein the surgical escalation comprises mastectomy.

248. The method of arrangement 246, wherein the surgical escalation further comprises wider resection margins.

249. The method of arrangement 247 or 248, wherein the treatment further comprises systemic therapy escalation.

250. The method of arrangement 245, wherein the treatment comprises systemic therapy escalation.

251. The method of any one of arrangements 240-250, wherein the background population is age-matched.

252. The method of any one of arrangements 240-250, wherein the background population is age-matched with the subject.

253. A method for treating breast cancer, comprising:
building an Integrated model from a sample provided by a subject to generate an Integrated score, wherein the Integrated model includes a Proliferative Index and an Immunescore; and
building a Final model based on the Integrated model and an age of the subject to generate a Final score,
providing a treatment based on an Integrated/Final score comprising the Integrated score or the Final score,
wherein the subject is greater than 55 years old.

254. The method of arrangement 253, wherein the Integrated/Final score is above a 70th percentile of a representative background population of patients and tumors.

255. The method of arrangement 254, wherein the treatment comprises RT escalation.

256. The method of arrangement 255, wherein the RT escalation comprises RT boost.

257. The method of arrangement 253, wherein the Integrated/Final score is above an 85th percentile of a representative background population of patients and tumors.

258. The method of arrangement 257, wherein the treatment comprises mastectomy.

259. The method of arrangement 257, wherein the treatment comprises escalating systemic therapy.

260. The method of any one of arrangements 253-259, wherein the background population is age-matched.

261. The method of any one of arrangements 253-259, wherein the background population is age-matched with the subject.

262. The method of any one of the preceding arrangements regarding treatment, wherein the subject is administered or receives the indicated or disclosed treatment or therapy.

### Example 1

The local immune infiltrate's influence on tumor progression is closely linked to tumor-intrinsic factors. Genomically unstable tumors with a high tumor mutational burden benefit more from an immune infiltrate. The aim was to investigate whether integrating immunological and tumor-intrinsic factors can identify clinically high-risk patients who may be candidates for de-escalation of radiotherapy (RT).

The SweBCG91RT trial included 1178 patients with stage I-IA breast cancer, randomized to breast-conserving surgery with or without adjuvant RT, and followed for a median time of 15.2 years. In total, 8% were treated with systemic therapy. Using publicly available cohorts, two models were trained where they were designed to capture immune activity and immunomodulatory tumor-intrinsic qualities, respectively. Afterwards, analysis was performed to determine if combining these two variables could identify high-risk tumors with a favorable prognosis without RT boost.

The immunological (Immunescore) and tumor-intrinsic (Proliferative Index) models showed a strong interaction effect (p=0.01). Aggressive tumors (i.e., high scores of the tumor-intrinsic model called Proliferative Index) with low immunological activity had the highest risk of ipsilateral breast tumor recurrence (IBTR). By integrating the immunological and tumor-intrinsic models, irradiated patients with grade III tumors aged <70 years and patients aged <60 years regardless of histological grade with a 10-year IBTR risk below 10% despite a lack of RT boost and a low frequency of systemic therapy were identified.

### Model Training

### Training Population

Publicly available breast cancer data sets were downloaded using the R package MetaGxBreast[1], Table 3. For the TCGA cohort, updated follow-up data was retrieved using the R package curatedTCGAData[2]. The cohorts with available outcome data (n=21) were selected as training cohorts. Pam50 subtypes were inferred using the genefu package[3]. The endpoints used were chosen in the following order based on availability: 1. Distant metastasis. 2. Any recurrence. 3. Overall survival. Age was included as a covariate for overall survival. The follow-up time used was 10 years for distant metastasis and any recurrence, and 15 years for overall survival.

The rank-based single-sample Gene-Set Enrichment Analysis (ssGSEA) method was chosen to minimize batch effects, as rank-based methods show robustness for analyses across different array platforms. HGNChelper was used to harmonize the names of genes between the different training cohorts. Without being bound by theory, it is hypothesized that biological processes, rather than individual genes, drive prognosis and treatment prediction, so gene sets from msigdb was used as features. Unlike individual genes, which can be involved in several different biological pathways and expressed in various cell types, a gene set is more specific for a given underlying biological process, which is important because testing the hypothesis required an accurate assessment of the immune infiltrate. Finally, because enrichment scores are calculated based on available genes in each gene set, the analysis does not need to be limited to genes profiled in all 21 training cohorts. In other words, an enrichment score is calculated for the respective included gene sets using the ssGSEA method. The gene sets and coefficients are presented in Tables 4 and/or 6. The enrichment score for each gene set is then centered to have a mean of 0 and a standard deviation of 1. The enrichment score is then multiplied with the respective coefficient and the values are summed to obtain a score for Immunescore or Proliferative Index. The Integrated score is obtained by centering the Immunescore and Proliferative Index to have a mean 0 and a standard deviation 1. The formula in Table 8 is then used to obtain the Integrated score.

TABLE 3
Demographics of the training cohorts

| | | | | | | | | Median |
|---|---|---|---|---|---|---|---|---|
| | Lum A | Lum B | Her2 | Basal | Normal | Total | Median | tumor |
| Cohort | n | n | n | n | n | n | age | size |
| CAL | 34 | 45 | 10 | 27 | 2 | 118 | 51 | 2.3 |
| | (28.81%) | (38.14%) | (8.47%) | (22.88%) | (1.69%) | | (missing = | (missing = |
| | | | | | | | 1) | 2) |
| DFHCC | 39 | 30 | 18 | 28 | 0 | 115 | 53 | 2.1 |
| | (33.91%) | (26.09%) | (15.65%) | (24.35%) | (0%) | | (missing = | (missing = |
| | | | | | | | 0) | 0) |
| DUKE | 43 | 48 | 37 | 24 | 15 | 167 | NA | 2.3 |
| | (25.75%) | (28.74%) | (22.16%) | (14.37%) | (8.98%) | | (missing = | (missing = |
| | | | | | | | 167) | 79) |
| EMC2 | 66 | 44 | 34 | 55 | 5 | 204 | NA | NA |
| | (32.35%) | (21.57%) | (16.67%) | (26.96%) | (2.45%) | | (missing = | (missing = |
| | | | | | | | 204) | 204) |
| GSE58644 | 111 | 118 | 37 | 48 | 1 | 315 | 58 | 2.1 |
| | (35.24%) | (37.46%) | (11.75%) | (15.24%) | (0.32%) | | (missing = | (missing = |
| | | | | | | | 0) | 0) |
| GSE25066 | 124 | 136 | 61 | 170 | 17 | 508 | 49 | NA |
| | (24.41%) | (26.77%) | (12.01%) | (33.46%) | (3.35%) | | (missing = | (missing = |
| | | | | | | | 0) | 508) |
| UNC4 | 59 | 54 | 50 | 86 | 54 | 303 | 55 | 3 |
| | (19.47%) | (17.82%) | (16.5%) | (28.38%) | (17.82%1 | | (missing = | (missing = |
| | | | | | | | 59) | 72) |
| MAINZ | 85 | 61 | 18 | 24 | 12 | 200 | 60 | 2 |
| | (42.5%) | (30.5%) | (9%) | (12%1 | (6%) | | (missing = | (missing = |
| | | | | | | | 0) | 0) |
| METABRIC | 501 | 921 | 249 | 275 | 144 | 2090 | 61.77 | 2.3 |
| | (23.97%) | (44.07%) | (11.91%) | (13.16%) | (6.89%) | | (missing = | (missing = |
| | | | | | | | 10) | 135) |
| MSK | 20 | 27 | 16 | 29 | 6 | 98 | 56.5 | 3.2 |
| | (20.41%) | (27.55%) | (16.33%) | (29.59%) | (6.12%) | | (missing = | (missing = |
| | | | | | | | 0) | 0) |
| NCI | 13 | 31 | 18 | 29 | 8 | 99 | 57 | 2.5 |
| | (13.13%) | (31.31%) | (18.18%) | (29.29%) | (8.08%) | | (missing = | (missing = |
| | | | | | | | 0) | 0) |
| NKI | 121 | 110 | 39 | 62 | 5 | 337 | 45 | 2 |
| | (35.91%) | (32.64%) | (11.57%) | (18.4%) | (1.48%) | | (missing = | (missing = |
| | | | | | | | 0) | 0) |
| UNT | 60 | 37 | 12 | 18 | 6 | 133 | 53 | 1.9 |
| | (45.11%) | (27.82%) | (9.02%) | (13.53%) | (4.51%) | | (missing = | (missing = |
| | | | | | | | 0) | 0) |
| PMC | 20 | 30 | 17 | 23 | 1 | 91 | 53.98 | 2.5 |
| | (21.98%) | (32.97%) | (18.68%) | (25.27%) | (1.1%) | | (missing = | (missing = |
| | | | | | | | 4) | 6) |
| STK | 56 | 50 | 23 | 18 | 12 | 159 | 56 | NA |
| | (35.22%) | (31.45%) | (14.47%) | (11.32%) | (7.55%) | | (missing = | (missing = |
| | | | | | | | 0) | 159) |
| STNO2 | 28 | 30 | 23 | 31 | 6 | 118 | 58 | 3 |
| | (23.73%) | (25.42%) | (19.49%) | (26.27%) | (5.08%) | | (missing = | (missing = |
| | | | | | | | 0) | 5) |
| TCGA | 379 | 312 | 101 | 175 | 32 | 999 | 58 | NA |
| | (37.94%) | (31.23%) | (10.11%) | (17.52%) | (3.2%) | | (missing = | (missing = |
| | | | | | | | 0) | 999) |
| TRANSBIG | 82 | 44 | 25 | 44 | 3 | 198 | 46 | 2 |
| | (41.41%) | (22.22%) | (12.63%) | (22.22%) | (1.52%) | | (missing = | (missing = |
| | | | | | | | 0) | 0) |
| UCSF | 25 | 48 | 22 | 54 | 12 | 161 | 53.5 | 2 |
| | (15.53%) | (29.81%) | (13.66%) | (33.54%) | (7.45%) | | (missing = | (missing = |
| | | | | | | | 9) | 7) |
| UPP | 112 | 63 | 36 | 27 | 13 | 251 | 64 | 2 |
| | (44.62%) | (25.1%) | (14.34%) | (10.76%) | (5.18%) | | (missing = | (missing = |
| | | | | | | | 0) | 0) |
| VDX | 95 | 98 | 41 | 96 | 14 | 344 | 52 | 1 |
| | (27.62%) | (28.49%) | (11.92%) | (27.91%) | (4.07%) | | (missing = | (missing = |
| | | | | | | | 58) | 58) |

| | Median | | Chemo- | Chemo- | Hormono- | |
|---|---|---|---|---|---|---|
| Cohort | LNM | Untreated | therapy | hormono | therapy | Endpoint |
| CAL | 1 | 14 | 40 | 36 | 25 | Distant |
| | (missing = | (12.17%) | (34.78%) | (31.3%) | (21.74%) | metastasis |
| | 0) | | | | | |
| DFHCC | 0 | 7 | 22 | 38 | 42 | Distant |
| | (missing = | (6.42%) | (20.18%) | (34.86%) | (38.53%) | metastasis |
| | 0) | | | | | |
| DUKE | 0 | NA | NA | NA | NA | Overall |
| | (missing = | | | | | survival |
| | 78) | | | | | |
| EMC2 | 0 | 48 | NA | 156 | NA | Distant |
| | (missing = | (23.53%) | | (76.47%) | | metastasis |
| | 156) | | | | | |
| GSE58644 | 1 | 10 | 65 | 29 | 86 | Distant |
| | (missing = | (5.26%) | (34.21%) | (15.26%) | (45.26%) | metastasis |
| | 32) | | | | | |
| GSE25066 | 1 | NA | NA | NA | NA | Distant |
| | (missing = | | | | | metastasis |
| | 0) | | | | | |
| UNC4 | 1 | NA | NA | NA | NA | Any |
| | (missing = | | | | | recurrence |
| | 44) | | | | | |
| MAINZ | 0 | NA | NA | NA | NA | Distant |
| | (missing = | | | | | metastasis |
| | 0) | | | | | |
| METABRIC | 0 | 653 | 1023 | 223 | 191 | Overall |
| | (missing = | (31.24%) | (48.95%) | (10.67%) | (9.14%) | survival |
| | 116) | | | | | |
| MSK | 1 | NA | NA | NA | NA | Distant |
| | (missing = | | | | | metastasis |
| | 0) | | | | | |
| NCI | 1 | 11 | 78 | 10 | NA | Any |
| | (missing = | (11.11%) | (78.79%) | (10.1%) | | recurrence |
| | 0) | | | | | |
| NKI | 0 | 207 | 40 | 90 | NA | Distant |
| | (missing = | (61.42%) | (11.87%) | (26.71%) | | metastasis |
| | 0) | | | | | |
| UNT | 0 | NA | NA | NA | NA | Distant |
| | (missing = | | | | | metastasis |
| | 0) | | | | | |
| PMC | 0 | NA | NA | NA | NA | Any |
| | (missing = | | | | | recurrence |
| | 9) | | | | | |
| STK | NA | 22 | 48 | 89 | NA | Any |
| | (missing = | (13.84%) | (30.19%) | (55.97%) | | recurrence |
| | 159) | | | | | |
| STNO2 | 1 | 22 | 73 | 23 | NA | Any |
| | (missing = | (18.64%) | (61.86%) | (19.49%) | | recurrence |
| | 5) | | | | | |
| TCGA | 1 | NA | NA | NA | NA | Overall |
| | (missing = | | | | | survival |
| | 0) | | | | | |
| TRANSBIG | 0 | NA | NA | NA | NA | Distant |
| | (missing = | | | | | metastasis |
| | 0) | | | | | |
| UCSF | 1 | 22 | 61 | 38 | 30 | Distant |
| | (missing = | (14.57%) | (40.4%) | (25.17%) | (19.87%) | metastasis |
| | 13) | | | | | |
| UPP | 0 | 142 | 80 | NA | NA | Any |
| | (missing = | (63.96%) | (36.04%) | | | recurrence |
| | 9) | | | | | |
| VDX | 0 | NA | NA | NA | NA | Distant |
| | (missing = | | | | | metastasis |
| | 0) | | | | | |

### Immunescore

In a non-limiting example, the immune model was trained within Basal and HER2⁺ tumors (n=2230) because less aggressive subtypes exhibit a more heterogeneous immunological prognostic signal. ssGSEA scores were obtained using the msigdb and GSVA packages for gene sets from the C7 category (immunological signature gene set) and all additional gene sets, including any of the keywords “LYMPHOCYTE|T_CELL|PD1|PD-1|PDL1|PD-L1|LAG3|CHECKPOINT_RECEPTOR|B_CELL|PERFORIN|GRANZYME|NK_CELL|CD 8|CYTOTOXIC” (cell type signature gene sets) (N=5661 gene sets, which altogether include over 20,000 genes).

The features of the gene sets were analyzed in Cox regression models, and the p-values were saved as two-tailed (each p-value was multiplied with the sign of the respective coefficients) and converted to one-sided p-values using the metap package[4]. A meta-analysis of the 21 p-values for each feature was then performed using the weighted sum Z (Stouffer's) method[5]. The square roots of the number of observations in each cohort was used as weights. The top 50 gene sets were selected, and Spearman correlations were computed for each possible pairwise combination of the 50 gene sets within each cohort. A mean value for each pairwise correlation was generated using all 22 cohorts. To reduce variable multicollinearity, the gene set with the lowest p-value for all gene sets with a mean pairwise correlation ≥0.7 or <−0.7 was removed, indicating a strong association. This resulted in 22 remaining gene sets as shown in Table 4, which were then scaled and centered within each cohort, and the cohorts were merged. An elastic net model was fit using the caret package[6], FIG. 1. The resulting model was named Immunescore, FIG. 1. In some embodiments, this model is based on immunological model, the immunological model including genes from immunological gene sets, some of which are shown in Table 4. Regarding the correlations, in some embodiments, a different threshold may be selected to perform the collinearity filtering (i.e., removing highly correlated gene sets). In some embodiments, the top number of gene sets that are selected may be similar to that of the general model above: in some embodiments, the top 5 gene sets were selected; in some embodiments, the top 200 gene sets were selected; or, in some embodiments, any number from the top 5 gene sets to the top 200 gene sets were selected (e.g., top 10 gene sets, top 15 gene sets, . . . top 100 gene sets, . . . etc.).

Since the model was trained against prognosis, the model measures the quantity and quality of the local immune response. However, since direct measurements of the immune infiltrate were not used when developing the model, the association was validated by comparing it to immunohistochemistry assessments of stromal tumor-infiltrating lymphocytes (TILs) on whole-tissue sections scored as previously described[7], which is incorporated by reference in its entirety herein. Furthermore, xCell[8], and ESTIMATE[9], which are deconvolutional methods developed to quantify immune infiltrates from gene expression data, were used as controls for the correlation with TILs. Our results show that Immunescore performs better than ESTIMATE and xCell as indicated by a stronger Spearman correlation with TILs (rho 0.42, p<0.001 vs rho 0.33, p<0.001 and rho 0.27, p<0.001, respectively). In other words, the immunological model (Immunescore) disclosed herein performs better than the already established methods xCell and ESTIMATE. This is reflected by the stronger correlation between Immunescore and TILs vs. that of ESTIMATE/xCell and TILs.

TABLE 4
Selected gene sets and genes used to determine Immunescore

| Gene Set | Genes | Coefficient |
|---|---|---|
| (Intercept) | — | −0.558318003922337 |
| AIZARANI_LIVER_C24_EPCAM_{—} | AGR2, AQP3, ATP1B1, BIRC3, | 0.00777260484210833 |
| POS_BILE_DUCT_CELLS_3 | CAMK2N1, CAPN2, CCND1, | |
| | CD24, CDH1, CEACAM6, | |
| | DUSP4, GSTP1, KLF5, LMO4, | |
| | MMP7, MUC1, SHROOM3, | |
| | SOX4, SPP1, TFF3, TM4SF1, | |
| | TMC5, TXLNG | |
| GAUCHER_PBMC_YF_VAX_{—} | ADA, CCNB2, CD38, CDCA7, | −0.0101018153391127 |
| STAMARIL_UNKNOWN_AGE_{—} | COBLL1, DUSP5, FAM30A, | |
| 14DY_UP | GLDC, JCHAIN, MZB1, | |
| | NUSAP1, PDIA4, PRDX4, | |
| | PTTG1, RTCB, SEL1L3, SP140, | |
| | SRM, TENT5C, TNFRSF17, | |
| | TXNDC5, UBE2C, XBP1 | |
| GOBP_ACTIVATED_T_CELL_{—} | BTN2A2, BTN3A1, CASP3, | −0.00888976110078319 |
| PROLIFERATION | CD274, CLC, CRTAM, FADD, | |
| | FYN, HMGB1, ICOSLG, IGF1, | |
| | IGFBP2, IL23A, IL23R, IL27RA, | |
| | IL2RA, LILRB4, LRRC32, | |
| | PRNP, PYCARD, RIPK3, RPS3, | |
| | STAT5B | |
| GOBP_THYMIC_T_CELL_{—} | AIRE, ATG5, CARD11, CCR7, | −0.00433600098576297 |
| SELECTION | CD28, CD3D, CD3E, CD3G, | |
| | CD74, DOCK2, FOXN1, | |
| | GATA3, GLI3, ITPKB, JAG2, | |
| | PTPN2, PTPRC, SHH, SRF, | |
| | STK11, TOX, ZAP70 | |
| GSE1460_CD4_THYMOCYTE_{—} | ACTN1, BMERB1, CDC42BPA, | 0.00438292213983279 |
| VS_THYMIC_STROMAL_{—} | CDH2, COPB2, DERL1, DHRS7, | |
| CELL_DN | DLG5, IGFBP5, KCNK1, | |
| | LARGE1, ME1, MMP1, MT1E, | |
| | PHLDA1, PON2, RBP1, STAM2, | |
| | STK3, TMEM100, UAP1, UCK2, | |
| | UST | |
| GSE1460_CD4_THYMOCYTE_{—} | ATP6V0A4, CELSR2, CRYBG1, | −0.000733659085694323 |
| VS_THYMIC_STROMAL_{—} | IFI44L, ISG20, KLF2, | |
| CELL_UP | PCDHB13, PLIN1, POMC, | |
| | PTDSS1, RSRP1, SDK2, | |
| | SLC9A3R1, SNX11, SP140L, | |
| | TGIF2, TMEM131L, TNFSF10, | |
| | TRANK1, TRIM14, VAV3, XPA, | |
| | ZNF271P | |
| GSE1460_DP_THYMOCYTE_{—} | C3orf14, CHPT1, CTTN, | 0.00672357926688569 |
| VS_THYMIC_STROMAL_CELL_{—} | DNAJC12, DNAJC15, | |
| DN | FAM171A1, FTO, HIGD1A, | |
| | IFT46, KLF9, LAMP2, | |
| | MANSC1, MORF4L2, MYO5A, | |
| | NBN, PLGRKT, PYGL, REXO2, | |
| | RHOBTB3, RPS18, STAT1, | |
| | TMX1, USP13 | |
| GSE1740_MCSF_VS_MCSF_{—} | ACO2, B2M, BTN3A2, CDH3, | −0.00458406229490887 |
| AND_IFNG_DAY2_DERIVED_{—} | CHST3, ERAP2, FHL2, FNDC5, | |
| MACROPHAGE_UP | GBP1, GBP4, GBP5, LAMA3, | |
| | MDGA1, NLRC5, PARP14, | |
| | PLAAT4, POLB, PSMB8, | |
| | RAB30, TAP1, TOX3, UBE2L6, | |
| | ZNF239 | |
| GSE22886_NAIVE_CD4_{—} | AP3M2, CD69, CD96, CDC25B, | −0.00355796524313593 |
| TCELL_VS_MONOCYTE_UP | CHD7, ECHDC2, EPHX2, | |
| | GAMT, GZMM, ICOS, INPP4B, | |
| | KIF5C, LEF1, MGAT4A, | |
| | NUCB2, PILRB, PLEKHA1, | |
| | PRKCA, RETREG1, SIDT1, | |
| | SKAP1, TNIK, ZNF551 | |
| GSE24142_EARLY_THYMIC_{—} | AP3S1, ARHGAP9, AXIN2, | −0.00843862054872214 |
| PROGENITOR_VS_{—} | BNIPL, CARHSP1, CCNA2, | |
| DNHYMOCYTE_DN | CTLA4, DEGS2, DGKE, FKBP5, | |
| | FYB1, GRAP2, GSR, HMGCS2, | |
| | IL17RB, LPXN, MCOLN2, | |
| | NCKAP1, PDLIM1, RAB27A, | |
| | RHOH, SEC24D, TMEM50B | |
| GSE26488_WT_VS_HDAC7_{—} | CP, ERGIC1, FGF11, HOXA2, | 0.0079331267035272 |
| DELTAP_TG_OT2_THYMOCYTE_{—} | IER3, IF127, IL6ST, KDM5B, | |
| WITH_PEPTIDE_INJECTION_{—} | KIF16B, MOCOS, OAT, PLK2, | |
| DN | PROM1, PRSS23, SLC1A4, | |
| | SLC39A8, SLC4A7, SMO, | |
| | SYTL4, TPBG, TSPAN6, | |
| | TXNDC16, YBX3 | |
| GSE29618_BCELL_VS_{—} | AFF3, BCL11A, CKAP2, | −0.0053536894785983 |
| MONOCYTE_UP | CSNK1G3, CYFIP2, E2F5, EVL, | |
| | GABPB1, GGA2, H2BC5, | |
| | KCNA3, MPP6, PARP1, PHTF2, | |
| | PIM2, PPP1R16B, RABEP1, | |
| | RASGRP1, RPS6, SH3BP5, | |
| | TMEM156, TSPAN13, ZHX2 | |
| GSE29618_MONOCYTE_VS_{—} | AHNAK2, APMAP, CCT2, | −0.00808644523138692 |
| MDC_DN | CD1C, CD2, DTNA, FAM162A, | |
| | FLT3, GATM, GSN, H2BC10, | |
| | HIP1, IRF4, ITGB7, PPA1, | |
| | RGS1, RGS10, RPS2, SCD, | |
| | SCRN1, SH3BP4, SUB1, TOB1 | |
| GSE3039_ALPHAALPHA_CD8_{—} | APOC1, ASAP1, C2, CDC40, | −0.00458749986713501 |
| TCELL_VS_B2_BCELL_UP | CYTIP, DMKN, EPB41L3, | |
| | FABP3, FAM241A, GP2, | |
| | GPR160, LRG1, MSN, NTNG1, | |
| | PLA2G7, PMM1, PTPN1, | |
| | SFT2D2, SMPDL3A, SPTB, | |
| | ST3GAL4, TESMIN, TSPYL4 | |
| GSE40666_UNTREATED_VS_{—} | AGPAT5, CCNG2, CDCA7L, | −0.00400399595864113 |
| IFNA_STIM_STAT1_KO_CD8_{—} | CERS4, DPYD, ENPP1, | |
| TCELL_90MIN_DN | FAM234B, FOXP1, GCNT1, | |
| | GLCCI1, MAML3, NRIP1, | |
| | P2RX1, SERPINI1, SLC4A8, | |
| | SNX27, ST3GAL1, SYT17, | |
| | TCTN1, TRPS1, UBE2E2, UBL3, | |
| | ZNF135 | |
| GSE42021_CD24HI_VS_{—} | AP1M2, CAP2, CBX6, CLIP4, | 0.00372214341549843 |
| CD24INT_TREG_THYMUS_UP | FECH, GDE1, GNPAT, GPC1, | |
| | IMPDH2, IPO9, MPC2, NRCAM, | |
| | PALLD, PDGFC, PDGFD, | |
| | PLCE1, PXDN, SCARA3, SRI, | |
| | TBL1X, TMF1, TMX4, USP34 | |
| HP_T_CELL_LYMPHOMA | CASP10, FAS, FASLG, MSH6, | −0.00649229114288465 |
| | POLE, PRKCD, TNFRSF1B | |
| LI_PBMC_MENACTRA_AGE_{—} | IGHE, IGHG3, IGLL1, PDIA6 | −0.00616710150938698 |
| 18_45YO_7DY_UP | | |
| PATEL_SKIN_OF_BODY_ZOSTAVAX_{—} | CCL5, CD8A, CLEC12A, | −0.00693772335418378 |
| AGE_70_93YO_VZV_{—} | CLEC4E, CXCL10, CXCL9, | |
| CHALLENGED_POST_VACCINATION_{—} | EOMES, FCGR1A, FCGR1B, | |
| VS_UNCHALLENGED_{—} | FPR1, GNLY, GZMB, IDO1, | |
| 72HR_TOP_30_DEG_UP | ITGAL, KLHDC7B, KLRC2, | |
| | LYZ, SELL, SERPINA1, UBD, | |
| | XCL1 | |
| SCHERER_PBMC_APSV_{—} | ABCC5, ADSL, B4GALT3, | −0.00486094466565956 |
| WETVAX_AGE_18_32YO_5_TO_{—} | CRIP1, EXPH5, GZMH, GZMK, | |
| 7DY_DN | KCNN4, KIF22, LIMCH1, | |
| | MRPS21, OSER1, PDE3B, | |
| | PELP1, POC1B, RPL12, RPL13, | |
| | RPL13A, RPL14, RPL23A, | |
| | RPS15A, RPS27, TRAF3IP3 | |
| SHIN_B_CELL_LYMPHOMA_{—} | CADM1, CD86, CXCL13, | −0.00439236059907722 |
| CLUSTER_3 | CXCR6, FGR, FZD1, HHEX, | |
| | ID2, JAK2, LFNG, MAF, | |
| | NFATC1, NFKB2, TGFB1, | |
| | TNFRSF1A, TNFSF13, WNT5A, | |
| | ZEB2 | |
| SHIN_B_CELL_LYMPHOMA_{—} | GATA1, HMGB3, HOXA4, | −0.012608144870638 |
| CLUSTER_9 | IKZF3, IRF8, MMP13, REL, | |
| | TAL1, TLX1, WNT2 | |

TABLE 5
Immunescore Model Calculations

| Variable | Coefficient |
|---|---|
| (Intercept) | −0.558318003922337 |
| AIZARANI_LIVER_C24_EPCAM_POS_{—} | 0.00777260484210833 |
| BILE_DUCT_CELLS_3 | |
| GAUCHER_PBMC_YF_VAX_STAMARIL_UNKNOWN_{—} | −0.0101018153391127 |
| AGE_14DY_UP | |
| GOBP_ACTIVATED_T_CELL_PROLIFERATION | −0.00888976110078319 |
| GOBP_THYMIC_T_CELL_SELECTION | −0.00433600098576297 |
| GSE1460_CD4_THYMOCYTE_VS_THYMIC_STROMAL_{—} | 0.00438292213983279 |
| CELL_DN | |
| GSE1460_CD4_THYMOCYTE_VS_THYMIC_STROMAL_{—} | −0.000733659085694323 |
| CELL_UP | |
| GSE1460_DP_THYMOCYTE_VS_THYMIC_STROMAL_{—} | 0.00672357926688569 |
| CELL_DN | |
| GSE1740_MCSF_VS_MCSF_AND_IFNG_DAY2_{—} | −0.00458406229490887 |
| DERIVED_MACROPHAGE_UP | |
| GSE22886_NAIVE_CD4_TCELL_VS_MONOCYTE_UP | −0.00355796524313593 |
| GSE24142_EARLY_THYMIC_PROGENITOR_VS_DN2_{—} | −0.00843862054872214 |
| THYMOCYTE_DN | |
| GSE26488_WT_VS_HDAC7_DELTAP_TG_OT2_{—} | 0.0079331267035272 |
| THYMOCYTE_WITH_PEPTIDE_INJECTION_DN | |
| GSE29618_BCELL_VS_MONOCYTE_UP | −0.0053536894785983 |
| GSE29618_MONOCYTE_VS_MDC_DN | −0.00808644523138692 |
| GSE3039_ALPHAALPHA_CD8_TCELL_VS_B2_BCELL_{—} | −0.00458749986713501 |
| UP | |
| GSE40666_UNTREATED_VS_IFNA_STIM_STAT1_{—} | −0.00400399595864113 |
| KO_CD8_TCELL_90MIN_DN | |
| GSE42021_CD24HI_VS_CD24INT_TREG_THYMUS_UP | 0.00372214341549843 |
| HP_T_CELL_LYMPHOMA | −0.00649229114288465 |
| LI_PBMC_MENACTRA_AGE_18_45YO_7DY_UP | −0.00616710150938698 |
| PATEL_SKIN_OF_BODY_ZOSTAVAX_AGE_70_93YO_{—} | −0.00693772335418378 |
| VZV_CHALLENGED_POST_VACCINATION_VS_{—} | |
| UNCHALLENGED_72HR_TOP_30_DEG_UP | |
| SCHERER_PBMC_APSV_WETVAX_AGE_18_32YO_5_{—} | −0.00486094466565956 |
| TO_7DY_DN | |
| SHIN_B_CELL_LYMPHOMA_CLUSTER_3 | −0.00439236059907722 |
| SHIN_B_CELL_LYMPHOMA_CLUSTER_9 | −0.012608144870638 |

### Calculating Risk Score

In this non-limiting example to calculate the risk score, here Table 4 serves an example of how the risk score is calculated from the values provided by the Table. The mean of one or more genes per gene set is multiplied with the respective coefficient. The products (mean of genes×coefficient) are then summed to obtain a risk score. The intercept is a constant and never changes. Providing an example for the first two gene sets, AGR2 and AQP3 from the first gene set and ADA and CCNB2 from the second gene set are used. By way of example only, the gene expression measurements are as follows: AGR2: 0.5; AQP3: 1; ADA: 0.25; CCNB2: 0.35. Based on these gene expression measurements, the mean of the genes of the first gene set (AGR2, AQP3) is 0.75 ((0.5+1)/2). The mean of the genes of the second gene set (ADA, CCNB2) is 0.3. The values provided by these two gene sets for the risk score calculation are 0.75×0.00777260484210833 and 0.3×−0.0101018153391127. The values would then be summed together with the values from all other gene sets (calculated using the same method) and the intercept to produce a final risk score. It is determined if the risk score is above/below a certain threshold to determine the appropriate therapy by, for example, one of the following methods: 1) Comparison to a representative background population using percentiles as cut-offs (e.g., values above the 75th percentile are considered high-risk); and 2) If the genes are measured by methods producing absolute values (e.g., qPCR with normalization/standardization against housekeeping genes), an absolute threshold would be used and the value would not necessarily have to be compared to a background population.

If one were to choose only one gene from each gene set, then the gene would be multiplied by its respective coefficient. If one were to choose multiple genes from each gene set, the mean value of the genes corresponding to a specific gene set would be multiplied by the respective coefficient. To determine a high/low score without relating it to percentiles from a background population, one would use any method capable of measuring and normalizing gene expression on a single-sample level. This can be achieved with the qPCR method, where absolute transcript counts are measured and normalized against housekeeping genes. To accomplish this, one would need to perform a qPCR analysis on a sufficiently large cohort (once we have identified the final set of genes to be included in an upcoming study) and set absolute thresholds based on the distribution of values. These thresholds could then be applied to a new cohort on a single sample level (provided exactly the same method is used) without the need to relate the values to a background population. Alternatively, microarrays and the single-channel array normalization (SCAN) method can be used to provide absolute risk scores. If an Affymetrix microarray platform is used on a new population and the SCAN normalization method is used, set absolute thresholds could theoretically be set, which can guide decisions about an appropriate therapy without relating the values to a background population. When using the Final model, age would be factored in. However, this would not be the optimal method in clinical practice, where methods such as qPCR with housekeeping gene would be more appropriate than microarray and the SCAN normalization method.

### Proliferative Index

In this non-limiting example, gene sets from the H (hallmark), C2 (curated gene sets), and C6 (oncogenic signature gene set) categories were chosen as potential features for the tumor-intrinsic model (n=6529 gene sets, which altogether include over 9,000 genes). To identify biological processes that act as effect modifiers of an active immune infiltrate, cox regression analyses, including all tumors (n=7008), were performed with an interaction term between each gene set and the created immunologic signature, Immunescore. A meta-analysis of interaction p-values for all gene sets, selecting the 50 top-ranked gene sets and removing highly correlated processes (rho >0.7 or <−0.7), was performed using the same method as for the Immunescore. A total of 3 gene sets were included in the resulting model, Table 6. Without being bound by theory, it is believed that biological processes associated with tumor aggressiveness could be regarded as effect modifiers of the prognostic effects of a local immune infiltrate. Consequently, to isolate the prognostic and immunomodulatory signal of aggressive tumor-intrinsic pathways, the model was trained within tumors with an Immunescore in the lowest third (n=2312) because the prognostic signal would not be attenuated by an immune infiltrate. The model showed a clear dominance of proliferation-related processes, which, without being bound by theory, is hypothesized to correlate with genomic instability. Therefore, the resulting model is called Proliferative Index, as shown in FIG. 1. In some embodiments, the model is based on a tumor-intrinsic model, the tumor-intrinsic model including genes from tumor-intrinsic gene sets, some of the gene sets and their associated genes are shown in Table 6.

TABLE 6
Selected gene sets and genes used to determine Proliferative Index

| Gene Set | Genes | Coefficient |
|---|---|---|
| (Intercept) | — | −0.733732436115213 |
| SMID_BREAST_CANCER_{—} | ABCA8, ADH1B, ADIRF, | −0.0773241028664877 |
| LUMINAL_A_UP | ADRA2A, AREG, ATP1A2, | |
| | ATRNL1, BTG2, C1orf21, | |
| | CIDEC, CILP, COL14A1, CPA3, | |
| | CXCL14, CYP4B1, DHRS2, DPT, | |
| | DUSP1, ELOVL2, ENPP2, ESR1, | |
| | FABP4, FBLN1, FHL1, G0S2, | |
| | GFRA1, GPD1, HMGCS2, | |
| | HOXB2, IGF1, ITIH5, LAMA2, | |
| | LAMP5, LRRC17, MAOA, | |
| | MAP3K12, MFAP4, MYB, | |
| | NAT1, OGN, OMD, PCSK5, | |
| | PDCD4, PGR, PLIN1, PPARG, | |
| | PTGER3, PTHLH, PTN, RAI2, | |
| | RBP4, RTN1, RUNX1, SCUBE2, | |
| | SFRP4, SLC26A3, STC2, SVEP1, | |
| | TAT, TNN | |
| SOTIRIOU_BREAST_CANCER_{—} | AASS, ANG, BBS1, CFAP69, | −0.22031312657708 |
| GRADE_1_VS_3_DN | CIRBP, CX3CR1, CYBRD1, | |
| (Intercept) | — | −0.733732436115213 |
| | DIXDC1, DYNC2H1, DZANK1, | |
| | ECHDC2, FOS, FRY, IFT46, | |
| | KIF13B, LTBP3, NME5, | |
| | NYNRIN, SESN1, STAT5B, | |
| | SYNC, TP53BP1, WDR19, | |
| | ZNF395 | |
| WP_GASTRIC_CANCER_NETWORK1 | ACTL6A, AURKA, CENPF, | 0.0808874859443737 |
| | CEP192, ECT2, ESM1, KIF15, | |
| | KIF20B, LIN9, MCM4, NOTCH1, | |
| | NUP107, RUVBL1, S100P, | |
| | SMOC2, TOP2A, TPX2, UBE2C | |

TABLE 7
Proliferative Index Model

| Variable | Coefficient |
|---|---|
| (Intercept) | −0.733732436115213 |
| SMID_BREAST_CANCER_{—} | −0.0773241028664877 |
| LUMINAL_A_UP | |
| SOTIRIOU_BREAST_CANCER_{—} | −0.22031312657708 |
| GRADE_1_VS_3_DN | |
| WP_GASTRIC_CANCER_{—} | 0.0808874859443737 |
| NETWORK_1 | |

### Integrated Model

To answer the clinical question of whether the integration of tumor-intrinsic and immunological factors allows the downgrading of high-risk tumors, an Integrated model was created that weighs these dimensions together. This was done by scaling, centering, and calculating each training cohort's Immunescore and Proliferative Index. The cohorts were then merged, and a new model was trained where the included variables were Immunescore. Proliferative Index, and an interaction term according to the expression: (Immunescore+Proliferative Index)\hat{ }2, Table 8. The integrated model was used to stratify patients aged <70 with grade III tumors or patients aged <60 based on the median. This subgroup was chosen as it may be recommended RT boost based on today's guidelines. Without being bound by theory, it is hypothesized that this would allow the identification of a high-risk clinical group that may be omitted RT boost.

TABLE 8
Integrated Model

| Variable | Coefficient |
|---|---|
| (Intercept) | −0.78299480 |
| Immunescore | −0.09315584 |
| Proliferative Index | 0.29267438 |
| Immunescore: Proliferative Index | −0.19197174 |

### Validation Cohorts

The Servant and Sjöström cohorts. The well-annotated publicly available Servant (n=341)[10] and Sjöström (n=172)[11] cohorts include irradiated patients followed for local recurrence, Table 9. These cohorts were used to evaluate the implications of the Immunescore on the risk of local recurrence based on the Proliferative Index of the tumor, among irradiated patients.

TABLE 9
Clinical characteristics of the Sjöström and Servant cohorts

| Variables | | Servant | Sjöström* |
|---|---|---|---|
| ER status | Negative | 75 (22%) | 49 (29%) |
| | Positive | 268 (78%) | 119 (69%) |
| | Missing | 0 (0%) | 4 (2%) |
| Histological | Grade I | 55 (16%) | 16 (9%) |
| grade | Grade II | 147 (43%) | 46 (27%) |
| | Grade III | 137 (40%) | 30 (17%) |
| | Missing | 4 (1%) | 80 (47%) |
| Age (years) | <50 | 327 (95%) | NA |
| | ≥50 | 16 (5%) | NA |
| | Missing | 0 (0%) | NA |
| Lymph node | Negative | 215 (63%) | 125 (73%) |
| status | Positive | 122 (36%) | 35 (20%) |
| | Missing | 6 (2%) | 12 (7%) |
| Chemotherapy | Yes | 133 (39%) | 34 (20%) |
| | No | 209 (61%) | 133 (77%) |
| | Missing | 1 (0.3%) | 5 (3%) |
| Anti-hormonal | Yes | 31 (9%) | 60 (35%) |
| therapy | No | 311 (91%) | 108 (63%) |
| | Missing | 1 (0.3%) | 5 (3%) |
| Local | No event | 224 (65%) | 104 (60%) |
| recurrence | Event | 119 (35%) | 68 (40%) |
| | Missing | 0 (0%) | 0 (0%) |
| | Median follow-up | 10.0 | 13.2 |
| | time no event | | |
| | (years) | | |
| | Median follow-up | 3.3 | 3.7 |
| | time event | | |
| | (years) | | |

*Information of clinical variables were retrieved from the original publication (Sjostrom et al., 2018). Clinical data from the Sjöström data set was not available for the analyses of the present study.

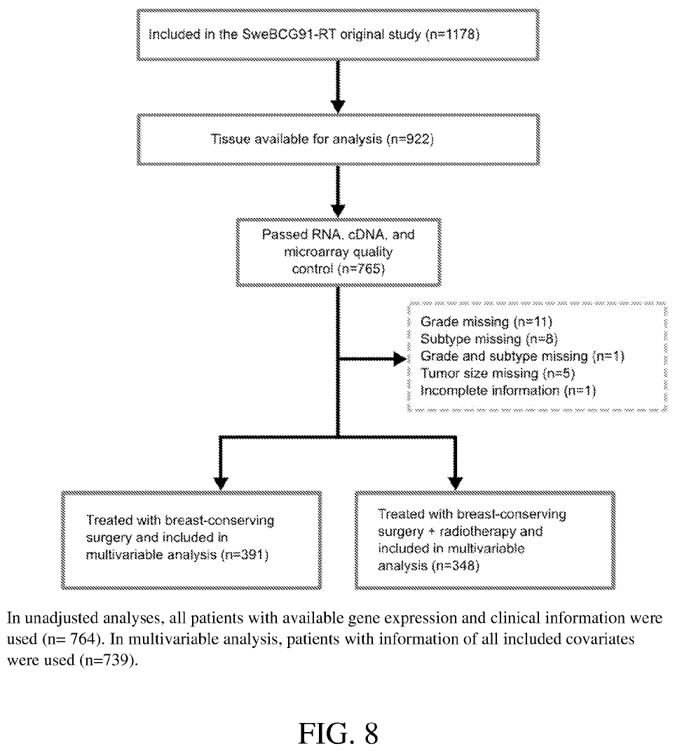

The SweBCG91RT cohort. To understand how the genomic instability of the tumor influences the RT benefit associated with an active immune infiltrate, the randomized SweBCG91RT cohort was used. In summary, 1178 patients with lymph-node negative (NO) stage I or IIA breast cancer were randomly assigned between 1991 and 1997 to breast-conserving surgery with or without whole-breast RT and followed for a median time of 15.2 years, FIG. 8, Table 1. GeneChip Human Exon 1.0 ST Arrays (Thermo Fisher Scientific, South San Francisco, CA) were used to obtain gene expression data (GEO GSE119295). Altogether, 7% of patients received endocrine treatment, 1% received chemotherapy, and 0.4% received both endocrine therapy and chemotherapy. Analyses were performed on treatment naïve tumor samples. Invasive carcinoma was histologically confirmed by a board-certified pathologist. Clinical variables did not differ significantly between included and excluded patients except for tumor size and histological grade. Excluded patients had slightly smaller (median size: 11 mm versus 12 mm) tumors and the tumors were of a lower histological grade compared to included patients, Table 18. Gene expression data have been deposited in the Gene Expression Omnibus under accession number GSE119295. Due to the ethical review board's regulations and laws related to patient privacy, all clinical information has not been made publicly available.

The trial and follow-up study were approved by a Regional Ethical Review Board (approval numbers 2010/127 and 2015/548) and conducted per the declaration of Helsinki.

### Statistical Methods

Time to ipsilateral breast tumor recurrence (IBTR) as the first event within 10 years from the date of diagnosis was used as the primary endpoint. Other recurrences and death were considered competing risks for IBTR. Multivariable regressions in the framework of flexible parametric survival analysis (Stata macro stpm2[12]) were used to estimate Royston-Parmer models and hazard ratios (HR) and to predict the 10-year cumulative incidence of IBTR. Time was measured from the date of randomization (SwcBCG91RT cohort) or the date of diagnosis (Servant and Sjöström cohorts) to an IBTR event, censoring due to a competing event or death, or last follow-up. Analyses were performed with up to 10 years of follow-up. The continuous values of Immunescore, Proliferative Index, and Integrated score were used for interaction analyses [16]. The regression models included time-dependent effects for covariates that did not fulfill the proportional hazards assumption. Interactions were tested by comparing models with and without an interaction term using the likelihood ratio test. Covariates included in the SweBCG91RT cohort were age, ER status, histological grade, and tumor size. The Immunescore and Proliferative Index variables were standardized and rescaled to have a mean of zero and a standard deviation of one. The predicted cumulative incidence for different percentiles of Immunescore and Proliferative Index from the regression models were plotted with 95% confidence intervals. A p-value <0.05 was considered statistically significant. The full models are provided in Table 19. Figures of cumulative incidence were created according to the method of Fine and Gray[14] and based on the multivariable Cox models of subhazards for the different endpoints. P values for the subhazard ratio between compared groups were denoted P_{CIF} in the plots, which were calculated by using a weighted log-rank test as described by Geskus[15](using the sterprep command in Stata). R version 4.1.2 and Stata/MP 17.0 for Mac were used for the statistical analysis.

### Results

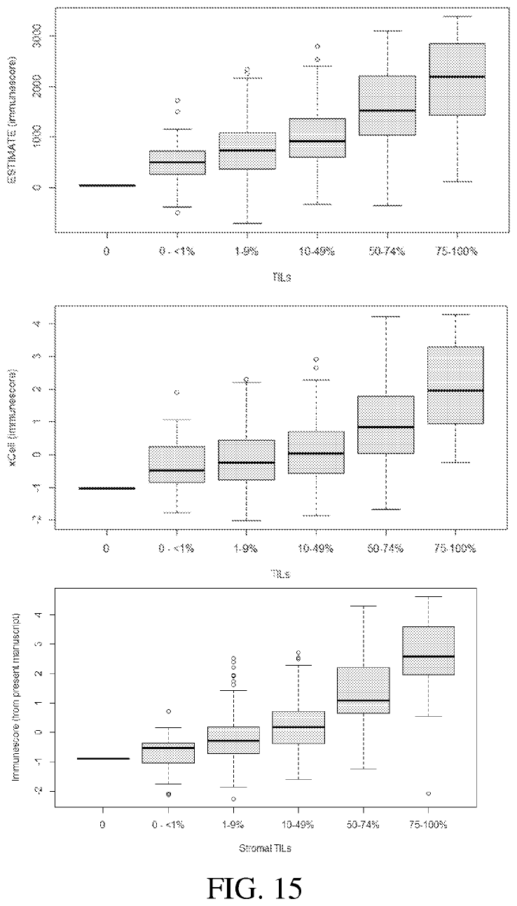

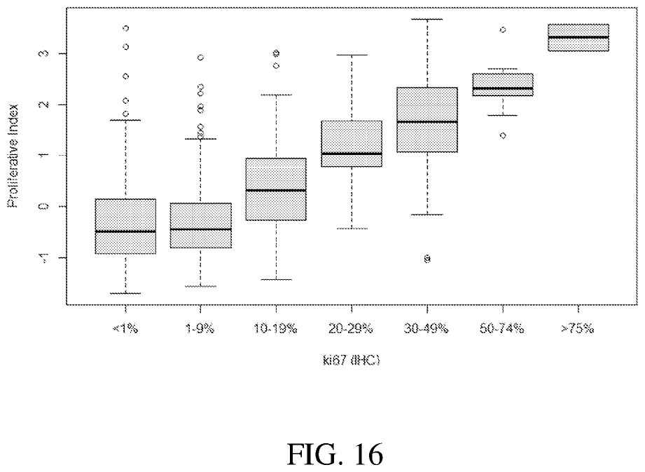

Correlations. Immunescore correlated with TILs (rho 0.42, p<0.001), FIG. 15. The Y-axis is standardized to have a mean of 0 and a standard deviation of 1. ESTIMATE and xCell exhibited correlations with TILs of 0.33 (p<0.001), and 0.27 (p<0.001), respectively. Furthermore, Immunescore correlated with histological grade (rho 0.25, p<0.001), and was inversely correlated with ER status (rho −0.26, p<0.001) and age (rho −0.076, p=0.038), Table 10. As shown in FIG. 15, Immunescore showed a stronger correlation with tumor-infiltrating lymphocytes than other developed methods to quantify the degree of immune infiltration. Also, Proliferative Index has been shown to correlate with Ki67 in the SweBCG91RT cohort, FIG. 16. Ki67 levels were evaluated by a board-certified pathologist on TMAs.

The Immunescore and Proliferative Index correlated (rho 0.23, p<0.001) and were generally enriched among tumors with unfavorable characteristics. Immunescore correlated with TILs (rho 0.36, p<0.001) and histological grade (rho 0.25, p<0.001). Tt was inversely correlated with ER status (rho −0.26, p<0.001) and age (rho −0.076, p=0.038), Table 10. Proliferative Index showed similar correlations with TILs (rho 0.40, p<0.001), histological grade (rho 0.55, p<0.001) and ER status (rho −0.46, p<0.001), Table 10.

TABLE 10
Spearman correlation table between PI, IS, and clinical variables

| | | | Tumor- | | | | |
|---|---|---|---|---|---|---|---|
| | Proliferative | | infiltrating | Histological | Tumor | ER | |
| Variables | Index | Immunescore | lymphocytes | grade | size | status | Age |
| Proliferative | 1.0 | 0.2339* | 0.4003* | 0.5471* | 0.2139* | −0.4636* | 0.0218 |
| Index | | | | | | | |
| Immunescore | 0.2339* | 1.0 | 0.3615* | 0.2553* | 0.0294 | −0.2554* | −0.0761 |
| TILs | 0.4003* | 0.3615* | 1.0 | 0.3571* | 0.2137* | −0.3113* | −0.1065* |
| Histological | 0.5471* | 0.2553* | 0.3571* | 1.0 | 0.2730* | −0.3956* | −0.0270 |
| grade | | | | | | | |
| Tumor size | 0.2139* | 0.0294 | 0.2137* | 0.2730* | 1.0 | −0.1374* | −0.1350* |
| ER status | −0.4636* | −0.2554* | −0.3113* | −0.3956* | −0.1374* | 1.0 | 0.0495 |
| Age | 0.0218 | −0.0761 | −0.1065* | −0.0270 | −0.1350* | 0.0495 | 1.0 |

*P < 0.05

Prognostic implications of the interplay between immune activity and tumor aggressiveness for IBTR. Proliferative Index was associated with an unfavorable prognosis in unadjusted analysis in the Sjöström (HR 1.98, CI 95% 1.49-1.62, p<0.001), Servant (HR 1.30, CI 95% 1.08-1.57, p=0.006) and SweBCG91RT (HR 1.36, CI 95% 1.14-1.63, p=0.001) cohorts, Table 11. The significance remained in the Sjöström cohort with adjustment for subtype (HR 1.60, CI 95% 1.01-2.54, p=0.04) and in the SweBCG91RT cohort with adjustment for RT, ER status, histological grade, age, and tumor size (HR 1.32, CI 95% 1.03-1.70, p=0.031) but not in the Servant cohort with adjustment for age and subtype (HR 0.96, CI 95% 0.69-1.32, p=0.791).

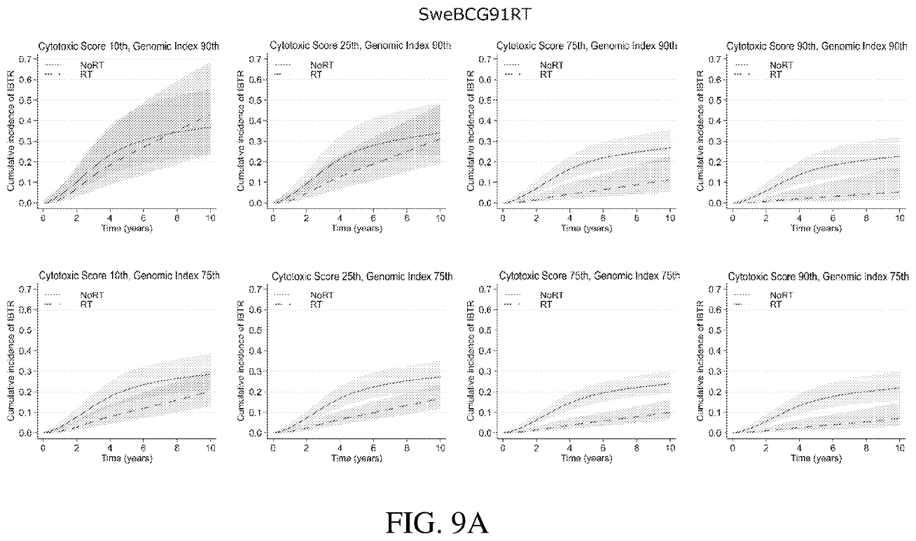

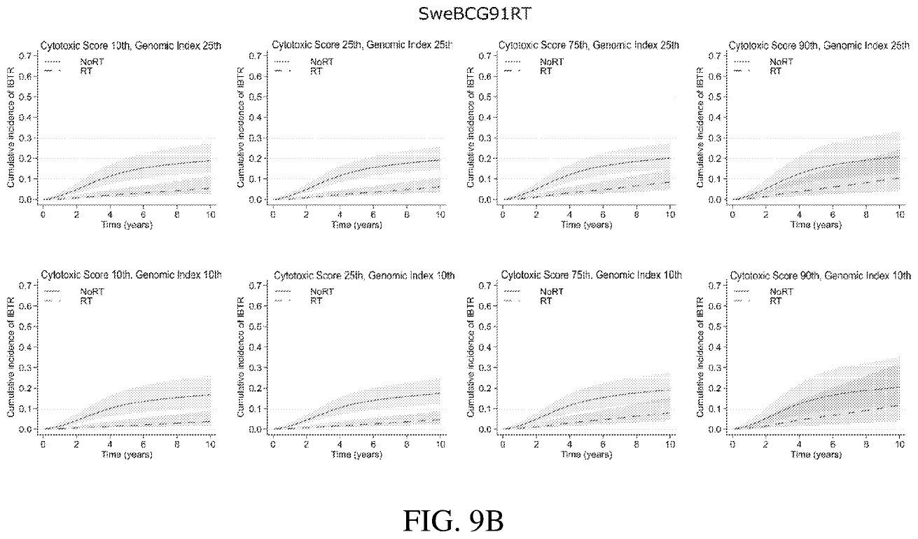

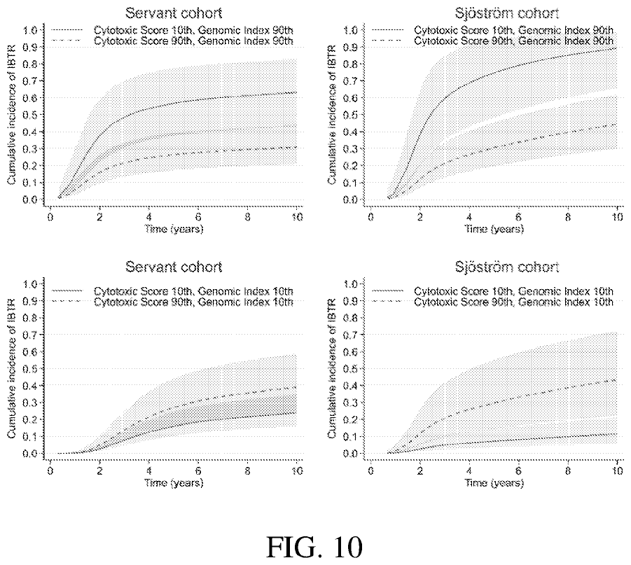

The prognostic effect of the Immunescore varied depending on Proliferative Index, FIGS. 9A, 9B, and 10. Among tumors with a low Proliferative Index, an increased Immunescore was associated with a less favorable prognosis. The opposite was seen for tumors with a high Proliferative Index, FIGS. 9A, 9B, and 10, and Table 11. As shown in FIG. 9A, tumors with a high Proliferative Index and a low Immunescore had the highest risk of IBTR. An unadjusted interaction test between Immunescore and Proliferative Index to IBTR was significant in the Sjöström (HR 0.61, CI 95% 0.47-0.81, p<0.001), Servant (HR 0.78, CI 95% 0.65-0.95, p=0.018) and SweBCG91RT (HR 0.78, CI 95% 0.64-0.95, p=0.013) cohorts, which is shown in Table 11. The significance remained in the Sjöström cohort with adjustment for subtype (HR 0.63, CI 95% 0.47-0.84, p=0.002), in the Servant cohort with adjustment for subtype and age (HR 0.77, CI 95% 0.62-0.96, p=0.021) and the SweBCG91RT cohort with adjustment for RT. ER status, histological grade, age and tumor size (HR 0.75, CI 95% 0.61-0.94, p=0.012).

TABLE 11
Unadjusted and adjusted flexible parametric survival analysis with Royston-
Parmar (RP) regression models of local recurrence (IBTR) within 10 years
in the Sjöström, Servant, and SweBCG91RT cohorts

| | No adjustment for | | | Adjustment for | | |
|---|---|---|---|---|---|---|
| | other covariates | | | other^{#} covariates | | |
| | HR | 95% CI | p | HR | 95% CI | p |
| Sjöström (n = 172) | | | | | | |
| Immunescore | 1.14 | 0.83-1.55 | 0.418 | 1.03 | 0.73-1.45 | 0.878 |
| Proliferative Index | 1.98 | 1.49-2.62 | <0.001 | 1.61 | 1.01-2.56 | 0.044 |
| Proliferative Index × | 0.61 | 0.47-0.81 | <0.001 | 0.63 | 0.47-0.84 | 0.002 |
| Immunescore | | | | | | |
| Servant (n = 343) | | | | | | |
| Immunescore | 0.97 | 0.80-1.18 | 0.768 | 0.86 | 0.69-1.07 | 0.178 |
| Proliferative Index* | 1.46 | 1.20-1.78 | <0.001 | 1.06 | 0.76-1.48 | 0.716 |
| Proliferative Index × | 0.79 | 0.65-0.96 | 0.018 | 0.78 | 0.63-0.97 | 0.029 |
| Immunescore* | | | | | | |
| SweBCG91RT total | | | | | | |
| cohort (n = 764) | | | | | | |
| Immunescore | 0.91 | 0.72-1.15 | 0.437 | 0.81 | 0.64-1.04 | 0.096 |
| Proliferative Index | 1.36 | 1.14-1.63 | 0.001 | 1.32 | 1.03-1.70 | 0.031 |
| Proliferative Index × | 0.78 | 0.64-0.95 | 0.016 | 0.75 | 0.61-0.94 | 0.012 |
| Immunescore | | | | | | |
| RT vs no RT | 0.41 | 0.27-0.61 | <0.001 | 0.41 | 0.28-0.62 | <0.001 |

*The proportional hazards assumption was not fulfilled for Proliferative Index in the Servant cohort. Therefore, time-dependence för Proliferative Index was allowed using splines.
^{#}For the Sjöström cohort, adjustment for subtype was performed.
^{#}For the Servant cohort, adjustment for age and subtype was performed.
^{#}For the SweBCG91RT cohort, adjustment for age, histologic grade, tumor size, and ER status was performed.

Radiotherapy benefit. To investigate how the benefits of RT varied depending on Immunescore and Proliferative Index, the SweBCG91RT cohort was then analyzed. Patients were stratified based on RT, and a model with an interaction term between Proliferative Index and Immunescore was created for irradiated (p_{interaction}=0.013) and unirradiated (p_{interaction}=0.17) patients, Table 11. The prognosis of irradiated and unirradiated patients at different quantiles of Proliferative Index and Immunescore was then compared, as shown in FIGS. 9A and 9B. It was determined that highly aggressive tumors could be stratified based on immune infiltrates regarding RT benefit. Accordingly, tumors with a Proliferative Index in the highest quartile were studied. Tumors with a high Proliferative Index and a low Immunescore had an increased risk of IBTR regardless of RT treatment, and the benefit from RT was reduced, FIG. 9A. In total, 10.9% of irradiated patients with tumors in the highest quartile of Proliferative Index and an Immunescore above the median suffered an IBTR within 10 years, FIG. 9A. This can be contrasted against tumors with a Proliferative Index in the highest quartile but an Immunescore below the median, where 30.3% of irradiated patients had an IBTR, as shown in FIG. 9A. Aggressive tumors with a high Immunescore also appeared to benefit more from RT than high-risk tumors with a low Immunescore, FIGS. 9A and 9B. Among tumors with a low Proliferative Index, Immunescore was not associated with a favorable prognosis, FIG. 9B. The largest benefit from RT among these tumors was seen with a low Immunescore, FIG. 9B. As shown in FIGS. 9A and 9B, in some embodiments, the ranges of the thresholds for IS and PI scores would be: threshold where a score below indicates a low score—10th percentile≤x<25th percentile; a lower (x) and an upper (y) threshold where a score within the range indicates a medium score—25th percentile≤x<y<75th percentile; threshold where a score above indicates a high score—75th percentile≤x≤90th percentile. In some embodiments, the ranges of the thresholds of the IS and PI scores would be: threshold where a score below indicates a low score—x<25th percentile; a lower (x) and an upper (y) threshold where a score within indicates a medium score—25th percentile≤x<y<75th percentile; threshold where a score above indicates a high score—75th percentile≤x. In some embodiments, the ranges of the thresholds of the IS and PI scores would be: threshold where a score below indicates a low score—10th percentile≤x<40th percentile; threshold where a score within indicates a medium score—30th percentile≤x<70th percentile; threshold where a score above indicates a high score—60th percentile≤x≤90th percentile. In some embodiments, if patients with grade III tumors serve as a representative background population, the ranges of the scores of the IS and PI would be: threshold where a score below indicates a low score—10th percentile≤x<33th percentile; threshold where a score within indicates a medium score—33th percentile≤x<67th percentile; threshold where a score above indicates a high score—67th≤x≤90th percentile. In some embodiments, the ranges of the scores of the IS and PI scores would be: low score—0th percentile≤x<25th percentile; medium score—25th percentile≤x<75th percentile; high score—75th percentile≤x≤100th percentile.

Identification of patients with high-risk tumors that can be omitted from the RT boost. A model was then used that integrates the Immunescore and Proliferative Index, called the Integrated model as shown in Table 8, to try to downgrade high-risk tumors in terms of necessary treatment intensity. As shown in Table 8, the model contains an interaction term between the Immunescore and the Proliferative Index. The coefficient of the interaction term is negative, which means that among tumors with a high Proliferative Index, a high value on the Immunescore can downgrade the predicted probability of recurrence. The highest predicted risk of recurrence is therefore observed in tumors with a high Proliferative Index and low Immunescore. Patients from the SweBCG91RT cohort were divided into groups with high or low scores based on the median score as shown in FIGS. 11A and 11B. Patients aged 51-70 with grade III tumors and patients aged <60 years were then selected. High-risk patents are defined as those aged 51-70 with grade III tumors or aged <60 years with any histological grade. The distribution of subtypes in the different tertiles can be seen in Table 24. All subtypes were represented in the different textiles, and Luminal B was the subtype most equally distributed. These patients may be recommended an RT boost according to current guidelines. Additional clinical considerations for patients with a high Proliferative Index and a low Immunescore includes escalated therapy. One example already mentioned is the RT boost. Other escalated therapies include dose-dense chemotherapy, antibody drug conjugates, and prolonged endocrine treatment. As shown in FIGS. 11A and 11B, the low-score group had a 10-year incidence of IBTR of 17.3% (95% CI: 12.2-26.9) without RT and 7.9% (CI 95% 4.2-14.6) with RT, and appeared to benefit from standard RT (SHR 0.39, CI 95% 0.18-0.85, p=0.015). The high-score group had a 10-year IBTR incidence of 25.7% (CI 95% 18.9-34.3) unirradiated and 16.2% (CI 95% 10.3-25.1) irradiated, and had a reduced benefit from RT (SHR 0.64, CI 95% 0.32-1.03, p=0.064). For FIGS. 11C, 11D, and 11E, patients were divided into three groups using tertiles as cut-offs instead of the median score shown in FIGS. 11A and 11B. Using tertiles as cut-offs allows for a dose-dependent gradient to be observed.

Patients from the lowest tertile had a 10-year incidence of IBTR of 17.1% (95% CI: 10.3-27.7) without RT and 5.4% (CI95% 2.1-13.7) with RT, and appeared to benefit from standard RT (SHR 0.29, CI 95% 0.10-0.87, p=0.028), FIGS. 11C, 11D, and 11E. Patients within the medium tertile had a 10-year IBTR incidence of 20.3% (CI 95% 12.8-31.4) unirradiated and 9.8% (CI 95% 4.5-20.5) irradiated, and a non-significant benefit from RT (SHR 0.42, CI 95% 0.17-1.07, p=0.069). The group in the highest tertile was most likely to suffer an IBTR, with a 10-year incidence of 28.7% (CI 95% 20.3-39.7) without RT and 19.5% (CI 95% 12.2-30.3) with RT. This group did not significantly benefit from RT (SHR 0.61, CI 95% 0.32-1.15, p=0.13).

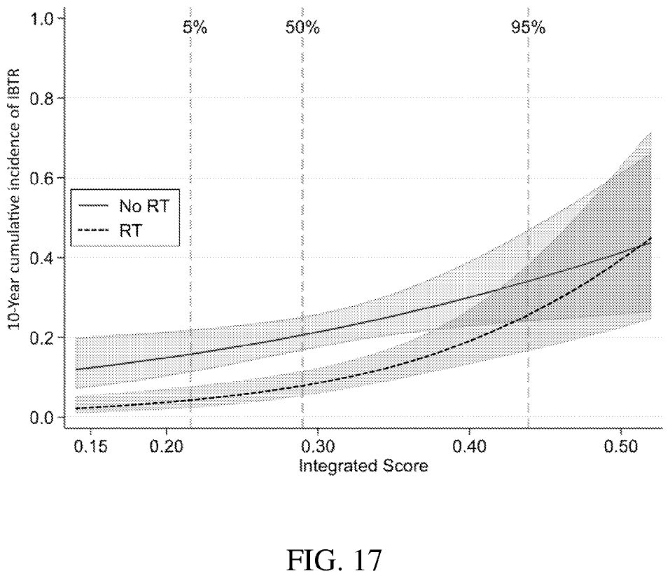

To investigate if the Integrated model was predictive of RT benefit, an interaction analysis in the whole SweBCG91RT cohort was performed. The interaction was significant in an unadjusted analysis (p=0.004) and an analysis adjusted for histological grade, age, ER status, and tumor size (p=0.008), see Table 22 and FIG. 17. As shown in FIG. 17, the predicted 10-year cumulative incidence of IBTR with 95% confidence intervals stratified by proposed menopausal status, by Integrated model score based on Royston-Parmar models with estimates from Table 22. The stpm2 macro for Stata was used for the calculations. The dotted vertical lines represent percentiles of the Integrated score.

Final Model with a Reduced Number of Genes

Finally, patient age was integrated into the model by retraining Integrated model in the Servant cohort using the following expression: Integrated model+patient age. An example of which is shown in Table 12. To improve the clinical utility of the model, the number of genes included were then reduced as illustrated by FIG. 2. Stably expressed genes across different samples including, but not limited to, tumor cores and tissue types (formalin-fixed paraffin-embedded and fresh-frozen cores) were selected and used. This allowed for the creation of what is called the Final Reduced Model in FIG. 2. The ability of the model to separate patients based on prognosis and radiotherapy benefit can be illustrated by FIGS. 6 and 7. The Final Reduced Model allows for an improved individualization of breast cancer therapy by classifying patients into different risk groups. The various risk groups derived from the Final Reduced Model are summarized in Table 13.

If age is considered as a separate prognostic variable to identify high-risk groups (similarly to histological grade III or ER negativity, etc., as indicated in Table 13), then the age of 60 would a reasonable highest threshold to identify a high-risk group of young patients, and somewhere around 40 years would be a suitable threshold on the lower end of the range (i.e., <60, <55, <50, <45, <40 can be used as thresholds to identify young patients). Even if age is used to define high-risk groups, the final model will still distinguish between, relatively speaking, younger and older patients. For example, the final model ran on a high-risk group of patients below 55 years, patients aged 40 years will be classified as being of higher risk than patients aged 45 years. The same principle would apply for older patients. Low-risk groups can be defined as having ages above 60-75 (any threshold in between is possible). While setting thresholds for age might be relevant if age is used as a prognostic variable to identify high-/low-risk groups, such thresholds are not necessary for the final model itself because age is included as a continuous variable in the final model. Therefore, no thresholds need to be defined for the final model to classify patients into different risk groups.

TABLE 12
Final Model
Final model

| | |
|---|---|
| (Intercept) | 0.85423832 |
| Patient age | −0.03471772 |
| Integrated model | 0.19541174 |

Immunological and Tumor-Intrinsic Signature Outcomes with Respect to Menopausal Status

Given that premenopausal patients more often have highly proliferative and/or ER-negative tumors, it follows that they generally derive a greater prognostic benefit from an immune infiltrate. Identifying immune responsive tumors also among postmenopausal patients is possible, which is demonstrated by additional adjusted analyses. In Table 23, patients are stratified by proposed menopausal status (the age of 55 was used as cut-off). For women <55 years, the combined immune-tumor cell proliferation signature (called Integrated model/score) and RT violated the proportional hazards assumption. Therefore, time-varying coefficients (tvc) were included for these variables. For patients >=55 years, only RT and Integrated score were significant and included in the final model. In summary, the Integrated score is prognostic in both groups of patients.

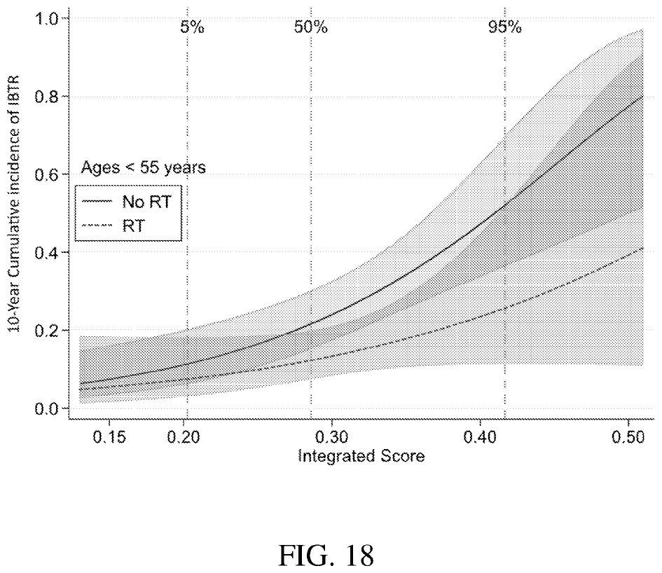

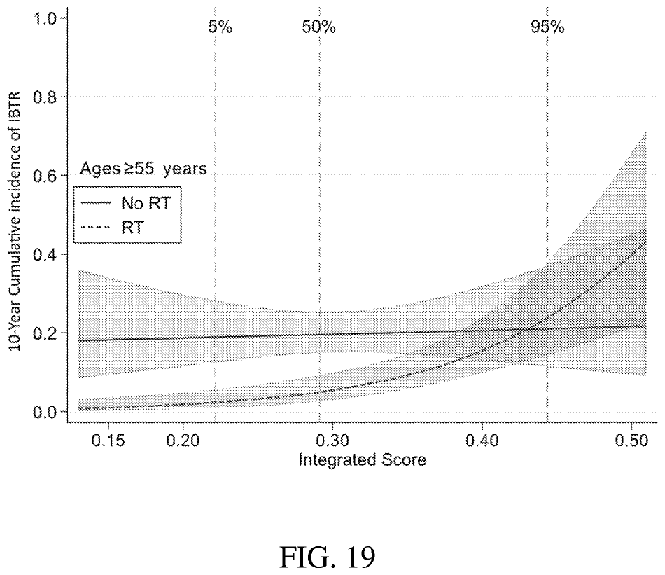

FIGS. 18 and 19 predicted 10-year cumulative incidence with 95% confidence intervals stratified by proposed menopausal status, by Integrated model/score based on Royston-Parmar models with estimates from Table 23. The stpm2 macro for Stata was used for the calculations. The dotted vertical lines represent percentiles of the Integrated score within the respective menopausal groups. It appears that a prognostic effect dominates among younger women (<55 years of age, FIG. 18), while a predictive effect may dominate among older women (≥55 years of age, FIG. 19). Furthermore, FIG. 18 for premenopausal patients supports the conclusion that not all need an RT boost despite a young age. In FIG. 18, the younger women (<55 years of age) with <5th percentile of the Integrated Score would be recommended a de-escalation of RT (omission or shorter course/lower dose, e.g., IORT); those with >50th percentile of the Integrated Score would be recommended an escalation of RT compared to standard RT (i.e., RT+boost); those >95th percentile would be recommended for surgical escalation (mastectomy, wider resection margins) and/or systemic therapy escalation. In FIG. 19, the older population of women (≥55 years of age) with >70^{th} percentile of the Integrated Score would be recommended RT escalation (e.g., RT+boost) and those with >85th percentile of the Integrated Score would be recommended escalation to mastectomy or escalation of systemic therapy as shown in FIG. 30. The same or substantially the same thresholds described here for FIGS. 18 and 19 would likely apply to the Final Score.

Further Age-stratified Analyses. As shown in Table 12A, estimates remain similar in different age groups, meaning the methods should work well among both younger (<50 years old) and older patients (>65 years old). The interaction effect between immunological (TILs, immunescore, etc.) and tumor-intrinsic variables (Proliferative Index, histological grade, Ki67, etc.) may be strongest among younger individuals. Due to the strong association with distant metastasis in the group aged 65 or older, reliable analyses with local recurrences as endpoint need further refinements because patients are censored for local recurrences when they develop a distant metastasis. Therefore, if a variable strongly increases the risk of distant metastasis, it may wrongly give the impression of also reducing the risk of local recurrences. The strong association between Proliferative Index, Integrated, and Final scores and distant metastasis among patients >=65 years of age further shows that these can be used as improved alternatives to the commonly used 21-gene recurrence score or 70-gene score to determine the need for chemotherapy among such patients. In Table 12B, the performance of Proliferative Index, the Integrated model, and the Final model remain superior to the 21-gene recurrence score and the 70-gene score in an analysis of patients aged >=65 years with ER-positive tumors from the SweBCG91RT cohort.

While 50 and 65 years of age are clinically useful and common endpoints for various clinical studies, treatment plans, and interventions, endpoints for under 50 and over 65 years of age remain less studied. Typically, patients under 50 years of age are treated with RT boost after receiving a diagnosis of cancer from their tumor biopsy. However, there is a severe problem with the assumption that patients under 50 years of age being diagnosed with cancer should be defaulted into RT boost treatment. However, when integrated into the model by retraining Integrated model in the Servant cohort using the following expression: Integrated model+patient age, the Final Reduced Model allows for an improved individualization of breast cancer therapy by classifying patients in this age group into different risk groups. Rather than having all of the patients under 50 years of age receive RT boost after a cancer diagnosis, the ≤50 year old group can be further classified into different risk groups as shown in FIGS. 30 and 31, which improves individualization of breast cancer therapy.

With respect to systemic therapy shown in FIG. 30, different risk assessments may be performed for <50 and >=65 year old patients such as tumor-intrinsic aggressivity and immune infiltrate, or Integrated/Final Score. If the risk assessment finds a high-tumor intrinsic risk and absence of activated immune infiltrate (with optional integration of additional clinical variables (e.g., lymph node status, tumor size, lymphovascular invasion, age), preferably in borderline cases), the treatment plan includes chemotherapy intensification and/or immunotherapy omission. Here, the high tumor-intrinsic risk includes any or a combination of the following: 1. Proliferative Index above a threshold above the 60th percentile compared to a background population of representative tumors and patients; 2. Histological grade 3 or histological grade 2 and a Proliferative Index equal to or above the median of a background population of grade 3 tumors; 3. HER2-amplification; 4. High Ki67 (e.g., above a threshold higher than 20% scored as a global assessment); 5. ER-negative tumor. Also, Integrated or Final score above a threshold above the 60th percentile of a representative background population of patients and tumors. If the risk assessment finds a high tumor-intrinsic risk and activated immune infiltrate, or a high tumor-intrinsic risk and a low Integrated/Final Score, then the treatment plan includes immunotherapy (regardless of the subtype) and/or chemotherapy de-escalation or omission. If the risk assessment finds a low tumor-intrinsic risk, then the immune infiltrate is assessed treatment plan includes immunotherapy (regardless of the subtype) and/or chemotherapy de-escalation or omission. As shown, multiple types of assessments may be provided to assess treatment options for those outside of the more common 50 to 65 years old group.

Further, similar can be said of the radiotherapy flow diagram shown in FIG. 31, where different risk assessments are used to determine at least the tumor-intrinsic risk from the tumor biopsy to determine a treatment plan that includes radiotherapy. As shown, different risk assessments may be performed for <50 and >65 year old patients such as tumor-intrinsic aggressivity and immune infiltrate, or Integrated/Final Score. If the risk assessment finds a high-tumor intrinsic risk and absence of activated immune infiltrate (with optional integration of additional clinical variables (e.g., lymph node status, tumor size, lymphovascular invasion, age), preferably in borderline cases), the treatment plan includes intensification of the radiotherapy treatment. Here, the high tumor-intrinsic risk includes any or a combination of the following: 1. Proliferative Index above a threshold above the 60th percentile compared to a background population of representative tumors and patients; 2. Histological grade 3 or histological grade 2 and a Proliferative Index equal to or above the median of a background population of grade 3 tumors; 3. HER2-amplification; 4. High Ki67 (e.g., above a threshold higher than 20% scored as a global assessment); 5. ER-negative tumor. Alternatively, Integrated or Final score above a threshold above the 60th percentile of a representative background population of patients and tumors. If the risk assessment finds a high tumor-intrinsic risk and activated immune infiltrate, or a high tumor-intrinsic risk and a low Integrated/Final Score, then the treatment plan includes any of or a combination of radiotherapy de-escalation or omission, preoperative radiotherapy, preoperative systemic therapy, and/or omission or de-escalation of regional nodal irradiation regardless of positive lymph node status. If the risk assessment finds a low tumor-intrinsic risk, then the immune infiltrate is assessed. If the immune infiltrate is absent, then radiotherapy omission will be recommended if other low-risk clinical features are present (e.g., lymph node-negativity, older age (e.g., >=65), small tumor (e.g., <20 mm)), endocrine therapy). If the immune infiltrate is activated, the standard radiotherapy will be planned. As shown, multiple types of assessments may be provided to assess treatment options for those outside of the more common 50 to 65 years old group.

TABLE 12A
Age-stratified analyses

| | 50 years or younger | 50 years or younger | 65 years or older |
|---|---|---|---|
| | (Servant)* | (SweBCG91RT)* | (SweBCG91RT) |
| Variable | HR [CI 95%], P | HR [CI 95%], P | HR [CI 95%], P |
| Proliferative | 1.16 [0.98-1.37], | 1.20 [0.92-1.58], | 1.92 [1.29-2.86], |
| Index | p = 0.087 | p = 0.18 | p = 0.001** |
| Integrated | 1.31 [1.11-1.53], | 1.47 [1.08-2.0], | 1.75 [1.26-2.42], |
| score | p < 0.001 | p = 0.015 | p < 0.001** |
| Final | 1.46 [1.25-1.72], | 1.43 [1.0-2.0], | 2.88 [1.52-5.45], |
| score | p < 0.001 | p = 0.051 | p = 0.001** |
| Proliferative | 0.78 [0.65-0.95], | 0.70 [0.50-0.98], | 1.24 [0.79-1.94], |
| Index × | p = 0.014 | p = 0.040 | p = 0.36** |
| Immunescore | | | |
| Grade III | NA | 0.70 [0.21-2.34], | 0.70 [0.078-6.29], |
| activated vs non- | | p = 0.57 | p = 0.75* |
| activated immune | | | |
| infiltrate | | | |
| Grade I-II | NA | 3.46 [1.22-9.77], | 1.43 [0.34-5.95], |
| activated vs non- | | p = 0.019 | p = 0.62* |
| activated immune | | | |
| infiltrate | | | |
| Grade × activated/non- | NA | 4.77 [0.98-23.3], | 2.02 [0.15-27.5], |
| activated immune | | p = 0.053 | p = 0.60* |
| infiltrate | | | |
| 21-gene recurrence | NA | NA | 1.37 [0.84-2.26], |
| score | | | p = 0.21*** |
| 70-gene score | NA | NA | 1.66 [1.12-2.45], |
| | | | p = 0.011*** |

*Local recurrence (IBTR) used as endpoint
**Distant metastasis used as endpoint due to competing risk problems in patients of age 65 and older

TABLE 12B
Risk of distant metastasis in patients >=65 years
with Luminal A or Luminal B tumors in the SweBCG91RT
cohort comparing Proliferative Index, the Integrated
model, and the Final model to established methods

| Method | HR [CI 95%], P |
|---|---|
| Proliferative Index | 1.99 [1.35-2.93], p < 0.001 |
| Integrated score | 1.75 [1.27-2.41], p < 0.001 |
| Final score | 2.93 [1.57-5.47], p < 0.001 |
| 21-gene recurrence score | 1.51 [0.91-2.51], p = 0.11 |
| 70-gene score | 1.69 [1.16-2.47], p = 0.006 |

TABLE 12C
Additional considerations for patients >=65 years

| Group | Recommended Treatment |
|---|---|
| High tumor-intrinsic risk + absent immune | RT boost (generally not given to older patients). |
| activation or high Integrated/Final score | |
| No co-morbidity + high tumor-intrinsic risk + | Chemotherapy omission or de-escalation |
| activated immune infiltrate or low Integrated/Final | (chemotherapy would be recommended to patients |
| score + activated immune infiltrate | with high tumor-intrinsic risk) and/or |
| | immunotherapy. Also, endocrine therapy if ER- |
| | positive |
| No co-morbidity + low tumor-intrinsic risk + absent | RT omission, chemotherapy omission or de- |
| immune infiltrate or low Integrated/Final score + | escalation, give endocrine therapy.* This group |
| absent immune infiltrate | probably does not differ from current guidelines. |
| No co-morbidity + low tumor-intrinsic risk + | RT (would have been omitted according to current |
| activated immune infiltrate or low Integrated/Final | guidelines) and/or chemotherapy escalation (would |
| score + activated immune infiltrate | have been omitted according to current guidelines), |
| | give endocrine therapy. |
| Co-morbidity | Chemotherapy omission and RT-de-escalation |
| | (boost --> standard or standard --> omission)* |

*Group probably does not differ from current guidelines

While the same thresholds in terms of representative background populations can be used as stated throughout, the representative background population of patients and tumor can be an age-matched group instead of a general group that includes all age groups. In other words, for patients >=65 years, the representative background population of tumors would include only patients >=65 years. The same could be said for patients <50 years and 50-64 years. The recommended options for escalation/standard/de-escalation can then depend on the age group. As provided by Table 12C, for patients of >=65 years of age (any of or a combination of the listed alternatives), Escalation RT: Standard RT. RT boost; Escalation systemic therapy: Standard chemotherapy or dose-dense chemotherapy; De-escalation RT: Omission; De-escalation systemic therapy: Omission of chemotherapy. For patients of <50 years of age (any of or a combination of the listed alternatives), Escalation RT: RT boost, wider surgical resection margins, intensified systemic therapy; Escalation systemic therapy: dose-dense chemotherapy, longer duration of chemotherapy, ovarian suppression (as an escalated alternative to endocrine therapy for ER-positive tumors); De-escalation RT: Omission of RT boost or complete omission of RT; and De-escalation systemic therapy: Standard chemotherapy, shorter duration of chemotherapy or omission of chemotherapy. Escalation systemic therapy for >=65 years of age may mean going from no chemotherapy to some kind of chemotherapy treatment. For Table 12C, some examples of co-morbidities include (but are not limited to) coronary heart disease, heart failure, chronic obstructive pulmonary disease, previous stroke (ischemic or hemorrhagic), uncontrolled hypertension, diabetes mellitus, osteoporosis, one or more cancers other than breast cancer, or a combination thereof.

In the above non-limiting examples, an integrated assessment of immunological biomarkers and tumor-intrinsic factors can enable the downgrading of RT treatment in early-stage highly aggressive tumors—a group that research on de-intensification of RT has not focused on so far. The data presented in the non-limiting examples suggest that this may be possible, paving the way for continued individualization of postoperative RT treatment in breast cancer.

The key mechanism for immunological tumor rejection is activating neoantigen-specific T cells. However, only a minority of tumor mutations give rise to neoantigen-specific T-cell responses. Consequently, with a higher neoantigen load, genomically unstable tumors are associated with a higher probability of being infiltrated by an activated antitumor immune response and of benefit from immunotherapy in several cancers. The signature designed to measure tumor-intrinsic qualities, which is called Proliferative Index, included gene sets capturing ER status and histological grade, and correlated strongly with proliferation. Without being bound by theory, these characteristics are associated with tumor mutational burden (TMB) and, consequently, likely the number of available immunogenic neoantigens. Therefore, an association between Proliferative Index and TMB may at least, in part, explain the above data.

Aggressive tumor characteristics may be associated with a more immunogenic tumor microenvironment. A high proliferation rate increases replication stress. This results in the accumulation of genomic stress resulting in a buildup of DNA in the cytosol, activating the cGAS-STING pathway, which is central to the activation of antigen-presenting cells. Without being bound by theory, an association between tumor proliferation and a favorable tumor microenvironment for T cell activation, not limited to TMB, is another plausible explanation for the data. Proliferation has previously been hypothesized to determine the significance of the immune infiltrate, which is in line with the presented data.

An active immune response in the primary tumor appears to be associated with an improved prognosis and a lower need for RT. The data here shows that these findings primarily apply to patients in high-risk clinical groups. Despite highly aggressive characteristics, no RT boost, and a low frequency of systemic therapy, these patients have a 10-year IBTR risk of well below 10% with standard RT treatment if they have an active immune infiltrate. In contrast, aggressive tumors without an immune infiltrate constitute the group with the highest risk of IBTR, and these patients may also derive the least benefit from RT. These tumors are enriched for immunosuppressive mechanisms, and treatments targeted at immunosuppressive signaling pathways and radiosensitization, e.g., with TGFβ inhibitors, may be an appropriate strategy for eradicating residual tumor cells after surgery. Given that tumor aggressiveness and immune infiltration are predictive of checkpoint inhibitor therapy and chemotherapy, the prognosis in clinical high-risk groups with and without an effective immune response in the modern setting diverges even more. This further strengthens the indication for individualized RT treatment based on immunological biomarkers among patients with aggressive tumors. Other approximations of immune activation and tumor proliferation may provide equally useful information for treatment individualization. Therefore, future studies should investigate if integrating biomarkers already used in clinical practice, such as TILs, immune checkpoint receptor expression, Ki67, and histological grade, allows for an improved treatment individualization of high-risk tumors. Several prospective studies have been conducted regarding the individualization of RT in breast cancer, but these have mainly focused on de-escalation in clinically low-risk groups where the absolute benefit of RT is limited by the favorable prognosis. Low Proliferative Index scores most likely reflect this group, and there are no signs of a favorable immunological effect found here. Instead, tendencies towards the opposite were seen, in line with other studies that associate immune infiltrates with poorer prognosis in clinical low-risk groups. This indicates that prerequisites for immune activation may be lacking among slowly proliferating tumors. Furthermore, immune infiltration in low-risk groups may represent dysfunctional inflammation or correlate with unfavorable tumor-intrinsic characteristics, warranting caution regarding the omission of RT among such patients. The less aggressive subgroup of tumors, as represented by a low Proliferative Index, explains the findings of an increased RT benefit among immune-depleted tumors. The methods of this study were based on assumptions from previous literature to analyze how tumor-intrinsic and immunological factors interact regarding IBTR risk and RT benefit. The examples include large training material and three independent validation cohorts.

The randomized setting of SweBCG91RT makes it a suitable cohort for investigating the effect of RT based on biomarkers. However, several aspects should be taken into consideration. The SweBCG91RT cohort is underpowered to demonstrate differences in RT effect along the two axes of tumor-intrinsic factors and immune activation. Several subgroup analyses were included in the present study and no adjustments were made for multiple hypothesis testing. Nevertheless, the consistent findings in the SweBCG91RT, Sjöström, and Servant cohorts are reassuring. Patients of the SweBCG91RT cohort largely lacked systemic treatment, indicating that the risk of relapse would likely have been lower in the modern setting. At the same time, immunotherapy and chemotherapy may have further accentuated the difference in IBTR rates between highly proliferative tumors with and without immune infiltration due to the treatment-predictive effects of an immune infiltrate. In addition, high-risk patients would likely have received an RT boost if they were diagnosed today and were, therefore, also undertreated with RT. This may limit the generalizability. At the same time, these results provide an opportunity to study which patients do well without systemic therapy and an RT boost, and who may safely be omitted from intensified RT treatment. Finally, the immunological model was not solely based on direct measurements of an activated immune response. However, despite this, the correlation of the immunological model with stromal TILs measured on whole-tissue sections was stronger than that of other established deconvolutional methods for estimating immune infiltration based on bulk RNA data indicating that the methods disclosed herein allowed for an accurate assessment of the local immune response.

RT can affect the tumor microenvironment differently depending on the dose. Preclinical studies suggest high RT doses can turn an immunosuppressive tumor microenvironment proinflammatory. In addition, combining RT with immunotherapy may further favor RT-induced immune activation, and clinical trials investigating this are ongoing. Finally, without being bound by theory, neoadjuvant RT, which entails a high tumor burden, can be hypothesized to elicit RT-induced cell death more effectively than adjuvant RT. It remains to be determined if alternative fractionation schemes, the addition of immunotherapy, or neoadjuvant RT is more effective in inducing an antitumoral immune response compared to adjuvant RT with standard fractionation used as described herein.

In summary, implementing the immune system as a biomarker for treatment individualization of RT in breast cancer requires consideration of tumor-intrinsic characteristics. Patients with aggressive tumors derive strong protection from an activated immune infiltrate regarding IBTR and may be candidates for RT de-escalation.

TABLE 13
Risk Groups and Therapy to be Recommended and/or Administered

| | | Prognosis compared to | | | |
|---|---|---|---|---|---|
| | | tumors from the same | | | |
| | | grade category or a | | | |
| | | group defined by other | | Risk-benefit | |
| | | clinical/pathological | | profile | |
| | | variables or gene | Predicted | (compared to | |
| | | expression with a | benefit | tumors from | |
| | | corresponding | from RT | the same | Therapy to be |
| Risk | Tumor | recurrence | (standard | grade | recommended and/or |
| group | Grade*** | risk\hat{ } | of care) | category) # | administered |
| High-risk† | all | Unfavorable | Low | Residual | Intensified relative to |
| | | | | | standard of care (where |
| | | | | | standard of care depends |
| | | | | | on tumor- and patient |
| | | | | | characteristics (see |
| | | | | | Tables 14 and 15) |
| | III | Unfavorable | Low | Residual | Intensified relative to |
| | | | | | standard of care (where |
| | | | | | standard of care is standard |
| | | | | | radiotherapy with the |
| | | | | | addition of a boost**) |
| | II | Unfavorable | Low | Residual | Intensified relative to |
| | | | | | standard of care (where |
| | | | | | standard of care is standard |
| | | | | | radiotherapy without the |
| | | | | | addition of a boost*) |
| | I | Unfavorable | Low | Residual | Intensified relative to |
| | | | | | standard of care (where |
| | | | | | standard of care is standard |
| | | | | | radiotherapy without the |
| | | | | | addition of a boost*) |
| Medium- | all | Neither | High | High | Standard of care (where |
| risk†† | | favorable nor | | | standard of care depends |
| | | unfavorable | | | on tumor- and patient |
| | | | | | characteristics (see |
| | | | | | Tables 14 and 15) |
| | III | Neither | High | High | Standard of care (where |
| | | favorable nor | | | standard of care is standard |
| | | unfavorable | | | radiotherapy with the |
| | | | | | addition of a boost**) |
| | II | Neither | High | High | Standard of care is standard |
| | | favorable nor | | | radiotherapy without the |
| | | unfavorable | | | addition of a boost*) |
| | I | Neither | High | High | Standard of care is standard |
| | | favorable nor | | | radiotherapy without the |
| | | unfavorable | | | addition of a boost*) |
| Low-risk††† | all | Favorable | Low | Low | De-intensified relative to |
| | | | | | standard of care (where |
| | | | | | standard of care depends |
| | | | | | on tumor- and patient |
| | | | | | characteristics (see |
| | | | | | Tables 14 and 15) |
| | III | Favorable | Low | Low | De-intensified relative to |
| | | | | | standard of care (where |
| | | | | | standard of care is standard |
| | | | | | radiotherapy with the |
| | | | | | addition of a boost**) |
| | II | Favorable | Low | Low | De-intensified relative to |
| | | | | | standard of care (where |
| | | | | | standard of care is standard |
| | | | | | radiotherapy without the |
| | | | | | addition of a boost*) |
| | I | Favorable | Low | Low | De-intensified relative to |
| | | | | | standard of care (where |
| | | | | | standard of care is standard |
| | | | | | radiotherapy without the |
| | | | | | addition of a boost*) |

*Standard dosing (as defined in Tables 14 and 15): The whole breast should receive a hypofractionated dose of 40-42.5 Gy in 15-16 fractions; in selected cases 45-50.4 Gy in 25-28 fractions may be considered.
**A boost is recommended to patients with an increased risk of recurrence. A boost dose is typically 10-16 Gy in 4-8 fractions or it can be simultaneously integrated into the standard radiotherapy.
***Higher tumor grade is predictive of an unfavorable prognosis.
\hat{ }The risk groups depending on grade represent high risk (grade III), moderate risk (grade II), and low risk (grade I). Other methods than tumor grade can be used to define the risk groups:
Grade III: Considered a marker of poor prognosis. Other markers of poor prognosis which can be used instead of grade III are negative Estrogen Receptor (ER) status, negative Progesterone Receptor (PgR) status, young age, large tumor size, lymphovascular tumor invasion, high Oncotype Dx score, high MammaPrint score, high EndoPredict score, unfavorable Prosigna score.
Grade I: Considered a marker of good prognosis. Other markers of poor prognosis which can be used instead of grade I are positive Estrogen Receptor (ER) status, positive Progesterone Receptor (PgR) status, old age, small tumor size, lack of lymphovascular tumor invasion, low Oncotype Dx score, low MammaPrint score, low EndoPredict score, favorable Prosigna score.
Grade II: Better prognosis than grade I but worse prognosis than grade III.
# Risk-benefit profile
Residual: High risk of recurrence despite standard radiotherapy
High: High risk of recurrence without standard radiotherapy. Low risk of recurrence with standard radiotherapy.
Low: Low risk of recurrence without standard radiotherapy. Low risk of recurrence with standard radiotherapy.
†High-risk: Defined as a high score compared to a background population of representative tumors and patients§. The threshold may be anywhere above the 60^{th} percentile (e.g., the 67^{th} percentile, the 75^{th} percentile, the 80^{th} percentile, et cetera).
††Medium-risk: Defined as a medium score compared to a background population of representative tumors and patients§. The threshold may be anywhere from the 30^{th} to the 70^{th} percentile (e.g., the 45^{th} percentile, the 50^{th} percentile, the 55^{th} percentile, et cetera).
†Low-risk: Defined as a low score compared to a background population of representative tumors and patients§. The threshold may be anywhere below 40^{th} percentile (e.g., the 5^{th} percentile, the 15^{th} percentile, the 25^{th} percentile, et cetera).
These risk groups (high-risk, medium-risk, low-risk) can be defined by either of the following models:
Proliferative Index (can be regarded as a gene expression-based equivalent of histological grade)
Integrated model (an improvement compared to Proliferative Index because immune activation is also integrated)
Final (reduced) model (a further improvement because patient age is also integrated)
While the Final model is likely the best performer of the three, each of the other models are still capable of identifying high-, medium-, and low-risk tumors.
§Definition of a background population of representative tumors and patients
Tumor grade category: all. A sample, of preferably at least 100 tumors, with tumor and patient characteristics comparable to that of a general breast cancer population[1].
Tumor grade category: Grade III: A sample, of preferably at least 100 tumors, with demographic characteristics comparable to that of the breast cancer population with grade III tumors.
Tumor grade category: Grade II: A sample, of preferably at least 100 tumors, with demographic characteristics comparable to that of the breast cancer population with grade II tumors.
Tumor grade category: Grade I: A sample, of preferably at least 100 tumors, with demographic characteristics comparable to that of the breast cancer population with grade I tumors.

TABLE 14
Radiotherapy Guidelines

| | N- | | | | Boost |
|---|---|---|---|---|---|
| Guideline | status | Surgery | Volume | Fractionation | (tumor bed) |
| NCCN | N0 | BCS | WBRT * | 40-42.5 Gy in 15-16 | 10-16 Gy in 4-8 |
| | | | and ** | fractions (or 45-50.5 | fraction if high |
| | | | | Gy in 25-28 Fr.) | risk *** |
| NCCN | N+ | BCS | WBRT + | 40-42.5 Gy in 15-16 | 10-16 Gy in 4-8 |
| | | | RNI | fractions (or 45-50.5 | fraction if high |
| | | | | Gy in 25-28 Fr.) | risk *** |
| ESMO | N0 | BCS | WBRT § | 45-50 Gy in 25-28 | 10-16 Gy in 4-8 |
| | | | | fractions (or 2.5-2.67 | fraction if high |
| | | | | Gy × 15-16 Fr) | risk *** |
| ESMO | N+ | BCS | WBRT + | 45-50 Gy in 25-28 | 10-16 Gy in 4-8 |
| | | | RNI | fractions (or 2.5-2.67 | fraction if high |
| | | | | Gy × 15-16 Fr) | risk *** |
| NICE | N0 | BCS | WBRT § | 40 Gy in 15 | 10 Gy in 5 fractions |
| | | | | fractions | if high risk z |
| NICE | N+ | BCS | WBRT + | 40 Gy in 15 | 10 Gy in 5 fractions |
| | | | RNI | fractions | if high risk z |

```
WBRT = Whole breast radiotherapy
RNI = regional nodal irradiation
BCS = breast-conserving surgery
N0 = no lymph node involvement
N+ = lymph node involvement
* APBI if low risk
** omission considered if >70 ER+, T1, Endocrine Treatment (ET)
*** >2 cm, younger age, LVI (lymphovascular invasion)
§ APBI if age <50, T <= 3, ER-positive, HER2-negative and grade 1 to 2, non-lobular
z Risk can be estimated using a range of standardised tools and clinical expertise
NCCN Guidelines Version 4.2021 (Invasive Breast Cancer)
PRINCIPLES OF RADIATION THERAPY
Optimizing Delivery of Individual Therapy
  It is important to individualize RT planning and delivery.
      CT-based treatment planning should be routinely utilized to delineate target volumes and
      adjacent organs at risk.
      Radiation to the breast/chest wall and nodal regions is generally delivered with single
      energy or mixed energy photons ± electrons.
      Improved homogeneity of the target dose and sparing of normal tissues can be
      accomplished using compensators such as wedges, forward planning using segments,
      and intensity-modulated RT (IMRT).
      Additional techniques such as respiratory control (deep inspiration breath-hold), prone
      positioning, cardiac blocks may also be used to try to further reduce dose to heart, lung,
      and adjacent normal tissue.
      Verification of treatment setup consistency is done with weekly imaging. When using
      certain techniques (i.e., prone breast), more frequent imaging may be appropriate.
      Standard utilization of daily imaging is not recommended.
      When treating the internal mammary nodes, dose-volume histograms (DVHs) should be
      used to evaluate dose constraints, dose to normal tissues (i.e., heart, lung), and planning
      target volumes (PTVs).
  It is common for RT to follow chemotherapy when chemotherapy is indicated.
Whole Breast Radiation
  Target definition is the breast tissue in entirety.
  RT dosing:
      The whole breast should receive a hypofractionated dose of 40-42.5 Gy in 15-16
      fractions; in selected cases 45-50.4 Gy in 25-28 fractions may be considered.
      A boost to the tumor bed is recommended in patients at higher risk for recurrence.
      Typical boost doses arc 10-16 Gy in 4-8 fractions.
  Lumpectomy cavity boost can bedelivered using enface electrons, photons, or
  brachytherapy.
  For patients who require a more limited number of treatment visits for WBRT delivery,
  ultra-hypofractionated WBRT of 28.5 Gy delivered as 5 (once-a-week) fractions, may
  be considered in selected patients aged ≥50 years following BCS with pTis/T1/T2/N0
  tumor. However, late toxicity effects beyond 10 years are not currently defined.
      The optimal fractionation for the delivery of a boost is not known for this regimen.
      3-D planning o minimize inhomogeneity and exposure to heart and lung is essential
      when using this regimen.
ESMO guidelines
Whole-breast radiotherapy.
RT after BCS: Postoperative RT is strongly recommended after BCS [I, A]. WBRT alone reduces
the 10-year risk of any first recurrence (including locoregional and distant) by 15% and the 15-year
risk of breast cancer-related mortality by 4%. Boost RT gives a further 50% RR reduction and is
indicated for most patients who have unfavourable risk factors for local control such as age <50
years, grade 3 tumours, presence of vascular invasion or extensive intraductal component and non-
radical tumour excision (focally-otherwise further surgery should be advocated) [I, A].
Recommendations:
```

Postoperative RT is strongly recommended after BCS [I, A].
Boost RT is recommended to reduce the risk of in-breast re-
lapse in patients at higher risk of local recurrence [I, A].

Accelerated partial-breast RT after BCS: The concept of accelerated partial-breast irradiation (APBI)
is an appealing approach to substantially shorten the overall treatment time. The rationale for APBI
is that the majority of local failures occur in the vicinity of the primary tumour site, while so-called
‘elsewhere’ in-breast failures may represent a new primary tumour. Excellent results with low local
recurrence rates equivalent to WBRT are reported for partial-breast irradiation (accelerated and
conventionally fractionated) using external beam techniques and brachytherapy. However, for
intraoperative RT, as used in the ELIOT (single dose with electrons) and TARGIT (single dose with
50-kV X- rays) randomised trials, the ipsilateral breast cancer recurrence rate was significantly
higher in the APBI groups, compared with the WBRT. Based on these results, APBI might be
considered an acceptable treatment option in patients with a low risk for local recurrence, for example
those who are at least 50years old, with unicentric, unifocal, node-negative, non- lobular breast
cancer, up to 3 cm without the presence of extensive intraductal components or vascular invasion
and with negative margins, especially if they will receive adjuvant endocrine treatment [III, C]. APBI
may also be considered for low-grade DCIS [III, C]. More and long-term results of several past and
ongoing prospective randomised APBI trials are awaited.

Recommendation:
APBI is an acceptable treatment option in patients with a low risk for local recurrence [III,
C].

```
Early and locally advanced breast cancer: diagnosis and management
NICE guideline [NG101] Published: 18 Jul. 2018
Radiotherapy after breast-conserving surgery
1.10.3 Offer whole-breast radiotherapy to women with invasive breast cancer who have had
    breast-conserving surgery with clear margins. [2018]
1.10.4 Consider partial breast radiotherapy (as an alternative to whole-breast radiotherapy) for
    women who have had breast-conserving surgery for invasive cancer (excluding lobular type)
    with clear margins and who:
```

have a low absolute risk of local recurrence (defined as women aged 50 and over with
tumours that are 3 cm or less, N0, ER-positive, HER2-negative and grade 1 to 2) and
have been advised to have adjuvant endocrine therapy for a minimum of 5 years. [2018]

1.10.5 When considering partial breast radiotherapy (see recommendation 1.10.4), discuss the
benefits and risks, and explain that:

local recurrence with partial breast radiotherapy at 5 years is equivalent to that with
whole-breast radiotherapy
the risk of local recurrence beyond 5 years is not yet known
there is a potential reduction in late adverse effects. [2018]

1.10.6 When delivering partial breast radiotherapy, use external beam radiotherapy. [2018]
1.10.7 Consider omitting radiotherapy for women who:

have had breast-conserving surgery for invasive breast cancer with clear margins and
have a very low absolute risk of local recurrence (defined as women aged 65 and over with
tumours that are T1N0, ER-positive, HER2-negative and grade 1 to 2) and
are willing to take adjuvant endocrine therapy for a minimum of 5 years. [2018]

1.10.8 When considering omitting radiotherapy for the population in recommendation 1.10.7,
discuss the benefits and risks . . . and explain that:

without radiotherapy, local recurrence occurs in about 50 women per 1,000 at 5 years, and
with radiotherapy, occurs in about 10 women per 1,000 at 5 years
overall survival at 10 years is the same with or without radiotherapy
there is no increase in serious late effects if radiotherapy is given (for example, congestive
cardiac failure, myocardial infarction or secondary cancer). [2018]

```
Dose fractionation
    1.10.13 Use external beam radiotherapy giving 40 Gy in 15 fractions as standard practice for
    women with invasive breast cancer after breast-conserving surgery or mastectomy. [2009]
Breast boost following breast-conserving surgery
    1.10.14 Offer an external beam boost to the tumour bed for women with invasive breast cancer
    and a high risk (www.nice.org.uk/guidance/ng101/chapter/Recommendations) of local
    recurrence, following whole-breast radiotherapy. [2009, amended 2018]
    1.10.15 Inform women of the risk of side effects associated with an external beam boost to the
    tumour bed following whole-breast radiotherapy. [2009, amended 2018]
Radiotherapy to nodal areas
    1.10.16 Do not offer adjuvant radiotherapy to regional lymph nodes to people with invasive
    breast cancer who have been shown to have histologically lymph node-negative breast
    cancer. [2009, amended 2018]
    1.10.17 Do not offer adjuvant radiotherapy to the axilla after axillary clearance for invasive
    breast cancer. [2009, amended 2018]
    1.10.18 Offer adjuvant radiotherapy to the supraclavicular fossa to people with invasive breast
    cancer and 4 or more involved axillary lymph nodes. [2009]
    1.10.19 Offer adjuvant radiotherapy to the supraclavicular fossa to people with invasive breast
    cancer and 1 to 3 positive lymph nodes if they have other poor prognostic factors (for example,
    T3 and/or histological grade 3 tumours) and good performance status. [2009]
    1.10.20 Consider including the internal mammary chain within the nodal radiotherapy target for
    people with node-positive (macrometastases) invasive breast cancer. [2018]
ESTRO
Ultrahypofractionation (26 Gy in five fractions) for chest wall irradiation without breast
reconstruction can be offered as standard of care or within a randomised controlled trial or
prospective registration cohort.
International guidelines
The following international guidelines have been identified which include guidance on the use of
hypofractionated radiotherapy for early breast cancer:
```

The American Society for Radiation Oncology (ASTRO) guidelines on fractionation for
whole breast irradiation, 2010
The New Zealand Ministry of Health Guidelines for Management of Early Breast Cancer,
2009
NICE Guidelines for early and locally advanced breast cancer, 2009
Scottish Intercollegiate Guidelines Network (SIGN) guidelines, 2009
BC Cancer Agency Breast cancer management consensus guidelines 2013
European Journal of Medical Oncology (ESMO) guidelines on primary breast cancer
diagnosis, treatment and follow-up, 2013
German Society of Radiation Oncology (DEGRO) guidelines on radiotherapy of breast
cancer, 2013
Nice-Saint-Paul de Vence guidelines on adjuvant radiotherapy in the management of axillary
node negative invasive breast cancer, 2013

The BC Cancer Agency consensus-based guidelines for the management of early breast cancer
include recommendations on the use of radiotherapy and recommend a hypofractionated
radiotherapy regimen as standard. The guideline recommends the following dose fractionation for
radiotherapy following breast conserving therapy (T1, T2; N0):

| | |
|---|---|
| 1. | Standard whole breast dose is 42.5 Gray (Gy) in 16 daily fractions |
| 2. | Certain patients are at risk for inferior cosmetic outcome from the 16-fraction course. |
| | Extended fractionation should be considered for patients with very large breast size, and |
| | those with significant post-operative induration, oedema, erythema, hematoma or infection. |
| | Patients with these indications for extended fractionation should receive 45 Gy in 25 daily |
| | fractions plus a boost dose of 10 Gy in 5 fractions or 50.4 Gy in 28 daily fractions. |
| 3. | If a boost is used, an additional dose of 6-16 Gy in 3-8 fractions is recommended. |

TABLE 15
ESTRO Radiotherapy Guidelines (2022)
Panel: Final consensus statements

1. Whole breast irradiation

| | |
|---|---|
| a | Moderate hypofractionated whole breast irradiation should be offered regardless of age at breast |
| | cancer diagnosis, pathological tumour stage, breast cancer biology, surgical margins status, |
| | tumour bed boost, breast size, invasive or pre-invasive ductal carcinoma in situ (DCIS) disease, |
| | oncoplastic breast conserving surgery, and use of systemic therapy |
| b | Ultrahypofractionated (26 Gy in five fractions) whole breast irradiation can be offered as (1) |
| | standard of care or (2) within a randomised controlled trial or prospective registration cohort |

2. Chest wall irradiation

| | |
|---|---|
| a | Moderate hypofractionation can be offered for chest wall irradiation without breast |
| | reconstruction |
| b | Moderate hypofractionation can be offered for chest wall irradiation regardless of time and type |
| | of breast reconstruction |
| c | Ultrahypofractionation (26 Gy in five fractions) for chest wall irradiation without breast |
| | reconstruction can be offered as (1) standard of care or (2) within a randomised controlled trial |
| | or prospective registration cohort |
| d | Ultrahypofractionation (26 Gy in five fractions) for chest wall irradiation after breast |
| | reconstruction can be offered within a randomised controlled trial or prospective registration |
| | cohort |

3. Nodal irradiation

| | |
|---|---|
| a | Moderate hypofractionation should be offered for nodal irradiation |
| b | Ultrahypofractionation (26 Gy in five fractions) should not be offered for nodal irradiation until |
| | ongoing trials results are reported |

4. Partial breast irradiation-patient selection for external beam radiotherapy

Low risk-features suitable for partial breast irradiation are: luminal-like subtypes small tumour
(≤3 cm), absence of lymph vascular space invasion, non-lobular invasive carcinoma, tumour
grade 1-2, low-to-intermediate grade DCIS (sized ≤2.5 cm with clear surgical margins ≥3 mm),
age at diagnosis 50 years or more, unicentric or unifocal lesion, clear surgical margins (>2 mm),
node negative (including isolated tumour cells), and no use of primary systemic therapy and
neoadjuvant chemotherapy

5. Partial breast irradiation-dose and fractionation

| | |
|---|---|
| a | Moderate hypofractionation (40 Gy in 15 fractions) and ultrahypofractionation (26-30 Gy in |
| | five fractions) represent acceptable schedules for external beam partial breast irradiation |
| b | Twice a day external beam partial breast irradiation dose and fractionations similar to those used |
| | in the RAPID trial should not be offered |

DCIS: ductal carcinoma in situ.

TABLE 16A
Any Recurrence (Grade I/II/III)

| | | All Patients (Grade I/II/III tumors) | | | | |
|---|---|---|---|---|---|---|
| Marker | Variable | HR | CI Low | CI High | P-value | Interpretation |
| PD-L1 RT | PD-L1 <1% no RT | Reference | Reference | Reference | Reference | Patients with high PD-L1 protein expression |
| | | group | group | group | group | show a reduced benefit from standard |
| | | (HR 1) | | | | radiotherapy treatment — (HR decreases |
| | | | | | | from 1.12 to 0.77 with radiotherapy compared |
| | | | | | | to patients with low PD-L1 where HR |
| | | | | | | decreases from 1 to 0.476 with radiotherapy. If |
| | | | | | | the patient has a good prognosis --> de- |
| | | | | | | intensify. If the patient has a poor prognosis --> |
| | | | | | | intensify.) |
| | PD-L1 >=1% no RT | 1.12 | 0.749 | 1.675 | 0.581 | — |
| | PD-L1 <1% RT | 0.476 | 0.346 | 0.655 | <0.001 | Standard of care |
| | PD-L1 >=1% RT | 0.77 | 0.473 | 1.251 | 0.291 | Intensify |
| PD-1 RT | PD-1 <1% no RT | Reference | Reference | Reference | Reference | — (Patients with high PD-1 protein |
| | | group | group | group | group | expression show a reduced benefit from |
| | | (HR 1) | | | | standard radiotherapy treatment. If they have a |
| | | | | | | good prognosis --> de-intensify. If they have a |
| | | | | | | poor prognosis --> intensify.) |
| | PD-1 >=1% no RT | 0.866 | 0.522 | 1.437 | 0.577 | — |
| | PD-1 <1% RT | 0.479 | 0.354 | 0.649 | <0.001 | Standard of care |
| | PD-1 >=1% RT | 0.684 | 0.386 | 1.211 | 0.193 | Intensify |
| PD-1 | PD-1 <1% PD- | Reference | Reference | Reference | Reference | — (Patients with high PD-1 and PD-L1 |
| PD-L1 RT | L1 <1% no RT | group | group | group | group | protein expression derive less benefit from |
| | | (HR 1) | | | | standard radiotherapy treatment. In particular |
| | | | | | | the group with high expression of both proteins |
| | | | | | | show a poor benefit.) |
| | PD-1 >=1% PD- | 0.707 | 0.311 | 1.609 | 0.409 | — |
| | L1 <1% no RT | | | | | |
| | PD-1 <1% PD- | 1.145 | 0.703 | 1.865 | 0.587 | — |
| | L1 >=1% no RT | | | | | |
| | PD-1 >=1% PD- | 1.021 | 0.549 | 1.899 | 0.948 | — |
| | L1 >=1% no RT | | | | | |
| | PD-1 <1% PD- | 0.459 | 0.33 | 0.64 | <0.001 | Standard of care |
| | L1 <1% RT | | | | | |
| | PD-1 >=1% PD- | 0.548 | 0.224 | 1.344 | 0.189 | Standard of care |
| | L1 <1% RT | | | | | |
| | PD-1 <1% PD- | 0.7 | 0.376 | 1.303 | 0.261 | Intensify |
| | L1 >=1% RT | | | | | |
| | PD-1 >=1% PD- | 0.84 | 0.41 | 1.723 | 0.635 | Intensify |
| | L1 >=1% RT | | | | | |
| PD-L1 | PD-L1 <1% TILsLow | Reference | Reference | Reference | Reference | Patients with high PD-L1 and high TILs have |
| TILs | | group | group | group | group | an increased risk of recurrence in the whole |
| | | (HR 1) | | | | patient population |
| | | | | | | Standard of care |
| | PD-L1 <1% TILsHigh | 1.032 | 0.709 | 1.5 | 0.870 | See above |
| | PD-L1 >=1% TILsLow | 1.068 | 0.625 | 1.824 | 0.810 | See above |
| | PD-L1 >=1% TILsHigh | 1.562 | 1.065 | 2.292 | 0.023 | Intensify |
| PD-1 | PD-1 <1% TILsLow | Reference | Reference | Reference | Reference | Standard of care |
| TILs | | group | group | group | group | |
| | | (HR 1) | | | | |
| | PD-1 <1% TILsHigh | 1.305 | 0.948 | 1.798 | 0.103 | Standard of care |
| | PD-1 >=1% TILsLow | 1.207 | 0.682 | 2.135 | 0.519 | Standard of care |
| | PD-1 >=1% TILsHigh | 1.076 | 0.63 | 1.837 | 0.789 | Standard of care |
| PD-L1 | PD-L1 <1% | Reference | Reference | Reference | Reference | — (Patients with high PD-L1 protein |
| TILs RT | TILsLow no RT | group | group | group | group | expression and high TILs show a decreased |
| | | (HR 1) | | | | benefit from standard radiotherapy treatment.) |
| | PD-L1 <1% | 0.749 | 0.456 | 1.232 | 0.256 | — |
| | TILsHigh no RT | | | | | |
| | PD-L1 >=1% | 0.797 | 0.386 | 1.644 | 0.538 | — |
| | TILsLow no RT | | | | | |
| | PD-L1 >=1% | 1.274 | 0.79 | 2.054 | 0.321 | — |
| | TILsHigh no RT | | | | | |
| | PD-L1 <1% | 0.434 | 0.299 | 0.63 | <0.001 | Standard of care/de-intensify |
| | TILsLow RT | | | | | |
| | PD-L1 <1% | 0.696 | 0.408 | 1.187 | 0.183 | Standard of care |
| | TILsHigh RT | | | | | |
| | PD-L1 >=1% | 0.702 | 0.325 | 1.517 | 0.368 | Standard of care |
| | TILsLow RT | | | | | |
| | PD-L1 >=1% | 0.882 | 0.482 | 1.614 | 0.684 | Intensify |
| | TILsHigh RT | | | | | |
| PD-L1 high | PD-L1 <1% Low/ | Reference | Reference | Reference | Reference | — (Patients with very high stromal TIL |
| TILs RT | moderate TILs no RT | group | group | group | group | levels (>50%) have a high risk of recurrence |
| | | (HR 1) | | | | despite standard radiotherapy treatment) |
| | PD-L1 >=1% Low/ | 0.915 | 0.548 | 1.526 | 0.733 | — |
| | moderate no RT | | | | | |
| | PD-L1 <1% Very | 2.107 | 0.926 | 4.797 | 0.076 | — |
| | high TILs no RT | | | | | |
| | PD-L1 >=1% Very | 1.745 | 0.981 | 3.103 | 0.058 | — |
| | high TILs no RT | | | | | |
| | PD-L1 <1% TILs | 0.473 | 0.341 | 0.656 | <0.001 | Standard of care/de-intensify |
| | Low/moderate RT | | | | | |
| | PD-L1 >=1% TILs | 0.763 | 0.437 | 1.332 | 0.342 | Standard of care |
| | Low/moderate RT | | | | | |
| | PD-L1 <1% TILs | 1.372 | 0.435 | 4.321 | 0.589 | Intensify |
| | Very high RT | | | | | |
| | PD-L1 >=1% TILs | 0.884 | 0.36 | 2.167 | 0.787 | Intensify |
| | Very high RT | | | | | |
| PD-1 | PD-1 <1% | Reference | Reference | Reference | Reference | — (PD-1 protein expression can be a marker |
| TILs RT | TILsLow no RT | group | group | group | group | of radioresistance. Among grade III tumors, |
| | | (HR 1) | | | | high PD-1 in combination with low TILs |
| | | | | | | indicates a poor prognosis and radioresistance. |
| | | | | | | Low PD-1 and low TILs indicates a poor |
| | | | | | | prognosis. Among grade I and grade II tumors, |
| | | | | | | high groups with high PD-1 expression benefit |
| | | | | | | from intensified therapy regardless of TILs. |
| | | | | | | However, the group with high TILs and high |
| | | | | | | PD-1 show the greatest need for |
| | | | | | | intensification.) |
| | PD-1 <1% | 1.038 | 0.692 | 1.558 | 0.857 | — |
| | TILsHigh no RT | | | | | |
| | PD-1 >=1% | 0.936 | 0.433 | 2.022 | 0.866 | — |
| | TILsLow no RT | | | | | |
| | PD-1 >=1% | 0.775 | 0.375 | 1.599 | 0.490 | — |
| | TILsHigh no RT | | | | | |
| | PD-1 <1% | 0.442 | 0.306 | 0.639 | <0.001 | De-intensify |
| | TILsLow RT | | | | | |
| | PD-1 <1% | 0.789 | 0.49 | 1.272 | 0.331 | Standard of care |
| | TILsHigh RT | | | | | |
| | PD-1 >=1% | 0.773 | 0.338 | 1.769 | 0.543 | Standard of care |
| | TILsLow RT | | | | | |
| | PD-1 >=1% | 0.745 | 0.345 | 1.61 | 0.454 | Intensify |
| | TILsHigh RT | | | | | |
| PD-1 High | PD-1 <1% Low/ | Reference | Reference | Reference | Reference | — (PD-1 protein expression can be a marker |
| TILs RT | moderate TILs no RT | group | group | group | group | of radioresistance. Among grade III tumors, |
| | | (HR 1) | | | | high PD-1 and low very high TILs indicates a |
| | | | | | | favorable prognosis and these patients do not |
| | | | | | | benefit from standard radiotherapy. Very high |
| | | | | | | PD-1 and low/moderate TILs indicates poorer |
| | | | | | | prognosis and these patients benefit from |
| | | | | | | intensification. Low PD-1 in combination with |
| | | | | | | very high TILs (>50%) indicates a poor |
| | | | | | | prognosis and benefit from intensification. |
| | | | | | | Among grade I and grade II tumors, very high |
| | | | | | | TIL groups with high PD-1 expression benefit |
| | | | | | | from intensified therapy regardless of TILs. |
| | | | | | | However, the group with high TILs and high |
| | | | | | | PD-1 show the greatest need for |
| | | | | | | intensification.) |
| | PD-1 >=1% Low/ | 0.867 | 0.478 | 1.573 | 0.639 | — |
| | moderate no RT | | | | | |
| | PD-1 <1% Very | 2.415 | 1.386 | 4.211 | 0.002 | — |
| | high TILs no RT | | | | | |
| | PD-1 >=1% Very | 1.106 | 0.451 | 2.709 | 0.826 | — |
| | high TILs no RT | | | | | |
| | PD-1 <1% TILs | 0.475 | 0.346 | 0.654 | <0.001 | Standard of care/de-intensify |
| | Low/moderate RT | | | | | |
| | PD-1 >=1% TILs | 0.851 | 0.458 | 1.581 | 0.610 | Standard of care |
| | Low/moderate RT | | | | | |
| | PD-1 <1% TILs | 1.991 | 0.875 | 4.526 | 0.100 | Intensify |
| | Very high RT | | | | | |
| | PD-1 >=1% TILs | 0.413 | 0.102 | 1.671 | 0.215 | Standard of care |
| | Very high RT | | | | | |
| TILs RT | TILs <10% no RT | Reference | Reference | Reference | Reference | — (Patients with high TIL levels (>=10%) |
| | | group | group | group | group | show a reduced benefit from standard |
| | | (HR 1) | | | | radiotherapy treatment) |
| | TILs >=10% no RT | 0.977 | 0.675 | 1.415 | 0.903 | — |
| | TILs <10% RT | 0.469 | 0.331 | 0.665 | <0.001 | Standard of care |
| | TILs >=10% RT | 0.782 | 0.512 | 1.193 | 0.254 | Intensify |
| High | TILs Low/moderate | Reference | Reference | Reference | Reference | — (Patients with very high stromal TIL |
| TILs RT | no RT | group | group | group | group | levels (>50%) have a high risk of recurrence |
| | | (HR 1) | | | | despite standard radiotherapy treatment) |
| | TILs Very high no RT | 1.868 | 1.153 | 3.028 | 0.011 | — |
| | TILS Low/moderate RT | 0.52 | 0.387 | 0.699 | <0.001 | Standard of care |
| | TILs Very high RT | 1.033 | 0.505 | 2.111 | 0.929 | Intensify |

TABLE 16B
Any Recurrence (Grade III)

| | | Grade III Tumors | | | | |
|---|---|---|---|---|---|---|
| Marker | Variable | HR | CI Low | CI High | P-value | Interpretation |
| PD-L1 RT | PD-L1 <1% no RT | Reference | Reference | Reference | Reference | — |
| | | group | group | group | group | |
| | PD-L1 >=1% no RT | 0.713 | 0.388 | 1.309 | 0.275 | — |
| | PD-L1 <1% RT | 0.616 | 0.336 | 1.132 | 0.119 | Intensify |
| | PD-L1 >=1% RT | 0.541 | 0.274 | 1.069 | 0.077 | De-intensify |
| PD-1 RT | PD-1 <1% no RT | Reference | Reference | Reference | Reference | — |
| | | group | group | group | group | |
| | PD-1 >=1% no RT | 0.423 | 0.18 | 0.995 | 0.049 | — |
| | PD-1 <1% RT | 0.613 | 0.364 | 1.033 | 0.066 | Intensify |
| | PD-1 >=1% RT | 0.413 | 0.163 | 1.042 | 0.061 | De-intensify |
| PD-1 | PD-1 <1% PD- | Reference | Reference | Reference | Reference | — |
| PD-L1 RT | L1 <1% no RT | group | group | group | group | |
| | PD-1 >=1% PD- | 0.263 | 0.036 | 1.925 | 0.189 | — |
| | L1 <1% no RT | | | | | |
| | PD-1 <1% PD- | 0.847 | 0.417 | 1.718 | 0.645 | — |
| | L1 >=1% no RT | | | | | |
| | PD-1 >=1% PD- | 0.456 | 0.178 | 1.168 | 0.102 | — |
| | L1 >=1% no RT | | | | | |
| | PD-1 <1% PD- | 0.639 | 0.342 | 1.194 | 0.161 | Intensify |
| | L1 <1% RT | | | | | |
| | PD-1 >=1% PD- | 0.225 | 0.031 | 1.647 | 0.142 | De-intensify |
| | L1 <1% RT | | | | | |
| | PD-1 <1% PD- | 0.507 | 0.224 | 1.146 | 0.102 | De-intensify |
| | L1 >=1% RT | | | | | |
| | PD-1 >=1% PD- | 0.488 | 0.173 | 1.379 | 0.176 | De-intensify |
| | L1 >=1% RT | | | | | |
| PD-L1 | PD-L1 <1% TILsLow | Reference | Reference | Reference | Reference | Intensify |
| TILs | | group | group | group | group | |
| | PD-L1 <1% TILsHigh | 0.666 | 0.365 | 1.213 | 0.184 | De-intensify |
| | PD-L1 >=1% TILsLow | 1.039 | 0.458 | 2.353 | 0.928 | Standard of care |
| | PD-L1 >=1% TILsHigh | 0.583 | 0.33 | 1.028 | 0.062 | De-intensify |
| PD-1 | PD-1 <1% TILsLow | Reference | Reference | Reference | Reference | Intensify |
| TILs | | group | group | group | group | |
| | PD-1 <1% TILsHigh | 0.767 | 0.466 | 1.264 | 0.298 | De-intensify |
| | PD-1 >=1% TILsLow | 1.17 | 0.36 | 3.799 | 0.794 | Intensify |
| | PD-1 >=1% TILsHigh | 0.377 | 0.175 | 0.811 | 0.013 | De-intensify |
| PD-L1 | PD-L1 <1% | Reference | Reference | Reference | Reference | — |
| TILs RT | TILsLow no RT | group | group | group | group | |
| | PD-L1 <1% | 0.414 | 0.193 | 0.892 | 0.024 | — |
| | TILsHigh no RT | | | | | |
| | PD-L1 >=1% | 0.971 | 0.337 | 2.799 | 0.957 | — |
| | TILsLow no RT | | | | | |
| | PD-L1 >=1% | 0.466 | 0.228 | 0.951 | 0.036 | — |
| | TILsHigh no RT | | | | | |
| | PD-L1 <1% | 0.383 | 0.172 | 0.853 | 0.019 | Intensify |
| | TILsLow RT | | | | | |
| | PD-L1 <1% | 0.582 | 0.251 | 1.351 | 0.208 | De-intensify |
| | TILsHigh RT | | | | | |
| | PD-L1 >=1% | 0.563 | 0.169 | 1.869 | 0.348 | Standard of care |
| | TILsLow RT | | | | | |
| | PD-L1 >=1% | 0.361 | 0.162 | 0.803 | 0.013 | De-intensify |
| | TILsHigh RT | | | | | |
| PD-L1 high | PD-L1 <1% Low/ | Reference | Reference | Reference | Reference | — |
| TILs RT | moderate TILs no RT | group | group | group | group | |
| | PD-L1 >=1% Low/ | 0.695 | 0.32 | 1.509 | 0.358 | — |
| | moderate no RT | | | | | |
| | PD-L1 <1% Very | 0.68 | 0.163 | 2.836 | 0.596 | — |
| | high TILs no RT | | | | | |
| | PD-L1 >=1% Very | 0.692 | 0.305 | 1.567 | 0.377 | — |
| | high TILs no RT | | | | | |
| | PD-L1 <1% TILs | 0.579 | 0.304 | 1.103 | 0.097 | Intensify |
| | Low/moderate RT | | | | | |
| | PD-L1 >=1% TILs | 0.554 | 0.244 | 1.255 | 0.157 | Standard of care |
| | Low/moderate RT | | | | | |
| | PD-L1 <1% TILs | 0.782 | 0.187 | 3.265 | 0.736 | Intensify |
| | Very high RT | | | | | |
| | PD-L1 >=1% TILs | 0.485 | 0.172 | 1.373 | 0.173 | De-intensify |
| | Very high RT | | | | | |
| PD-1 | PD-1 <1% | Reference | Reference | Reference | Reference | — |
| TILs RT | TILsLow no RT | group | group | group | group | |
| | PD-1 <1% | 0.541 | 0.29 | 1.01 | 0.054 | — |
| | TILsHigh no RT | | | | | |
| | PD-1 >=1% | 0.982 | 0.233 | 4.139 | 0.980 | — |
| | TILsLow no RT | | | | | |
| | PD-1 >=1% | 0.255 | 0.089 | 0.732 | 0.011 | — |
| | TILsHigh no RT | | | | | |
| | PD-1 <1% | 0.407 | 0.196 | 0.844 | 0.016 | Intensify |
| | TILsLow RT | | | | | |
| | PD-1 <1% | 0.552 | 0.273 | 1.118 | 0.099 | De-intensify |
| | TILsHigh RT | | | | | |
| | PD-1 >=1% | 0.638 | 0.087 | 4.704 | 0.659 | Intensify |
| | TILsLow RT | | | | | |
| | PD-1 >=1% | 0.282 | 0.098 | 0.808 | 0.018 | De-intensify |
| | TILsHigh RT | | | | | |
| PD-1 High | PD-1 <1% Low/ | Reference | Reference | Reference | Reference | — |
| TILs RT | moderate TILs no RT | group | group | group | group | |
| | PD-1 >=1% Low/ | 0.529 | 0.188 | 1.488 | 0.228 | — |
| | moderate no RT | | | | | |
| | PD-1 <1% Very | 1.061 | 0.472 | 2.385 | 0.885 | — |
| | high TILs no RT | | | | | |
| | PD-1 >=1% Very | 0.308 | 0.074 | 1.28 | 0.105 | — |
| | high TILs no RT | | | | | |
| | PD-1 <1% TILs | 0.537 | 0.298 | 0.967 | 0.038 | De-intensify |
| | Low/moderate RT | | | | | |
| | PD-1 >=1% TILs | 0.75 | 0.267 | 2.107 | 0.585 | Intensify |
| | Low/moderate RT | | | | | |
| | PD-1 <1% TILs | 1.208 | 0.474 | 3.079 | 0.692 | Intensify |
| | Very high RT | | | | | |
| | PD-1 >=1% TILs | 0.15 | 0.021 | 1.092 | 0.061 | De-intensify |
| | Very high RT | | | | | |
| TILs RT | TILs <10% no RT | Reference | Reference | Reference | Reference | — |
| | | group | group | group | group | |
| | TILs >=10% no RT | 0.443 | 0.249 | 0.787 | 0.006 | — |
| | TILs <10% RT | 0.421 | 0.21 | 0.847 | 0.015 | Standard of care |
| | TILs >=10% RT | 0.44 | 0.235 | 0.825 | 0.010 | De-intensify |
| High | TILs Low/moderate | Reference | Reference | Reference | Reference | — |
| TILs RT | no RT | group | group | group | group | |
| | TILs Very high no RT | 0.749 | 0.364 | 1.544 | 0.434 | — |
| | TILS Low/moderate RT | 0.62 | 0.362 | 1.06 | 0.081 | Standard of care |
| | TILs Very high RT | 0.604 | 0.256 | 1.426 | 0.250 | De-intensify |

TABLE 16C
Any Recurrence (Grade I/II)

| | | Grade I/II Tumors | | | | |
|---|---|---|---|---|---|---|
| Marker | Variable | HR | CI Low | CI High | P-value | Interpretation |
| PD-L1 RT | PD-L1 <1% no RT | Reference | Reference | Reference | Reference | — |
| | | group | group | group | group | |
| | PD-L1 >=1% no RT | 1.267 | 0.714 | 2.247 | 0.419 | — |
| | PD-L1 <1% RT | 0.51 | 0.348 | 0.747 | 0.001 | De-intensified |
| | PD-L1 >=1% RT | 0.861 | 0.396 | 1.872 | 0.706 | Intensify |
| PD-1 RT | PD-1 <1% no RT | Reference | Reference | Reference | Reference | — |
| | | group | group | group | group | |
| | PD-1 >=1% no RT | 1.103 | 0.553 | 2.201 | 0.781 | — |
| | PD-1 <1% RT | 0.492 | 0.337 | 0.717 | <0.001 | De-intensified |
| | PD-1 >=1% RT | 0.95 | 0.438 | 2.06 | 0.897 | Intensify |
| PD-1 | PD-1 <1% PD- | Reference | Reference | Reference | Reference | — |
| PD-L1 RT | L1 <1% no RT | group | group | group | group | |
| | PD-1 >=1% PD- | 0.863 | 0.315 | 2.368 | 0.775 | — |
| | L1 <1% no RT | | | | | |
| | PD-1 <1% PD- | 1.16 | 0.578 | 2.326 | 0.676 | — |
| | L1 >=1% no RT | | | | | |
| | PD-1 >=1% PD- | 1.474 | 0.594 | 3.657 | 0.403 | — |
| | L1 >=1% no RT | | | | | |
| | PD-1 <1% PD- | 0.485 | 0.325 | 0.721 | <0.001 | De-intensified |
| | L1 <1% RT | | | | | |
| | PD-1 >=1% PD- | 0.861 | 0.314 | 2.36 | 0.771 | Intensify |
| | L1 <1% RT | | | | | |
| | PD-1 <1% PD- | 0.714 | 0.26 | 1.959 | 0.513 | Intensify |
| | L1 >=1% RT | | | | | |
| | PD-1 >=1% PD- | 1.154 | 0.363 | 3.667 | 0.809 | Intensify |
| | L1 >=1% RT | | | | | |
| PD-L1 | PD-L1 <1% TILsLow | Reference | Reference | Reference | Reference | De-intensified |
| TILs | | group | group | group | group | |
| | PD-L1 <1% TILsHigh | 0.989 | 0.604 | 1.62 | 0.966 | Standard of care |
| | PD-L1 >=1% TILsLow | 0.865 | 0.42 | 1.779 | 0.693 | Standard of care |
| | PD-L1 >=1% TILsHigh | 2.649 | 1.483 | 4.732 | 0.001 | Intensify |
| PD-1 | PD-1 <1% TILsLow | Reference | Reference | Reference | Reference | De-intensified |
| TILs | | group | group | group | group | |
| | PD-1 <1% TILsHigh | 1.295 | 0.838 | 2.001 | 0.245 | Intensify |
| | PD-1 >=1% TILsLow | 1.315 | 0.685 | 2.526 | 0.410 | Intensify |
| | PD-1 >=1% TILsHigh | 1.888 | 0.827 | 4.312 | 0.132 | Intensify |
| PD-L1 | PD-L1 <1% | Reference | Reference | Reference | Reference | — |
| TILs RT | TILsLow no RT | group | group | group | group | |
| | PD-L1 <1% | 0.82 | 0.42 | 1.6 | 0.561 | — |
| | TILsHigh no RT | | | | | |
| | PD-L1 >=1% | 0.581 | 0.211 | 1.599 | 0.293 | — |
| | TILsLow no RT | | | | | |
| | PD-L1 >=1% | 2.216 | 1.135 | 4.326 | 0.020 | — |
| | TILsHigh no RT | | | | | |
| | PD-L1 <1% | 0.47 | 0.307 | 0.717 | <0.001 | De-intensified |
| | TILsLow RT | | | | | |
| | PD-L1 <1% | 0.615 | 0.305 | 1.239 | 0.174 | Intensify |
| | TILsHigh RT | | | | | |
| | PD-L1 >=1% | 0.661 | 0.241 | 1.819 | 0.423 | Intensify |
| | TILsLow RT | | | | | |
| | PD-L1 >=1% | 1.284 | 0.403 | 4.092 | 0.673 | Intensify |
| | TILsHigh RT | | | | | |
| PD-L1 high | PD-L1 <1% Low/ | Reference | Reference | Reference | Reference | — |
| TILs RT | moderate TILs no RT | group | group | group | group | |
| | PD-L1 >=1% Low/ | 0.851 | 0.409 | 1.772 | 0.666 | — |
| | moderate no RT | | | | | |
| | PD-L1 <1% Very | 4.639 | 1.69 | 12.735 | 0.003 | — |
| | high TILs no RT | | | | | |
| | PD-L1 >=1% Very | 5.162 | 2.237 | 11.91 | <0.001 | — |
| | high TILs no RT | | | | | |
| | PD-L1 <1% TILs | 0.524 | 0.356 | 0.774 | 0.001 | De-intensified |
| | Low/moderate RT | | | | | |
| | PD-L1 >=1% TILs | 0.933 | 0.428 | 2.033 | 0.862 | Intensify |
| | Low/moderate RT | | | | | |
| | PD-L1 <1% TILs | 1.754 | 0.243 | 12.633 | 0.577 | Intensify |
| | Very high RT | | | | | |
| | PD-L1 >=1% TILs | — | — | — | — | Intensify |
| | Very high RT | | | | | |
| PD-1 | PD-1 <1% | Reference | Reference | Reference | Reference | — |
| TILs RT | TILsLow no RT | group | group | group | group | |
| | PD-1 <1% | 1.177 | 0.678 | 2.042 | 0.563 | — |
| | TILsHigh no RT | | | | | |
| | PD-1 >=1% | 0.926 | 0.372 | 2.306 | 0.869 | — |
| | TILsLow no RT | | | | | |
| | PD-1 >=1% | 1.599 | 0.581 | 4.4 | 0.364 | — |
| | TILsHigh no RT | | | | | |
| | PD-1 <1% | 0.468 | 0.305 | 0.719 | 0.001 | De-intensified |
| | TILsLow RT | | | | | |
| | PD-1 <1% | 0.694 | 0.355 | 1.355 | 0.284 | Intensify |
| | TILsHigh RT | | | | | |
| | PD-1 >=1% | 0.96 | 0.386 | 2.392 | 0.931 | Intensify |
| | TILsLow RT | | | | | |
| | PD-1 >=1% | 1.036 | 0.253 | 4.24 | 0.960 | Intensify |
| | TILsHigh RT | | | | | |
| PD-1 High | PD-1 <1% Low/ | Reference | Reference | Reference | Reference | — |
| TILs RT | moderate TILs no RT | group | group | group | group | |
| | PD-1 >=1% Low/ | 0.851 | 0.37 | 1.961 | 0.706 | — |
| | moderate no RT | | | | | |
| | PD-1 <1% Very | 4.685 | 2.152 | 10.201 | <0.001 | — |
| | high TILs no RT | | | | | |
| | PD-1 >=1% Very | 5.761 | 1.81 | 18.335 | 0.003 | — |
| | high TILs no RT | | | | | |
| | PD-1 <1% TILs | 0.521 | 0.354 | 0.767 | 0.001 | De-intensified |
| | Low/moderate RT | | | | | |
| | PD-1 >=1% TILs | 1.066 | 0.49 | 2.32 | 0.872 | Intensify |
| | Low/moderate RT | | | | | |
| | PD-1 <1% TILs | 1.761 | 0.245 | 12.684 | 0.574 | Intensify |
| | Very high RT | | | | | |
| | PD-1 >=1% TILs | — | — | — | — | Intensify |
| | Very high RT | | | | | |
| TILs RT | TILs <10% no RT | Reference | Reference | Reference | Reference | — |
| | | group | group | group | group | |
| | TILs >=10% no RT | 1.25 | 0.757 | 2.063 | 0.383 | — |
| | TILs <10% RT | 0.506 | 0.338 | 0.758 | 0.001 | De-intensify |
| | TILs >=10% RT | 0.739 | 0.399 | 1.368 | 0.335 | Intensify |
| High | TILs Low/moderate | Reference | Reference | Reference | Reference | — |
| TILs RT | no RT | group | group | group | group | |
| | TILs Very high no RT | 5.032 | 2.598 | 9.744 | <0.001 | — |
| | TILS Low/moderate RT | 0.571 | 0.397 | 0.82 | 0.002 | De-intensified |
| | TILs Very high RT | 1.203 | 0.167 | 8.651 | 0.855 | Intensify |

TABLE 17
Various Explanations for Tables 16A-16C

| | |
|---|---|
| PD-L1 | PD-L1 measured as the proportion of lymphocytes expressing PD-L1 protein. |
| RT | Low defined as below 1%, high defined as equal to or above 1% |
| PD-1 | PD-1 measured as the proportion of lymphocytes expressing PD-1 protein. |
| RT | Low defined as below 1%, high defined as equal to or above 1% |
| PD-1 | PD-1 measured as the proportion of lymphocytes expressing PD-1 protein. |
| PD-L1 | Low defined as below 1%, high defined as equal to or above 1% |
| RT | PD-L1 measured as the proportion of lymphocytes expressing PD-L1 protein. |
| | Low defined as below 1%, high defined as equal to or above 1% |
| PD-L1 | PD-L1 measured as the proportion of lymphocytes expressing PD-L1 protein. |
| TILs | Low defined as below 1%, high defined as equal to or above 1% |
| | TILs cells measured as the absolute stromal area occupied by TILs. |
| | Low defined as below 10%, high defined as equal to or above 10% |
| PD-1 | PD-1 measured as the proportion of lymphocytes expressing PD-1 protein. |
| TILs | Low defined as below 1%, high defined as equal to or above 1% |
| | TILs cells measured as the absolute stromal area occupied by TILs. |
| | Low defined as below 10%, high defined as equal to or above 10% |
| PD-L1 | TILs cells measured as the absolute stromal area occupied by TILs. |
| TILs RT | Low defined as below 10%, high defined as equal to or above 10% |
| | PD-L1 measured as the proportion of lymphocytes expressing PD-L1 protein. |
| | Low defined as below 1%, high defined as equal to or above 1% |
| PD-L1 | PD-L1 measured as the proportion of lymphocytes expressing PD-L1 protein. |
| high | Low defined as below 1%, high defined as equal to or above 1% |
| TILs RT | TILs high = stromal TILs above or equal to 50% (this variable |
| | uses a higher threshold than the ordinary TILs variable) |
| | TILs low/moderate = stromal TILs below 50% |
| PD-1 | PD-1 measured as the proportion of lymphocytes expressing PD-1 protein. |
| TILs RT | Low defined as below 1%, high defined as equal to or above 1% |
| | TILs cells measured as the absolute stromal area occupied by TILs. |
| | Low defined as below 10%, high defined as equal to or above 10% |
| PD-1 | TILs high = stromal TILs above or equal to 50% (this variable |
| High | uses a higher threshold than the ordinary TILs variable) |
| TILs RT | TILs low/moderate = stromal TILs below 50% |
| | PD-1 measured as the proportion of lymphocytes expressing PD-1 protein. |
| | Low defined as below 1%, high defined as equal to or above 1% |
| TILs RT | TILs cells measured as the absolute stromal area occupied by TILs. |
| | Low defined as below 10%, high defined as equal to or above 10% |
| High | TILs high = stromal TILs above or equal to 50% (this variable |
| TILs RT | uses a higher threshold than the ordinary TILs variable) |
| | TILs low/moderate = stromal TILs below 50% |

TABLE 18
Comparison of included versus excluded patients

| Variable | Included | Excluded | P value* |
|---|---|---|---|
| Age median | 59 | 60 | |
| Age mean | 58.40 | 59.31 | |
| Age min | 31 | 32 | |
| Age max | 78 | 78 | 0.11 |
| Missing | 0 | 0 | |

| | | |
|---|---|---|
| RT treated no | 402 (53%) | 213 (50%) |

| | | | |
|---|---|---|---|
| RT treated yes | 362 (47%) | 210 (50%) | 0.46 |
| Missing | 0 | 0 | |

| | | |
|---|---|---|
| 1-10 mm | 169 (22%) | 140 (33%) |
| 11-15 mm | 388 (51%) | 193 (46%) |
| 16-20 mm | 92 (12%) | 33 (8%) |

| | | | |
|---|---|---|---|
| >20 mm | 110 (14%) | 53 (13%) | 0.0003 |
| Missing | 5 | 4 | |

| | | |
|---|---|---|
| Histological grade I | 104 (14%) | 44 (21%) |
| Histological grade II | 457 (61%) | 116 (56%) |

| | | | |
|---|---|---|---|
| Histological grade III | 191 (25%) | 46 (22%) | 0.038 |
| Missing | 12 | 217 | |

| | | |
|---|---|---|
| Systemic treatment yes | 702 (92%) | 384 (91%) |

| | | | |
|---|---|---|---|
| Systemic treatment no | 62 (8%) | 39 (9%) | 0.51 |
| Missing | 0 | 0 | |

*Calculated using the Wilcoxon rank sum test

TABLE 19
10-year follow-up for local recurrence (IBTR). Flexible
parametric survival analysis with Royston-Parmar (RP)
regression models used in FIGS. 9A, 9B, and 11.

| | No adjustment for | | |
|---|---|---|---|
| | other covariates | | |
| | HR | 95% CI | p |
| Sjöström (n = 172) | | | |
| Immunescore | 1.14 | 0.83-1.55 | 0.418 |
| Proliferative Index | 1.98 | 1.49-2.62 | <0.001 |
| Proliferative | 0.61 | 0.47-0.81 | <0.001 |
| Index × | | | |
| Immunescore | | | |
| Servant (n = 343) | | | |
| Immunescore | 0.97 | 0.80-1.18 | 0.768 |
| Proliferative Index | 1.46 | 1.20-1.78 | <0.001 |
| Spline point 1 | 0.72 | 0.62-0.85 | <0.001 |
| Spline point 2 | 0.88 | 0.79-0.97 | 0.012 |
| Proliferative | 0.79 | 0.65-0.96 | 0.018 |
| Index × | | | |
| Immunescore | | | |
| SweBCG91RT, no RT | | | |
| Immunescore | 0.94 | 0.72-1.23 | 0.658 |
| Proliferative Index | 1.25 | 1.00-1.55 | 0.048 |
| Proliferative | 0.86 | 0.70-1.07 | 0.173 |
| Index × | | | |
| Immunescore | | | |
| SweBCG91RT, no RT | | | |
| Immunescore | 0.85 | 0.54-1.34 | 0.491 |
| Proliferative Index | 1.54 | 1.07-2.21 | 0.021 |
| Proliferative | 0.52 | 0.31-0.87 | 0.013 |
| Index × | | | |
| Immunescore | | | |

TABLE 20
10-year follow-up for local recurrence (IBTR). Flexible
parametric survival analysis with Royston-Parmar (RP)
regression models with adjustment for other covariates.

| | | Adjustment for | | |
|---|---|---|---|---|
| | No. of | other covariates | | |
| | IBTR/patients | HR | 95% CI | p |
| Sjöström (n = 172) | | | | |
| Immunescore | 172 | 1.03 | 0.73-1.45 | 0.875 |
| Proliferative Index | 172 | 1.60 | 1.01-2.54 | 0.045 |
| Proliferative | 172 | 0.63 | 0.47-0.84 | 0.002 |
| Index × Immunescore | | | | |
| Histologic subtype | | | | |
| Normal | 4/7 | 3.37 | 1.11-10.2 | 0.031 |
| Luminal A | 17/88 | 1.00 | | |
| Luminal B | 16/39 | 1.51 | 0.65-3.50 | 0.335 |
| Basal | 12/26 | 2.44 | 0.66-9.05 | 0.182 |
| Her2 | 6/12 | 2.34 | 0.70-7.78 | 0.167 |
| Servant (n = 343) | | | | |
| Immunescore | 119/343 | 0.86 | 0.69-1.07 | 0.178 |
| Proliferative Index | 119/343 | 1.06 | 0.76-1.48 | 0.716 |
| Spline point 1 | 119/343 | 0.73 | 0.62-0.85 | <0.001 |
| Spline point 2 | 119/343 | 0.88 | 0.79-0.97 | 0.013 |
| Proliferative | 119/343 | 0.79 | 0.65-0.96 | 0.018 |
| Index × Immunescore | | | | |
| Age | 119/343 | 0.94 | 0.91-0.98 | 0.001 |
| Histologic subtype | | | | |
| Normal | 1/7 | 0.44 | 0.06-3.23 | 0.420 |
| Luminal A | 38/138 | 1.00 | | |
| Luminal B | 47/119 | 1.52 | 0.90-2.55 | 0.118 |
| Basal | 20/58 | 2.43 | 0.96-6.16 | 0.062 |
| Her2 | 13/21 | 4.02 | 1.82-8.86 | 0.001 |
| SweBCG91RT (n = 744) | | | | |
| Immunescore | 112/744 | 0.81 | 0.64-1.04 | 0.096 |
| Proliferative Index | 112/744 | 1.32 | 1.03-1.70 | 0.031 |
| Proliferative | 112/744 | 0.75 | 0.61-0.94 | 0.012 |
| Index × Immunescore | | | | |
| RT | | | | |
| No | 79/394 | 1.00 | | |
| Yes | 33/350 | 0.41 | 0.28-0.62 | <0.001 |
| Age | 112/744 | 0.97 | 0.95-0.98 | <0.001 |
| Tumor size (mm) | 112/744 | 0.97 | 0.94-1.01 | 0.134 |
| Histologic grade | | | | |
| 1 | 9/102 | 1.00 | | |
| 2 | 67/453 | 1.78 | 0.88-3.61 | 0.108 |
| 3 | 36/189 | 2.20 | 0.98-4.96 | 0.058 |
| ER positive | | | | |
| No | 14/78 | 1.00 | | |
| Yes | 98/666 | 0.85 | 0.41-1.78 | 0.674 |

TABLE 21
Absolute number of events within different tertiles of Immunescore and Proliferative Index and depending on RT treatment

| | No RT | | | | | | RT | | | | | |
|---|---|---|---|---|---|---|---|---|---|---|---|---|
| | No | | | Competing | | | No | | | Competing | | |
| Group | recurrence | IBTR | | event | | Total | recurrence | IBTR | | event | | Total |
| PILowIsLow | 39 (76.5%) | 9 | (17.6%) | 3 | (5.9%) | 51 (100.0%) | 42 (87.5%) | 2 | (4.2%) | 4 | (8.3%) | 48 (100.0%) |
| PILowIsMed | 37 (78.7%) | 7 | (14.9%) | 3 | (6.4%) | 47 (100.0%) | 44 (81.5%) | 3 | (5.6%) | 7 | (13.0%) | 54 (100.0%) |
| PILowIsHigh | 21 (65.6%) | 7 | (21.9%) | 4 | (12.5%) | 32 (100.0%) | 18 (78.3%) | 4 | (17.4%) | 1 | (4.3%) | 23 (100.0%) |
| PIMedIsLow | 29 (56.9%) | 14 | (27.5%) | 8 | (15.7%) | 51 (100.0%) | 32 (78.0%) | 3 | (7.3%) | 6 | (14.6%) | 41 (100.0%) |
| PIMedIsMed | 33 (76.7%) | 5 | (11.6%) | 5 | (11.6%) | 43 (100.0%) | 30 (69.8%) | 3 | (7.0%) | 10 | (23.3%) | 43 (100.0%) |
| PIMedIsHigh | 28 (71.8%) | 6 | (15.4%) | 5 | (12.8%) | 39 (100.0%) | 31 (83.8%) | 2 | (5.4%) | 4 | (10.8%) | 37 (100.0%) |
| PIHighIsLow | 19 (59.4%) | 6 | (18.8%) | 7 | (21.9%) | 32 (100.0%) | 19 (59.4%) | 7 | (21.9%) | 6 | (18.8%) | 32 (100.0%) |
| PIHighIsMed | 16 (50.0%) | 10 | (31.2%) | 6 | (18.8%) | 32 (100.0%) | 23 (65.7%) | 4 | (11.4%) | 8 | (22.9%) | 35 (100.0%) |
| PIHighIsHigh | 42 (56.0%) | 17 | (22.7%) | 16 | (21.3%) | 75 (100.0%) | 32 (65.3%) | 5 | (10.2%) | 12 | (24.5%) | 49 (100.0%) |

PI = Proliferative Index.
Is = Immunescore.
IBTR = Ipsilateral breast tumor recurrence

TABLE 22
Unadjusted and adjusted analysis of the interaction between
the Integrated model and RT in the SweBCG91RT cohort

| | | Unadjusted | | | Adjusted | | |
|---|---|---|---|---|---|---|---|
| Variables | | HR | CI 95% | P | HR | CI 95% | P |
| Integrated | | 1.98 | 1.49-2.64 | <0.001 | 2.29 | 1.67-3.15 | <0.001 |
| model | | | | | | | |
| RT | No | 1.0 | | | 1.0 | | |
| | | (reference) | | | (reference) | | |
| | Yes | 0.16 | 0.08-0.35 | <0.001 | 0.18 | 0.08-0.40 | <0.001 |
| Integrated | | 1.69 | 1.18-2.41 | 0.004 | 1.65 | 1.14-2.38 | 0.008 |
| model × RT | | | | | | | |
| Integrated | | 0.88 | 0.82-0.94 | <0.001 | 0.86 | 0.80-0.93 | <0.001 |
| model (tvc) | | | | | | | |
| Hist grade | 1 | | | | 1.0 | | |
| | | | | | (reference) | | |
| | 2 | | | | 1.79 | 0.88-3.61 | 0.107 |
| | 3 | | | | 1.81 | 0.82-4.00 | 0.142 |
| Age | | | | | 0.96 | 0.94-0.98 | <0.001 |
| ER status | Pos | | | | 1.0 | | |
| | | | | | (reference) | | |
| | Neg | | | | 0.85 | 0.45-0.61 | 0.613 |
| Tumor size | | | | | 0.97 | 0.94-1.01 | 0.101 |

Estimates were calculated using Royston-Parmar models. The stpm2 macro for Stata was used for the calculations. Tvc = time-varying coefficient.

TABLE 23
Prognostic effect of Integrated model regarding IBTR depending
on premenopausal status in the SweBCG91RT cohort

| | | <55 years | | >=55 years | |
|---|---|---|---|---|---|
| | | HR | P | HR | P |
| Variables | | (CI 95%) | value | (CI 95%) | value |
| Integrated | | 2.08 | <0.001 | 1.35 | 0.008 |
| score | | (1.48-2.93) | | (1.08-1.67) | |
| Integrated | | 0.99 | 0.041 | | |
| score (tvc) | | (0.98-1.00) | | | |
| RT | No | 1.0 (ref) | | 1.0 (ref) | |
| | Yes | 0.25 | 0.003 | 0.37 | <0.001 |
| | | (0.10-0.61) | | (0.21-0.65) | |
| RT (tvc) | | 1.02 | 0.035 | | |
| | | (1.00-1.05) | | | |
| Hist grade | 1 | 1 (ref) | | | |
| | 2 | 4.88 | 0.031 | | |
| | | (1.16-20.52) | | | |
| | 3 | 3.59 | 0.10 | | |
| | | (0.78-16.43) | | | |

TABLE 24
Distribution of subtypes in the tertiles of
the Integrated score among high-risk patients

| Subtype | Lowest | Middle | Highest |
|---|---|---|---|
| (column %/row %) | tertile | tertile | tertile |
| HER2+ | 10 (6.8%/24.4%) | 7 (4.6%/17.1%) | 24 (16.4%/58.5%) |
| Triple-negative | 16 (10.9%/30.8%) | 8 (5.3%/15.4%) | 28 (19.2%/53.4%) |
| Luminal B | 32 (21.8%/27.6%) | 36 (23.7%/31.0%) | 48 (32.9%/41.4%) |
| Luminal A | 89 (60.5%/37.7%) | 101 (66.4%/42.8%) | 46 (31.5%/19.5%) |

Patients were defined as high-risk if aged <60 or having histological grade III and aged <70.

### Example 2

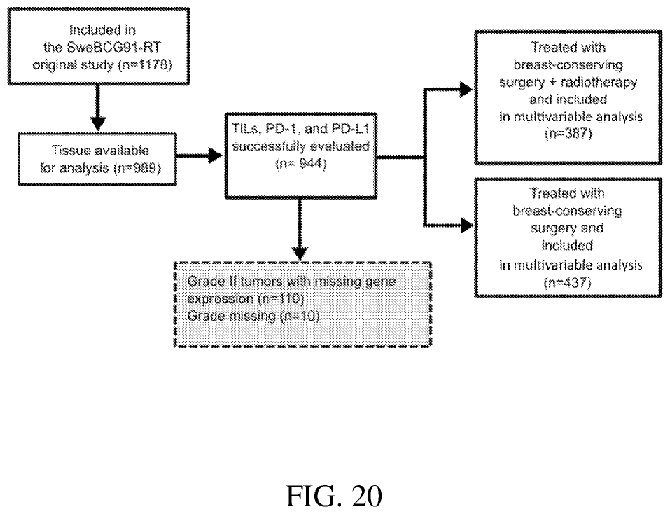

Patients from the SweBCG91RT trial were analyzed. In this example, 1178 patients with lymph-node negative (NO) stage I or IIA breast cancer were randomly assigned between 1991 and 1997 to breast-conserving surgery with or without whole-breast RT and followed for a median time of 15.2 years, FIG. 20. No patient had a positive surgical margin. Systemic adjuvant therapy was given in accordance with regional guidelines at the time. In total, 7% of patients received endocrine treatment, 1% received chemotherapy, and 0.4% received both endocrine therapy and chemotherapy. Tumor subtyping was performed according to the St Gallen international Breast Cancer Conference (2013) Expert Panel on tissue microarray (TMA) slides. In short, tumors were classified as luminal A-like (ER-positive, PgR positive, HER2 negative, and Ki-67 low), luminal B-like (ER-positive, PgR negative or Ki-67 high, and HER2 negative), HER2 positive (HER2 positive, any ER and PgR status, any Ki-67) and triple negative (ER-negative, PgR negative, HER2 negative, and any Ki-67). Analyses were performed on treatment-naîve FFPE tumor samples. Invasive carcinoma was histologically confirmed by a board-certified pathologist.

The original trial and follow-up study were approved by a Regional Ethical Review Board (approval numbers 2010/127 and 2015/548) and conducted per the declaration of Helsinki. Oral informed consent was obtained from all patients before performing human investigations for the original trial and this follow-up study, and was determined appropriate and approved by the Ethical Review Board.

Data Sharing. Gene expression data has been deposited in the Gene Expression Omnibus under accession number GSE119295. Due to regulations of the ethical review board and of laws related to patient privacy, all clinical information has not been made publicly available.

IHC Evaluation. TILs were evaluated on whole tissue H&E sections by two board-certified pathologists. In short, stromal tumor-infiltrating lymphocytes (TILs) were evaluated as semicontinuous values (0%, 1-9%, 10-49%, 50-74%, 75-100%) by two board-certified pathologists, who were blinded to the outcome, until consensus was reached[16]. Evaluations of PD-1 and PD-L1 were performed on TMAs by two board-certified pathologists using the Cell Marque 315M-95 (NAT105) and Ventana (SP142) (RTU) (ref. no. 740-4859) antibodies respectively. Alternatively, the IHC evaluation may be done by automated image analysis as a continuous variable (i.e., not intervals) of the proportion of stroma occupied by lymphocytes as a value between 0-100%. The basic principle is to evaluate the proportion of the tumor stroma occupied by lymphocytes according to the principles outlined by the TILs working group. First, TILs should be reported for the stromal compartment (=% stromal TILs). The denominator used to determine the % stromal TILs is the area of stromal tissue (i.e. area occupied by mononuclear inflammatory cells over total intratumoral stromal area), not the number of stromal cells (i.e., fraction of total stromal nuclei that represent mononuclear inflammatory cell nuclei). Second, TILs should be evaluated within the borders of the invasive tumor. Third, exclude TILs outside of the tumor border and around DCIS and normal lobules. Fourth, exclude TILs in tumor zones with crush artifacts, necrosis, regressive hyalinization as well as in the previous core biopsy site. Fifth, all mononuclear cells (including lymphocytes and plasma cells) should be scored, but polymorphonuclear leukocytes are excluded. Sixth, one section (4-5 μm, magnification 200×-400×) per patient is currently considered to be sufficient. Seventh, full sections are preferred over biopsies whenever possible; cores can be used in the pretherapeutic neoadjuvant setting; currently no validated methodology has been developed to score TILs after neoadjuvant treatment. Eighth, a full assessment of average TILs in the tumor area by the pathologist should be used with care not to focus on hotspots. Ninth, the working group's consensus is that TILs may provide more biological relevant information when scored as a continuous variable, since this will allow more accurate statistical analyses, which can later be categorized around different thresholds. However, in daily practice. most pathologists will rarely report for example 13.5% and will round up to the nearest 5%-10%, in this example thus 15%. The pathologist should report their scores in as much detail as the pathologist feels comfortable with. Tenth, TILs should be assessed as a continuous parameter. The percentage of stromal TILs is a semiquantitative parameter for this assessment, for example, 80% stromal TILs means that 80% of the stromal area shows a dense mononuclear infiltrate. For assessment of percentage values, the dissociated growth pattern of lymphocytes needs to be taken into account. Lymphocytes typically do not form solid cellular aggregates; therefore, the designation “100% stromal TILs” would still allow some empty tissue space between the individual lymphocytes. Eleventh, no formal recommendation for a clinically relevant TIL threshold(s) can be given at this stage. The consensus was that a valid methodology is currently more important than issues of thresholds for clinical use, which will be determined once a solid methodology is in place. lymphocyte-predominant breast cancer can be used as a descriptive term for tumors that contain ‘more lymphocytes than tumor cells’. However, the thresholds vary between 50% and 60% stromal lymphocytes. The above principles outlined by the TILs working group is performed using automated image analysis. The automated method first segregates the image into tumor and stroma. Then, lymphocytes are identified within the respective compartments and proportions of the area within the respective compartment occupied by lymphocytes are calculated. The value for the stromal compartment is most useful for evaluating the level of TILs.

For immunostaining PD-1, the NAT105 is diluted in a concentration of 1:50. A tissue block of the sample is cut in 4 μm sections and then dried in 60° C. for 1 hour. Deparaffinization and pretreatment was performed in a pressure cooker with buffer at pH 6. The following steps were performed in Autostainer plus, DAKO staining equipment with Dako kit K8010 solutions, (except for the primary antibody), the steps include: a peroxidase block (5 min.); primary antibody (30 min.); EnVision (HRP-conjugated polymers) (30 min.); DAB Substrate-chromogen solution (2×5 min); Hematoxylin (counterstain) (4 min.); and dehydrate and coverslip, wherein a rinse was performed with wash buffer between every step. Alternatively, quantifying mRNA levels (e.g., RT-PCR, qPCR, Northern Blotting, microarray analysis, RNA-seq) may be used to approximate protein levels as measured by IHC.

For immunostaining PD-L1, a Benchmark Ultra from Ventana is used to analyze the stained tissue sample. As indicated above, the SP142 is used as the antibody against PD-L1. The pre-treatment buffer is ULTRA Cell Conditioning Solution (ULTRA CC1) from Ventata, a tris-based buffer, to condition the sample for 43 minutes in 100° C. An OptiView DAB IHC Detection kit (ref. no. 760-700) and OptiView Amplification Kit (ref. no. 860-099) are used. Another round of pretreatment buffer wash for 48 minutes in 100° C. The SP142 is incubated for 16 mins at 37° C. Background staining via hematoxlyin II (ref. no. 740-4859) is performed for 8 minutes. Other markers/kits/assays to detect PD-L1 in a sample may be used. In the alternative, the example can instead use Ventana SP263 as the antibody against PD-L1. In the alternative, the example can instead use PD-L1 IHC 28-8 or PD-L1 IHC 22C3 pharmDx assays to detect PD-L1.

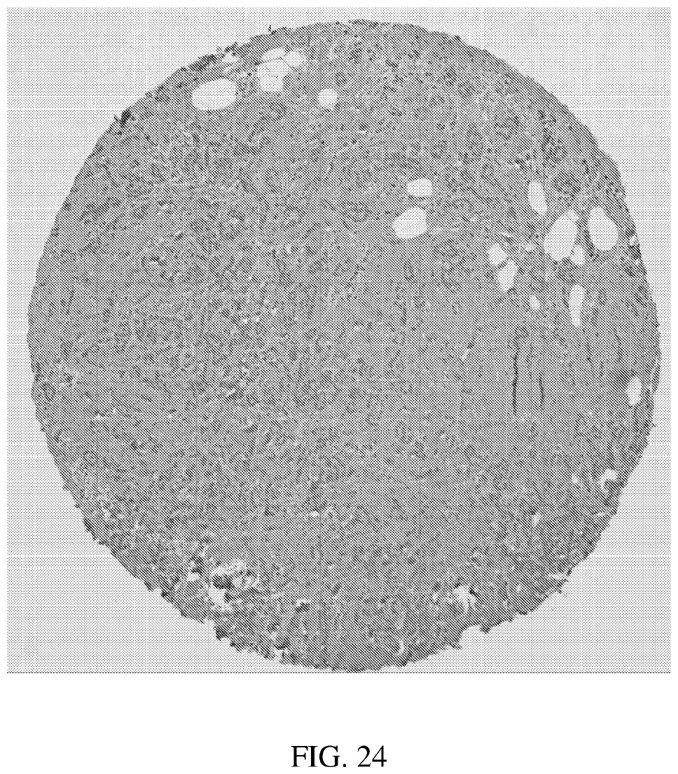

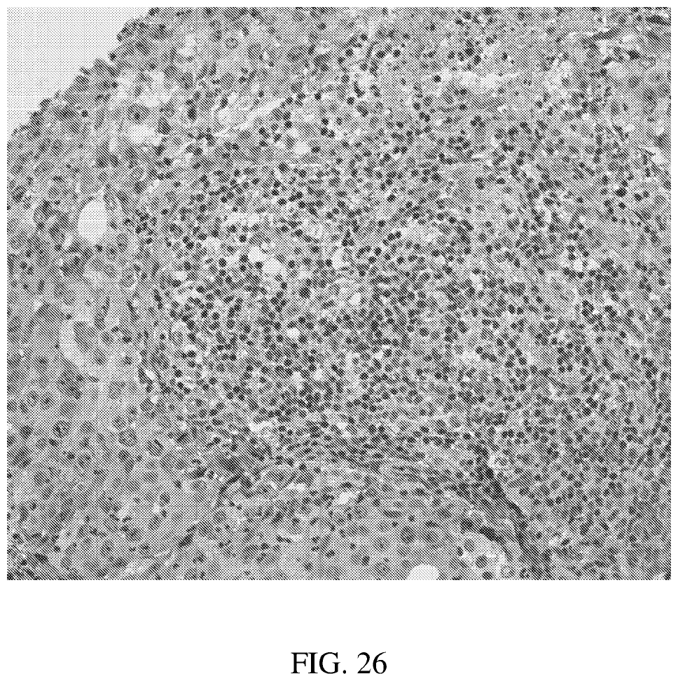

Two cores per marker were evaluated, and the highest value per marker was chosen, given that TMA evaluations of immune checkpoint proteins tend to underestimate the degree of positive staining [17]. Staining of >=1% of lymphocytes was defined as positive, as this is the cut-off used in clinical practice to determine PD-L1 positivity[18]. Images of various staining results are shown in FIGS. 24, 25A, 25B, and 26. FIG. 24 shows a negative staining of hematoxylin of a tissue sample. PD-1 and/or PD-L1 can also be analyzed on whole-tissue sections. Furthermore, the analysis can be performed using an automated image analysis.

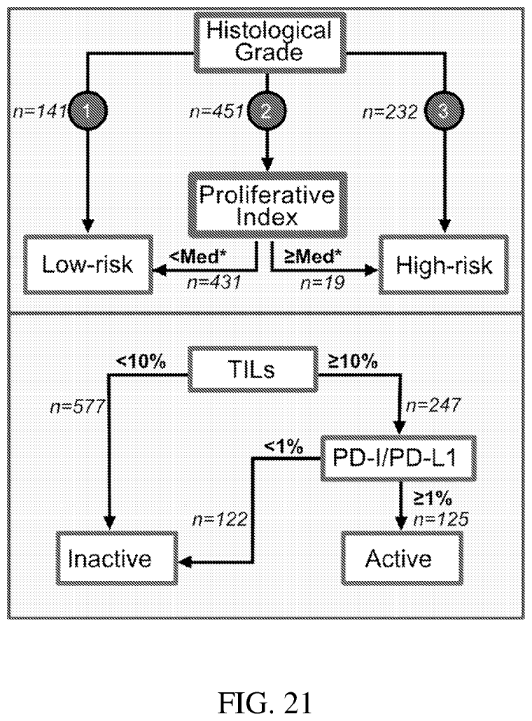

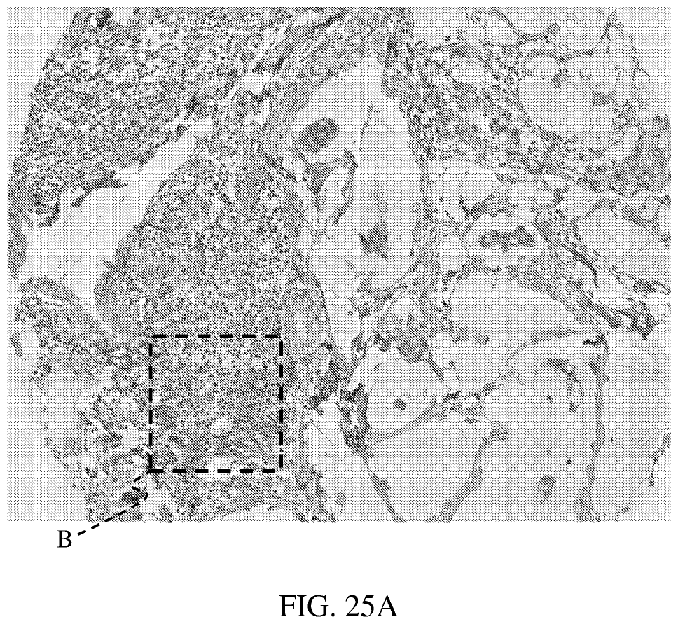

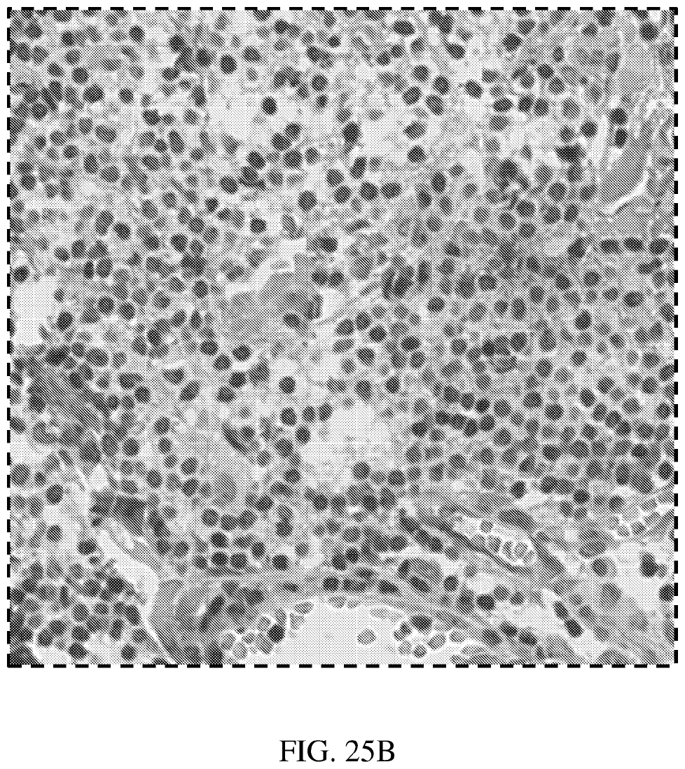

An activated immune infiltrate is defined as TILs>=10% and positive staining for at least one of PD-1 or PD-L1, FIGS. 21 and 25A-B. The activated immune infiltrate in this example is based on evidence that TILs and immune checkpoint molecule expression provide independent information, thereby complementing each other [19]. Consequently, combining TILs with checkpoint molecule expression measurements allows for identifying the most immunogenic tumors compared to either marker alone [20].

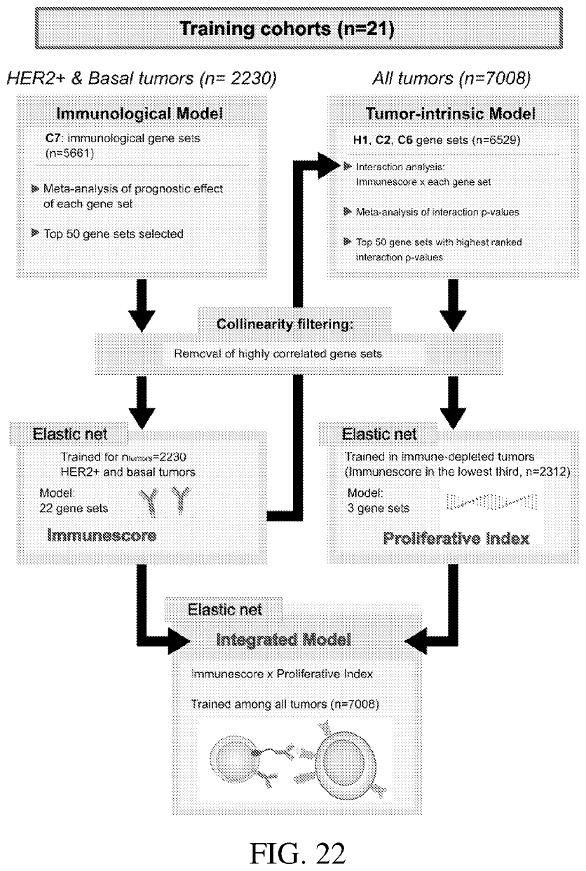

Tumor-intrinsic risk group assessment. Patients were divided into low- and high-risk groups depending on histological grade and the signature Proliferative Index (PI). Histological grade I was classified as low-risk and grade III as high-risk. In the previous example, Proliferative Index demonstrated a strong correlation with histological grade and proliferation, and could predict the immune responsiveness of tumors. Without being bound by theory, grade II tumors are predicted to be heterogeneous and can be reclassified into high- or low-risk as previously suggested [21]. Most tumors of the SweBCG91RT cohort were previously classified as grade II. Because an immune infiltrate's prognostic effect in low-risk, ER-dominated, cohorts is either absent or unfavorable, the majority of grade II tumors should be classified as low-risk and not immune-responsive[22-24]. This determined is further supported by the fact that the Proliferative Index of grade II tumors resembled grade I tumors more than grade III tumors, FIG. 22 (as shown gene sets from the molecular signatures database were used to create a model measuring immunological activity and immunomodulatory tumor-intrinsic factors). These were merged to create a model considering the interaction between the antitumoral immune response and tumor-intrinsic immunomodulatory factors. Therefore, grade II tumors are more similar to grade I tumors regarding the biological implications of an immune infiltrate. To accurately reclassify grade II tumors based on their immune responsiveness, the cut-off for high-risk grade II tumors was set at the median Proliferative Index of grade III tumors. The remainder of grade II tumors were classified as low-risk, FIGS. 21 and 22. The high cut-off was to prevent diluting the effect size of the high-risk group.

The Proliferative Index can be used stratify clinical groups, Table 25. For example, stratifying tumors of histological grade II to high-risk and low-risk. In another example, stratifying Luminal A and Luminal B tumors into high- and low-risk. This means that Proliferative Index can be used to improve predictions decision-making in cases with borderline indication. High-risk tumors should receive chemotherapy (especially relevant to Luminal A where patients with low-risk Luminal A tumors can be spared chemotherapy).

The following are examples of thresholds to upgrade to high-risk. For Luminal A, a value above anywhere between 60th to 95th percentile compared to a background population of representative Luminal A tumors. For Histological Grade II, a value above anywhere between 60th to 95th percentile compared to a background population of representative grade II tumors. For Luminal B, a value above anywhere between 60th to 95th percentile compared to a background population of representative Luminal B tumors. The above high-risk groups may be further classified based on immune activation (Immunescore or PD-1/PD-L1/TILs), most relevant to Luminal B high-risk tumors where the lack of immune activation predicts a poor prognosis.

TABLE 25
Risk of any recurrence depending on Proliferative Index within
Luminal A, Luminal B, and histological grade II tumors

| | Luminal A | | Luminal B | | Histological grade II | |
|---|---|---|---|---|---|---|
| Variable | HR (CI 95%) | P value | HR (CI 95%) | P value | HR (CI 95%) | P value |
| Proliferative | 1.75 | <0.001 | 1.50 | 0.003 | 1.34 | 0.0238 |
| Index* | (1.254-2.44) | | (1.153-1.952) | | (1.04-1.73) | |

*Each unit increase represents a standard deviation in the whole cohort (i.e., not just the examined subtype)

Statistical Methods. Time to ipsilateral breast tumor recurrence (TBTR) as the first event within 10 years from diagnosis was used as the primary endpoint. The aims were to analyze the interaction between an activated immune response and tumor-intrinsic risk group (high-risk or low-risk) on the risk of IBTR and its implications for the benefit from RT. A likelihood-ratio test between regression models with and without an interaction term was used to test the interaction effect. A p-value<0.05 was considered significant. P values reported for other analyses, which were not part of the main hypothesis, were not adjusted for multiple hypothesis testing and should be interpreted with caution. Hazard ratios (HR) presented in tables and the results section were calculated with cause-specific Cox proportional hazards regression to reflect the biological effect of an activated immune infiltrate depending on tumor-intrinsic risk groups in the presence of competing risks. Other recurrences and deaths were considered competing risks for IBTR. Cumulative incidences were used to describe 10-year IBTR rates. Figures of cumulative incidences were created according to the method of Fine and Gray[30] and based on the Cox models of subhazards, producing subdistribution hazard ratios (SHR). P-values for differences in cumulative incidences between compared groups were denoted as PCIF in the plots. Age, tumor size, ER status, and RT were tested in univariable analysis and, if significant, in multivariable analysis.

STATA 17.0 was used for analysis (StataCorp. 2017, Stata: Release 17, Statistical Software, StataCorp LLC).

### Results

Demographics. In total, 148 (15.4%) tumors were classified as grade I, 573 (59.8%) as grade II, and 237 (24.7%) as grade III. The Proliferative Index was calculated, and the scores were centered and standardized to have a mean of 0 and a standard deviation of 1. The Proliferative Index was used to classify grade II tumors as high-risk or low-risk, see FIG. 21. Grade I tumors had a median Proliferative Index of −0.70, grade I tumors −0.43, and grade III tumors 1.03, FIG. 22. A total of 19 (3.3%) of the 573 grade II tumors had a Proliferative Index equal to or higher than the median of grade III tumors and were classified as high-risk.

In total, 139 (55.4%) of high-risk tumors had high TILs, 62 (24.7%) had a high PD-1 expression (>=1%), and 101 (40.2%) had a high PD-L1 expression (>=1%), Table 26. A total of 96 (38.2%) were classified as having an activated immune response. A total of 75 (36.1%) high-risk tumors were ER-negative, and 232 (92.4%) tumors were of grade III, while 19 (7.6%) were of grade II.

In the low-risk group, high TILs were seen among 108 tumors (18.8%), high PD-1 expression among 48 (8.4%) tumors, and high PD-L1 expression among 62 (10.8%) tumors, Table 26. In total, 29 (5.1%) tumors were classified as having an activated immune response. Among low-risk tumors, 12 (2.3%) were ER-negative, 141 (24.6%) of grade I, and 432 (75.4%) of grade II.

TABLE 26
Demographics of included patients

| | | Low-risk group | | | High-risk group | | |
|---|---|---|---|---|---|---|---|
| Variables | | No RT | RT | Total | No RT | RT | Total |
| TILs | Low | 240 | 225 | 465 | 63 | 49 | 112 |
| | | (81.4%) | (80.9%) | (81.2%) | (44.4%) | (45%) | (44.6%) |
| | High | 55 | 53 | 108 | 79 | 60 | 139 |
| | | (18.6%) | (19.1%) | (18.8%) | (55.6%) | (55%) | (55.4%) |
| PD-1 | Low | 267 | 258 | 525 | 109 | 80 | 189 |
| | | (90.5%) | (92.8%) | (91.6%) | (76.8%) | (73.4%) | (75.3%) |
| | High | 28 | 20 | 48 | 33 | 29 | 62 |
| | | (9.5%) | (7.2%) | (8.4%) | (23.2%) | (26.6%) | (24.7%) |
| PD-L1 | Low | 255 | 256 | 511 | 89 | 61 | 150 |
| | | (86.4%) | (92.1%) | (89.2%) | (62.7%) | (56%) | (59.8%) |
| | High | 40 | 22 | 62 | 53 | 48 | 101 |
| | | (13.6%) | (7.9%) | (10.8%) | (37.3%) | (44%) | (40.2%) |
| Immune | Active* | 20 | 9 | 29 | 50 | 46 | 96 |
| activation | | (6.8%) | (3.2%) | (5.1%) | (35.2%) | (42.2%) | (38.2%) |
| | Inactive/absent** | 275 | 269 | 544 | 92 | 63 | 155 |
| | | (93.2%) | (96.8%) | (94.9%) | (64.8%) | (57.8%) | (61.8%) |
| Subtype | HER2 positive | 6 | 9 | 15 | 19 | 19 | 38 |
| | | (2.2%) | (3.5%) | (2.8%) | (16.2%) | (21.6%) | (18.5%) |
| | Luminal A | 193 | 175 | 368 | 26 | 18 | 44 |
| | | (71%) | (67.8%) | (69.4%) | (22.2%) | (20.5%) | (21.5%) |
| | Luminal B | 70 | 72 | 142 | 37 | 27 | 64 |
| | | (25.7%) | (27.9%) | (26.8%) | (31.6%) | (30.7%) | (31.2%) |
| | Triple negative | 3 | 2 | 5 | 35 | 24 | 59 |
| | | (1.1%) | (0.8%) | (0.9%) | (29.9%) | (27.3%) | (28.8%) |
| ER status | Negative | 7 | 5 | 12 | 42 | 33 | 75 |
| | | (2.6%) | (1.9%) | (2.3%) | (35.3%) | (37.1%) | (36.1%) |
| | Positive | 266 | 254 | 520 | 77 | 56 | 133 |
| | | (97.4%) | (98.1%) | (97.7%) | (64.7%) | (62.9%) | (63.9%) |
| PgR status | Negative | 41 | 48 | 89 | 59 | 48 | 107 |
| | | (15%) | (18.5%) | (16.7%) | (49.6%) | (53.9%) | (51.4%) |
| | Positive | 232 | 211 | 443 | 60 | 41 | 101 |
| | | (85%) | (81.5%) | (83.3%) | (50.4%) | (46.1%) | (48.6%) |
| Histological | Grade I | 68 | 73 | 141 | 0 | 0 | 0 |
| grade | | (23.1%) | (26.3%) | (24.6%) | (0%) | (0%) | (0%) |
| | Grade II | 227 | 205 | 432 | 10 | 9 | 19 |
| | | (76.9%) | (73.7%) | (75.4%) | (7%) | (8.3%) | (7.6%) |
| | Grade III | 0 | 0 | 0 | 132 | 100 | 232 |
| | | (0%) | (0%) | (0%) | (93%) | (91.7%) | (92.4%) |
| Endocrine | No hormone | 254 | 249 | 503 | 105 | 80 | 185 |
| therapy | therapy | (92.7%) | (95.8%) | (94.2%) | (88.2%) | (88.9%) | (88.5%) |
| | Hormone | 20 | 11 | 31 | 14 | 10 | 24 |
| | therapy | (7.3%) | (4.2%) | (5.8%) | (11.8%) | (11.1%) | (11.5%) |
| Chemotherapy | No | 274 | 259 | 533 | 113 | 87 | 200 |
| | chemotherapy | (100%) | (99.6%) | (99.8%) | (95%) | (96.7%) | (95.7%) |
| | Chemotherapy | 0 | 1 | 1 | 6 | 3 | 9 |
| | | (0%) | (0.4%) | (0.2%) | (5%) | (3.3%) | (4.3%) |
| IBTR within | No IBTR | 240 | 255 | 495 | 96 | 86 | 182 |
| 5 years*** | | (81.4%) | (91.7%) | (86.4%) | (67.6%) | (78.9%) | (72.5%) |
| | IBTR | 37 | 7 | 44 | 27 | 8 | 35 |
| | | (12.5%) | (2.5%) | (7.7%) | (19%) | (7.3%) | (13.9%) |
| | Censored | 18 | 16 | 34 | 19 | 15 | 34 |
| | | (6.1%) | (5.8%) | (5.9%) | (13.4%) | (13.8%) | (13.5%) |
| IBTR within | No IBTR | 206 | 221 | 427 | 80 | 69 | 149 |
| 10 years*** | | (69.8%) | (79.5%) | (74.5%) | (56.3%) | (63.3%) | (59.4%) |
| | IBTR | 54 | 23 | 77 | 33 | 10 | 43 |
| | | (18.3%) | (8.3%) | (13.4%) | (23.2%) | (9.2%) | (17.1%) |
| | Censored | 35 | 34 | 69 | 29 | 30 | 59 |
| | | (11.9%) | (12.2%) | (12%) | (20.4%) | (27.5%) | (23.5%) |

*Defined as TILs >=10% and PD-L1 and/or PD-1 >=1%
**Defined as TILs <10% or TILs >=10% but PD-L1 and PD-1 <1%
***Reported as absolute frequencies rather than cumulative incidences

Prognostic Effect. In total, 17.2% (13.1-22.5) of patients in the high-risk group and 13.7% (11.1-16.8) of patients in the low-risk group developed an IBTR within 10 years. High-risk tumors with an active immune response had an IBTR rate of 8.4% (4.3-16.1) while high-risk tumors without an active immune infiltrate had an IBTR rate of 22.8% (16.9-30.2). Among high-risk tumors, an activated immune infiltrate was associated with a reduced risk of IBTR in univariable (HR 0.34, CI 95% 0.16-0.73, p=0.006) and multivariable (HR 0.33. CI 95% 0.15-0.72, p=0.005) analysis, Table 28. The interaction between immune activity and risk group was significant in univariable (p=0.005) and multivariable (p=0.007) analysis, Table 27.

TABLE 27
Cox proportional hazard rate regression. 10-year follow-up
of ipsilateral breast tumor recurrence (IBTR)

| | | | Univariable Cox | | Multivariable Cox | |
|---|---|---|---|---|---|---|
| | | No. of | regression | | regression | |
| | | IBTRs/No. | HR | | HR | |
| Variables | | of patients | (95% CI) | P | (95% CI) | P |
| Combination | Not | 71/544 | 1.0 | | 1.0 | |
| of immune | activated, | | | | | |
| group and | low risk | | | | | |
| risk group | Not | 35/155 | 2.1 | <0.001 | 1.9 | <0.002 |
| | activated, | | (1.4-3.1) | | (1.3-2.9) | |
| | high risk | | | | | |
| | Activated, | 6/29 | 2.0 | 0.105 | 1.8 | 0.189 |
| | low risk | | (0.87-4.6) | | (0.76-4.0) | |
| | Activated, | 8/96 | 0.68 | 0.306 | 0.63 | 0.217 |
| | high risk | | (0.33-1.4) | | (0.30-1.3) | |
| | Interaction | | | 0.005* | | 0.007* |
| Age (cont.) | | 120/824 | 0.97 | 0.005 | 0.97 | 0.003 |
| | | | (0.95-0.99) | | (0.96-0.99) | |
| Tumor size | | 110/738 | 1.01 | 0.761 | — | |
| (cont.) | | | (0.97-1.04) | | | |
| ER status | Negative | 15/94 | 1.0 | | — | |
| | Positive | 105/726 | 0.78 | 0.376 | — | |
| | | | (0.46-1.35) | | | |
| RT | No | 87/437 | 1.0 | | 1.0 | |
| | Yes | 33/387 | 0.39 | <0.001 | 0.41 | <0.001 |
| | | | (0.26-0.58) | | (0.27-0.61) | |

*Likelihood-ratio test.

TABLE 28
Cox proportional hazard rate regression. 10-year follow-up of ipsilateral
breast tumor recurrence (IBTR) among low-risk- and high-risk patients

| | Low-risk group (n = 573) | | | | | High-risk group (n = 251) | | | | |
|---|---|---|---|---|---|---|---|---|---|---|
| | | | | Multiv. | | | | | Multiv. | |
| | # of | Univ. Cox | | Cox | | # of | Univ. Cox | | Cox | |
| | IBTR/ | regression | | regression | | IBTR/ | regression | | regression | |
| | # of | HR | | HR | | # of | HR | | HR | |
| Variable | patients | (95% CI) | P | (95% CI) | P | patients | (95% CI) | P | (95% CI) | P |
| Immune | | | | | | | | | | |
| system | | | | | | | | | | |
| Not | 71/544 | 1.0 | | 1.0 | | 35/155 | 1.0 | | 1.0 | |
| activated | | | | | | | | | | |
| Activated | 6/29 | 2.0 | 0.100 | 1.8 | 0.159 | 8/96 | 0.34 | 0.006 | 0.33 | 0.005 |
| | | (0.87-4.6) | | (0.79-4.2) | | | (0.16-0.73) | | (0.15-0.72) | |
| Age | 77/573 | 0.98 | 0.102 | — | — | 43/251 | 0.97 | 0.019 | 0.97 | 0.016 |
| (cont.) | | (0.95-1.0) | | | | | (0.94-0.99) | | (0.94-0.99) | |
| Tumor | 71/530 | 1.0 | 0.739 | — | — | 39/208 | 0.98 | 0.518 | — | — |
| size | | (0.96-1.1) | | | | | (0.93-1.0) | | | |
| (cont.) | | | | | | | | | | |
| ER^{#} | | | | | | | | | | |
| Negative | 4/9 | 1.0 | | 1.0 | | 11/85 | 1.0 | | | |
| Positive | 73/561 | 0.21 | 0.002 | 0.17 | 0.001 | 32/165 | 1.4 | 0.302 | — | — |
| | | (0.08-0.56) | | (0.06-0.46) | | | (0.72-2.8) | | | |
| RT^{#} | | | | | | | | | | |
| No | 54/295 | 1.0 | | 1.0 | | 33/142 | 1.0 | | 1.0 | |
| Yes | 23/278 | 0.41 | <0.001 | 0.40 | <0.001 | 10/109 | 0.36 | 0.005 | 0.42 | 0.017 |
| | | (0.25-0.67) | | (0.25-0.66) | | | (0.18-0.74) | | (0.20-0.85) | |

Low-risk tumors with an active immune infiltrate had a 10-year IBTR rate of 20.9% (10.0-40.7) compared to an IBTR rate of 13.3% (10.7-16.5) among low-risk tumors without an active immune infiltrate. No significant difference in risk IBTR among low-risk tumors was seen for an active immune infiltrate (univariable: 2.0, CI 95% 0.87-4.6, p=0.100, multivariable: HR 1.8. CI 95% 0.79-4.2, p=0.159) compared to not having an active immune infiltrate (HR 1.0), Table 28.

Benefit from RT. A non-significant benefit from RT was seen among high-risk tumors with an activated immune infiltrate (HR 0.34, CI 95% 0.07-1.67, p=0.182), while a significant benefit was observed among high-risk tumors without an activated immune infiltrate (HR 0.40, CI 95% 0.18-0.88, p=0.022). Among low-risk tumors with an activated immune infiltrate, the estimates for RT benefit (HR 0.40, CI 95% 0.05-3.44, p=0.403) were similar to those of low-risk tumors without an activated immune infiltrate (HR 0.42, CI 95% 0.25-0.69, p=0.001).

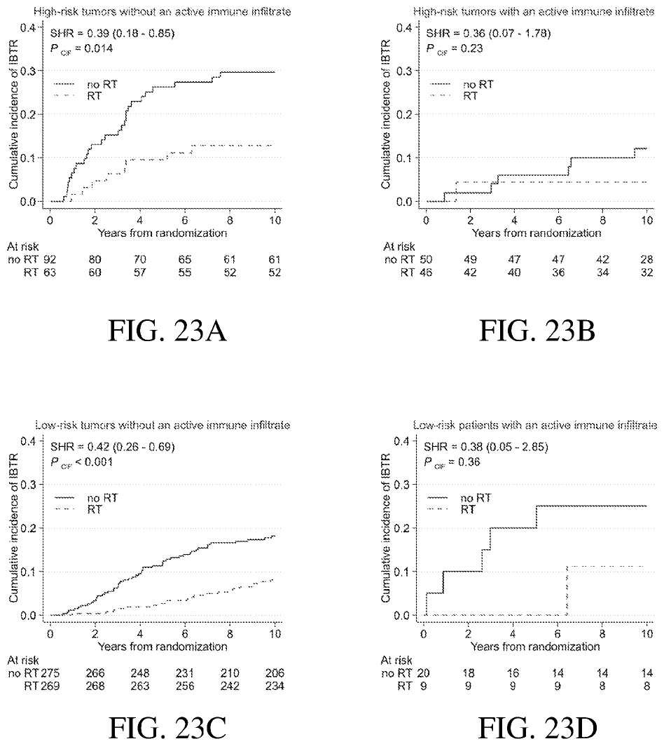

FIGS. 23A, 23B, 23C, and 23D illustrate the cumulative incidences depending on RT, immune activation, and tumor-intrinsic risk group. High-risk tumors with an activated immune response had a 10-year incidence of IBTR of 12.1% (5.6-25.0) without RT and 4.4% (1.1-16.3) with RT. This can be contrasted against high-risk tumors with an absent immune response where the 10-year incidence of IBTR was 29.6% (21.4-40.2) without RT and 12.8% (6.6-23.9) with RT. Low-risk tumors with an activated immune response had a 10-year IBTR incidence of 25.0% (11.2-50.0) without RT and 11.1% (1.6-56.7) with RT, while low-risk tumors without an active immune infiltrate had a 10-year incidence of IBTR of 18.1% (14.0-23.3) without RT and 8.4% (5.6-12.5) with RT.

### Example 3

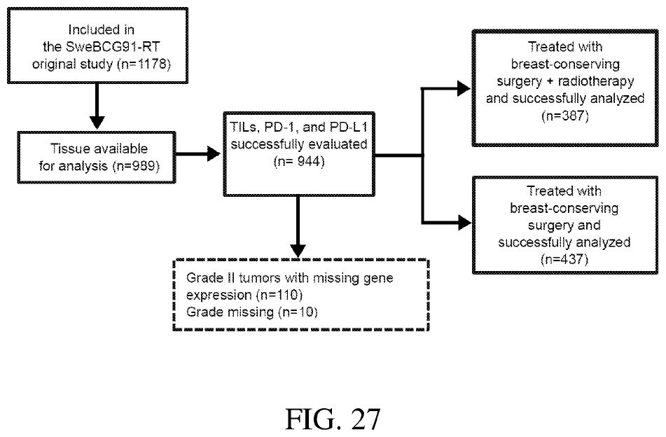

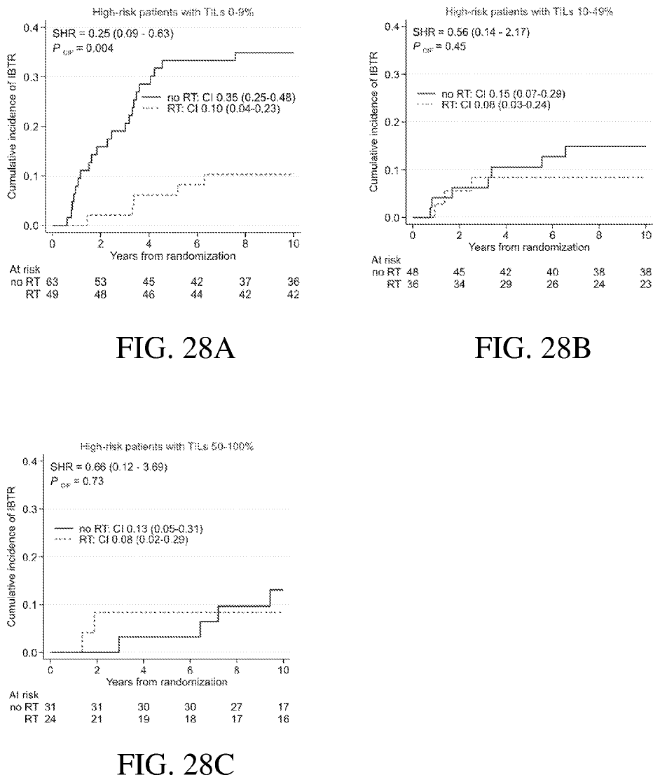

Assessing Immunological Activity via level of TILs. To investigate a potential dose-response relationship, high-risk groups with TILs 10-49% and 50-100% were compared. The consort of patients is depicted in FIG. 27. As shown in FIGS. 28A-C. unirradiated patients with TILs 10-49% had a 10-year cumulative IBTR incidence of 15% (0.07-0.29); (RT=Radiotherapy, SHR=Subhazard ratio for RT vs. no RT; and CI=Cumulative incidence). Also shown, unirradiated patients with TILs 50-100% had a lower, but not significantly different, cumulative IBTR incidence of 13% (0.05-0.31). From these results, a trend of an inverse dose-dependent relationship is shown between TILs and RT benefit. Considering low recurrence rates among unirradiated patients with high-risk tumors and high TILs despite lack of systemic therapy, it seems that these patients can be omitted from RT. Accordingly, immune-responsive tumors with very high TIL levels (e.g., ≥50%) may represent an RT omission group, while moderately increased TILs (e.g., 10-49%) could justify RT boost omission. Based on this finding, TILs levels are linearly protective among tumors that benefit from an immune response (e.g., high-risk tumors). High-risk tumors may be identified by a high grade. Alternatively, high-risk tumors are identified by a high PI score. Alternatively, high-risk tumors are identified as being ER-negative. Alternatively, high-risk tumors are identified by being HER2-positive. Alternatively, high-risk tumors are identified by high Ki67 levels. Alternatively, high-risk tumors are identified by high TMB. Alternatively, high-risk tumors are identified by any high grade, high PI score, ER negative, HER2-positive, high Ki67 levels, high TMB, or any combination thereof.

The same linear relationship principle can be used for combination of markers. For example, in tumors that benefit from an immune response, TILs above a threshold (preferably in a range of 10% to 75%), high PD-1, and high PD-L1 can identify tumors for RT omission, which indicated in Table 28. Additionally, samples with level of TILs above a threshold but low PD-1 and PD-L1 can be identified as tumors for RT boost omission. While using a TILs threshold of 10% is fine, the treatment indication would be stronger if the threshold is set higher. To improve the treatment indication, a TILs threshold of above 10% such 10.1%, 20%, 30%, 40%, 50%, or any numbers in between, may be considered. An added value to TILs of additional immunological markers, such as PD-1/PD-L1 expression, may be partly or entirely explained by an association with even higher TILs. Therefore, assessing TILs as a continuous variable on whole sections can be a sufficiently robust measurement to identify tumors with different immune activation degrees and tailor therapy accordingly.

TABLE 28
Levels of TILs among patients with TILs ≥10%
with and without PD-1/PD-L1 expression

| | | TILs | TILs | TILs | |
|---|---|---|---|---|---|
| Groups | | 10-49% | 50-74% | 75-100% | P* |
| PD-1/PD-L1 | No | 97 (89.0%) | 10 (9.2%) | 2 (1.8%) | <0.001 |
| expression | Yes | 66 (58.9%) | 29 (25.9%) | 17 (15.2%) | |

*Fisher's exact test
PD-1/PD-L1 expression was defined as the expression of PD-1 or PD-L1 in ≥1% of lymphocytes in at least one TMA. Among tumors with TILs ≥10%, PD-1/PD-L1 expression was associated with higher TILs levels.

TABLE 29
Example of scenarios when combining the high-risk group with different immune activation categories

| | | | | | Prognosis compared to tumors | | | |
|---|---|---|---|---|---|---|---|---|
| | | | | | from the same grade category | Predicted | Risk-benefit | |
| | | | | | or a group defined by other | benefit | profile (compared | Therapy to be |
| | | | | | clinical/pathological variables | from RT | to tumors from | recommended |
| Risk | | | | | or gene expression with a | (standard | the same risk | and/or |
| group | TILs | PD-1 | PD-L1 | Immunescore | corresponding recurrence risk\hat{ } | of care) | group category) | administered** |
| High- | 0-9% | <1% or | <1% or | <25th | Unfavorable | High | Residual | Standard RT |
| risk* | | unknown | unknown | percentile | | | | with RT boost |
| | | | | or unknown | | | | |
| | 10-49% | <1% or | <1% or | <25th | Similar | High | High | Standard RT |
| | | unknown | unknown | percentile | | | | with or |
| | | | | or unknown | | | | without boost |
| | 10-49% | >=1% | >=1% | >=75th | Favorable | Low | Low | Standard RT |
| | | | | percentile | | | | without RT |
| | | | | | | | | boost |
| | 50-74% | <1% or | <1% or | <75th | Favorable | Low | Low | Standard RT |
| | | unknown | unknown | percentile | | | | without RT |
| | | | | or unknown | | | | boost |
| | 50-74% | >=1% | >=1% | >=75th | Very | Low | Low | Standard RT |
| | | | | percentile | favorable | | | without RT |
| | | | | | | | | boost or RT |
| | | | | | | | | omission |
| | 75-100% | >=1% | >=1% | >=75th | Very | Low | Low | RT omission |
| | | | | percentile | favorable | | | |

*Preferably defined by a combination of histological grade and Proliferative Index. Can be defined as:
1. Tumors of histological grade III and/or a Proliferative Index above a threshold above the 60^{th} percentile of a representative background population of tumors.
2. Tumors of histological grade II and a Proliferative Index above the 60^{th} percentile (preferably above the 75^{th} percentile or even more preferably above the 85^{th} percentile) of a representative background population of tumors.
**Age can optionally be used to up- or downgrade borderline cases, whereby an age above a threshold indicates that the less intensive therapy is indicated. Suitable thresholds may be 60 years, 55 years or 50 years of age.
# Risk-benefit profile
Residual: High risk of recurrence despite standard radiotherapy
High: High risk of recurrence without standard radiotherapy. Low risk of recurrence with standard radiotherapy.
Low: Low risk of recurrence without standard radiotherapy. Low risk of recurrence with standard radiotherapy.

### Example 4

A subject with breast cancer is provided treatment plan based on determining an Immunescore (IS) based on an immunological model comprised of genes from immunological gene sets and determining a Proliferative Index (PI) based on a tumor-intrinsic model comprised of genes from tumor-intrinsic gene sets. The IS and PI are integrated into an Integrated model. The Integrated model and an age of the subject are integrated into a Final model to identify if the subject falls into a specific-risk clinical group. Based on the specific-risk clinical group, an appropriate therapy is performed on the subject. The immunological model is trained in basal and HER2+ tumors. The tumor intrinsic model is trained in immune-depleted tumors.

### Example 5

A subject with breast cancer is provided a treatment plan based on determining an IS based on an immunological model comprised of genes from immunological gene sets and determining a PI based on a tumor-intrinsic model comprised of genes from tumor-intrinsic gene sets. The IS and the PI are integrated into an Integrated model. The Integrated model and an age of a subject are integrated into a Final model to identify if the subject falls into a specific-risk clinical group based on a risk score from the Final model. The immunological model is trained in basal and HER2+ tumors. The tumor intrinsic model is trained in immune-depleted tumors. The high risk score groups the subject into a high-risk clinical group.

A treatment based on the treatment plan for the subject is intensified when the risk score for the subject is a high risk score. The treatment plan in this example includes treating the subject with intensified radiotherapy including a dose of at least one of: 67 Gy or more, add a boosting dose to a standard recommended treatment for the subject when the standard recommended treatment does not include a boosting dose, increase a boosting dose beyond the standard amount for the subject, increase the fraction dose on a per fraction basis above the standard for the subject, increase the number of fractions of a recommended dose above the standard for the subject.

The intensified treatment plan denotes at least one of: intensified radiotherapy treatment, systemic therapy, mastectomy, the additional use of a sensitizer to another therapy; a therapy above a level set by at least one of: the NCCN, ESMO, ESTRO, Clinical Practice Recommendations Australia, and/or NICE guidelines for the subject's remaining indicators, or any combination thereof.

### Example 6

A subject with breast cancer is provided a treatment plan based on determining expression levels of genes that are included in a model. The expression levels of the genes are adjusted by scaling or normalizing to a background population of representative tumors. The adjusted expression levels of the genes are summed to determine enrichment scores of gene sets included in the model, wherein the gene sets include the genes. The enrichment scores are standardized by comparing the enrichment scores to the background population. The IS and PI models are used to calculate IS and PI scores respectively. The IS and PT scores are standardized by comparing the IS and PT scores to the background population. The integrated model is used to calculate an integrated score. The integrated score is used by comparing the score to the background population. A final model is used to determine a risk score of a subject, where the final model includes an age of the subject.

The risk score of the subject is compared to the background population to determine if the risk score of the subject falls into a risk category, the risk category including a low-risk group, a medium risk group, or a high risk group. A therapy is performed on the subject based on the risk category of the subject.

### Example 7

A subject with breast cancer is provided a treatment plan by determining an IS from a tumor sample from the subject based on an immunological model that includes genes from immunological gene sets and determining a PI from the tumor sample based on a tumor-intrinsic model comprised of genes from tumor-intrinsic gene sets. The IS and the PI are integrated into an Integrated model. The Integrated model and an age of a subject are integrated into a Final model to identify if the subject falls into a specific-risk clinical group. An appropriate therapy is performed on the subject based on the specific-risk clinical group.

### Example 8

A subject with breast cancer is provided a treatment plan based on identifying relevant gene sets from immunological gene sets and tumor-intrinsic gene sets. A plurality of the relevant gene sets are selected. From these selected gene sets, an IS and a PI are created. The IS corresponds to the immunological gene sets, and the PI corresponds to the tumor-intrinsic gene sets. The IS and the PI are integrated into an integrated model. The integrated model and an age of a subject are integrated to create a Final model. The Final Model identifies if the subject falls into a specific-risk clinical group. An appropriate therapy is performed on the subject based on the specific-risk clinical group.

### Example 9

A subject with breast cancer is provided a treatment plan based on determining a tumor aggressivity, the tumor aggressivity including a histological grade of a tumor from at least a portion of a sample of the tumor provided by the subject, and the histological grade including Grades I, H, and III. The sample is classified, when determined as a Grade I tumor, as low-risk. The sample is classified, when determined as a Grade III tumor, as high-risk. When the sample is determined as a Grade II tumor, the tumor aggressivity further includes a Proliferative Index score of the sample. The Grade I tumor is classified as: a) high-risk when the Proliferative Index score is greater or equal to a median score of a background population of Grade III tumors, or b) low-risk when the Proliferative Index score is less than the median score of the background population of Grade III tumors.

An Immunescore of the sample is determined based on a level of tumor infiltrating lymphocytes (TILs) in the sample, a level of checkpoint molecules in the sample, or any combination thereof to determine an immunological activity.

The tumor aggressivity and the immunological activity are integrated by training an elastic net having an interaction term between the tumor aggressivity and the immunological activity. A treatment plan is determined based on an integration of tumor aggressivity and immunological activity.

In this example, the Proliferative Index score is based on expression of a first group of genes, and the Immunescore is based on expression of a second group of genes. The checkpoint molecules include programmed cell death protein-1 (PD-1) and programmed death-ligand 1 (PD-L1). The immunological activity is determined to be: i) active when the TILs score is ≥10% and the checkpoint molecules score is ≥1% of lymphocytes with positive staining for PD-1 or PD-L1; or ii) inactive when the TILs score is less than 10% and the checkpoint molecules score is less than 1% of lymphocytes with positive staining for both PD-1 and PD-L1, or when the TILs score is ≥10%, and the checkpoint molecules score is less than 1% of lymphocytes with positive staining for both PD-1 and PD-L1.

The active immunological activity indicates an activated immune infiltrate. The inactive immunological activity indicates an inactive immune infiltrate. The first group of genes comprises one or more genes listed in Table 6. The second group of genes comprises one or more genes listed in Table 4.

The treatment that is performed on the subject is: c) standard radiotherapy, radiotherapy de-intensification, or radiotherapy omission, when the sample is classified as the high-risk Grade II tumor and the immunological activity is active; d) radiotherapy intensification when sample is classified as the high-risk Grade II tumor and the immunological activity is inactive; c) radiotherapy intensification when the sample is classified as the low-risk Grade II tumor and the immunological activity is active; or f) radiotherapy de-intensification, or radiotherapy omission, when the sample is classified as the low-risk Grade II tumor and the immunological activity is inactive.

### Example 10

A subject with breast cancer is provided a treatment plan based on determining tumor aggressivity, the tumor aggressivity including a histological grade of a tumor from at least a portion of a sample of the tumor provided by a subject. The histological grade includes Grade I, Grade II, and Grade III. The sample is classified, when determined as a Grade I tumor, as low-risk. The sample is classified, when determined as a Grade III tumor, as high-risk. To determine the tumor aggressivity of the sample, when determined as a Grade II tumor, the tumor aggressivity further comprising a Proliferative Index score of the sample. The Grade II tumor is classified as: a) high-risk when the Proliferative Index score is above a threshold between 60th to 95th percentile compared to a background population of representative Grade II tumors, or b) low-risk when the Proliferative Index score is below a threshold below the 60th percentile to the background population of representative Grade II tumors.

To determine an Immunescore of the sample, a level of TILs in the sample, a level of checkpoint molecules in the sample, or any combination thereof is used. The tumor aggressivity and immunological activity are integrated by training an elastic net having an interaction term between the tumor aggressivity and the immunological activity. A treatment plan is determined based on the tumor aggressivity, the immunological activity, and the interaction term. The Proliferative Index score is based on expression of a first group of genes. The Immunescore is based on expression of a second group of genes. The checkpoint molecules include PD-1 and PD-L1.

The immunological activity is determined to be: i) active when the TILs score is ≥10% and the checkpoint molecules score is ≥1% of lymphocytes with positive staining for PD-1 or PD-L1, or ii) inactive when the TILs score is less than 10% and/or the checkpoint molecules score is less than 1% of lymphocytes with positive staining for both PD-1 and PD-L1. The active immunological activity indicates an activated immune infiltrate. The inactive immunological activity indicates an inactive immune infiltrate. The first group of genes includes one or more genes listed in Table 6. The second group of genes includes one or more genes listed in Table 4.

The treatment that is performed on the subject is: c) standard radiotherapy, radiotherapy de-intensification, or radiotherapy omission, when the sample is classified as the high-risk Grade II tumor and the immunological activity is active; d) radiotherapy intensification when sample is classified as the high-risk Grade I tumor and the immunological activity is inactive; e) the radiotherapy intensification when the sample is classified as the low-risk Grade II tumor and the immunological activity is active; f) radiotherapy de-intensification, or the radiotherapy omission, when the sample is classified as the low-risk Grade II tumor and the immunological activity is inactive; g) the radiotherapy intensification when the sample is classified as a Grade III tumor and the immunological activity is inactive; h) radiotherapy de-intensification, or the radiotherapy omission, when the sample is classified as a Grade III tumor and the immunological activity is active; i) the radiotherapy intensification when the sample is classified as a Grade I tumor and the immunological activity is active; or j) radiotherapy de-intensification, or the radiotherapy omission, when the sample is classified as a Grade I tumor and the immunological activity is inactive.

### Example 11

A subject with breast cancer is provided a treatment plan based on subtyping a tumor from at least a portion of a sample of the tumor provided by a subject, wherein the subtype includes Luminal A, Luminal B, HER2+, and triple negative/basal. A tumor aggressivity of the sample is determined, when the sample is a Luminal A tumor or a Luminal B tumor. The tumor aggressivity includes a Proliferative Index score of the tumor sample.

The sample is classified, when the sample is subtyped as the Luminal A tumor, as: a) high-risk when the Proliferative Index score is above a threshold between 60th to 95th percentile compared to a background population of representative Luminal A tumors, or b) low-risk when the Proliferative Index score is below a threshold below the 60th percentile to the background population of representative Luminal A tumors. The sample is classified, when the sample is subtyped as the Luminal B tumor, as: c) high-risk, when the Proliferative index score is above a threshold between 60th to 95th percentile compared to a background population of representative Luminal B tumors, or d) low-risk when the Proliferative Index score is below a threshold below the 60th percentile to the background population of representative Luminal B tumors.

An immunological activity is determined by scoring an Immunescore of the sample, scoring a level of TILs in the sample, scoring a level of checkpoint molecules in the sample, or any combination thereof. The tumor aggressivity and the immunological activity are integrated by training an elastic net having an interaction term between the tumor aggressivity and the immunological activity. A treatment plan is determined based on the integrated tumor aggressivity, immunological activity, and the interaction term. The scoring of the Proliferative Index is based on expression of a first group of genes. The scoring of the Immunescore is based on expression of a second group of genes. The checkpoint molecules include PD-1 and PD-L1.

The immunological activity is determined to be: i) active when the TILs score is ≥10% and either of the checkpoint molecules score is ≥1%, or ii) inactive when the TILs score is less than 10% and the checkpoint molecules score is less than 1%, where the active immunological activity indicates an activated immune infiltrate, and the inactive immunological activity indicates an inactive immune infiltrate. The first group of genes includes one or more genes listed in Table 6. The second group of genes includes one or more genes listed in Table 4.

The treatment that is performed on the subject is: e) radiotherapy de-intensification or omission when the tumor sample is classified as the low-risk Luminal A tumor, f) standard radiotherapy, radiotherapy de-intensification, or the radiotherapy omission when the sample is classified as the high-risk Luminal B tumor and the immunological activity is active; or g) radiotherapy intensification when tumor sample is classified as the high-risk Luminal B tumor and the immunological activity is inactive.

### Example 12

A subject with breast cancer is provided a treatment plan based on determining a tumor aggressivity of a tumor from at least a portion of a sample of the tumor provided by a subject. The tumor aggressivity includes a Proliferative Index that classifies the tumor as: a) high-risk when a Proliferative Index score of the sample is above a threshold between 60th to 95th percentile compared to a background population of representative tumors, or b) low-risk when the Proliferative Index score of the sample is below a threshold below the 60th percentile compared to the background population of representative tumors. An immunological activity of the tumor is determined based on an Immunescore of the sample. The tumor aggressivity and immunological activity are integrated based on an interaction term between the tumor aggressivity and the immunological activity. A treatment that is performed on the subject is determined based on the tumor aggressivity, immunological activity, and the interaction term.

### Example 13

A subject with breast cancer is provided a treatment plan based on determining a tumor aggressivity of a tumor from at least a portion of a sample of the tumor provided by a subject. The tumor aggressivity includes a Proliferative Index score that classifies the tumor as: a) high-risk when the Proliferative Index score of the sample is greater or equal to a median score of a background population of representative tumors, or b) low-risk when the Proliferative Index score of the sample is less than the median score of the background population of representative tumors. An immunological activity of the tumor is determined based on an Immunescore of the sample. The tumor aggressivity and immunological activity are integrated based on an interaction term between the tumor aggressivity and the immunological activity. A treatment that is performed on the subject is determined based on the tumor aggressivity, immunological activity, and the interaction term.

### Example 14

A subject with breast cancer is provided a treatment plan based on the benefits of the treatment plan. A histological grade of a tumor is determined from at least a sample of the tumor provided by a subject. The histological grade for the tumor includes Grade I, Grade II, or Grade III. A tumor aggressivity of the sample is assigned as: a) low-risk when the sample is determined as a Grade I tumor, b) high-risk when the sample is determined as a Grade III tumor, c) low-risk when the sample is determined as a Grade II tumor and a Proliferative Index score of the sample is less than a median score of a background population of tumors; or d) high-risk when the sample is determined as the Grade II tumor and a Proliferative Index score of the sample is greater or equal to a median score of the background population of tumors. An immunological activity is classified as active or inactive, wherein this classification is determined by: e) an Immunescore of the sample; f) a level of TILs in the sample; g) a level of checkpoint molecules in the sample; or h) any combination of e)-f).

The tumor aggressivity and the immunological activity are integrated by using an interaction term to determine the benefit of the treatment plan.

### Example 15

A subject with breast cancer is provided a therapy. To determine which therapy to provide, a sample of a tumor from the subject is supplied. A therapy is provided to the subject based on an analysis of the supplied sample, the analysis including determining a tumor aggressivity of the tumor from the sample, determining an immunological activity of the tumor from sample, and integrating the tumor aggressivity and the immunological activity based on an interaction term. The tumor aggressivity includes a Proliferative Index score that classifies the tumor as high-risk or low-risk. The immunological activity includes: a) an Immunescore of the sample; b) a level of TILs in the sample; c) a level of checkpoint molecules in the sample; or d) any combination of a)-c).

### Example 16

A subject with breast cancer is provided a therapy based on determining a Proliferative Index based on a level of one or more of the genes in Table 6 and determining an Immunescore based on expression of one or more of the genes in Table 4. The Proliferative Index and Immunescore are combined, and optionally factoring in age of the subject, to determine if the subject will respond to the cancer therapy. and administering the cancer therapy if the Proliferative Index and Immunescore, and optionally the age of the subject, indicates that the therapy will be successful.

### Example 17

A subject with breast cancer is provided a treatment plan based on factoring in a level of one or more genes from Table 6, factoring in a level of one or more genes from Table 4, and factoring in an age of the subject, thereby determining if the subject should receive standard radiation therapy, radiotherapy intensifications, radiotherapy de-intensification or radiotherapy omission. The determined therapy is performed on the subject.

### Example 18

A subject with breast cancer is provided a treatment plan based on determining an Immunescore of a core biopsy without determining a level of TILs surrounding a tumor from which the core biopsy was obtained, using the Immunescore to determine an amount of radiotherapy to administer to a subject, without factoring in the level of TILs. The determined amount of therapy is given to the subject.

### Example 19

A subject with breast cancer is provided a treatment plan based on a method of predicting the effectiveness of a cancer therapy, the method including analyzing a sample for a presence of one or more of the genes in Table 4 and/or one or more of the genes in Table 6, wherein a variation in a level of the gene indicates an effectiveness of the cancer therapy.

### Example 20

A subject with breast cancer is provided a treatment plan based on determining a histological grade of a tumor from at least a sample of the tumor provided by a subject. The histological grade for the tumor includes Grade I, Grade II, or Grade III. A tumor aggressivity of the sample is assigned as: a) low-risk when the sample is determined as a Grade I tumor, b) low-risk when the sample is determined as a Grade II tumor and a Proliferative Index score of the sample is less than a median score of a background population of tumors; c) high-risk when the sample is determined as the Grade II tumor and a Proliferative Index score of the sample is greater or equal to a median score of the background population of tumors; or d) high-risk when the sample is determined as: i) a Grade III tumor, ii) the Proliferative Index score of the sample is greater or equal to a median score of the background population of tumors, iii) ER-negative, iv) HER2-positive, v) the level of Ki67 is high, vi) mutational burden is high, or vii) any combination of i)-vi).

An immunological activity is classified by a level of TILs in the sample. The tumor aggressivity and the immunological activity are integrated using an interaction term to determine the treatment plan. The determined treatment plan is performed on the subject.

### Example 21

A subject is diagnosed for breast cancer based on determining a histological grade of a tumor from at least a sample of the tumor provided by a subject. The histological grade for the tumor includes Grade I, Grade II, or Grade III. A tumor aggressivity of the sample is assigned as: a) low-risk when the sample is determined as a Grade I tumor; b) low-risk when the sample is determined as a Grade II tumor and a Proliferative Index score of the sample is less than a median score of a background population of tumors; c) high-risk when the sample is determined as the Grade II tumor and a Proliferative Index score of the sample is greater or equal to a median score of the background population of tumors; or d) high-risk when the sample is determined as: i) a Grade III tumor, ii) the Proliferative Index score of the sample is greater or equal to a median score of the background population of tumors, iii) ER-negative, iv) HER2-positive, v) the level of Ki67 is high, vi) mutational burden is high, or vii) any combination of i)-vi).

An immunological activity is classified by a level of TILs in the sample. The tumor aggressivity and the immunological activity are integrated using an interaction term.

### Example 22

A subject with breast cancer is provided a treatment plan based on determining a histological grade of a tumor from at least a sample of the tumor provided by a subject. The histological grade for the tumor includes Grade I, Grade II, or Grade III. A tumor aggressivity of the sample is assigned. An immunological activity is classified by including a level of TILs in the sample. The tumor aggressivity and the immunological activity are integrated using an interaction term to determine a treatment plan. The determined treatment plan is performed on the subject.

The described embodiments and examples of the present disclosure are intended to be illustrative rather than restrictive, and are not intended to represent every embodiment or example of the present disclosure, and thus, are not to be limited in scope by the specific embodiments and examples described herein. While the fundamental novel features of the disclosure as applied to various specific embodiments thereof have been shown, described, and pointed out, it will also be understood that various omissions, substitutions, and changes in the details of the methods that are disclosed, may become apparent and may be made by those skilled in the art without departing from the spirit of the disclosure. For example, it is expressly intended that all combinations of those method steps that perform substantially the same function in substantially the same way to achieve the same results are within the scope of the disclosure. Additionally, one or more of the disclosed method steps may be repeated any number of times. Moreover, it should be recognized that method steps shown and/or described in connection with any disclosed form or embodiment of the disclosure may be incorporated in any other disclosed or described or suggested form or embodiment as a general matter of design choice. Further, in at least some of the previously described embodiments, one or more elements used in an embodiment can interchangeably be used in another embodiment unless such a replacement is not technically feasible. Further, various modifications and variations can be made without departing from the spirit or scope of the disclosure as set forth in the following claims both literally and in equivalents recognized in law.

### REFERENCES

1. Zon M, G. D., Haibe-Kains B, MetaGxBreast: Transcriptomic Breast Cancer Datasets. 2021.
2. Ramos M, G. L., Oh S, Schiffer L, Azhar R, Kodali H, de Bruijn I, Gao J, Carey V J, Morgan M, Waldron L, Multiomic Integration of Public Oncology Databases in Bioconductor. JCO Clinical Cancer Informatics, 958-971, 2020.
3. Deena M. A. Gendoo, N. R., Markus S. Schroeder, Laia Pare, Joel S Parker, Aleix Prat, Benjamin Haibe-Kains, genefu: Computation of Gene Expression-Based Signatures in Breast Cancer. 2021.
4. Dewey, M., metap: meta-analysis of significance values. 2022.
5. Stouffer, S. A., et al., The American soldier: Adjustment during army life. (Studies in social psychology in World War II), Vol. 1. The American soldier: Adjustment during army life. (Studies in social psychology in World War II), Vol. 1. 1949, Oxford, England: Princeton Univ. Press. xii, 599-xii, 599.
6. Kuhn, M., caret: Classification and Regression Training. 2021.
7. Kovács, A., et al., Effect of Radiotherapy After Breast-Conserving Surgery Depending on the Presence of Tumor-Infiltrating Lymphocytes: A Long-Term Follow-Up of the SweBCG91RT Randomized Trial. Journal of Clinical Oncology, 2019. 37(14): p. 1179-1187.
8. Aran, D., Z. Hu, and A. J. Butte, xCell: digitally portraying the tissue cellular heterogeneity landscape. Genome Biol, 2017. 18(1): p. 220.
9. Yoshihara, K., et al., Inferring tumour purity and stromal and immune cell admixture from expression data. Nature Communications, 2013. 4(1): p. 2612
10. Servant, N., et al., Search for a gene expression signature of breast cancer local recurrence in young women. Clin Cancer Res, 2012. 18(6): p. 1704-15.
11. Sjostrom, M., et al., Identification and validation of single-sample breast cancer radiosensitivity gene expression predictors. Breast Cancer Res, 2018. 20(1): p. 64.
12. P Royston, P. L., Stata Press, FlexibleParametric Survival Analysis Using Stata: Beyond the Cox Model.
13. Thoresen, M., Spurious interaction as a result of categorization. BMC Med Res Methodol, 2019. 19(1): p. 28.
14. Fine, J. P. and R. J. Gray, A Proportional Hazards Model for the Subdistribution of a Competing Risk. Journal of the American Statistical Association, 1999. 94(446): p. 496-509.
15. Geskus, R. B., Cause-specific cumulative incidence estimation and the fine and gray model under both left truncation and right censoring. Biometrics, 2011. 67(1): p. 39-49.
16. Kovacs, A., A. Stenmark Tullberg, E. Werner Ronnerman, E. Holmberg, L. Hartman, M. Sjostrom, D. Lundstedt, P. Malmstrom, M. Ferno, and P. Karlsson, Effect of Radiotherapy After Breast-Conserving Surgery Depending on the Presence of Tumor-Infiltrating Lymphocytes: A Long-Term Follow-Up of the SweBCG91RT Randomized Trial. J Clin Oncol, 2019. 37(14): p. 1179-1187.
17. Sobral-Leite, M., K. Van de Vijver, M. Michaut, R. van der Linden, G. K. J. Hooijer, H. M. Horlings, T. M. Severson, A. M. Mulligan, N. Weerasooriya, J. Sanders, A. M. Glas, D. Wehkamp, L. Mittempergher, K. Kersten, A. Cimino-Mathews, D. Peters, E. Hooijberg, A. Broeks, M. J. van de Vijver, R. Bernards, I. L. Andrulis, M. Kok, K. E. de Visser, and M. K. Schmidt, Assessment of PD-L1 expression across breast cancer molecular subtypes, in relation to mutation rate, BRCA1-like status, tumor-infiltrating immune cells and survival. Oncoimmunology, 2018. 7(12): p. e1509820.
18. Schmid, P., S. Adams, H. S. Rugo, A. Schneeweiss, C. H. Barrios, H. Iwata, V. Dieras, R. Hegg, S. A. Im, G. Shaw Wright, V. Henschel, L. Molinero, S. Y. Chui, R. Funke, A. Husain, E. P. Winer, S. Loi, L. A. Emens, and I. M. T. Investigators, Atezolizumab and Nab-Paclitaxel in Advanced Triple-Negative Breast Cancer. N Engl J Med, 2018. 379(22): p. 2108-2121.
19. Loi, S., S. Michiels, S. Adams, S. Loibl, J. Budczies, C. Denkert, and R. Salgado, The journey of tumor-infiltrating lymphocytes as a biomarker in breast cancer: clinical utility in an era of checkpoint inhibition. Ann Oncol, 2021. 32(10): p. 1236-1244.
20. Emens, L. A., C. Cruz, J. P. Eder, F. Braiteh, C. Chung, S. M. Tolaney, I. Kuter, R. Nanda, P. A. Cassier, J. P. Delord, M. S. Gordon, E. ElGabry, C. W. Chang, I. Sarkar, W. Grossman, C. O'Hear, M. Fasso, L. Molinero, and P. Schmid, Long-term Clinical Outcomes and Biomarker Analyses of Atezolizumab Therapy for Patients With Metastatic Triple-Negative Breast Cancer: A Phase 1 Study. JAMA Oncol, 2019. 5(1): p. 74-82.
21. Sotiriou, C., P. Wirapati, S. Loi, A. Harris, S. Fox, J. Smeds, H. Nordgren, P. Farmer, V. Praz, B. Haibe-Kains, C. Desmedt, D. Larsimont, F. Cardoso, H. Peterse, D. Nuyten, M. Buyse, M. J. Van de Vijver, J. Bergh, M. Piccart, and M. Delorenzi, Gene expression profiling in breast cancer: understanding the molecular basis of histologic grade to improve prognosis. J Natl Cancer Inst, 2006. 98(4): p. 262-72.
22. Sobral-Leite, M., I. Salomon, M. Opdam, D. T. Kruger, K. J. Beelen, V. van der Noort, R. L. P. van Vlierberghe, E. J. Blok, D. Giardiello, J. Sanders, K. Van de Vijver, H. M. Horlings, P. J. K. Kuppen, S. C. Linn, M. K. Schmidt, and M. Kok, Cancer-immune interactions in ER-positive breast cancers: PI3K pathway alterations and tumor-infiltrating lymphocytes. Breast Cancer Res, 2019. 21(1): p. 90.
23. Liu, S., W. D. Foulkes, S. Leung, D. Gao, S. Lau, Z. Kos, and T. O. Nielsen, Prognostic significance of FOXP3+ tumor-infiltrating lymphocytes in breast cancer depends on estrogen receptor and human epidermal growth factor receptor-2 expression status and concurrent cytotoxic T-cell infiltration. Breast Cancer Res, 2014. 16(5): p. 432.
24. Johansson, A., N. Y. Yu, A. Iftimi, N. P. Tobin, L. van't Veer, B. Nordenskjold, C. C. Benz, T. Fornander, G. Perez-Tenorio, O. Stal, L. J. Esserman, C. Yau, and L. S. Lindstrom, Clinical and molecular characteristics of estrogen receptor-positive ultralow risk breast cancer tumors identified by the 70-gene signature. Int J Cancer, 2022.

What is claimed is:

1. A method of treating a subject, comprising:
factoring in a level of one or more genes from Table 6:
factoring in a level of one or more genes from Table 4; and
factoring in an age of the subject, thereby determining if the subject should receive standard radiation therapy, radiotherapy intensification, radiotherapy de-intensification, or radiotherapy omission.

2. The method of claim 1, wherein the expression of one or more genes for Immunescore and/or Proliferative Index is measured from a tissue sample provided by the subject.

3. The method of claim 2, wherein the sample comprises a formalin fixed paraffin embedded tissue sample.

4. The method of claim 2 or 3, wherein the sample is collected prior to a treatment of the subject for the cancer.

5. The method of any one of claims 2-4, wherein the sample is collected prior to a therapeutic surgery of the subject for the cancer.

6. The method of any one of the preceding claims, wherein the subject is treated with neoadjuvant immunotherapy.

7. The method of any one of the preceding claims, wherein stromal tumor-infiltrating lymphocytes around the tumor are not used in and/or available for analysis or used as part of the method.

8. The method of any one of the preceding claims, wherein the subject is 55 years or older.

9. The method of any one of claims 1-7, wherein the subject is 65 years or older.

10. The method of any one of claims 1-7, wherein the subject is 55 years or younger.

11. The method of any one of claims 1-7, wherein the subject is between 50 and 65 years of age.

12. The method of any one of claims 1-7, wherein the subject is less than 50 years of age.

13. The method of any one of claims 1-12, wherein the level of one or more genes of Table 6 includes one or more levels of the one or more genes of Table 6, and
wherein the level of one or more genes of Table 4 includes one or more levels of the one or more genes of Table 4.

14. A method for treating breast cancer comprising:
determining a histological grade of a tumor from at least a sample of the tumor provided by a subject, wherein the histological grade for the tumor includes Grade I, Grade IT, or Grade III;
assigning a tumor aggressivity of the sample as:
a) low-risk when the sample is determined as a Grade I tumor;
b) low-risk when the sample is determined as a Grade II tumor and a Proliferative Index score of the sample is less than a median score of a background population of high-risk tumors;
c) high-risk when the sample is determined as the Grade II tumor and a Proliferative Index score of the sample is greater or equal to a median score of the background population of high-risk tumors; or
d) high-risk when the sample is determined as:
i) a Grade III tumor,
ii) the Proliferative Index score of the sample is greater or equal to a median score of the background population of high-risk tumors,
iii) ER-negative,
iv) HER2-positive,
v) the level of Ki67 is high,
vi) mutational burden is high, or
vii) any combination of i)-vi);
classifying an immunological activity includes a level of tumor-infiltrating lymphocytes (TILs) in the sample; and
integrating the tumor aggressivity and the immunological activity using an interaction term to determine a treatment plan.

15. A method for diagnosing breast cancer comprising:
determining a histological grade of a tumor from at least a sample of the tumor provided by a subject, wherein the histological grade for the tumor includes Grade I, Grade II, or Grade III;
assigning a tumor aggressivity of the sample as:
a) low-risk when the sample is determined as a Grade I tumor;
b) low-risk when the sample is determined as a Grade II tumor and a Proliferative Index score of the sample is less than a median score of a background population of high-risk tumors;
c) high-risk when the sample is determined as the Grade II tumor and a Proliferative Index score of the sample is greater or equal to a median score of the background population of high-risk tumors; or
d) high-risk when the sample is determined as:
i) a Grade III tumor,
ii) the Proliferative Index score of the sample is greater or equal to a median score of the background population of high-risk tumors,
iii) ER-negative,
iv) HER2-positive,
v) the level of Ki67 is high,
vi) mutational burden is high, or
vii) any combination of i)-vi);
classifying an immunological activity includes a level of TILs in the sample; and integrating the tumor aggressivity and the immunological activity using an interaction term.

16. The method of claim 14 or 15, wherein vii) includes a combination of three or more selected from i)-vi).

17. The method of claim 14 or 15, wherein the level of Ki67 is ≥10%.

18. The method of claim 14 or 15, wherein the level of Ki67 is ≥20%.

19. The method of claim 14 or 15, wherein the level of Ki67 is ≥30%.

20. The method of any one of claims 14-19, wherein the mutational burden is ≥5 mutations per genomic megabase (mut/MB).

21. The method of any one of claims 14-19, wherein the mutational burden is ≥7 mut/MB.

22. The method of any one of claims 14-19, wherein the mutational burden is ≥10 mut/MB.

23. The method of any one of claims 14-22, wherein the subject is 55 years or older.

24. The method of any one of claims 14-22, wherein the subject is 65 years or older.

25. The method of any one of claims 14-22, wherein the subject is 55 years or younger.

26. The method of any one of claims 14-22, wherein the subject is between 50 and 65 years of age.

27. The method of any one of claims 14-22, wherein the subject is less than 50 years of age.

28. The method of any one of claims 14-22, wherein the background population is age-matched.

29. The method of any one of claims 14-28, wherein the Proliferative Index score is based on one or more levels of one or more genes from a tumor-intrinsic gene set.


SEQUENCE LISTING (5 sequences)

SEQ ID NO: 1 - protein, 288 aa, Homo sapiens
  source 1..288: note = PD-1
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - protein, 290 aa, Homo sapiens
  source 1..290: note = PD-L1
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 3 - protein, 176 aa, Homo sapiens
  source 1..176: note = PD-L1
MRIFAVFIFMTYWHLLNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 4 - RNA, 3634 nt, Homo sapiens
  source 1..3634: mol_type = mRNA; note = PD-L1
agttctgcgcagcttcccgaggctccgcaccagccgcgcttctgtccgcctgcagggcattccagaaagatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatccca

SEQ ID NO: 5 - RNA, 3292 nt, Homo sapiens
  source 1..3292: mol_type = mRNA; note = PD-L1
agttctgcgcagcttcccgaggctccgcaccagccgcgcttctgtccgcctgcagggcattccagaaagatgaggatatttgctgtctttatattcatgacctactggcatttgctgaacgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatccca